SEQUENCE LISTING (509 sequences)

SEQ ID NO: 1 - DNA, 23 bp, synthetic construct
  source 1..23: mol_type = other DNA
ccattgttttgtcgttcctgatc

SEQ ID NO: 2 - DNA, 26 bp, synthetic construct
  source 1..26: mol_type = other DNA
caattgcgtgtatcatagatgttttg

SEQ ID NO: 3 - DNA, 35 bp, synthetic construct
  source 1..35: mol_type = other DNA
gatatttacacagaccgagatagtcatggaattcg

SEQ ID NO: 4 - DNA, 27 bp, synthetic construct
  source 1..27: mol_type = other DNA
ctgaccgagcccgctgttgtcagagat

SEQ ID NO: 5 - DNA, 20 bp, synthetic construct
  source 1..20: mol_type = other DNA
gccccagtagcgcattcagc

SEQ ID NO: 6 - DNA, 20 bp, synthetic construct
  source 1..20: mol_type = other DNA
gcccggttagcgcctcttcc

SEQ ID NO: 7 - DNA, 27 bp, synthetic construct
  source 1..27: mol_type = other DNA
gctggaaaccattagtacgcgataacc

SEQ ID NO: 8 - DNA, 24 bp, synthetic construct
  source 1..24: mol_type = other DNA
gctcaatctgtagggtttcgcagg

SEQ ID NO: 9 - DNA, 39 bp, synthetic construct
  source 1..39: mol_type = other DNA
gataatattgaggctcagctccggtagaatggcggagac

SEQ ID NO: 10 - DNA, 32 bp, synthetic construct
  source 1..32: mol_type = other DNA
ggacgcgttagcacgctggtacgctcatgcac

SEQ ID NO: 11 - DNA, 27 bp, synthetic construct
  source 1..27: mol_type = other DNA
gccggttaccaggctacggatggtacg

SEQ ID NO: 12 - DNA, 23 bp, synthetic construct
  source 1..23: mol_type = other DNA
tgggataggctctaagtccaacg

SEQ ID NO: 13 - DNA, 22 bp, synthetic construct
  source 1..22: mol_type = other DNA
gttggtatcgttcgggtcgtaa

SEQ ID NO: 14 - DNA, 25 bp, synthetic construct
  source 1..25: mol_type = other DNA
agtctccattcttgacgaaaacatc

SEQ ID NO: 15 - DNA, 23 bp, synthetic construct
  source 1..23: mol_type = other DNA
caaactctccactcgtcctgaaa

SEQ ID NO: 16 - DNA, 25 bp, synthetic construct
  source 1..25: mol_type = other DNA
ccggagtagacttagtcgagatcag

SEQ ID NO: 17 - DNA, 23 bp, synthetic construct
  source 1..23: mol_type = other DNA
aaaaggtaacgcaagcaatgaat

SEQ ID NO: 18 - DNA, 23 bp, synthetic construct
  source 1..23: mol_type = other DNA
cagtttttctttcatccacagcg

SEQ ID NO: 19 - DNA, 28 bp, synthetic construct
  source 1..28: mol_type = other DNA
gccataagacataacaattacagaagga

SEQ ID NO: 20 - DNA, 23 bp, synthetic construct
  source 1..23: mol_type = other DNA
atacgggcatagaatccgctctc

SEQ ID NO: 21 - DNA, 22 bp, synthetic construct
  source 1..22: mol_type = other DNA
ggcgttgattttacggtgactc

SEQ ID NO: 22 - DNA, 23 bp, synthetic construct
  source 1..23: mol_type = other DNA
actcaacctcattcagtttaccg

SEQ ID NO: 23 - DNA, 23 bp, synthetic construct
  source 1..23: mol_type = other DNA
ggtgatagacagcgggctaaaga

SEQ ID NO: 24 - DNA, 24 bp, synthetic construct
  source 1..24: mol_type = other DNA
tgttatttcaatcggttattcgct

SEQ ID NO: 25 - DNA, 24 bp, synthetic construct
  source 1..24: mol_type = other DNA
gataaactgctcggtaataaacgc

SEQ ID NO: 26 - DNA, 22 bp, synthetic construct
  source 1..22: mol_type = other DNA
ccgcaaatgaggataaagcaaa

SEQ ID NO: 27 - DNA, 21 bp, synthetic construct
  source 1..21: mol_type = other DNA
ccagccattgcgactgttcac

SEQ ID NO: 28 - DNA, 18 bp, synthetic construct
  source 1..18: mol_type = other DNA
caggaagcggggcaggtt

SEQ ID NO: 29 - DNA, 22 bp, synthetic construct
  source 1..22: mol_type = other DNA
caatcgtccttagtttggcagc

SEQ ID NO: 30 - DNA, 24 bp, synthetic construct
  source 1..24: mol_type = other DNA
atggtttttatcagtcacttccgg

SEQ ID NO: 31 - DNA, 19 bp, synthetic construct
  source 1..19: mol_type = other DNA
cgtgacctcaatgccgacc

SEQ ID NO: 32 - DNA, 25 bp, synthetic construct
  source 1..25: mol_type = other DNA
cacaactcaccacagaagaggattt

SEQ ID NO: 33 - DNA, 25 bp, synthetic construct
  source 1..25: mol_type = other DNA
gcattatacgagaaatctttccctc

SEQ ID NO: 34 - DNA, 23 bp, synthetic construct
  source 1..23: mol_type = other DNA
ccattgttttgtcgttcctgatc

SEQ ID NO: 35 - DNA, 26 bp, synthetic construct
  source 1..26: mol_type = other DNA
caattgcgtgtatcatagatgttttg

SEQ ID NO: 36 - DNA, 27 bp, synthetic construct
  source 1..27: mol_type = other DNA
gcgaaggcggccgttagccaggcgtca

SEQ ID NO: 37 - DNA, 27 bp, synthetic construct
  source 1..27: mol_type = other DNA
gctggtgcggttgtcaacgaagcgaaa

SEQ ID NO: 38 - DNA, 27 bp, synthetic construct
  source 1..27: mol_type = other DNA
gcggcggaaacggttgatgtctctctg

SEQ ID NO: 39 - DNA, 27 bp, synthetic construct
  source 1..27: mol_type = other DNA
ccaggtcgtcgcgttgaaaacggcggt

SEQ ID NO: 40 - DNA, 36 bp, synthetic construct
  source 1..36: mol_type = other DNA
gttacccgtaccgtcgaccgtgtcgaaagtttcttc

SEQ ID NO: 41 - DNA, 27 bp, synthetic construct
  source 1..27: mol_type = other DNA
accgggccggaagtcgaagacgattat

SEQ ID NO: 42 - DNA, 27 bp, synthetic construct
  source 1..27: mol_type = other DNA
gatgctctgaacgttcctggtcaccac

SEQ ID NO: 43 - DNA, 27 bp, synthetic construct
  source 1..27: mol_type = other DNA
tctggcgtacaggtccgcaccatgaaa

SEQ ID NO: 44 - DNA, 36 bp, synthetic construct
  source 1..36: mol_type = other DNA
cagcagccaccggttcgtgtcgtcgcgcctggccgt

SEQ ID NO: 45 - DNA, 42 bp, synthetic construct
  source 1..42: mol_type = other DNA
atggaaggtctggttgttgataccaacgtcagctttaccaac

SEQ ID NO: 46 - DNA, 27 bp, synthetic construct
  source 1..27: mol_type = other DNA
gaagatttgcaggttcgcttccgtcct

SEQ ID NO: 47 - DNA, 27 bp, synthetic construct
  source 1..27: mol_type = other DNA
cgtaacgttggcgtcgacccggaagtt

SEQ ID NO: 48 - DNA, 27 bp, synthetic construct
  source 1..27: mol_type = other DNA
tttacacagaccgagatagtcatggaa

SEQ ID NO: 49 - DNA, 25 bp, synthetic construct
  source 1..25: mol_type = other DNA
gaccgagcccgctgttgtcagagat

SEQ ID NO: 50 - DNA, 21 bp, synthetic construct
  source 1..21: mol_type = other DNA
gccccagtagcgcattcagca

SEQ ID NO: 51 - DNA, 21 bp, synthetic construct
  source 1..21: mol_type = other DNA
cgcccggttagcgcctcttcc

SEQ ID NO: 52 - DNA, 36 bp, synthetic construct
  source 1..36: mol_type = other DNA
tggctggaaaccattagtacgcgataaccgatattg

SEQ ID NO: 53 - DNA, 27 bp, synthetic construct
  source 1..27: mol_type = other DNA
cggctcaatctgtagggtttcgcaggc

SEQ ID NO: 54 - DNA, 50 bp, synthetic construct
  source 1..50: mol_type = other DNA
ggtcgataatattgaggctcagctccggtagaatggcggagacaatgaca

SEQ ID NO: 55 - DNA, 36 bp, synthetic construct
  source 1..36: mol_type = other DNA
cggacgcgttagcacgctggtacgctcatgcaccgc

SEQ ID NO: 56 - DNA, 32 bp, synthetic construct
  source 1..32: mol_type = other DNA
cgccggttaccaggctacggatggtacggcga

SEQ ID NO: 57 - DNA, 25 bp, synthetic construct
  source 1..25: mol_type = other DNA
ggtattggatagactttaagcaaac

SEQ ID NO: 58 - DNA, 28 bp, synthetic construct
  source 1..28: mol_type = other DNA
cttgactccttagagagtcctactaatg

SEQ ID NO: 59 - DNA, 38 bp, synthetic construct
  source 1..38: mol_type = other DNA
actttttcgcagtttacttggtttcagtacattactag

SEQ ID NO: 60 - DNA, 37 bp, synthetic construct
  source 1..37: mol_type = other DNA
aattttgagattatacgctacttaatgatttttatat

SEQ ID NO: 61 - DNA, 36 bp, synthetic construct
  source 1..36: mol_type = other DNA
gttatagggtggcgccctcgctttaccggtttattt

SEQ ID NO: 62 - DNA, 27 bp, synthetic construct
  source 1..27: mol_type = other DNA
caaactagtgctttaactatcgatggt

SEQ ID NO: 63 - DNA, 37 bp, synthetic construct
  source 1..37: mol_type = other DNA
acctgttacattattagatagtcgacgaaatcattgg

SEQ ID NO: 64 - DNA, 47 bp, synthetic construct
  source 1..47: mol_type = other DNA
gtttttatcatttatctcaacgcagctctcgagcgattgaaaaataa

SEQ ID NO: 65 - DNA, 47 bp, synthetic construct
  source 1..47: mol_type = other DNA
gtgtttggactccctgaatatcaacttaacctcatgaatccactact

SEQ ID NO: 66 - DNA, 37 bp, synthetic construct
  source 1..37: mol_type = other DNA
gcttgatcatgatatcagcactcattatatatctgca

SEQ ID NO: 67 - DNA, 22 bp, synthetic construct
  source 1..22: mol_type = other DNA
ggacgtaacgtcaccgataggg

SEQ ID NO: 68 - DNA, 19 bp, synthetic construct
  source 1..19: mol_type = other DNA
gctccgcagccacttttca

SEQ ID NO: 69 - DNA, 22 bp, synthetic construct
  source 1..22: mol_type = other DNA
tctcaagcactagaggctgtgc

SEQ ID NO: 70 - DNA, 20 bp, synthetic construct
  source 1..20: mol_type = other DNA
ccacaccagtccggtgcagg

SEQ ID NO: 71 - DNA, 25 bp, synthetic construct
  source 1..25: mol_type = other DNA
cctgacccacagcccgatgttccac

SEQ ID NO: 72 - DNA, 24 bp, synthetic construct
  source 1..24: mol_type = other DNA
ggaaggcctgctagtggacgaggg

SEQ ID NO: 73 - DNA, 25 bp, synthetic construct
  source 1..25: mol_type = other DNA
caccgagcccagcgccgaagtggac

SEQ ID NO: 74 - DNA, 28 bp, synthetic construct
  source 1..28: mol_type = other DNA
ggacaattccgctaagccagaacgcaac

SEQ ID NO: 75 - DNA, 30 bp, synthetic construct
  source 1..30: mol_type = other DNA
cttatcgaaattaatacgactcactatagg

SEQ ID NO: 76 - DNA, 23 bp, synthetic construct
  source 1..23: mol_type = other DNA
ggatccactagtccagtgtggtg

SEQ ID NO: 77 - DNA, 28 bp, synthetic construct
  source 1..28: mol_type = other DNA
gtttgtgttcttcgtcttgttgcctttg

SEQ ID NO: 78 - DNA, 29 bp, synthetic construct
  source 1..29: mol_type = other DNA
ctttggtttcaattcagtgcgtgaatttg

SEQ ID NO: 79 - DNA, 51 bp, synthetic construct
  source 1..51: mol_type = other DNA
gtgcgtgaatttcactaaccgaactcagctgccgtctgcctacaccaattc

SEQ ID NO: 80 - DNA, 27 bp, synthetic construct
  source 1..27: mol_type = other DNA
gcgtgaatttgcgtacacgaactcagc

SEQ ID NO: 81 - DNA, 26 bp, synthetic construct
  source 1..26: mol_type = other DNA
gcgtgaatttcactacacgaactcag

SEQ ID NO: 82 - DNA, 48 bp, synthetic construct
  source 1..48: mol_type = other DNA
gtcacatggttccatgtaataagcgggacgaatgggaccaaacgcttc

SEQ ID NO: 83 - DNA, 44 bp, synthetic construct
  source 1..44: mol_type = other DNA
catggttccatgcaataagcgggacgaatgggaccaaacgcttc

SEQ ID NO: 84 - DNA, 28 bp, synthetic construct
  source 1..28: mol_type = other DNA
catgcaatacactttagcgggacgaatg

SEQ ID NO: 85 - DNA, 33 bp, synthetic construct
  source 1..33: mol_type = other DNA
ggacgaatgtgatcaaacgcttcgataatccgg

SEQ ID NO: 86 - DNA, 26 bp, synthetic construct
  source 1..26: mol_type = other DNA
caaacgcttcgctaatccggtactcc

SEQ ID NO: 87 - DNA, 25 bp, synthetic construct
  source 1..25: mol_type = other DNA
caaacgcttcggtaatccggtactc

SEQ ID NO: 88 - DNA, 37 bp, synthetic construct
  source 1..37: mol_type = other DNA
ctacttcgcttctattgagaagtcaaacattatccgc

SEQ ID NO: 89 - DNA, 26 bp, synthetic construct
  source 1..26: mol_type = other DNA
attttgcaattacccgttcctgggcg

SEQ ID NO: 90 - DNA, 47 bp, synthetic construct
  source 1..47: mol_type = other DNA
ttgcaatgacccgttctattatcataagaataacaagagctggatgg

SEQ ID NO: 91 - DNA, 44 bp, synthetic construct
  source 1..44: mol_type = other DNA
gcaatgacccgttcctggacgtttattatcataagaataacaag

SEQ ID NO: 92 - DNA, 52 bp, synthetic construct
  source 1..52: mol_type = other DNA
caatgacccgttcctgggcgtttatcataagaataacaagagctggatggag

SEQ ID NO: 93 - DNA, 74 bp, synthetic construct
  source 1..74: mol_type = other DNA
gacccgttcctggacgtttattatcataagaataacaagagctggatgaagtctgagttccgcgtatactcttc

SEQ ID NO: 94 - DNA, 70 bp, synthetic construct
  source 1..70: mol_type = other DNA
gttcctgggcgtttatcataagaataacaagagctggatggagtctgagtcccgcgtatactcttctgcc

SEQ ID NO: 95 - DNA, 30 bp, synthetic construct
  source 1..30: mol_type = other DNA
ataacaagagctgcatggagtctgagttcc

SEQ ID NO: 96 - DNA, 46 bp, synthetic construct
  source 1..46: mol_type = other DNA
taacaagagctggatgaagtctgagttccgcgtatactcttctgcc

SEQ ID NO: 97 - DNA, 60 bp, synthetic construct
  source 1..60: mol_type = other DNA
acaagagctggatggagtctggggtatactcttctgccaataattgtaccttcgaatacg

SEQ ID NO: 98 - DNA, 48 bp, synthetic construct
  source 1..48: mol_type = other DNA
caagagctggatggagtctggcgtatactcttctgccaataattgtac

SEQ ID NO: 99 - DNA, 34 bp, synthetic construct
  source 1..34: mol_type = other DNA
gatggagtctgagtcccgcgtatactcttctgcc

SEQ ID NO: 100 - DNA, 48 bp, synthetic construct
  source 1..48: mol_type = other DNA
caaggcaatttcaaaaacctcagcgagttcgtttttaagaatatcgat

SEQ ID NO: 101 - DNA, 29 bp, synthetic construct
  source 1..29: mol_type = other DNA
cttgtccgaggtctgccgcaaggatttag

SEQ ID NO: 102 - DNA, 37 bp, synthetic construct
  source 1..37: mol_type = other DNA
caaggatttagtgtacttgaaccgctggtcgatcttc

SEQ ID NO: 103 - DNA, 52 bp, synthetic construct
  source 1..52: mol_type = other DNA
cattacccggtttcaaactttgcacatcagttatcttacgccaggcgacagc

SEQ ID NO: 104 - DNA, 49 bp, synthetic construct
  source 1..49: mol_type = other DNA
cattacccggtttcaaactttgcaccggagttatcttacgccaggcgac

SEQ ID NO: 105 - DNA, 64 bp, synthetic construct
  source 1..64: mol_type = other DNA
gtttcaaactctgttggctttgcacaacagctccagtggatggacggcaggggctgctgcatac

SEQ ID NO: 106 - DNA, 23 bp, synthetic construct
  source 1..23: mol_type = other DNA
acgccaggcggcagctccagtgg

SEQ ID NO: 107 - DNA, 28 bp, synthetic construct
  source 1..28: mol_type = other DNA
gctccagtggattgacggcaggggctgc

SEQ ID NO: 108 - DNA, 36 bp, synthetic construct
  source 1..36: mol_type = other DNA
gggcagacgggaaatatagccgattataactataag

SEQ ID NO: 109 - DNA, 36 bp, synthetic construct
  source 1..36: mol_type = other DNA
gggcagacgggaacaatagccgattataactataag

SEQ ID NO: 110 - DNA, 40 bp, synthetic construct
  source 1..40: mol_type = other DNA
gaggaaactataactatcagtaccggctctttcgcaagtc

SEQ ID NO: 111 - DNA, 40 bp, synthetic construct
  source 1..40: mol_type = other DNA
gaggaaactataactatcgctaccggctctttcgcaagtc

SEQ ID NO: 112 - DNA, 54 bp, synthetic construct
  source 1..54: mol_type = other DNA
ccaagcaggtaatacgccgtgcaacggggtaaaagggtttaattgttacttccc

SEQ ID NO: 113 - DNA, 32 bp, synthetic construct
  source 1..32: mol_type = other DNA
ccaagcaggtaatacgccgtgcaacggggtag

SEQ ID NO: 114 - DNA, 29 bp, synthetic construct
  source 1..29: mol_type = other DNA
ccaagcaggtagtaagccgtgcaacgggg

SEQ ID NO: 115 - DNA, 39 bp, synthetic construct
  source 1..39: mol_type = other DNA
agtacgccgtgcaacggggtaaaagggtttaattgttac

SEQ ID NO: 116 - DNA, 39 bp, synthetic construct
  source 1..39: mol_type = other DNA
ccgtgcaacggggtacaagggtttaattgttacttcccg

SEQ ID NO: 117 - DNA, 34 bp, synthetic construct
  source 1..34: mol_type = other DNA
gggtttaattgttactccccgttgcagagttacg

SEQ ID NO: 118 - DNA, 61 bp, synthetic construct
  source 1..61: mol_type = other DNA
ttcccgttgcagccttacggatttcaaccaacctacggagtagggtatcagccgtaccgcg

SEQ ID NO: 119 - DNA, 41 bp, synthetic construct
  source 1..41: mol_type = other DNA
ggatttcaaccaacctacggagtagggtatcagccgtaccg

SEQ ID NO: 120 - DNA, 32 bp, synthetic construct
  source 1..32: mol_type = other DNA
gccgttccagcagttggggagggatattgctg

SEQ ID NO: 121 - DNA, 31 bp, synthetic construct
  source 1..31: mol_type = other DNA
gggagggatattgatgacactaccgacgcag

SEQ ID NO: 122 - DNA, 41 bp, synthetic construct
  source 1..41: mol_type = other DNA
gtttgattggtgctgagtacgtaaataactcatacgaatgc

SEQ ID NO: 123 - DNA, 30 bp, synthetic construct
  source 1..30: mol_type = other DNA
ctaccaaactcataccaattcaccgaggcg

SEQ ID NO: 124 - DNA, 31 bp, synthetic construct
  source 1..31: mol_type = other DNA
caaaccaattcacacaggcgcgcacgatccg

SEQ ID NO: 125 - DNA, 29 bp, synthetic construct
  source 1..29: mol_type = other DNA
caaaccaattcacggaggcgcgcacgatc

SEQ ID NO: 126 - DNA, 28 bp, synthetic construct
  source 1..28: mol_type = other DNA
gagcctcggcgtagaaaactcagttgcg

SEQ ID NO: 127 - DNA, 46 bp, synthetic construct
  source 1..46: mol_type = other DNA
aacagcattgcaatccctataaattttacgatcagtgttacgaccg

SEQ ID NO: 128 - DNA, 36 bp, synthetic construct
  source 1..36: mol_type = other DNA
gttcaatgggctgaacgtgctgcccccgctgcttac

SEQ ID NO: 129 - DNA, 22 bp, synthetic construct
  source 1..22: mol_type = other DNA
ggtggacgttgggcgcaggagc

SEQ ID NO: 130 - DNA, 60 bp, synthetic construct
  source 1..60: mol_type = other DNA
cctggggaagctgcaacatgtcgtaaaccagaacgctcgagcgcttaatacattggttaa

SEQ ID NO: 131 - DNA, 41 bp, synthetic construct
  source 1..41: mol_type = other DNA
cctggggaagctgcaacatgtcgtaaaccagaacgctcaag

SEQ ID NO: 132 - DNA, 41 bp, synthetic construct
  source 1..41: mol_type = other DNA
cctggggaagctgcaaaatgtcgtaaaccagaacgctcaag

SEQ ID NO: 133 - DNA, 50 bp, synthetic construct
  source 1..50: mol_type = other DNA
gtcgtaaaccagaacgctcgagcgcttaatacattggttaaacaattatc

SEQ ID NO: 134 - DNA, 37 bp, synthetic construct
  source 1..37: mol_type = other DNA
cttaacgacattttggctcggcttgacaaggttgagg

SEQ ID NO: 135 - DNA, 38 bp, synthetic construct
  source 1..38: mol_type = other DNA
cgcaaaccttgcggccataaaaatgagtgaatgtgtac

SEQ ID NO: 136 - DNA, 44 bp, synthetic construct
  source 1..44: mol_type = other DNA
gtaacttatgtacctgcgcatgaaaaaaatttcacgactgcgcc

SEQ ID NO: 137 - DNA, 53 bp, synthetic construct
  source 1..53: mol_type = other DNA
cacttcccaaggaaaggcgtctttgtatcaaatgggacgtattggttcgtcac

SEQ ID NO: 138 - DNA, 47 bp, synthetic construct
  source 1..47: mol_type = other DNA
ccagattataactacccacaacacattcgtcagcggcaattgtgatg

SEQ ID NO: 139 - DNA, 68 bp, synthetic construct
  source 1..68: mol_type = other DNA
cggtgatatatcaggtataaatgcgtcattcgtaaacatccagaaggagattgatcgcttgaacgagg

SEQ ID NO: 140 - DNA, 68 bp, synthetic construct
  source 1..68: mol_type = other DNA
ggagattgatcgcttgaacgaggttgctaacaatttgaatgaaagcctcatcgacctccaggaattag

SEQ ID NO: 141 - DNA, 27 bp, synthetic construct
  source 1..27: mol_type = other DNA
gcgtgaatttgnnsacacgaactcagc

SEQ ID NO: 142 - DNA, 28 bp, synthetic construct
  source 1..28: mol_type = other DNA
catgcaatacacnnsagcgggacgaatg

SEQ ID NO: 143 - DNA, 41 bp, synthetic construct
  source 1..41: mol_type = other DNA
gcaatgacccgttcctgnnsgtttattatcataagaataac

SEQ ID NO: 144 - DNA, 39 bp, synthetic construct
  source 1..39: mol_type = other DNA
gcaaggatttagtnnscttgaaccgctggtcgatcttcc

SEQ ID NO: 145 - DNA, 36 bp, synthetic construct
  source 1..36: mol_type = other DNA
gggcagacgggannsatagccgattataactataag

SEQ ID NO: 146 - DNA, 40 bp, synthetic construct
  source 1..40: mol_type = other DNA
gaggaaactataactatnnstaccggctctttcgcaagtc

SEQ ID NO: 147 - DNA, 29 bp, synthetic construct
  source 1..29: mol_type = other DNA
ccaagcaggtagtnnsccgtgcaacgggg

SEQ ID NO: 148 - DNA, 29 bp, synthetic construct
  source 1..29: mol_type = other DNA
caaaccaattcannsaggcgcgcacgatc

SEQ ID NO: 149 - DNA, 43 bp, synthetic construct
  source 1..43: mol_type = other DNA
cctggggaagctgcaannsgtcgtaaaccagaacgctcaagcg

SEQ ID NO: 150 - DNA, 51 bp, synthetic construct
  source 1..51: mol_type = other DNA
aagagctggatggagtctnnsgtatactcttctgccaataattgtaccttc

SEQ ID NO: 151 - DNA, 22 bp, synthetic construct
  source 1..22: mol_type = other DNA
ccctcgatgttgacgttgcaaa

SEQ ID NO: 152 - DNA, 22 bp, synthetic construct
  source 1..22: mol_type = other DNA
gaaagcggcgaacatcctgatc

SEQ ID NO: 153 - DNA, 20 bp, synthetic construct
  source 1..20: mol_type = other DNA
aagcccagatcgtacatgcg

SEQ ID NO: 154 - DNA, 21 bp, synthetic construct
  source 1..21: mol_type = other DNA
gcgtttgtgctgattaccctt

SEQ ID NO: 155 - DNA, 22 bp, synthetic construct
  source 1..22: mol_type = other DNA
aaaatcgcctgctttggtctgg

SEQ ID NO: 156 - DNA, 22 bp, synthetic construct
  source 1..22: mol_type = other DNA
gcggatcgtatcaaccagcaac

SEQ ID NO: 157 - DNA, 22 bp, synthetic construct
  source 1..22: mol_type = other DNA
gctggcactaaaacgctatgga

SEQ ID NO: 158 - DNA, 22 bp, synthetic construct
  source 1..22: mol_type = other DNA
cgcccgtaataactcattgccc

SEQ ID NO: 159 - DNA, 22 bp, synthetic construct
  source 1..22: mol_type = other DNA
gacctgttttaacgcctgctcc

SEQ ID NO: 160 - DNA, 22 bp, synthetic construct
  source 1..22: mol_type = other DNA
gccgttatccagtttgctcagg

SEQ ID NO: 161 - DNA, 22 bp, synthetic construct
  source 1..22: mol_type = other DNA
ggatgttcgccgctttcagaat

SEQ ID NO: 162 - DNA, 21 bp, synthetic construct
  source 1..21: mol_type = other DNA
ttcaggtggtggtactggacg

SEQ ID NO: 163 - DNA, 19 bp, synthetic construct
  source 1..19: mol_type = other DNA
tgctgcggcaagggtaatc

SEQ ID NO: 164 - DNA, 21 bp, synthetic construct
  source 1..21: mol_type = other DNA
aggtcatcaatattcggttta

SEQ ID NO: 165 - DNA, 22 bp, synthetic construct
  source 1..22: mol_type = other DNA
gttgctggttgatacgatccgc

SEQ ID NO: 166 - DNA, 22 bp, synthetic construct
  source 1..22: mol_type = other DNA
gtatagaacgcttcatcgccgc

SEQ ID NO: 167 - DNA, 22 bp, synthetic construct
  source 1..22: mol_type = other DNA
taccgtggggcaatgagttatt

SEQ ID NO: 168 - DNA, 22 bp, synthetic construct
  source 1..22: mol_type = other DNA
ggagcaggcgttaaaacaggtc

SEQ ID NO: 169 - DNA, 22 bp, synthetic construct
  source 1..22: mol_type = other DNA
cctgagcaaactggataacggc

SEQ ID NO: 170 - DNA, 19 bp, synthetic construct
  source 1..19: mol_type = other DNA
cggcgaaaccttctgggat

SEQ ID NO: 171 - DNA, 20 bp, synthetic construct
  source 1..20: mol_type = other DNA
gggctacagccacggtttca

SEQ ID NO: 172 - DNA, 21 bp, synthetic construct
  source 1..21: mol_type = other DNA
tgctggaaagctccttcgttg

SEQ ID NO: 173 - DNA, 20 bp, synthetic construct
  source 1..20: mol_type = other DNA
cggtatgctacaggctggct

SEQ ID NO: 174 - DNA, 26 bp, synthetic construct
  source 1..26: mol_type = other DNA
catattatgaaagtctgcacgagctg

SEQ ID NO: 175 - DNA, 21 bp, synthetic construct
  source 1..21: mol_type = other DNA
gtgtgccgctactgaaaccgt

SEQ ID NO: 176 - DNA, 38 bp, synthetic construct
  source 1..38: mol_type = other DNA
caccgagatcaccgccgcgtttgccacttgggcaaatg

SEQ ID NO: 177 - DNA, 30 bp, synthetic construct
  source 1..30: mol_type = other DNA
gtcgcgagtggtactgagtggaacgacgat

SEQ ID NO: 178 - DNA, 41 bp, synthetic construct
  source 1..41: mol_type = other DNA
ttgagcacgacaacgagtcggttttttatcatttcaggagg

SEQ ID NO: 179 - DNA, 32 bp, synthetic construct
  source 1..32: mol_type = other DNA
cactttccatacgctagggctggatatcatca

SEQ ID NO: 180 - DNA, 39 bp, synthetic construct
  source 1..39: mol_type = other DNA
cttgggatgaaagcgaacttcagcctatttgacgatacc

SEQ ID NO: 181 - DNA, 51 bp, synthetic construct
  source 1..51: mol_type = other DNA
gatcagcttgctctacttaaagagctaaccgaggggctgattgaagatgac

SEQ ID NO: 182 - DNA, 43 bp, synthetic construct
  source 1..43: mol_type = other DNA
cctgcaacaactgattagcaccatctctaactggaagaatgat

SEQ ID NO: 183 - DNA, 54 bp, synthetic construct
  source 1..54: mol_type = other DNA
cttcgatgatctgattctcctaccgacgctactgctgcaacgcaatgaagaagt

SEQ ID NO: 184 - DNA, 38 bp, synthetic construct
  source 1..38: mol_type = other DNA
ccgtggtgggtgacgatgaccagagcatctactcctgg

SEQ ID NO: 185 - DNA, 24 bp, synthetic construct
  source 1..24: mol_type = other DNA
gctaaaagtactcagcgcgaataa

SEQ ID NO: 186 - DNA, 23 bp, synthetic construct
  source 1..23: mol_type = other DNA
aaccatcagagccgggtgtttga

SEQ ID NO: 187 - DNA, 28 bp, synthetic construct
  source 1..28: mol_type = other DNA
atatctggtggtacgagctttttctctc

SEQ ID NO: 188 - DNA, 30 bp, synthetic construct
  source 1..30: mol_type = other DNA
gtcctgaaatcaaggacctcctggcttatc

SEQ ID NO: 189 - DNA, 40 bp, synthetic construct
  source 1..40: mol_type = other DNA
gaagcactaacccgcttcactcactggctagcagaaatcc

SEQ ID NO: 190 - DNA, 29 bp, synthetic construct
  source 1..29: mol_type = other DNA
gctgtacgaaacaagccccagcccgaaag

SEQ ID NO: 191 - DNA, 29 bp, synthetic construct
  source 1..29: mol_type = other DNA
acgcgctttactctacgcgacatgatgga

SEQ ID NO: 192 - DNA, 29 bp, synthetic construct
  source 1..29: mol_type = other DNA
ctccacgcgagcaaagggctggagtttcc

SEQ ID NO: 193 - DNA, 37 bp, synthetic construct
  source 1..37: mol_type = other DNA
tatggaagaagggtttctaccgcaccagagcagcatc

SEQ ID NO: 194 - DNA, 33 bp, synthetic construct
  source 1..33: mol_type = other DNA
tacccgcgcccagaaggaactaacctttacgct

SEQ ID NO: 195 - DNA, 29 bp, synthetic construct
  source 1..29: mol_type = other DNA
ccgagccgctttctactggagctgccgca

SEQ ID NO: 196 - DNA, 51 bp, synthetic construct
  source 1..51: mol_type = other DNA
cggaagcgttagggttagcagttcatggttagcctggagtgtcatctctgg

SEQ ID NO: 197 - DNA, 30 bp, synthetic construct
  source 1..30: mol_type = other DNA
gccaggcgtaggagacggcagagttgttct

SEQ ID NO: 198 - DNA, 51 bp, synthetic construct
  source 1..51: mol_type = other DNA
ggcattttgctgatgcaggctactgagatcgaccggattagtctggttgag

SEQ ID NO: 199 - DNA, 34 bp, synthetic construct
  source 1..34: mol_type = other DNA
cagtagcgtcgccagtagttttttctgtgcgggg

SEQ ID NO: 200 - DNA, 34 bp, synthetic construct
  source 1..34: mol_type = other DNA
ggcgctgatgagtagatcgcggcttattgcttca

SEQ ID NO: 201 - DNA, 31 bp, synthetic construct
  source 1..31: mol_type = other DNA
gacggccagatgtagcatgccgtagaccagg

SEQ ID NO: 202 - DNA, 31 bp, synthetic construct
  source 1..31: mol_type = other DNA
ctcgggaacacgagcgcacgttccagcgtca

SEQ ID NO: 203 - DNA, 50 bp, synthetic construct
  source 1..50: mol_type = other DNA
gaatggctcgctgttttagttgctgtagcttttccagggtaatgcgttca

SEQ ID NO: 204 - DNA, 32 bp, synthetic construct
  source 1..32: mol_type = other DNA
gccatcatgatttcctggagcgcctgcacggt

SEQ ID NO: 205 - DNA, 42 bp, synthetic construct
  source 1..42: mol_type = other DNA
taccggagttcatcggcaaccggacggagcacttcgcgtgcc

SEQ ID NO: 206 - DNA, 58 bp, synthetic construct
  source 1..58: mol_type = other DNA
caggtgacgcagcccatgctcaggctgaataggctggtggtttgtgcacccgtgccgg

SEQ ID NO: 207 - DNA, 24 bp, synthetic construct
  source 1..24: mol_type = other DNA
cggcaaggcggagcgtcgccgtag

SEQ ID NO: 208 - DNA, 50 bp, synthetic construct
  source 1..50: mol_type = other DNA
cgacaacgcgaatttggctgggaggaatatcttgcagacgttcagcaaac

SEQ ID NO: 209 - DNA, 35 bp, synthetic construct
  source 1..35: mol_type = other DNA
aggcgtagacattgccaaccgcgctccattgcttc

SEQ ID NO: 210 - DNA, 83 bp, synthetic construct
  source 1..83: mol_type = other DNA
cgcgcacaaccagcatatgaaaactattgctaccgagatcaatggctgcatacaggctgctggtggaacccatactcttcatc

SEQ ID NO: 211 - DNA, 57 bp, synthetic construct
  source 1..57: mol_type = other DNA
gattttgagtatgacattttttatttaaccactacgacgacgattacgcggagcgcc

SEQ ID NO: 212 - DNA, 98 bp, synthetic construct
  source 1..98: mol_type = other DNA
cagatcggtcattagtgcgtccggattgtatttgcttaccggaatactgtgaccaatataggtctcaatagcaggtagatttagtgcatactcttcac

SEQ ID NO: 213 - DNA, 55 bp, synthetic construct
  source 1..55: mol_type = other DNA
ggggagatcgtagttaaagacgtgcgtcactgccggaatatgtagaccacgcgcg

SEQ ID NO: 214 - DNA, 45 bp, synthetic construct
  source 1..45: mol_type = other DNA
ctgcgcgacatcgcctgttaggagaccgacacgatgaccatctgc

SEQ ID NO: 215 - DNA, 46 bp, synthetic construct
  source 1..46: mol_type = other DNA
caacggtgtttggtgttggcgaaaataatcgcgcggtctggccact

SEQ ID NO: 216 - DNA, 75 bp, synthetic construct
  source 1..75: mol_type = other DNA
cttcttcgatcagcgtttgcagtagacgcattttttcttcgttagaagggtagaaaagctcttctttaatacggt

SEQ ID NO: 217 - DNA, 52 bp, synthetic construct
  source 1..52: mol_type = other DNA
ccagttcacgtacccggtagctaagcgtggcggagaacagcatgttgaggcg

SEQ ID NO: 218 - DNA, 20 bp, synthetic construct
  source 1..20: mol_type = other DNA
ccataatgagggcacgcggc

SEQ ID NO: 219 - DNA, 30 bp, synthetic construct
  source 1..30: mol_type = other DNA
taatgaaacgtactcgtaagaaacgccatc

SEQ ID NO: 220 - DNA, 31 bp, synthetic construct
  source 1..31: mol_type = other DNA
cttctgttctgtgagatgtgttttgctcata

SEQ ID NO: 221 - DNA, 29 bp, synthetic construct
  source 1..29: mol_type = other DNA
taaaggtcagctaaaagagttcctcgacg

SEQ ID NO: 222 - DNA, 34 bp, synthetic construct
  source 1..34: mol_type = other DNA
cagatttctggcagcagtctcaatccgtcttgtc

SEQ ID NO: 223 - DNA, 27 bp, synthetic construct
  source 1..27: mol_type = other DNA
tcctgtgacccgacagcgaaaagacat

SEQ ID NO: 224 - DNA, 30 bp, synthetic construct
  source 1..30: mol_type = other DNA
tcttaccgaactcaagaatacgccggtttc

SEQ ID NO: 225 - DNA, 32 bp, synthetic construct
  source 1..32: mol_type = other DNA
actggagatactacaggatggatttggtttcc

SEQ ID NO: 226 - DNA, 26 bp, synthetic construct
  source 1..26: mol_type = other DNA
ctttgagaacctcaccccgctgcacg

SEQ ID NO: 227 - DNA, 48 bp, synthetic construct
  source 1..48: mol_type = other DNA
ctgaagatctcactgctcgcgtactggatctggcaagtcctatcggtc

SEQ ID NO: 228 - DNA, 39 bp, synthetic construct
  source 1..39: mol_type = other DNA
ttgttccggcgagtggtaaagtgctaaccggtggtgtgg

SEQ ID NO: 229 - DNA, 32 bp, synthetic construct
  source 1..32: mol_type = other DNA
atgatgaaacgcagttaaatttgtcttatgcc

SEQ ID NO: 230 - DNA, 33 bp, synthetic construct
  source 1..33: mol_type = other DNA
cattgtgaatctcctgacagtgagtactgatct

SEQ ID NO: 231 - DNA, 36 bp, synthetic construct
  source 1..36: mol_type = other DNA
catcagtatttttctcttcacgacactgtttctgtt

SEQ ID NO: 232 - DNA, 34 bp, synthetic construct
  source 1..34: mol_type = other DNA
aaaagtcggtctcgtggataaaccaaacttccgc

SEQ ID NO: 233 - DNA, 47 bp, synthetic construct
  source 1..47: mol_type = other DNA
accagggactaatacctctcgttggggggattagcgtttacgcaggg

SEQ ID NO: 234 - DNA, 51 bp, synthetic construct
  source 1..51: mol_type = other DNA
gcattgatggcctactgggcgggctatcctgcgtcagctttgcagcaatcg

SEQ ID NO: 235 - DNA, 27 bp, synthetic construct
  source 1..27: mol_type = other DNA
ggtgctctttctactagcattcttcct

SEQ ID NO: 236 - DNA, 34 bp, synthetic construct
  source 1..34: mol_type = other DNA
cgcgtaaaacgccgcctgcgtcgcaatcgtggtg

SEQ ID NO: 237 - DNA, 34 bp, synthetic construct
  source 1..34: mol_type = other DNA
gcagccccaatctcaccaaataaatgaggatgtg

SEQ ID NO: 238 - DNA, 43 bp, synthetic construct
  source 1..43: mol_type = other DNA
ggatattcgtgggctatttcgtaccctatgggctgggaagcta

SEQ ID NO: 239 - DNA, 25 bp, synthetic construct
  source 1..25: mol_type = other DNA
cgtttgcgctcatcgcgctggcgta

SEQ ID NO: 240 - DNA, 32 bp, synthetic construct
  source 1..32: mol_type = other DNA
gtggagcagcacggcgattaccgatcgtccaa

SEQ ID NO: 241 - DNA, 56 bp, synthetic construct
  source 1..56: mol_type = other DNA
cggtgaatatgctggggggatattacagccagcagcaatttctacgtaacctggat

SEQ ID NO: 242 - DNA, 50 bp, synthetic construct
  source 1..50: mol_type = other DNA
gtccgtagtaacatggcttctgccgaccaaccaagcgtcatggacgaagc

SEQ ID NO: 243 - DNA, 53 bp, synthetic construct
  source 1..53: mol_type = other DNA
tttgttatgcaactggccagctgggatacccgccgcgagttctggctgcaaac

SEQ ID NO: 244 - DNA, 43 bp, synthetic construct
  source 1..43: mol_type = other DNA
gccgatgcggcgctactggatgaaatgattaacaacatccagt

SEQ ID NO: 245 - DNA, 47 bp, synthetic construct
  source 1..47: mol_type = other DNA
cgctaataacctgctccgtcagtatgttgcttttgccagccagcgtg

SEQ ID NO: 246 - DNA, 44 bp, synthetic construct
  source 1..44: mol_type = other DNA
attgctgagcagcataatattagccgcagtgcgacagatgtacc

SEQ ID NO: 247 - DNA, 57 bp, synthetic construct
  source 1..57: mol_type = other DNA
tgccgaggaactccctgatagtgaaatgttcctgcttgggcgtccaatgcttcaggc

SEQ ID NO: 248 - DNA, 42 bp, synthetic construct
  source 1..42: mol_type = other DNA
tcgactggaaaatctccaggccgtcggtccggcctttgatct

SEQ ID NO: 249 - DNA, 50 bp, synthetic construct
  source 1..50: mol_type = other DNA
ctatgatcagaatcgggccatgctcaacaccctgaatgttggtccaaccc

SEQ ID NO: 250 - DNA, 50 bp, synthetic construct
  source 1..50: mol_type = other DNA
ctatcgctatctacgtacgccggaagaaccggtaaaacgcgatagcccac

SEQ ID NO: 251 - DNA, 58 bp, synthetic construct
  source 1..58: mol_type = other DNA
atcggggctggtgtcgcactcacccgccgttgcagcaaataacaacactgctgcggtg

SEQ ID NO: 252 - DNA, 62 bp, synthetic construct
  source 1..62: mol_type = other DNA
agccatcaagatggcgccgctagtgcatgcgctagcaaaagatcctttttttgaggctaaag

SEQ ID NO: 253 - DNA, 33 bp, synthetic construct
  source 1..33: mol_type = other DNA
cacgggcgatctctatagcccgtggccggaaga

SEQ ID NO: 254 - DNA, 40 bp, synthetic construct
  source 1..40: mol_type = other DNA
ggctaaccgtacactaaccgggcatctggcgatgtatcac

SEQ ID NO: 255 - DNA, 39 bp, synthetic construct
  source 1..39: mol_type = other DNA
ccggcaaaacctactgcgtgaaaacgttgcggatagccg

SEQ ID NO: 256 - DNA, 41 bp, synthetic construct
  source 1..41: mol_type = other DNA
tgatgcactgctctgggtgcgtgaccaggtgatgagcagcg

SEQ ID NO: 257 - DNA, 49 bp, synthetic construct
  source 1..49: mol_type = other DNA
acaagctgcgtagtgaactggcggcaaattacccgtttatcgaccccga

SEQ ID NO: 258 - DNA, 57 bp, synthetic construct
  source 1..57: mol_type = other DNA
gattctggtgaccggtcaccgacgtgagagtttcggtcgtggctttgaagaaatctg

SEQ ID NO: 259 - DNA, 41 bp, synthetic construct
  source 1..41: mol_type = other DNA
cccgaacgtccgcgaaccggtcaatcgcattctggggcatg

SEQ ID NO: 260 - DNA, 59 bp, synthetic construct
  source 1..59: mol_type = other DNA
aatgtcattctgatcgatccccaggagtatctcccgtttgtctggctgatgaaccacgc

SEQ ID NO: 261 - DNA, 82 bp, synthetic construct
  source 1..82: mol_type = other DNA
ctggctgattctaaccgacagtggcggcattcaggaagaagcgcctagcctggggaaacctgtgctggtgatgcgcgatacc

SEQ ID NO: 262 - DNA, 55 bp, synthetic construct
  source 1..55: mol_type = other DNA
cgaggaagtgacgcgtcttctcaaagacgaaaacgaatatcaagctatgagccgc

SEQ ID NO: 263 - DNA, 83 bp, synthetic construct
  source 1..83: mol_type = other DNA
ggcatgctctcgcattctggaagcgctcaaaaataatcggataagtctatgagttttgcgaccatttctgttatcggactggg

SEQ ID NO: 264 - DNA, 52 bp, synthetic construct
  source 1..52: mol_type = other DNA
caacggcagcagcgtttgccagtcggcaaaaacaggtaattggtgtcgatat

SEQ ID NO: 265 - DNA, 76 bp, synthetic construct
  source 1..76: mol_type = other DNA
tcgtcgaacctgatctagcgagtgtagtaaaaactgccgtagaaggcggttttctccgagcgagcacgacgccagt

SEQ ID NO: 266 - DNA, 42 bp, synthetic construct
  source 1..42: mol_type = other DNA
cctacgttgaaagcgctgctcgctccattgcgccagtgctga

SEQ ID NO: 267 - DNA, 77 bp, synthetic construct
  source 1..77: mol_type = other DNA
tgatccttgaatccaccagcccggtggggagtaccgagaagatggcagaatggctcgcagagatgcgtccggatctc

SEQ ID NO: 268 - DNA, 48 bp, synthetic construct
  source 1..48: mol_type = other DNA
cggaacgcgtgctcccaggacaggtaatggtcgagctgattaaaaacg

SEQ ID NO: 269 - DNA, 42 bp, synthetic construct
  source 1..42: mol_type = other DNA
gtatgacgccggtttgtagcgcccgcgccagcgaactgtaca

SEQ ID NO: 270 - DNA, 53 bp, synthetic construct
  source 1..53: mol_type = other DNA
aggtgagtgtgtcgtcactaacagccggacggcggaaatgtgtaagctcaccg

SEQ ID NO: 271 - DNA, 72 bp, synthetic construct
  source 1..72: mol_type = other DNA
cgcttttgctaatgaactaagcctgatttgtgccgatcaggggattaacgtctgggaactgattcgcctggc

SEQ ID NO: 272 - DNA, 22 bp, synthetic construct
  source 1..22: mol_type = other DNA
atccaccagctaccgaaaaaac

SEQ ID NO: 273 - DNA, 25 bp, synthetic construct
  source 1..25: mol_type = other DNA
cgctgatgcgcaaaattctgataac

SEQ ID NO: 274 - DNA, 22 bp, synthetic construct
  source 1..22: mol_type = other DNA
ttggcagcgcgctggtgcgtta

SEQ ID NO: 275 - DNA, 22 bp, synthetic construct
  source 1..22: mol_type = other DNA
ctgatgagcctggcaccggtcg

SEQ ID NO: 276 - DNA, 27 bp, synthetic construct
  source 1..27: mol_type = other DNA
ggacttatacactacttgaagcggcgc

SEQ ID NO: 277 - DNA, 39 bp, synthetic construct
  source 1..39: mol_type = other DNA
ggaagataaaaaaagcgcgttccgttttcatcatatctc

SEQ ID NO: 278 - DNA, 31 bp, synthetic construct
  source 1..31: mol_type = other DNA
gacctgcacagcacggatgatttcttcaccg

SEQ ID NO: 279 - DNA, 32 bp, synthetic construct
  source 1..32: mol_type = other DNA
ttattccgcgagtaaagccagcagcgaccatc

SEQ ID NO: 280 - DNA, 40 bp, synthetic construct
  source 1..40: mol_type = other DNA
gcttatcaccaactgcagcaataactacggcccttaccac

SEQ ID NO: 281 - DNA, 35 bp, synthetic construct
  source 1..35: mol_type = other DNA
ctggcgggtaaaagcctgccggtatatggcaacgg

SEQ ID NO: 282 - DNA, 35 bp, synthetic construct
  source 1..35: mol_type = other DNA
ccattgatgctagcaaaattgcccgtgaacttggc

SEQ ID NO: 283 - DNA, 29 bp, synthetic construct
  source 1..29: mol_type = other DNA
gggcgagcgtctcggtctgaaaggctaat

SEQ ID NO: 284 - DNA, 26 bp, synthetic construct
  source 1..26: mol_type = other DNA
cggcacccgactacatccgattacgc

SEQ ID NO: 285 - DNA, 54 bp, synthetic construct
  source 1..54: mol_type = other DNA
gcggcgtaagcaagcaactgctaccgatttacgataagccaatgatttactatc

SEQ ID NO: 286 - DNA, 31 bp, synthetic construct
  source 1..31: mol_type = other DNA
cgctgagcgtgctgatgctggccggtatccg

SEQ ID NO: 287 - DNA, 29 bp, synthetic construct
  source 1..29: mol_type = other DNA
cgcgctatcagcctggaagaaaagccaaa

SEQ ID NO: 288 - DNA, 27 bp, synthetic construct
  source 1..27: mol_type = other DNA
acagccgaagagtaactgggcggtgac

SEQ ID NO: 289 - DNA, 28 bp, synthetic construct
  source 1..28: mol_type = other DNA
ggtgaagccgagcgagcgtggtgaactg

SEQ ID NO: 290 - DNA, 32 bp, synthetic construct
  source 1..32: mol_type = other DNA
tgctgccagtagtctcgcgaaaactggctacg

SEQ ID NO: 291 - DNA, 29 bp, synthetic construct
  source 1..29: mol_type = other DNA
tctgctggagctccttcgtgcccgtccgc

SEQ ID NO: 292 - DNA, 31 bp, synthetic construct
  source 1..31: mol_type = other DNA
acgcgctcgcgccgtggagtcgggtgcaggc

SEQ ID NO: 293 - DNA, 37 bp, synthetic construct
  source 1..37: mol_type = other DNA
aattgcggcaagtaatacgggtgaactggatgccctg

SEQ ID NO: 294 - DNA, 44 bp, synthetic construct
  source 1..44: mol_type = other DNA
gtgaagttgatctagcgctacccgtgaacaatgccagtgatagc

SEQ ID NO: 295 - DNA, 44 bp, synthetic construct
  source 1..44: mol_type = other DNA
cgatattcccgcactccgtcagctcgccagcgccgcatttgcgc

SEQ ID NO: 296 - DNA, 37 bp, synthetic construct
  source 1..37: mol_type = other DNA
caatgtctgattctccgtgcggcgtccggcgatattc

SEQ ID NO: 297 - DNA, 42 bp, synthetic construct
  source 1..42: mol_type = other DNA
gcggctatgtctctctccgggaactcaatgcgacagatgcgc

SEQ ID NO: 298 - DNA, 37 bp, synthetic construct
  source 1..37: mol_type = other DNA
cggtaaaacaactctacgggtggcgacccaaatgggc

SEQ ID NO: 299 - DNA, 50 bp, synthetic construct
  source 1..50: mol_type = other DNA
cgcgtactggctctaccgatgatcacatgattccatttaacgcaccgccg

SEQ ID NO: 300 - DNA, 45 bp, synthetic construct
  source 1..45: mol_type = other DNA
ctatatgcagagcgcaatgggtagcggcaaactgtgtggcgatgg

SEQ ID NO: 301 - DNA, 51 bp, synthetic construct
  source 1..51: mol_type = other DNA
cgccaaagtgctcctgacgccgtcctgcaccgctagcctggagatggcggc

SEQ ID NO: 302 - DNA, 59 bp, synthetic construct
  source 1..59: mol_type = other DNA
ttatggcgctagcgaaaaagcataatctatttgtggtagaagatgccgctcagggcgtg

SEQ ID NO: 303 - DNA, 36 bp, synthetic construct
  source 1..36: mol_type = other DNA
cgataaagcgctcatcgaacgagccgagatcatccg

SEQ ID NO: 304 - DNA, 44 bp, synthetic construct
  source 1..44: mol_type = other DNA
ctccagctatctaatgtccgatctgcaagctgcatacctgtggg

SEQ ID NO: 305 - DNA, 18 bp, synthetic construct
  source 1..18: mol_type = other DNA
atgcgctcgcgcctctgg

SEQ ID NO: 306 - DNA, 22 bp, synthetic construct
  source 1..22: mol_type = other DNA
gagctgccgagcattcccgatg

SEQ ID NO: 307 - DNA, 23 bp, synthetic construct
  source 1..23: mol_type = other DNA
cggagcgcgctaattaactttct

SEQ ID NO: 308 - DNA, 23 bp, synthetic construct
  source 1..23: mol_type = other DNA
ctacaacctgagccccgtcaatc

SEQ ID NO: 309 - DNA, 32 bp, synthetic construct
  source 1..32: mol_type = other DNA
acggtaattgcgactctactaaactacttttc

SEQ ID NO: 310 - DNA, 39 bp, synthetic construct
  source 1..39: mol_type = other DNA
ctgatatgagcctagcaaaagcgtccctatggacggcgg

SEQ ID NO: 311 - DNA, 44 bp, synthetic construct
  source 1..44: mol_type = other DNA
cgggctcctggtcggtaagctactggcggtgagttttggtccgg

SEQ ID NO: 312 - DNA, 25 bp, synthetic construct
  source 1..25: mol_type = other DNA
gccagctaattaccgtgctcggcgt

SEQ ID NO: 313 - DNA, 32 bp, synthetic construct
  source 1..32: mol_type = other DNA
tcggcactagtagtgcgatggtacttggtttc

SEQ ID NO: 314 - DNA, 23 bp, synthetic construct
  source 1..23: mol_type = other DNA
gtgcgtctcgtggcgctggtgca

SEQ ID NO: 315 - DNA, 31 bp, synthetic construct
  source 1..31: mol_type = other DNA
aatcagccagggactatttggtaataccgac

SEQ ID NO: 316 - DNA, 46 bp, synthetic construct
  source 1..46: mol_type = other DNA
gcaggtaatgcgctcagcctgattgtcggcagcctaattggcgttc

SEQ ID NO: 317 - DNA, 39 bp, synthetic construct
  source 1..39: mol_type = other DNA
accgtctaggcggttatgaaggggcgctactgggtctgg

SEQ ID NO: 318 - DNA, 49 bp, synthetic construct
  source 1..49: mol_type = other DNA
ccgccatcatgctaatcaaacgtggtgtcatcccgctcagctatctgaa

SEQ ID NO: 319 - DNA, 82 bp, synthetic construct
  source 1..82: mol_type = other DNA
gcagggcagctaagcaaatttacgctcatggcgctaattacgagcgtgaccctacctgttgcttacatcatgatgcgtaaac

SEQ ID NO: 320 - DNA, 69 bp, synthetic construct
  source 1..69: mol_type = other DNA
cctgcaatttattacggcaagcttcagcgtatatctactgcccacgctaagccggctaacggaaaagcg

SEQ ID NO: 321 - DNA, 48 bp, synthetic construct
  source 1..48: mol_type = other DNA
ggaagtggttaaaagcctgaaattcgtcctcccggcagtggcggcggc

SEQ ID NO: 322 - DNA, 54 bp, synthetic construct
  source 1..54: mol_type = other DNA
ctgctgcgtgattttgctatctggctgctgctaagcaataaatttaccgctatg

SEQ ID NO: 323 - DNA, 54 bp, synthetic construct
  source 1..54: mol_type = other DNA
tctttgcctggcagctcgtgggtgatgtgctcaaagtgggcgcttatgtctttg

SEQ ID NO: 324 - DNA, 45 bp, synthetic construct
  source 1..45: mol_type = other DNA
ggtgatcgccaaagcgagtctgcggttttatattctggcggaagt

SEQ ID NO: 325 - DNA, 51 bp, synthetic construct
  source 1..51: mol_type = other DNA
gttcactctcctaatggtatttgcccactggctaatccctgcgcatggtgc

SEQ ID NO: 326 - DNA, 71 bp, synthetic construct
  source 1..71: mol_type = other DNA
acttatatcgtctatttttctctttgttgtggcgtgtttctcctctggcgtcgacgggcatgactgtactg

SEQ ID NO: 327 - DNA, 72 bp, synthetic construct
  source 1..72: mol_type = other DNA
attcacgtactgggaagcgatatccctcaccataaccgaaccgttctacggtttttcaatgacgcgctggcc

SEQ ID NO: 328 - DNA, 40 bp, synthetic construct
  source 1..40: mol_type = other DNA
gttggcaaggacgacggcctcagtgatagctgtccggcgc

SEQ ID NO: 329 - DNA, 63 bp, synthetic construct
  source 1..63: mol_type = other DNA
tttctgtgcaatttttccctgggaaaaaaagcctggcggaagcggtcatcgcgaaagcaaaag

SEQ ID NO: 330 - DNA, 55 bp, synthetic construct
  source 1..55: mol_type = other DNA
gagctttccagtggcctacgctataagcttttttacccactacgtcgcctggcgc

SEQ ID NO: 331 - DNA, 39 bp, synthetic construct
  source 1..39: mol_type = other DNA
cacccgcggcgatctaagcttttttgccaaaacgcaccc

SEQ ID NO: 332 - DNA, 42 bp, synthetic construct
  source 1..42: mol_type = other DNA
cgcggatggaccctagcctcaatacgatggcgaacgatcggc

SEQ ID NO: 333 - DNA, 40 bp, synthetic construct
  source 1..40: mol_type = other DNA
agcatattgctgccctacgcgccgttcatcagcaatttgg

SEQ ID NO: 334 - DNA, 43 bp, synthetic construct
  source 1..43: mol_type = other DNA
ggcggggctggagctcttcagcgaagaaaatctacaaattctg

SEQ ID NO: 335 - DNA, 36 bp, synthetic construct
  source 1..36: mol_type = other DNA
tacgctgtgcctcctgattcaggcgggcattccttg

SEQ ID NO: 336 - DNA, 43 bp, synthetic construct
  source 1..43: mol_type = other DNA
gatatgacggaacaacatctaccggtgctgtttactaccgacg

SEQ ID NO: 337 - DNA, 47 bp, synthetic construct
  source 1..47: mol_type = other DNA
gaagcgcagcgccagctagcgagcgtggataaaaacaccattgcctt

SEQ ID NO: 338 - DNA, 36 bp, synthetic construct
  source 1..36: mol_type = other DNA
gcgggcgctagcgattgccgcccgagaggtcgcatg

SEQ ID NO: 339 - DNA, 66 bp, synthetic construct
  source 1..66: mol_type = other DNA
cgctttaacttcaatgtcttctttagtctactgtttctactcacctttttcttcggcttcccgctg

SEQ ID NO: 340 - DNA, 44 bp, synthetic construct
  source 1..44: mol_type = other DNA
gcgccgccagaaatcctactacaggcgctactttctgcgggctg

SEQ ID NO: 341 - DNA, 36 bp, synthetic construct
  source 1..36: mol_type = other DNA
cacaacggctttctactgttccggcttaactcctac

SEQ ID NO: 342 - DNA, 37 bp, synthetic construct
  source 1..37: mol_type = other DNA
cggcgtggcgctcaaacgcttcttttactttttcatc

SEQ ID NO: 343 - DNA, 38 bp, synthetic construct
  source 1..38: mol_type = other DNA
gtcgcctttggcctactgacttatatgattgtcggcgg

SEQ ID NO: 344 - DNA, 38 bp, synthetic construct
  source 1..38: mol_type = other DNA
tcgcggctggattagcctatggatgctggcggcggcgg

SEQ ID NO: 345 - DNA, 20 bp, synthetic construct
  source 1..20: mol_type = other DNA
accttcagcccgtgggagaa

SEQ ID NO: 346 - DNA, 31 bp, synthetic construct
  source 1..31: mol_type = other DNA
tctggcgctactgctacagaactacgacaac

SEQ ID NO: 347 - DNA, 26 bp, synthetic construct
  source 1..26: mol_type = other DNA
ggtgctgaacagtgccaactacttta

SEQ ID NO: 348 - DNA, 41 bp, synthetic construct
  source 1..41: mol_type = other DNA
ggcgatcagccctacgcttataggcagtctggtggtgatgg

SEQ ID NO: 349 - DNA, 25 bp, synthetic construct
  source 1..25: mol_type = other DNA
gcggcgcgctattcatcccgctcgg

SEQ ID NO: 350 - DNA, 35 bp, synthetic construct
  source 1..35: mol_type = other DNA
aaagctgcgatactacacagtttctgctttggggc

SEQ ID NO: 351 - DNA, 55 bp, synthetic construct
  source 1..55: mol_type = other DNA
gcgtgaagggctggatagctttgtcagtcgcgtggtcttttttatcgtggtcttc

SEQ ID NO: 352 - DNA, 38 bp, synthetic construct
  source 1..38: mol_type = other DNA
atcgcaaaactgctatactggctttttgaaagcgccgg

SEQ ID NO: 353 - DNA, 64 bp, synthetic construct
  source 1..64: mol_type = other DNA
actcattcataaacgtacaaaaagtagcctccggacgcaggttgaaggataacaatgaataaca

SEQ ID NO: 354 - DNA, 50 bp, synthetic construct
  source 1..50: mol_type = other DNA
cctatacgctgcgtggcctccagctaattggttggcgtgatatgcagcac

SEQ ID NO: 355 - DNA, 65 bp, synthetic construct
  source 1..65: mol_type = other DNA
gctgactattgaagataacgccgaggtccgagagctcattaacgctgccgaatttaaatatgcgg

SEQ ID NO: 356 - DNA, 47 bp, synthetic construct
  source 1..47: mol_type = other DNA
atggcatcagcgttgtacgtagtgtacgtaaaaagtacccgcaggcg

SEQ ID NO: 357 - DNA, 37 bp, synthetic construct
  source 1..37: mol_type = other DNA
gcgatgggaagcccaaagcaggagatcatcatgcgcg

SEQ ID NO: 358 - DNA, 70 bp, synthetic construct
  source 1..70: mol_type = other DNA
gcctgcttagccagccgagccgcattaagcgtcagcttcgtctactgcgttatctccgctggcactacac

SEQ ID NO: 359 - DNA, 35 bp, synthetic construct
  source 1..35: mol_type = other DNA
gccatccgtactactagcctatatcatcgccgggc

SEQ ID NO: 360 - DNA, 63 bp, synthetic construct
  source 1..63: mol_type = other DNA
atgcgtagtatgggcgaaatgctattcctcgaaccggttaccggtagcttcgccgtttatgcg

SEQ ID NO: 361 - DNA, 42 bp, synthetic construct
  source 1..42: mol_type = other DNA
ccgcactaatcgcagtggcgctggtggcgctagcgaatctgg

SEQ ID NO: 362 - DNA, 35 bp, synthetic construct
  source 1..35: mol_type = other DNA
cggcggtgcggctatacggcgaaatcgagttctgg

SEQ ID NO: 363 - DNA, 45 bp, synthetic construct
  source 1..45: mol_type = other DNA
caatggcgggcagagcattggttttagcaatctcacagagcatgg

SEQ ID NO: 364 - DNA, 28 bp, synthetic construct
  source 1..28: mol_type = other DNA
aaaaaccgtcagctcccggcggcaatgg

SEQ ID NO: 365 - DNA, 35 bp, synthetic construct
  source 1..35: mol_type = other DNA
gctaattggcagttgcctgaactacatcattccca

SEQ ID NO: 366 - DNA, 34 bp, synthetic construct
  source 1..34: mol_type = other DNA
tttgtgatactaataagccagctgcgttttcggc

SEQ ID NO: 367 - DNA, 29 bp, synthetic construct
  source 1..29: mol_type = other DNA
tccgttccgcagtatcctgttcccgtggg

SEQ ID NO: 368 - DNA, 46 bp, synthetic construct
  source 1..46: mol_type = other DNA
attcctgatttgcgttctaatcggcatgtactttaatgaagatacg

SEQ ID NO: 369 - DNA, 42 bp, synthetic construct
  source 1..42: mol_type = other DNA
cgtatgagcctgtttgttggcatcatctttatgctggcggtg

SEQ ID NO: 370 - DNA, 22 bp, synthetic construct
  source 1..22: mol_type = other DNA
ggtgctggccgaaagataagta

SEQ ID NO: 371 - DNA, 26 bp, synthetic construct
  source 1..26: mol_type = other DNA
tcttgttggtctcagcctggatggcc

SEQ ID NO: 372 - DNA, 21 bp, synthetic construct
  source 1..21: mol_type = other DNA
ggtggccgccccacgcataag

SEQ ID NO: 373 - DNA, 37 bp, synthetic construct
  source 1..37: mol_type = other DNA
caaccgctacaggtatatgattttctatgcgatgcgg

SEQ ID NO: 374 - DNA, 25 bp, synthetic construct
  source 1..25: mol_type = other DNA
tggacttaagctccatgcgcctggt

SEQ ID NO: 375 - DNA, 21 bp, synthetic construct
  source 1..21: mol_type = other DNA
cggaatggagcgtgcgccccg

SEQ ID NO: 376 - DNA, 25 bp, synthetic construct
  source 1..25: mol_type = other DNA
tgtgggcgcagcgtgattgttgagc

SEQ ID NO: 377 - DNA, 24 bp, synthetic construct
  source 1..24: mol_type = other DNA
taagcagctcccggcgcagtgtcg

SEQ ID NO: 378 - DNA, 29 bp, synthetic construct
  source 1..29: mol_type = other DNA
ttgtaacgtgctcaaagcgtgctggggag

SEQ ID NO: 379 - DNA, 32 bp, synthetic construct
  source 1..32: mol_type = other DNA
ggactgaattatctatgtgccgggtatcagcg

SEQ ID NO: 380 - DNA, 27 bp, synthetic construct
  source 1..27: mol_type = other DNA
aatggctgatctactggcgcacggtcg

SEQ ID NO: 381 - DNA, 40 bp, synthetic construct
  source 1..40: mol_type = other DNA
gcatgcgcatctactggtggtgagtaagtaaaaatcggcg

SEQ ID NO: 382 - DNA, 37 bp, synthetic construct
  source 1..37: mol_type = other DNA
gaggttaaacacgctgcttcccgccgtttgcattact

SEQ ID NO: 383 - DNA, 28 bp, synthetic construct
  source 1..28: mol_type = other DNA
gttttcggcacgagattcgccactttcg

SEQ ID NO: 384 - DNA, 30 bp, synthetic construct
  source 1..30: mol_type = other DNA
ccttcccaggtgctacctttcgcaccacgg

SEQ ID NO: 385 - DNA, 28 bp, synthetic construct
  source 1..28: mol_type = other DNA
cagtcgccatagctggtgcgtgccggag

SEQ ID NO: 386 - DNA, 36 bp, synthetic construct
  source 1..36: mol_type = other DNA
ctttgctgaacgggagttgcttgatgtattcagaac

SEQ ID NO: 387 - DNA, 44 bp, synthetic construct
  source 1..44: mol_type = other DNA
cggtgtacatatcagacacgctgttaaagccacggaaatcatca

SEQ ID NO: 388 - DNA, 38 bp, synthetic construct
  source 1..38: mol_type = other DNA
cgttctgcggctggctctctttgttttcccccatatgc

SEQ ID NO: 389 - DNA, 36 bp, synthetic construct
  source 1..36: mol_type = other DNA
gtgcagtagctgcggcagcgtgggtgacccttgcag

SEQ ID NO: 390 - DNA, 49 bp, synthetic construct
  source 1..49: mol_type = other DNA
cttggttgagaatacgcgctagtgagaatcagcccctggctggcaacgg

SEQ ID NO: 391 - DNA, 43 bp, synthetic construct
  source 1..43: mol_type = other DNA
acatcgtccagtaggaaaacaaccacattcggtttcttaccgg

SEQ ID NO: 392 - DNA, 31 bp, synthetic construct
  source 1..31: mol_type = other DNA
gcggtggcagtactcggggtatctgctgcgc

SEQ ID NO: 393 - DNA, 70 bp, synthetic construct
  source 1..70: mol_type = other DNA
gcgacagcaacaacaagacgtttgggactaaagctaaattccatgcaaaatgctccggtttcatgtcatc

SEQ ID NO: 394 - DNA, 53 bp, synthetic construct
  source 1..53: mol_type = other DNA
aaggaattactgtggcgggttattctgtagcgtgagcattagaccgtcgcgac

SEQ ID NO: 395 - DNA, 24 bp, synthetic construct
  source 1..24: mol_type = other DNA
gcttgtgcaggcggtccagcgcgt

SEQ ID NO: 396 - DNA, 30 bp, synthetic construct
  source 1..30: mol_type = other DNA
gcgcggaaggcgaggcttgcttcctgccat

SEQ ID NO: 397 - DNA, 47 bp, synthetic construct
  source 1..47: mol_type = other DNA
gcttcatcagactctggcccagtgtgctccatagcagcgggcgatcg

SEQ ID NO: 398 - DNA, 30 bp, synthetic construct
  source 1..30: mol_type = other DNA
ttgccacctgtagcgctacctgatgacgcg

SEQ ID NO: 399 - DNA, 38 bp, synthetic construct
  source 1..38: mol_type = other DNA
caccagttacgtagaccttcgctaccgttatcggcacg

SEQ ID NO: 400 - DNA, 56 bp, synthetic construct
  source 1..56: mol_type = other DNA
gggctttcgccatacttgggataatatccagcaggctactccatgcacctgtgcgg

SEQ ID NO: 401 - DNA, 33 bp, synthetic construct
  source 1..33: mol_type = other DNA
ttccgccagacggagtacttccggatggcgtgg

SEQ ID NO: 402 - DNA, 31 bp, synthetic construct
  source 1..31: mol_type = other DNA
ccatcgccagaattagtatgatcgccaggcc

SEQ ID NO: 403 - DNA, 57 bp, synthetic construct
  source 1..57: mol_type = other DNA
ccccgcaatcagtagcacaaagagtaggagcacttttagcatggttattctccttgc

SEQ ID NO: 404 - DNA, 51 bp, synthetic construct
  source 1..51: mol_type = other DNA
ggaagatccatgctgatattttgctggctgagctggtccacctcgtcgagg

SEQ ID NO: 405 - DNA, 25 bp, synthetic construct
  source 1..25: mol_type = other DNA
tttggcgcgagcagcggtacggcgg

SEQ ID NO: 406 - DNA, 53 bp, synthetic construct
  source 1..53: mol_type = other DNA
gaaagctcttcaccgtcactatccatcgggctaccatcgctgtcattatcggc

SEQ ID NO: 407 - DNA, 55 bp, synthetic construct
  source 1..55: mol_type = other DNA
cagacgcaggttatctacctgattactaagctgattaagcttaaggatgatgccg

SEQ ID NO: 408 - DNA, 59 bp, synthetic construct
  source 1..59: mol_type = other DNA
tcataatccacctggcttactgcagaaaggctggcgatatcatcggtaattgcccgacg

SEQ ID NO: 409 - DNA, 36 bp, synthetic construct
  source 1..36: mol_type = other DNA
gcacttttcagtagcgctgcagcggtcgtgacgtcc

SEQ ID NO: 410 - DNA, 46 bp, synthetic construct
  source 1..46: mol_type = other DNA
tggacttcatctagctgttttgccagcgtttcttgctgacgattcg

SEQ ID NO: 411 - DNA, 39 bp, synthetic construct
  source 1..39: mol_type = other DNA
ggctctcctgggctttttgtagtgccgtcagttggttag

SEQ ID NO: 412 - DNA, 30 bp, synthetic construct
  source 1..30: mol_type = other DNA
cccagccatatagaccgatgcccgccgcca

SEQ ID NO: 413 - DNA, 55 bp, synthetic construct
  source 1..55: mol_type = other DNA
cgatagccaccgcgctgagaattagtgcggtattgttcttactctttttttctgt

SEQ ID NO: 414 - DNA, 52 bp, synthetic construct
  source 1..52: mol_type = other DNA
tgcgacaggttgactcgtggtgtccacggcctctcgggtctcttcaaccacg

SEQ ID NO: 415 - DNA, 64 bp, synthetic construct
  source 1..64: mol_type = other DNA
cttcctgttatgagagttattggagtgcccggagaagcgcatcgttgtcagcgttatcggcgac

SEQ ID NO: 416 - DNA, 71 bp, synthetic construct
  source 1..71: mol_type = other DNA
cgggcgagtttcgctagacgctcactgacgactagtagtcgacagtgtaggagccagtgctcacgatacca

SEQ ID NO: 417 - DNA, 72 bp, synthetic construct
  source 1..72: mol_type = other DNA
ttgtgggatcaggctccagagttgctgtagcatttcaccgctggtaacaacgaccatcgtcacctcgcgggc

SEQ ID NO: 418 - DNA, 39 bp, synthetic construct
  source 1..39: mol_type = other DNA
agctcacgaccaccattgccacggagtatcagcgcacgt

SEQ ID NO: 419 - DNA, 101 bp, synthetic construct
  source 1..101: mol_type = other DNA
ttgcccgcaatattttggagttcagggagttgtagtaggacttcgctgatttcccgatcctgcgggtagagaatcttctgtccacttacggtatgtagtgc

SEQ ID NO: 420 - DNA, 78 bp, synthetic construct
  source 1..78: mol_type = other DNA
tgatgcagctgactttgggcaaaagcaaccgcgtgttggctgagggcaaatagcagatcgctctcccccagcgctgcc

SEQ ID NO: 421 - DNA, 61 bp, synthetic construct
  source 1..61: mol_type = other DNA
agttgatcagcaagttgcgggagttgttgacccggagaaaactcaatcagcggaaaatgcc

SEQ ID NO: 422 - DNA, 43 bp, synthetic construct
  source 1..43: mol_type = other DNA
agacggctcacgagctcttctccagcgggagacgggcgggtga

SEQ ID NO: 423 - DNA, 54 bp, synthetic construct
  source 1..54: mol_type = other DNA
gtagctcttctgccaggctaatccccatttgttcggcatcttgcggcgcaccgc

SEQ ID NO: 424 - DNA, 45 bp, synthetic construct
  source 1..45: mol_type = other DNA
gaataatctggctaccgtccggcgcgccgaccagcgcacgcagcc

SEQ ID NO: 425 - DNA, 48 bp, synthetic construct
  source 1..48: mol_type = other DNA
gctcgcgagtgcgactatcatcaaggcggcattcaatacccaccgcac

SEQ ID NO: 426 - DNA, 83 bp, synthetic construct
  source 1..83: mol_type = other DNA
agagaaatctcgggtggtagcgcggcgcgaatacgactctccagaccgagacgttttagtccggctacggcaagaatgatggc

SEQ ID NO: 427 - DNA, 18 bp, synthetic construct
  source 1..18: mol_type = other DNA
cgacggagactggacgtc

SEQ ID NO: 428 - DNA, 18 bp, synthetic construct
  source 1..18: mol_type = other DNA
ccgggagcgcatccagac

SEQ ID NO: 429 - DNA, 27 bp, synthetic construct
  source 1..27: mol_type = other DNA
catctttcatactgtgtacggcgatat

SEQ ID NO: 430 - DNA, 24 bp, synthetic construct
  source 1..24: mol_type = other DNA
ttttacaaagaggccttttccgcc

SEQ ID NO: 431 - DNA, 24 bp, synthetic construct
  source 1..24: mol_type = other DNA
gctcgccattagcttgtctttgac

SEQ ID NO: 432 - DNA, 44 bp, synthetic construct
  source 1..44: mol_type = other DNA
gtgtggcaatgcggagaacattgtcgagcatgcttgttaccgtc

SEQ ID NO: 433 - DNA, 41 bp, synthetic construct
  source 1..41: mol_type = other DNA
gaaacagcgcctggatgccataaatcaactacgtgtggatc

SEQ ID NO: 434 - DNA, 31 bp, synthetic construct
  source 1..31: mol_type = other DNA
actgccgacactactacactatcaccatccg

SEQ ID NO: 435 - DNA, 28 bp, synthetic construct
  source 1..28: mol_type = other DNA
gtatcgtggaatgagtgtacaggatccg

SEQ ID NO: 436 - DNA, 25 bp, synthetic construct
  source 1..25: mol_type = other DNA
cagcaccagctccgtagggcaaagt

SEQ ID NO: 437 - DNA, 30 bp, synthetic construct
  source 1..30: mol_type = other DNA
agagcgccaactactacaacgtaaatgtag

SEQ ID NO: 438 - DNA, 24 bp, synthetic construct
  source 1..24: mol_type = other DNA
ctgggccgccagcctgggtgtgga

SEQ ID NO: 439 - DNA, 38 bp, synthetic construct
  source 1..38: mol_type = other DNA
tcggtggcctcagcagcctttctgctgaagagtacttt

SEQ ID NO: 440 - DNA, 41 bp, synthetic construct
  source 1..41: mol_type = other DNA
acagcgtctacacgacggcgagattgtaagctttggtctcg

SEQ ID NO: 441 - DNA, 48 bp, synthetic construct
  source 1..48: mol_type = other DNA
cgtctggatctcgtacgtcgctgcttctatctcaaagtgtgcgaaaag

SEQ ID NO: 442 - DNA, 38 bp, synthetic construct
  source 1..38: mol_type = other DNA
gtcgcgcagtgctaagccagctcgtgagcgagtggggt

SEQ ID NO: 443 - DNA, 32 bp, synthetic construct
  source 1..32: mol_type = other DNA
gcacaacgagctactcgacgcgatgatgcaga

SEQ ID NO: 444 - DNA, 32 bp, synthetic construct
  source 1..32: mol_type = other DNA
gccgcgtttgaagcactcccaggtaaagtgac

SEQ ID NO: 445 - DNA, 53 bp, synthetic construct
  source 1..53: mol_type = other DNA
gctggtaaacccgcagattagtcccgatctcagcgaaccgaatctgaccttta

SEQ ID NO: 446 - DNA, 37 bp, synthetic construct
  source 1..37: mol_type = other DNA
ccgggctaaccgtagtggttggtatctgtataaccgc

SEQ ID NO: 447 - DNA, 41 bp, synthetic construct
  source 1..41: mol_type = other DNA
gcgccaaatattgagagcatcatcagccatcagccgctgga

SEQ ID NO: 448 - DNA, 56 bp, synthetic construct
  source 1..56: mol_type = other DNA
gggcatggtttaacggcctgctgaccagccgcacccgtctatatattaaaggtaac

SEQ ID NO: 449 - DNA, 73 bp, synthetic construct
  source 1..73: mol_type = other DNA
gtcgatctacctaagctacaggagatggtcgccgacgtgagccaccatttcccgctgcgcctccctgcaccga

SEQ ID NO: 450 - DNA, 32 bp, synthetic construct
  source 1..32: mol_type = other DNA
gtaccgcaacagctggaacgaagtgcgtacgc

SEQ ID NO: 451 - DNA, 41 bp, synthetic construct
  source 1..41: mol_type = other DNA
cggcgagcaaagcatgatcgaagccctgaaaactattctcg

SEQ ID NO: 452 - DNA, 30 bp, synthetic construct
  source 1..30: mol_type = other DNA
tctgcgcggcctcattcgtactcgcgtgca

SEQ ID NO: 453 - DNA, 38 bp, synthetic construct
  source 1..38: mol_type = other DNA
tgtattgaactacgtctttccagcacccgccaggaaac

SEQ ID NO: 454 - DNA, 57 bp, synthetic construct
  source 1..57: mol_type = other DNA
aacctgggggctattcttcgaacgcctgaatgtaagcgtacagaaactggaaaacgc

SEQ ID NO: 455 - DNA, 54 bp, synthetic construct
  source 1..54: mol_type = other DNA
tggcgcgattagccataacaaactgcacggcctgagtgtgcaggttgaaaccaa

SEQ ID NO: 456 - DNA, 36 bp, synthetic construct
  source 1..36: mol_type = other DNA
tcacgtcaaactcccggcggtggtggacggctttgc

SEQ ID NO: 457 - DNA, 72 bp, synthetic construct
  source 1..72: mol_type = other DNA
gtgacgtcagtcgcttctacagcagtagccatgaccgttttacctacggcagtagcttcatcaacttcaacc

SEQ ID NO: 458 - DNA, 57 bp, synthetic construct
  source 1..57: mol_type = other DNA
tacgccgctactccagcaatattttagctgatgaacgtgccggaaagcgaggcttat

SEQ ID NO: 459 - DNA, 34 bp, synthetic construct
  source 1..34: mol_type = other DNA
cgcctgttccagtagatcccagaaggtttcgccg

SEQ ID NO: 460 - DNA, 38 bp, synthetic construct
  source 1..38: mol_type = other DNA
gattcgccggaattccaggtatggctgccgatggttat

SEQ ID NO: 461 - DNA, 57 bp, synthetic construct
  source 1..57: mol_type = other DNA
cccgacatcgtatttccgnnnnnnnnnnnncagagaccaaagttcaactgaaacgaa

SEQ ID NO: 462 - DNA, 48 bp, synthetic construct
  source 1..48: mol_type = other DNA
gatgccacccgtggatccnnnnnnnnnnnncccgacatcgtatttccg

SEQ ID NO: 463 - DNA, 25 bp, synthetic construct
  source 1..25: mol_type = other DNA
cgcagagcgggatcataaggacgac

SEQ ID NO: 464 - DNA, 25 bp, synthetic construct
  source 1..25: mol_type = other DNA
cgcagagcgggaacataaggacgac

SEQ ID NO: 465 - DNA, 30 bp, synthetic construct
  source 1..30: mol_type = other DNA
gttaagacggctgatccgggaaaaaagagg

SEQ ID NO: 466 - DNA, 30 bp, synthetic construct
  source 1..30: mol_type = other DNA
gttaagacggctgaaccgggaaaaaagagg

SEQ ID NO: 467 - DNA, 28 bp, synthetic construct
  source 1..28: mol_type = other DNA
cctctcggacaggatccaccagcagccc

SEQ ID NO: 468 - DNA, 28 bp, synthetic construct
  source 1..28: mol_type = other DNA
cctctcggacaggaaccaccagcagccc

SEQ ID NO: 469 - DNA, 43 bp, synthetic construct
  source 1..43: mol_type = other DNA
cctgtattacttgagcagagatacaaacactccaagtggaacc

SEQ ID NO: 470 - DNA, 43 bp, synthetic construct
  source 1..43: mol_type = other DNA
cctgtattacttgagcagagaaacaaacactccaagtggaacc

SEQ ID NO: 471 - DNA, 55 bp, synthetic construct
  source 1..55: mol_type = other DNA
ggataacaacaacagtgaatactcggattggactggagctaccaagtaccacctc

SEQ ID NO: 472 - DNA, 55 bp, synthetic construct
  source 1..55: mol_type = other DNA
ggataacaacaacagtgaatactcggaatggactggagctaccaagtaccacctc

SEQ ID NO: 473 - DNA, 48 bp, synthetic construct
  source 1..48: mol_type = other DNA
gaggcaacagacaagcagctgataccgcagatgtcaacacacaaggcg

SEQ ID NO: 474 - DNA, 48 bp, synthetic construct
  source 1..48: mol_type = other DNA
gaggcaacagacaagcagctgaaaccgcagatgtcaacacacaaggcg

SEQ ID NO: 475 - DNA, 3716 bp, Escherichia coli
  source 1..3716: mol_type = genomic DNA
ccctcgatgttgacgttgcaaaaattgcaatgtgacgttgtgaatatatcaatttccgcaataaatttcctgtcatatagtgaattcaatctcgcaaacgcgaaccgaacaataagaagcacaacatcacgaggaatcaccatggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaagtgcgcgctgatgccctcaccccgaccctctcccacagggagagggagaaaacactcaaggccttctcctggagaaggccttgctattagttgcggtacagcaccttaatgatgtgatatccgaactgggtgtgcagcgggccggtcggctccagtaccggacaagagaaaaccactttatcgaacgccggaaccatctgaccctggcggaattcacctaaatcaccgccgcgtttgcctgatgggcaaatggagtgtttcttcgccagcttgccgaaatcggccccgttcttaatctgctcgagaagatccagagccagtttctcttcttttacaaggatatgcagtgctgctgctgtttttgccatgatcgtgccttgagtgaataattgtcaggcggcgattttagcatgatccgcagatgctaacgtaattgtcagtcagggagtggctggtacatatatggcaaaagcccggaaaatccgggcttcgcaggtccctcaaggggagaaaacttagggcctctatgggctacaaagtcattatcggggatggttattttattgtcaatttatgagcgatatggcattgattgacttgatgaccgccctctgtatgtctgcaaccaactcatgtggcaaacattgattaagataccctctgtaagtccttgctctgtttaatctgtttcgactgacttgttgtaccaggtcggctttggcccaacgaatctgctgatcaaagaattgcaggtcattgataacattactggctatttcaacatgcattccacgttttaacttgtctggtcgcgagtggttgataatggaacgacgatgcatgcgttgccattaatttttccgttgagcacgacaactaacctgttttttatcatttcaggaggaatgcggccatcgtaatacgtcgtgctgaacggtccattttggctaacaggatagttaccaaagttgcattccagtatttcaccaaccttagggctgaaattaatcgccacgagtaaacctctccttagtaaactctgaaaaagtaataacacaacgttacgacccgatattttctaagtctaatggattcacgatgaactccgatttcggtcttctctctctgatttaaacatccgcagccaaccggttagcggcttacacgcggtcacattcaaatgcgattctgctacaatcctccccccgttcgaagattgagcaatacacctatgcgtctaaaccccggccaacaacaagctgtcgaattcgttaccggcccctgcctggtgctggcgggcgcgggttccggtaaaactcgtgttatcaccaataaaatcgcccatctgatccgcggttgcggttatcaggcgcggcacattgcggcggtgacctttactaataaagcagcgcgcgagatgaaagagcgtgtagggcagacgctggggcgcaaagaggcgcgtgggctgatgatctccactttccatacgttggggctggatatcatcaaacgcgagtatgcggcgcttgggatgaaagcgaacttctcgttgtttgacgataccgatcagcttgctttgcttaaagagttgaccgaggggctgattgaagatgacaaagttctcctgcaacaactgatttcgaccatctctaactggaagaatgatctcaaaacaccgtcccaggcggcagcaagtgcgattggcgagcgggaccgtatttttgcccattgttatgggctgtatgatgcacacctgaaagcctgtaacgttctcgacttcgatgatctgattttattgccgacgttgctgctgcaacgcaatgaagaagtccgcaagcgctggcagaacaaaattcgctatctgctggtggatgagtatcaggacaccaacaccagccagtatgagctggtgaaactgctggtgggcagccgcgcgcgctttaccgtggtgggtgacgatgaccagtcgatctactcctggcgcggtgcacgtccgcaaaacctggtgctgctgagtcaggattttccggcgctgaaggtgattaagcttgagcagaactatcgctcttccgggcgtattctgaaagcggcgaacatcc

SEQ ID NO: 476 - DNA, 3628 bp, Escherichia coli
  source 1..3628: mol_type = genomic DNA
gaaagcggcgaacatcctgatcgccaataacccgcacgtctttgaaaagcgtctgttctccgaactgggttatggcgcggagctaaaagtattaagcgcgaataacgaagaacatgaggctgagcgcgttactggcgagctgatcgcccatcacttcgtcaataaaacgcagtacaaagattacgccattctttatcgcggtaaccatcagtcgcgggtgtttgaaaaattcctgatgcaaaaccgcatcccgtacaaaatatctggtggtacgtcgtttttctctcgtcctgaaatcaaggacttgctggcttatctgcgcgtgctgactaacccggacgatgacagcgcatttctgcgtatcgttaacacgccgaagcgagagattggcccggctacgctgaaaaagctgggtgagtgggcgatgacgcgcaataaaagcatgtttaccgccagctttgatatgggcctgagtcagacgcttagcggacgtggttatgaagcattgacccgcttcactcactggttggcagaaatccagcgtctggcggagcgggagccgattgccgcggtgcgtgatctgatccatggcatggattatgaatcctggctgtacgaaacatcgcccagcccgaaagccgccgaaatgcgcatgaagaacgtcaaccaactgtttagctggatgacggagatgctggaaggcagtgaactggatgagccgatgacgctcacccaggtggtgacgcgctttactttgcgcgacatgatggagcgtggtgagagtgaagaagagctggatcaggtgcaactgatgactctccacgcgtcgaaagggctggagtttccttatgtctacatggtcggtatggaagaagggtttttgccgcaccagagcagcatcgatgaagataatatcgatgaggagcggcggctggcctatgtcggcattacccgcgcccagaaggaattgacctttacgctgtgtaaagaacgccgtcagtacggcgaactggtgcgcccggagccgagccgctttttgctggagctgccgcaggatgatctgatttgggaacaggagcgcaaagtggtcagcgccgaagaacggatgcagaaagggcaaagccatctggcgaatctgaaagcgatgatggcggcaaaacgagggaaataatcaaggccggaaaagatgcgtcagcatcgcatccggcacttactcattaatgcacttccagcggccagtggacatagctctgccactggctttcctgagcaataatctctttacccagcggatgttgggttagccaaccttgcggaagcgtcaaggtcaacagttcatggttagcctgtaatgtcatctctggcacgagatcgtcacggcgacggctggcaaaaatgatggccaggcgtagtaaacggcagagttgttctgcgacgcgcggcggtacggcattttgctgatgcagcgatgagagatcgaccggattagtctggttgagcagtagcgtcgccagcaattttttctgtgcgggggtaaaaccgggaagatccagattacgcaccagataagcagcgtgttgcggcgcttgtttgaagtcaacgctcaggccgatttcatgaagctgacaggcgctgatgagcaaatcgcggcttattgcttcaagatgccattcgttttccacctgatcgaagaagttagccgcaactttggctacgcgctgtgcctgatcaatatcgatcataaagcggcgttgaatattacgcagcgtacggctgcgaatatcctgctcgacggccagatgcaacatgccgtagaccaggccttcacgcagcgcaccgcccgccagggtcatacactgaatattcagttcggtaaaaatggcgatcaggatcgccagaccactcgggaacactaacgcacgttccagcgtcagcccgtcaatctccagttcctccagccgaccgcaatgaatggctcgctgtttcaattgctgcaacttttccagggtaatgcgttcatccatcccctgtgccatcatgatttcctgtaacgcctgcacggtgccggaagcaccaacgcacactttccagccgtgataccgtaattcatcggcaaccggacgtaacacttcgcgtgccgctttttctgcagcatcaaaattttcctgccccagattacgatcggcaaaatagcgttccagccaggtgacgcagcccatcgacaggctgaacaacgaggtggtttgtgcacccgtgccggttaccagttcagtactggctccgcctatatccaccaccaggcgctgatcggcaccaccagtggtgtgagcaacgccctgataaatcagacgtgcttcctcttcaccgctgatcacctgtaccggacaaccgagaatttcctgcgctttggcaataaaatcacccgcattgacggcaaggcgtaacgtcgccgtagcgacaacgcgaatttgcgagggaggaatatcttgcagacgttcagcaaacaggcgcaaacattgccaaccgcgctccattgcttcattggagagggcattttcgctattcaggccagcagccagacgcactttgcgttttattcgcgtcagcgtctggatgcttccagccacctcgcgcacaaccagcatatgaaaactattcgaaccgagatcaatggctgcatacagcgacgaggtggaacccatactcttcatccttcaatctgtctctgtgttggctgcattctttcacctgaatcacttactacagtaagctcatcgggattcattctcttgccatcttgatacagtttgaatgattttgagtatgacattttttatttaacctgaacgacgacgattacgcggagcgccagtacgacgcggaccattgcctgtgcgcgggcgcgtgaggcgcagcggttttggcagatcggtcatcaatgcgtccggattgtatttgcttaccggaattgagtgaccaatataggtctcaatagcaggcaaattcaatgcatactcttcacacgccaggctgatagagtgaccgcttgcgcctgcgcgacctgtacgaccaatacggtgaacgtaatcttcacagtcatcgggtaaatcgtagttaaagacgtgcgtcactgccggaatatgcaaaccacgcgcggcaacgtcggtggcaaccagaatatccagatcgccacgggtaaattcatcaagaatacgcagacgttttttctgcgcgacatcgcctgtcaataaaccgacacgatgaccatctgctgccaggtggccccagatctcttcacaacggtgtttggtgttggcgaaaataatcgctctgtctggccactcttcttcgatcagcgtttgcagcaaacgcattttttcttcgttagaagggtagaaaagctcttctttaatacggtggcccgttttctgttccggttccacttcaatatattcggcattgttcatctgctcgaacgccagttcacgtacccggtacgaaagcgtggcggagaacagcatgttgaggcgctggtttgcaggcggcatacggcggaacagccagcggatatctttaataaagcccagatcgtacatgcgatcggcttcgtccagtaccaccacctgaa

SEQ ID NO: 477 - DNA, 3718 bp, Escherichia coli
  source 1..3718: mol_type = genomic DNA
aagcccagatcgtacatgcgatcggcttcgtccagtaccaccacctgaatggcaccgaggttaatgtggttctgcttggcgtagtcaattaaacgccccgtggtgccaatcagaatgtcaacgccgctttccagcactttcagctgtttgtcgtagccatcaccaccgtaagccagacccagcttcaggccagtagcttccgccagcggttctgcgtcggcatggatctgcacggcaagttcacgcgtcggtgccataattaaggcacgcggctgattcaccttgcgatcggcaatcgcaggatgagagagaagataatgaaacgttgacgtaagaaacgccatcgttttcccggtaccggtttgcgcctgcccggctacgtcacgacccgccagcgtcagcggaagggccagtgcctgaatgggcgtacagttatgaaacccttttttttcaagggcttctacaaccttcggatgcagggcgaagtcggaaaacttctgttctgttaaatgtgttttgctcatagtgtggtagaatatcagcttactattgctttacgaaagcgtatccggtgaaataaagtcaacctttagttggttaatgttacaccaacaacgaaaccaacacgccaggcttattcctgtggagttatatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcgtaagggaatttcatgttcgggtgccccgtcgctaaaaactggacgcccggcgtgagtcatgctaacttagtgttgacttcgtattaaacataccttattaagtttgaatcttgtaatttccaacgcttcccgttttatcttaaatgcgaagtgaacagatttctggctcgtcactcaatccgtcttgtcgtttcagttctgcgtactctcctgtgaccaggcagcgaaaagacatgagtcgatgaccgtaaacaggcatggatgatcctgccataccattcacaacattaagttcgagatttaccccaagtttaagaactcacaccactatgaatcttaccgaattaaagaatacgccggtttctgagctgatcactctcggcgaaaatatggggctggaaaacctggctcgtatgcgtaagcaggacattatttttgccatcctgaagcagcacgcaaagagtggcgaagatatctttggtgatggcgtactggagatattgcaggatggatttggtttcctccgttccgcagacagctcctacctcgccggtcctgatgacatctacgtttcccctagccaaatccgccgtttcaacctccgcactggtgataccatctctggtaagattcgcccgccgaaagaaggtgaacgctattttgcgctgctgaaagttaacgaagttaacttcgacaaacctgaaaacgcccgcaacaaaatcctctttgagaacttaaccccgctgcacgcaaactctcgtctgcgtatggaacgtggtaacggttctactgaagatttaactgctcgcgtactggatctggcatcacctatcggtcgtggtcagcgtggtctgattgtggcaccgccgaaagccggtaaaaccatgctgctgcagaacattgctcagagcattgcttacaaccacccggattgtgtgctgatggttctgctgatcgacgaacgtccggaagaagtaaccgagatgcagcgtctggtaaaaggtgaagttgttgcttctacctttgacgaacccgcatctcgccacgttcaggttgcggaaatggtgatcgagaaggccaaacgcctggttgagcacaagaaagacgttatcattctgctcgactccatcactcgtctggcgcgcgcttacaacaccgttgttccggcgtcaggtaaagtgttgaccggtggtgtggatgccaacgccctgcatcgtccgaaacgcttctttggtgcggcgcgtaacgtggaagagggcggcagcctgaccattatcgcgacggcgcttatcgataccggttctaaaatggacgaagttatctacgaagagtttaaaggtacaggcaacatggaactgcacctctctcgtaagatcgctgaaaaacgcgtcttcccggctatcgactacaaccgttctggtacccgtaaagaagagctgctcacgactcaggaagaactgcagaaaatgtggatcctgcgcaaaatcattcacccgatgggcgaaatcgatgcaatggaattcctcattaataaactggcaatgaccaagaccaatgacgatttcttcgaaatgatgaaacgctcataaatttgtcttatgccaaaaacgccacgtgtttacgtggcgttttgcttttatatctgtaatcttaatgccgcgctggcgatgttaggaaaattcctggaatttgctggcatgttatgcaatttgcatatcaaatggttaatttttgcacaggactggtgggtttggaacggactttcccttctgaataaaggtcttcgtggttatacttctgctaataattttctctgagagcatgcattgtgaatttactgacagtgagtactgatctcatcagtatttttttattcacgacactgtttctgttttttgcccgtaaggtggcaaaaaaagtcggtttagtggataaaccaaacttccgcaaacgtcaccagggattgatacctctcgttggggggatttcggtttacgcagggatttgcttcacgttcggaattgtcgattactatattccgcatgcatctctctatctcgcttgtgccggtgtgcttgttttcattggcgcgctggatgaccgttttgatatcagcgtaaaaatccgtgccaccatacaggccgctgttggcattgttatgatggtgttcggcaagctttatctcagtagcctgggttatatctttggctcctgggagatggtgctcggaccgtttggttacttcctgacgctatttgccgtctgggcggccattaatgcgttcaacatggttgatggcattgatggcttgctgggcgggttgtcctgcgtctcgtttgcagcaatcggtatgattttgtggttcgacgggcaaaccagcctcgcaatctggtgctttgcgatgatcgccgccatcctgccatacatcatgcttaaccttggtatcctgggtcgccgctacaaagtctttatgggtgatgcgggcagtacgctgattggttttaccgttatctggatcctgctcgaaacgacccagggcaaaacccatcccatcagcccggttaccgctttgtggataatcgccattccgctaatggatatggtggcgattatgtaccgtcgcctgcgtaaaggcatgagcccattctctcctgaccgtcagcatattcaccatttgatcatgcgtgccgggtttacttcccgtcaggcgtttgtgctgattacccttgccgcagca

SEQ ID NO: 478 - DNA, 3475 bp, Escherichia coli
  source 1..3475: mol_type = genomic DNA
gcgtttgtgctgattacccttgccgcagcactgctcgcttccattggcgtgctggcagaatattctcattttgtcccggagtgggtcatgctggtgctctttttgctagcattcttcctctatggatattgcattaagcgtgcctggaaagttgctcgctttattaagcgcgtaaaacgcagactgcgtagaaatcgtggtggcagccccaatttaaccaaataaatgaggatgtgatgacacaaccaatgcctgggaaaccggccgaagacgctgaaaatgaactggatattcgtgggttgtttcgtaccttgtgggctgggaagctatggattattggcatggggctggcgtttgcgttaatcgcgctggcgtatacgttttttgctcgtcaggagtggagctcgacggcgattaccgatcgtccaacggtgaatatgctggggggatattactcgcagcagcaatttttgcgtaacctggatgtccgttcaaacatggcttctgccgaccaaccatcggtcatggacgaagcctataaagagtttgttatgcaactggcctcgtgggatacccgcagagagttctggctgcaaaccgactattacaaacagcggatggtgggcaacagcaaagccgatgcggcgttgctggatgaaatgattaacaacatccagtttatccccggagactttacccgcgcggtcaatgacagcgtgaagcttattgccgaaaccgcgcctgacgctaataacctgttacgtcagtatgttgcttttgccagccagcgtgcagccagccatctgaatgatgagctgaaaggcgcatgggcggcgcgtaccatccagatgaaagctcaggtgaagcgtcaggaagaggtggcgaaagccatctacgaccgccggatgaacagcattgagcaggcgctgaaaattgctgagcagcataatatttcgcgcagtgcgacagatgtacctgccgaggaattacctgattcagaaatgttcctgcttgggcgtccaatgcttcaggctcgactggaaaatttacaggccgtcggtccggcctttgatctcgactatgatcagaatcgggccatgttaaacaccctgaatgttggtccaaccctggatccgcgttttcagacctatcgctatttgcgtacgccggaagaaccggtaaaacgcgatagcccacgtcgtgccttcctgatgattatgtggggcattgtcggggggctgatcggggctggtgtcgcattaacccgccgttgctcgaaatagcaacactgctgcggtgagcgcaaaggcgctcgccgcttattcgaagagaatcgatgtgaaagtactgactgtatttggtacgcgcccggaagccatcaagatggcgccgttggtgcatgcgttggcaaaagatcctttttttgaggctaaagtttgcgtcactgcgcagcatcgggagatgctcgatcaggtgctgaaactcttttccattgtacctgactacgatctcaacataatgcagccaggacagggcctgacagagataacctgtcggattctggaagggctaaaacctattcttgccgagttcaaaccagacgtcgtgctggttcacggcgatacgacgacgacgctggcaaccagcctggcggcgttttatcagcgtattcctgttggtcacgttgaggctggtctgcgcacgggcgatctctattcgccgtggccggaagaggctaaccgtacattgaccgggcatctggcgatgtatcacttctctccaaccgaaacttcccggcaaaacttgctgcgtgaaaacgttgcggatagccgaatcttcattaccggtaatacagtcattgatgcactgttatgggtgcgtgaccaggtgatgagcagcgacaagctgcgttcagaactggcggcaaattacccgtttatcgaccccgataaaaagatgattctggtgaccggtcacaggcgtgagagtttcggtcgtggctttgaagaaatctgccacgcgctggcagacatcgccaccacgcaccaggacatccagattgtctatccggtgcatctcaacccgaacgtcagagaaccggtcaatcgcattctggggcatgtgaaaaatgtcattctgatcgatccccaggagtatttaccgtttgtctggctgatgaaccacgcctggctgattttgaccgactcaggcggcattcaggaagaagcgccttcgctggggaaacctgtgctggtgatgcgcgataccactgagcgtccggaagcggtgacggcgggtacggtgcgtctggtaggcacggataagcagcgaattgtcgaggaagtgacgcgtcttttaaaagacgaaaacgaatatcaagctatgagccgcgcccataacccgtatggtgatggtcaggcatgctctcgcattctggaagcgttaaaaaataatcggatatcactatgagttttgcgaccatttctgttatcggactgggttatatcgggctgccaacggcagcagcgtttgcctcacggcaaaaacaggtaattggtgtcgatatcaaccaacatgcggttgataccatcaatcgtggcgaaatccatatcgtcgaacctgatttggcgagtgtagtaaaaactgccgtagaaggcggttttttacgagcgagcacgacgccagttgaagcggatgcctggctgattgctgtacccacgccgtttaagggcgatcatgagccagatatgacctacgttgaatcggctgctcgctccattgcgccagtgctgaaaaaaggcgcgctggtgatccttgaatccacctcgccggtggggtcaaccgagaagatggcagaatggttagcagagatgcgtccggatctcactttcccgcagcaggtgggcgagcaggcggacgtcaacattgcttactgcccggaacgcgtgttaccaggacaggtaatggtcgagctgattaaaaacgatcgcgtgattggtggtatgacgccggtttgttcggcccgcgccagcgaactgtacaaaattttcctcgaaggtgagtgtgtcgtcactaactcgcggacggcggaaatgtgtaagctcaccgaaaacagcttccgcgatgtgaatatcgcttttgctaatgaattgtcgctgatttgtgccgatcaggggattaacgtctgggaactgattcgcctggcgaatcgtcaccctcgcgttaatattcttcagcctggccctggcgtgggcggtcactgcattgctgttgatccgtggtttatcgtggcacagaacccccagcaggcgcggcttatccgtaccgcgcgcgaagtgaacgatcacaaaccgttctgggttatcgatcaggtgaaagcggcggtggctgattgcctggcggctaccgataaacgcgccagtgaactgaaaatcgcctgctttggtctggcgtttaaaccgaatattgatgacct

SEQ ID NO: 479 - DNA, 3692 bp, Escherichia coli
  source 1..3692: mol_type = genomic DNA
aaaatcgcctgctttggtctggcgtttaaaccgaatattgatgacctgcgcgaaagcccggcgatggaaatcgctgaactgatcgcccagtggcatagcggcgaaactctggttgttgagcctaacatccaccagttgccgaaaaaactgaccgggctttgtactctggcgcagcttgacgaggcgctggcaacggcagacgtgctggtgatgctggtcgatcatagtcagttcaaagttatcaatggcgacaatgtccatcagcagtatgtcgtcgatgccaaaggagtctggcgctgatgagaaaaattctgataacaggtggtgccgggtttattggctcggcgctggtgcgttatatcatcaacgaaacgagcgacgcggtggtagtggtcgataagctgacctacgccggaaacctgatgtcgctggcaccggtcgcgcaaagcgagcgctttgcctttgagaaagttgatatctgcgatcgggcagaactggcacgcgtattcactgagcatcagccagactgtgtcatgcatctggcagccgaaagccatgttgaccgttctattgacggcccggcagcgtttattgaaaccaacattgtcgggacttatacattgcttgaagcggcgcgggcttactggaatgcgctgacggaagataaaaaatcagcgttccgttttcatcatatctccactgacgaagtatatggtgacctgcactcgacggatgatttcttcaccgaaaccacgccgtatgcgccgagcagcccttattccgcgtcaaaagccagcagcgaccatctggtgcgcgcctggctgcggacctatggtctgccgacgcttatcaccaactgctcgaataactacggcccttaccactttccggaaaaactgatcccgctgatgatcctcaacgcgctggcgggtaaatcgctgccggtatatggcaacgggcagcaaatccgtgactggctgtatgtggaagatcacgcccgcgcgctgtattgcgtggcgaccaccgggaaagtcggtgaaacctataatattggtggtcacaacgagcgtaagaatctcgatgttgtggaaaccatttgcgagctgctggaagaactggctccgaacaagccgcacggcgtggcgcactatcgtgacctgatcacctttgtcgctgaccgtccggggcatgatctgcgttatgccattgatgcttcgaaaattgcccgtgaacttggctggctgccgcaggaaacctttgaaagtggaatgcgtaaaacggttcagtggtatctggctaatgaaagctggtggaagcaggtgcaggacggcagctatcagggcgagcgtttaggtctgaaaggctaattttcagcggaggcaaacatgaaaggtattatcctggcgggcggttccggcacccgattgcatccgattacgcgcggcgtatcgaagcaactgttgccgatttacgataagccaatgatttactatccgctgtcggtgctgatgctggccggtatccgcgaaattctcatcatcactacgccggaagataaaggttatttccagcgcctgctgggcgatggtagtgagttcggtatccagctggaatatgccgaacagcccagcccggacggtctggcgcaggcctttatcatcggtgaaaccttccttaatggtgaaccttcttgtctggtgctgggcgataacatcttcttcggtcagggcttcagtccgaagctgcgtcatgttgcggcgcgcaccgaaggggcgacggtttttggctatcaggtgatggacccggaacgctttggcgtggtggagtttgacgacaatttccgcgctatctcgctggaagaaaagccaaaacagccgaagtcaaactgggcggtgaccgggctttatttctacgacagtaaagtcgtggagtacgcaaagcaggtgaagccgtcggagcgtggtgaactggagattacctccatcaaccagatgtacctcgaggcgggcaacctgaccgttgaactgctcgggcgcggatttgcctggctggataccggcactcacgacagcctgattgaagccagcacctttgtacagacggtggaaaaacgccagggctttaagattgcctgcctggaagagattgcctggcgtaacggctggctcgatgacgagggtgtgaagcgtgctgccagttcattagcgaaaactggctacggccaatatctgctggagttacttcgtgcccgtccgcgccagtattgagccactaacctgggaaaacgccttctttggtgttaacagcgccatcgtgcgcattacgtctgaagcaccgctcctgacgccagacgcgttagcgccgtggtcacgggtgcaggccaaaattgcggcatcaaatacgggtgaactggatgccctgcaacagctgggattctccctggtagaaggtgaagttgatttggcgctacccgtgaacaatgccagtgatagcggtgctgtagtggcacaagagaccgatattcccgcattacgtcagttagccagcgccgcatttgcgcaaagccgttttcgtgcgccgtggtatgcgcctgacgccagcagtcgcttttatgcacagtggattgaaaatgccgtgcgcggcacctttgatcatcaatgtctgattttacgtgcggcgtccggcgatattcgcggctatgtctctttacgggaactcaatgcgacagatgcgcgaattggcctgctggctggacgcggtgcaggtgctgagctgatgcaaacggcgctaaactgggcgtatgctcgcggtaaaacaactttgcgggtggcgacccaaatgggcaacaccgccgcgcttaaacgatacatacaaagtggtgcgaatgtagaaagcaccgcgtactggttatacaggtgatcacatgattccatttaacgcaccgccggtggtgggaaccgaactcgactatatgcagtcggcaatgggtagcggcaaactgtgtggcgatggcggttttacccgtcgctgccagcagtggctggagcaacgttttggcagcgccaaagtgttactgacgccgtcctgcaccgcttcgctggagatggcggcgctgctgctcgatatccagcctggcgatgaagtgatcatgccgagctacacctttgtctccaccgccaatgcctttgtgctgcgtggcgcaaaaatcgtttttgtggatgttcgcccggacaccatgaacatcgacgaaacgctgattgaagcggcgatcaccgacaaaacgcgcgttatcgtgccggtccattacgcgggtgtggcctgcgaaatggacaccattatggcgttggcgaaaaagcataatttgtttgtggtagaagatgccgctcagggcgtgatgtccacttacaaagggcgtgcactgggaaccattggtcatattggctgctttagcttccatgaaaccaaaaactacacggcgggcggtgaaggcggcgcgacgctgattaacgataaagcgttaatcgaacgagccgagatcatccgtgaaaagggcactaaccgcagccagttcttccgtggtcaggtcgataaatatacctggcgcgatattggctccagctatttgatgtccgatctgcaagctgcatacctgtgggcgcaactggaagcagcggatcgtatcaaccagcaac

SEQ ID NO: 480 - DNA, 3487 bp, Escherichia coli
  source 1..3487: mol_type = genomic DNA
gcggatcgtatcaaccagcaacgtctggcgctgtggcaaaactactacgatgcgttagcgcctctggcgaaagccgggcgtatcgagctgccgtcgattcccgatggctgcgtgcagaacgcgcatatgttctacattaaactgcgggatattgatgaccggagcgcgttgattaactttctgaaagaagcggaaatcatggcggtgtttcattacattccgctgcacggttgccctgcgggggaacactttggtgagttccacggtgaagatcgctacaccaccaaagagagcgagcgcctgctgcgcctgccgctgttctacaacctgtcgcccgtcaatcagcgtacggtaattgcgactttgttgaactacttttcctgatatgtcgttggcaaaagcgtccttgtggacggcggccagtacactggtcaagattggtgccgggttactggtcggtaagttgctggcggtgtcatttggtccggcggggcttgggctggcggcaaatttccgccagttgattaccgtgctcggcgtgcttgccggggctggcatctttaacggtgtaaccaaatacgttgcccagtaccatgataatccgcaacagctgcgccgcgtggtcggcacttcatcagcgatggtacttggtttctctacgctgatggcgctggtttttgtgctggcagctgcgccaatcagccagggattgtttggtaataccgactatcaggggctggtgcgtttagtggcgctggtgcaaatggggatcgcctggggcaacctgttactggcgctgatgaaaggctttcgcgatgccgcaggtaatgcgttatcgctgattgtcggcagcttgattggcgttctcgcgtactacgtcagttaccgtttgggcggttatgaaggggcgttgctgggtctggcgctgattcccgcgctggtggtaattcctgccgccatcatgttgatcaaacgtggtgtcatcccgttaagctatctgaaacccagctgggataacggtctggcagggcagttgagcaaatttacgctcatggcgttgattacgtcggtgaccttgcctgttgcttacatcatgatgcgtaaactgctggcggcgcagtatagctgggatgaggtggggatctggcaaggggtgagcagtatttccgatgcctacctgcaatttattacggcatcgttcagcgtatatttgctgcccacgttgtcgcggctaacggaaaagcgcgatatcacccgggaagtggttaaatcgctgaaattcgtcttaccggcagtggcggcggcgagttttaccgtctggctgctgcgtgattttgctatctggctgctgttgtcgaataaatttaccgctatgcgcgatctctttgcctggcagttagtgggtgatgtgttaaaagtgggcgcttatgtctttggttatctggtgatcgccaaagcgtcactgcggttttatattctggcggaagtcagccagttcactttattgatggtatttgcccactggctaatccctgcgcatggtgcactgggcgcggcgcaggcatatatggcaacttatatcgtctatttttctctttgttgtggcgtgtttttactctggcgtaggcgggcatgactgtactgattcacgtactgggatcggatatccctcaccataaccgaaccgttttgcggtttttcaatgacgcgctggccgcgacgagcgagcacgcgcgcgagtttatggttgttggcaaggacgacggcttaagtgatagctgtccggcgctttctgtgcaatttttccctgggaaaaaatcgctggcggaagcggtcatcgcgaaagcaaaagctaaccgtcagcagcgttttttcttccacggtcagttcaatcccacactgtggctggctctgctgagtggtggcattaagcccagccagtttttctggcatatctggggggcagacctgtatgagctttccagtggcttgagatataagcttttttacccactacgtcgcctggcgcaaaagcgagtcggctgtgtatttgccacccgcggcgatttgagcttttttgccaaaacgcacccaaaggtgcggggcgaactgctgttcttcccgacgcggatggacccttcgctcaatacgatggcgaacgatcggcaacgtgaagggaaaatgaccattctggtggggaactccggcgaccgcagcaatgagcatattgctgccttgcgcgccgttcatcagcaatttggcgatacggtaaaagtggtggtgccgatgggatatccgcctaataacgaagcgtacattgaggaagttcgtcaggcggggctggagttattcagcgaagaaaatctacaaattctgagcgaaaaactggaatttgacgcctatctggcgctacttcgtcagtgcgatcttggttactttatttttgcccgccagcagggcattggtacgctgtgcttactgattcaggcgggcattccttgtgtgcttaaccgggaaaatccgttctggcaggatatgacggaacaacatttgccggtgctgtttactaccgacgatctcaacgaggatattgtgcgtgaagcgcagcgccagttggcgtcggtggataaaaacaccattgccttctttagccctaactatctacaaggctggcagcgggcgttggcgattgccgccagggaggtcgcatgagtctgctgcaattcagtggcctgtttgttgtctggctgctctgcacgctgtttattgccacgctgacctggtttgagtttcgccgtgtgcgctttaacttcaatgtcttcttttcattgctgtttttgctcacctttttcttcggcttcccgctgaccagcgtgctggtatttcgctttgatgttggtgtcgcgccgccagaaatcttgttgcaggcgttgctttctgcgggctgcttctacgcggtttactatgtcacctacaaaacccgcctacgcaaacgcgttgctgatgtaccgcgccgtccgctgtttaccatgaaccgcgtggagaccaatcttacgtgggtgatcctgatgggtatcgcgctggtaagcgtcggcatcttcttcatgcacaacggctttttgctgttccggcttaactcctacagtcagatcttttccagtgaagtctccggcgtggcgttaaaacgcttcttttactttttcatcccggcgatgctggtggtctactttctgcgccaggacagcaaagcgtggctgtttttcctcgtcagcacggtcgcctttggcttgctgacttatatgattgtcggcggcactcgcgccaatatcatcatcgcattcgctatcttcctgtttattggcattattcgcggctggatttcgttgtggatgctggcggcggcgggcgtgctggggattgttggcatgttctggctggcactaaaacgctatggaatgaatgtgagcggcgatgaagcgttctatac

SEQ ID NO: 481 - DNA, 3583 bp, Escherichia coli
  source 1..3583: mol_type = genomic DNA
gctggcactaaaacgctatggaatgaatgtgagcggcgatgaagcgttctatacgtttctctatctcactcgcgacaccttctcgccgtgggagaatctggcgttgctgttgcagaactacgacaacatcgacttccagggcctggctccaattgtccgcgatttctatgtctttatcccttcctggctgtggccgggtcgcccgagtatggtgctgaactcagccaactactttacctgggaagtgctgaataaccactccggactggcgatctcgcctacgcttataggctcactggtggtgatgggcggcgcgttgttcatcccgctcggggcgatcgtggttggtctgatcatcaaatggttcgactggctgtatgagctgggcaaccgcgagcctaatcgctataaagctgcgatattgcacagtttctgctttggggcgatcttcaatatgatcgtgctggcgcgtgaagggctggattcgtttgtctcacgcgtggtcttttttatcgtggtcttcggcgcatgtctgatgatcgcaaaactgttgtactggctttttgaaagcgccggactcattcataaacgtacaaaatcatcgctccggacgcaggttgaaggataacaatgaataacaacaccacggcaccaacctatacgctgcgtggcttacagttgattggttggcgtgatatgcagcacgccctcgattatctgtttgctgacgggcagcttaagcagggaacgctggttgccattaatgctgaaaaaatgctgactattgaagataacgccgaggtcagggagttaattaacgctgccgaatttaaatatgcggatggcatcagcgttgtacgttcagtacgtaaaaagtacccgcaggcgcaggtttcccgcgttgccggtgccgatctctgggaagagctgatggcgcgcgcaggcaaagaagggacgccggtatttcttgtgggcggtaaacctgaagtgctggcgcaaactgaagctaaactgcgcaaccagtggaatgtgaatatcgttggcagtcaggatggttattttaaacccgagcagcgtcaggcgctgtttgaacgcattcatgccagcggtgcgcaaatcgtcaccgttgcgatgggatcgccaaagcaggagatcatcatgcgcgactgccgtctggtacatccagatgcgctgtatatgggcgttggcgggacttacgatgttttcaccggtcacgtaaaacgcgcaccgaaaatctggcaaacgctggggctggagtggctctaccgcctgctttcgcagccgagccgcattaagcgtcagcttcgtttgctgcgttatttacgctggcactacaccggcaacctatgattttcctctctttgtaaagcgacggagtggtcactccgtcgcctgtctgcttttttattacacaaagcattcaaatttttaatgctttatttgccatttctcctgaattacgaaaacatttgcaacactcgatgtacccataacgataaccggtaacaccggaaagcatgcaaacacaacacgaggatttatggcagataacaaaccagagctacagcgtgggctggaagctcgacatatcgaactcatcgccctggggggcaccattggcgtcggcctgtttatgggggccgccagtaccctgaaatgggccgggccatccgtattgttggcctatatcatcgccgggctgttcgtctttttcatcatgcgttcaatgggcgaaatgttgttcctcgaaccggttaccggttcgttcgccgtttatgcgcatcgttatatgagcccgttctttggctatctcaccgcctggtcttactggtttatgtggatggcggtggggatctctgaaatcaccgccattggcgtttatgtccagttctggttcccggagatggcgcagtggatacccgcattgatcgcagtggcgctggtggcgttggcgaatctggcggcggtgcggttgtacggcgaaatcgagttctggttcgcgatgatcaaagtcaccacgattatcgtgatgattgtcattggcctgggcgtgattttctttggctttggcaatggcgggcagtcgattggttttagcaatctcacagagcatggcggtttctttgcgggtggctggaaagggttcctgaccgctctgtgtattgtggtggcgtcctaccagggcgtggagctgattggcattactgccggtgaagcgaagaatccgcaggtgacgctgcgcagtgccgtaggcaaggtgctgtggcggatcctgattttctacgtaggcgcgattttcgttatcgtcaccatcttcccgtggaatgaaataggcagcaacggcagcccgttcgtactgacttttgccaaaatcggtattaccgcagcggcgggcattatcaactttgtggtgctgacggctgcgctctctggctgtaacagcggcatgtacagttgcggacgtatgctctacgcactggcgaaaaaccgtcagttaccggcggcaatggcgaaagtttcccgtcacggcgtaccggttgcgggtgtggcagtatctattgctattctgctaattggctcatgcctgaactacatcattcccaatccgcagcgtgtgtttgtctacgtctacagtgccagcgtgcttccggggatggtgccatggtttgtgatattgataagccagctgcgttttcggcgtgcacataaagcggcgattgccagccatccgttccgctcaatcctgttcccgtgggccaattacgtaacaatggcattcctgatttgcgttttgatcggcatgtactttaatgaagatacgcgtatgtcgctgtttgttggcatcatctttatgctggcggtgacggcgatttataaagtttttggccttaatcgccacgggaaagcgcataaactggaggaataagcagcaaaacgcacaaaccgtaaccaaacgcgcaatttatttaaaaagggactagacagaggggtgggaagtccgtattatccacccccgcaacggcgctaagcgcccgtagctcagctggatagagcgctgccctccggaggcagaggtctcaggttcgaatcctgtcgggcgcgccatttagtcccggcgcttgagctgcggtggtagtaataccgcgtaacaagatttgtagtggtggctatagctcagttggtagagccctggattgtgattccagttgtcgtgggttcgaatcccattagccaccccattattagaagttgtgacaatgcgaaggtggcggaattggtagacgcgctagcttcaggtgttagtgtccttacggacgtgggggttcaagtcccccccctcgcaccacgactttaaagaattgaactaaaaattcaaaaagcagtatttcggcgagtagcgcagcttggtagcgcaactggtttgggaccagtgggtcggaggttcgaatcctctctcgccgaccaattttgaaccccgcttcggcggggttttttgttttctgtgcatttcgtcaccctcccttcgcaataaacgcccgtaataactcattgccccacggta

SEQ ID NO: 482 - DNA, 3736 bp, Escherichia coli
  source 1..3736: mol_type = genomic DNA
cgcccgtaataactcattgccccacggtatgatttcgcccttaacgtattgaaggatgacttcaggcaaggagcgaccatgctgcaacaggttccaacgcgtgcttttcatgtgatggcgaaaccgagcggttccgattgtaatctgaactgtgactactgtttttatctcgaaaaacaatccctttaccgcgaaaagccagtcacgcatatggacgatgacacgctggaagcgtatgtccgtcactatatcgctgccagcgaaccgcaaaacgaagtggcttttacctggcagggcggcgaaccaacgctactcgggctggcgttttaccgccgtgccgtagcgctacaggcgaaatatggtgctggcaggaagataagtaacagcttccagactaacggcgtgctgctggatgacgaatggtgcgcgtttctcgcggagcatcattttcttgttggtttatcgctggatggcccgcctgagatccacaatcaatatcgcgtgactaaaggtggcagacccacgcataagctggtgatgcgtgccctgacgctcctgcaaaaacatcatgtcgactataacgtgctggtctgcgttaatcgcaccagcgcgcagcaaccgttgcaggtatatgattttttgtgcgatgcgggagtcgaattcatccagtttattccggtggtcgagcgcctggctgatgaaacaactgcccgcgatggacttaagttacatgcgcctggtgatattcagggtgagctaacggaatggtcggtgcgccccgaggagttcggtgagtttctggtggcgatattcgaccactggatcaaacgcgacgtcggcaagattttcgtgatgaatatcgaatgggcgtttgccaattttgtcggtgcgccgggtgcggtttgccatcatcagccaacctgtgggcgctcggtgattgttgagcacaacggcgacgtttacgcctgtgatcactatgtttatccgcaatatcggctggggaatatgcaccagcaaacaattgcagaaatgatcgattccccgcaacagcaggcgtttggtgaagataaatttaagcagttaccggcgcagtgtcgcagttgtaacgtgttaaaagcgtgctggggaggctgcccgaaacaccgcttcatgctcgatgccagcggcaaaccgggactgaattatttgtgtgccgggtatcagcgttatttccgccatctaccgccatatcttaaagcaatggctgatttgctggcgcacggtcgcccggccagcgacattatgcatgcgcatttgctggtggtgagtaagtagaaatcggcggccgcctgcggttgattgccggatgcggcgtaaacgccttatccggcctacatgatcgtgcaaattcaataaattgcagcgttctgtaggctggataagatgcgtcagcatcgcatccggcaaaggcagatctcagcgatagcgccggcttagtcagatttaatctgcgcgcgtggtggatattttttcaggatctccatatacgcgtgcatttcggtctgtagcggtacacccatcggaatatggcgcacgccgatggagtcgctttcctgcggatcggtgtagaggttaaacaccgacgatcccgccgtttgcattactgtgccggtgaatccaccctgatatccgctctgggtataagcgtaaggttgctgaatcaggacgtgatacttgaactcatccatacgcacagcagcgagtttaccgttgaggaagtagtgctcggccttacggttagactgaccatttgttcccaggaagaaggatgtctggtccacaccatcgataaaggtggttttcggcactaaattcgccactttcgctccaggatgccctgccagatccagcgcggtagggaagagatctgccagatcgacaataccgtcagatttacgcggttggatcatccctttccagtaaacgaaagtcggtacgcgaacgccgccttcccaggtcgaacctttcgcaccacggaacggggtgcgtccgtgcggcggtacttcggcttccggtccgttatcggaggtaaagacgatcagcgtgttatcaagctgaccgtttttctccagtgttttatacagattagcgaacacatcgttcatctccaccatgcagtcgccatacgaggtgcgtgccggagagctacccgcatatttcgcatttgggtagttatcgaagtggcagccacgagtgccgtagtagaggaagaatggtttatcgctcttcgccatcttgtcgaggaacttaacgccatagtccatccagcgttgatccagatcttccatatatttcggcgtaatgtcggcaatggcctgttgttcgccgccgcgcaccgcatgaacgtcatctttgctgaacggtaattgcttgatgtattcagaacggtccggactcagggccacttccggattgacgtgaacgtcgcgccattcggtgtacatatcagacaccgagttaaagccacggaaatcatcaaagccaacgttctgcggctgcgactctttgttttcccccatatgccattttccgatggcctgagtgacgtagccctgatcgtgcagcaactgcggcagcgtggttaacccttgcagcccgcccggttgcccgtacattggcggcatcagaatgccgtggtggatggagtattgtccggtgagaatggtggcgcgggttggggaagagcttggttgagaatacgccgaagttaaaatcagcccctggctggcaacggcgtcgatatctggtgtagggttacccaccgccacgccgccaccgttaaaaccgacgtccatccagcccacatcgtccagcaagaaaacaaccacattcggtttcttaccggtttttttctcaagttctgccagcttctgctgggtttctttatcctgcgccggatgctgcattactggcatcatattgtcggcaatagtggtcgccggtttaaccagatactggtttgggtgatcgtatccggcaaagcctttgcgtgcggtggcagttgacggggtatctgctgcgctggccatgagaggaagagcggcggcgacagcaacaacaagacgtttgggtgaaaacgaaaattccatgcaaaatgctccggtttcatgtcatcaaaatgatgacgtaattaagcattgataattgagatccctctccctgacaggatgattacataaataatagtgacaaaaataaattatttatttatccagaaaatgaattggaaaatcaggagagcgttttcaatcctacctctggcgcagttgatatgtaaggcaggtttattatatcgcgttgattattgatgctgtttttagttttaacggcaattaatatatgtgttattaattgaatgaattttatcattcataataagtatgtgtaggatcaagctcaggttaaatattcactcaggaagttattactcaggaagcaaagaggattacagaattatctcataacaagtgttaagggatgttatttcccgattctctgtggcataataaacgagtagatgctcattccatctcttatgttcgccttagtgcctcataaactccggaatgacgcagagccgtttacggtgcttatcgtccactgacagatgtcgcttatgcctcatcagacaccatggacacaacgttgagtgaagcacccacttgttgtcatacagacctgttttaacgcctgctcc

SEQ ID NO: 483 - DNA, 3709 bp, Escherichia coli
  source 1..3709: mol_type = genomic DNA
gacctgttttaacgcctgctccgtaataagagcaggcgtttttttatgtatcaggaaggccccggaggtgcttgcctccgggtgagaaggaactactgtggcgggttattctgcaacgttaacatcaaaccgtcgcgacgcatagctgcagcttcttccggcttgtgcagtctgtccagcgcgtcggcaagccatgcgtaatcgtaggcgtccggacgttgtttcagcgctgcgcggaaggcgagcgatgcttcctgccattctccgtgcttcatcagtgactggcccagtgtgctccacaacagcgggcgatcgccgacgtttttgatttgctggcgcagcactttttccagctgttccggattgtttgttttcagtcgaggaatcggcagcagtaggcgatcgtcgtactggcgtttcaggccatcgatgataatttgctgggcagtatcatgatcgtcacattcaataagatgttccgccattgccacctgcaacgctacctgatgacgcgttttccggctttggtttttccaccagttacgcaaaccttcgctaccgttatcggcacgcgcctgatccatcaggccaatccatgcctgttgttccagcattgcacgatgttcttcatcaccaacatgggctttcgccattgatgggataatatccagcagcgaactccatgcacctgtgcggatatacgcctgttccgccagacgtaatacttccggatggcgtggcgtaacttccagcagcttatccacgccgtggcgtgcagcatggttttcattacgggccagttgcagacgtacgcgggtgatttctaccggaatggtgtcgttgccggccagctccgctgcgcgttccagatgttggttggcgcgtgcttcatcaccacgttgttgcgcggcttcggcagccagtagatagttcaccaccggttgttccgcgtgatcggcatttttcgccatcagcttttcaacttgctgataatcgccttccgccagtttcagcagcgcctgttcggtctgcttacgtgcacggcgacgcttacgtccgacaaaccacccacgggtgtgcgcgccagtgcggaagatccgccgcagtagccactcaatggcaaacagcactaccatcgccagaatcaatatgatcgccaggcccgtgacgctggtttcgatattgtagttgtcggtctggatcagcacataaccctgatggccggcaatcatcgggccaaccacgatccccgcaatcagcaacacaaagagcaataacacttttagcatggttattctccttgcggcgcggctgccggagtatcagcttgcggtgcaggtgcaggtttagcttccgttgtccccgctgccggttgtgccagcaggttacgcacgcgagtctgcatcagtttttccagcatcgcctggctttgcagggtttccggaagatccatcgagatattttgctggcttaactggtccacctcgtcgaggaacgctttggtggtggcatcatcagtatcgtagtaagcacgtacccaggtggagacgttctccagcgcctggcgataagtctcttcctggtgacgcggtacagcttgtgctgcgaccagcaggcgagagcgaatattttcgcgcagatagatatcctgatttggcgctaacagcggtacggcggtgtcatcacgacggcgaatcgtaatgaagttgtccataaagttctgccagcttttttgcagattgatacgccattcgctgatggaactggaaagctcttcaccgtctgaatccatcggcgaaccatcgctgtcattatcggccagacgcaggttatctacctgatttgaaagctgattaagcttaaggatgatgccgtcataatccacctgcgatactgcagaaaggctggcgatatcatcggtaattgcccgacgaacggtaatcagactcgggtcattcatatccgccaggctggcgtctgcacttttcagcaacgctgcagcggtcgtgacgtcctgatcgctccacagcttccgtccggcgagtttcaccagaaaatcggcctgagccagcagccaggttttagcatcgctgccggaaatggtggcgaccttttgttggacttcatccaactgttttgccagcgtttcttgctgacgattcgcctgcttaagttgtgcagcttgttgcttaataatgccttccagctcggctttttggctctcctgggctttttgcaatgccgtcagttggttagccagggcatcgctggtggcggtctgattgacggcctgttgtttaccccagccatacaaaccgatgcccgccgccagagcaatagcgatagccaccgcgctgagaatcaatgcggtattgttcttactctttttttctgttgcgacaggttgtgacgtggtgtccacggcctccctggtctcttcaaccacggcggaggttttttcttgttccgtcattatggcttcctgttatgagagttattgtaatgcccgtaaaagcgcatcgttgtcagcgttatcggcgaccttaatgtcttgccagcccagttcccgggcgagtttcgccaaacgctcactgacgaccaatagtcgacagtgtagtaaccagtgctcacgataccattgtgggatcagcgaccagagttgctgcaacatttcaccgctggtaacaacgaccatcgtcacctcgcgggcttgccagcgcatcgcttcttctgcaccatcgtaatggattgcgcatcgttgataacattcacaaaaagtgacctcagcaccgcgcgccgtcagggtatccccaattagctcacgaccaccattgccacgtaatatcagcgcacgtttgcccgcaatattttgtaattcaggtaattgtagcaagacttcgctgatttcccgatcctgcgggtagagaatcttctgtccacttacggtatgtagtgccagtgcggtggtgcgtccaatggcgaaataatcaggtagtcggggccatttacgatcttgctgatgcagctgtgattgggcaaaagcaaccgcgtgttgcgagagggcaaacaacagatcgctctcccccagcgctgccagttgatcagcaagttgcggtaattgttgacccggagaaaactcaatcagcggaaaatgccaggccacctgccccagtgtgcgcagacggctcactaactcttctccagcgggagacgggcgggtgacaaggatactcatgccggggcgtctccgttatagacttcagcgaggatctcgcgcgcgccgttattcagtagctcttctgccagcgaaatccccatttgttcggcatcttgcggcgcaccgcggcgttcaccgcgaataatctgcgaaccgtccggcgcgccgaccagcgcacgcagccagatttcgccatcaataagctcggcgtagctaccaattggcacctgacatccgccttcgagacgggtattcatggcgcgttctgcggtaacgcgcagtgcagtttcgtggtgattcagcgcggcaagcagctcgcgagtgcgtgaatcatcaaggcggcattcaatacccaccgcaccttgtcctaccgccggaagagaaatctcgggtggcaacgcggcgcgaatacgtgactccagacctaaacgttttagtccggctacggcaagaatgatggcatcgtattcgccgttatccagtttgctcagg

SEQ ID NO: 484 - DNA, 3531 bp, Escherichia coli
  source 1..3531: mol_type = genomic DNA
gccgttatccagtttgctcaggcgagtgccgacgttgccgcgcagggagcggataatcagatccggacggcgttcagccagttggcactggcgacgtaaactggacgtcccgacgatactgcctgccggtaacgcatccagactgtcatagttattggacacaaaggcatcgcgaggatcttcacgctcacaaatagtgaccagtcccagaccttgcgggaattcaaccggcacatctttcattgagtgtacggcgatatcggcgcgattttcgaggagcgcgacttccagctcttttacaaataagccttttccgcctactttcgccagcggcgtatcaagaatcacatcgccgcgcgtcaccatcggtaccagttcaacgaccaggcccggatggctcgccatcaacttgtctttgacatagtgtgcctgccagagtgcaagtgggctttggcgtgtggcaattcttaaaacattgtctaacatgcttgttaccgtcattatcatccgtggtccatcctaacatccttgccagagtgatgtcagtgttgtggtgaaacgtagacgcctgcgcaaaccgtaaaatgaggtctggcagtggatcctgacaggcgtttcacgccgttgtaataaggaatttacagagaataaacggtgctacacttgtatgtagcgcatctttctttacggtcaatcagcaaggtgttaaattgatcacgttttagaccattttttcgtcgtgaaactaaaaaaaccaggcgcgaaaagtggtaacggttacctttgacatacgaaatatcccgaatgccgcgtgttaccgttgatgttggcggaatcacagtcatgacgggtagcaaatcaggcgatacgtcttgtacctctatattgagactctgaaacagagactggatgccataaatcaattgcgtgtggatcgcgcgcttgctgctatggggcctgcattccaacaggtctacagtctactgccgacattgttgcactatcaccatccgctaatgccgggttaccttgatggtaacgttcccaaaggcatttgcctttacacgcctgatgaaactcaacgccactacctgaacgagcttgaactgtatcgtggaatgtcagtacaggatccgccgaaaggtgagcttccaattactggtgtatacaccatgggcagcacctcgtccgtagggcaaagttgttcctctgacctggatatctgggtctgtcatcaatcctggctcgatagcgaagagcgccaattgctacaacgtaaatgtagcctgctggaaaactgggccgcctcgctgggtgtggaagtcagcttcttcctgattgatgaaaaccgcttccgtcataatgaaagcggcagcctggggggcgaagattgtggctccacccagcatatactgctgcttgacgaattttatcgtaccgccgtgcgtctcgccggtaagcgtattctgtggaatatggtgccgtgcgacgaagaagagcattacgacgactatgtgatgacgctttacgcgcagggcgtgctgacgccaaatgaatggctggatctcggtggcttaagctcgctttctgctgaagagtactttggtgccagcctttggcagctctacaagagtatcgattccccatacaaagcggtactgaaaacactgctgctggaagcctattcctgggaatacccgaacccacgtctgctggcgaaagatatcaaacagcgtttgcacgacggcgagattgtatcgtttggtctcgatccatactgcatgatgctggagcgtgttactgaatacctgacggcgattgaagattttacccgtctggatttagtacgtcgctgcttctatttaaaagtgtgcgaaaagctcagccgtgaacgcgcctgcgtaggctggcgtcgcgcagtgttgagccagttagtgagcgagtggggttgggacgaagctcgtctggcaatgctcgataaccgcgctaactggaagattgatcaggtgcgtgaggcgcacaacgagttgctcgacgcgatgatgcagagctaccgtaatctgatccgctttgcgcgtcgcaataaccttagcgtctccgccagtccgcaggatatcggcgtgctgacgcgtaagctgtatgccgcgtttgaagcattaccaggtaaagtgacgctggtaaacccgcagatttcacccgatctctcggaaccgaatctgacctttatttatgtgccgccgggccgggctaaccgttcaggttggtatctgtataaccgcgcgccaaatattgagtcgatcatcagccatcagccgctggaatataaccgttacctgaataaactggtggcgtgggcatggtttaacggcctgctgacctcgcgcacccgtttgtatattaaaggtaacggcattgtcgatttgcctaagttgcaggagatggtcgccgacgtgtcgcaccatttcccgctgcgcttacctgcaccgacaccgaaggcgctctacagcccgtgtgagatccgccatctggcgattatcgttaacctggaatatgacccgacagcggcgttccgcaatcaggtggtgcatttcgatttccgtaagctggatgtcttcagctttggcgagaatcaaaattgcctggtaggtagcgttgacctgctgtaccgcaactcgtggaacgaagtgcgtacgctgcacttcaacggcgagcaatcgatgatcgaagccctgaaaactattctcggcaaaatgcatcaggacgccgcaccgccagatagcgtggaagtcttctgttatagccagcatctgcgcggcttaattcgtactcgcgtgcagcaactggtttctgagtgtattgaattgcgtctttccagcacccgccaggaaaccgggcgtttcaaggcgctgcgcgtttctggtcaaacctgggggttgttcttcgaacgcctgaatgtatcggtacagaaactggaaaacgccatcgagttttatggcgcgatttcgcataacaaactgcacggcctgtcagtgcaggttgaaaccaatcacgtcaaattaccggcggtggtggacggctttgccagcgaagggatcatccagttctttttcgaagaaacgcaagacgagaatggctttaatatctacattctcgacgaaagcaaccgggttgaggtatatcaccactgcgaaggcagcaaagaggagctggtacgtgacgtcagtcgcttctactcgtcatcgcatgaccgttttacctacggctcaagcttcatcaacttcaacctgccgcagttctatcagattgtgaaggttgatggtcgtgaacaggtgattccgttccgcacaaaatctatcggtaacatgccgcctgccaatcaggatcacgatacgccgctattacagcaatatttttcgtgatgaacgtgccggaaagcgaggcttatccggcatgcaatcttagcggaaactgactgtttcacccgcctgctgcgtcgccgcctgttccagcaaatcccagaaggtttcgccg

SEQ ID NO: 485 - DNA, 3716 bp, synthetic construct
  source 1..3716: mol_type = other DNA
ccctcgatgttgacgttgcaaaaattgcaatgtgacgttgtgaatatatcaatttccgcaataaatttcctgtcatatagtgaattcaatctcgcaaacgcgaaccgaacaataagaagcacaacatcacgaggaatcaccatggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctacagccacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaaagctccttcgttgcggaagtgaaatctgacctgatgggcgagcaaaccatcctgtgcggtatgctacaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaaagtctgcacgagctgccgctgattgccaacaccatcgcccgtaagcgtctgtacgaaatgaacgtggttatctctgataccgctgagtacggtaactatctgttctcttacgcttgtgtgccgctactgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaagtgcgcgctgatgccctcaccccgaccctctcccacagggagagggagaaaacactcaaggccttctcctggagaaggccttgctattagttgcggtacagcaccttaatgatgtgatatccgaactgggtgtgcagcgggccggtcggctccagtaccggacaagagaaaaccactttatcgaacgccggaaccatctgaccctggcggaattcaccgagatcaccgccgcgtttgccacttgggcaaatggagtgtttcttcgccagcttgccgaaatcggccccgttcttaatctgctcgagaagatccagagccagtttctcttcttttacaaggatatgcagtgctgctgctgtttttgccatgatcgtgccttgagtgaataattgtcaggcggcgattttagcatgatccgcagatgctaacgtaattgtcagtcagggagtggctggtacatatatggcaaaagcccggaaaatccgggcttcgcaggtccctcaaggggagaaaacttagggcctctatgggctacaaagtcattatcggggatggttattttattgtcaatttatgagcgatatggcattgattgacttgatgaccgccctctgtatgtctgcaaccaactcatgtggcaaacattgattaagataccctctgtaagtccttgctctgtttaatctgtttcgactgacttgttgtaccaggtcggctttggcccaacgaatctgctgatcaaagaattgcaggtcattgataacattactggctatttcaacatgcattccacgttttaacttgtctggtcgcgagtggtactgagtggaacgacgatgcatgcgttgccattaatttttccgttgagcacgacaacgagtcggttttttatcatttcaggaggaatgcggccatcgtaatacgtcgtgctgaacggtccattttggctaacaggatagttaccaaagttgcattccagtatttcaccaaccttagggctgaaattaatcgccacgagtaaacctctccttagtaaactctgaaaaagtaataacacaacgttacgacccgatattttctaagtctaatggattcacgatgaactccgatttcggtcttctctctctgatttaaacatccgcagccaaccggttagcggcttacacgcggtcacattcaaatgcgattctgctacaatcctccccccgttcgaagattgagcaatacacctatgcgtctaaaccccggccaacaacaagctgtcgaattcgttaccggcccctgcctggtgctggcgggcgcgggttccggtaaaactcgtgttatcaccaataaaatcgcccatctgatccgcggttgcggttatcaggcgcggcacattgcggcggtgacctttactaataaagcagcgcgcgagatgaaagagcgtgtagggcagacgctggggcgcaaagaggcgcgtgggctgatgatctccactttccatacgctagggctggatatcatcaaacgcgagtatgcggcgcttgggatgaaagcgaacttcagcctatttgacgataccgatcagcttgctctacttaaagagctaaccgaggggctgattgaagatgacaaagttctcctgcaacaactgattagcaccatctctaactggaagaatgatctcaaaacaccgtcccaggcggcagcaagtgcgattggcgagcgggaccgtatttttgcccattgttatgggctgtatgatgcacacctgaaagcctgtaacgttctcgacttcgatgatctgattctcctaccgacgctactgctgcaacgcaatgaagaagtccgcaagcgctggcagaacaaaattcgctatctgctggtggatgagtatcaggacaccaacaccagccagtatgagctggtgaaactgctggtgggcagccgcgcgcgctttaccgtggtgggtgacgatgaccagagcatctactcctggcgcggtgcacgtccgcaaaacctggtgctgctgagtcaggattttccggcgctgaaggtgattaagcttgagcagaactatcgctcttccgggcgtattctgaaagcggcgaacatcc

SEQ ID NO: 486 - DNA, 3628 bp, synthetic construct
  source 1..3628: mol_type = other DNA
gaaagcggcgaacatcctgatcgccaataacccgcacgtctttgaaaagcgtctgttctccgaactgggttatggcgcggagctaaaagtactcagcgcgaataacgaagaacatgaggctgagcgcgttactggcgagctgatcgcccatcacttcgtcaataaaacgcagtacaaagattacgccattctttatcgcggtaaccatcagagccgggtgtttgaaaaattcctgatgcaaaaccgcatcccgtacaaaatatctggtggtacgagctttttctctcgtcctgaaatcaaggacctcctggcttatctgcgcgtgctgactaacccggacgatgacagcgcatttctgcgtatcgttaacacgccgaagcgagagattggcccggctacgctgaaaaagctgggtgagtgggcgatgacgcgcaataaaagcatgtttaccgccagctttgatatgggcctgagtcagacgcttagcggacgtggttatgaagcactaacccgcttcactcactggctagcagaaatccagcgtctggcggagcgggagccgattgccgcggtgcgtgatctgatccatggcatggattatgaatcctggctgtacgaaacaagccccagcccgaaagccgccgaaatgcgcatgaagaacgtcaaccaactgtttagctggatgacggagatgctggaaggcagtgaactggatgagccgatgacgctcacccaggtggtgacgcgctttactctacgcgacatgatggagcgtggtgagagtgaagaagagctggatcaggtgcaactgatgactctccacgcgagcaaagggctggagtttccttatgtctacatggtcggtatggaagaagggtttctaccgcaccagagcagcatcgatgaagataatatcgatgaggagcggcggctggcctatgtcggcattacccgcgcccagaaggaactaacctttacgctgtgtaaagaacgccgtcagtacggcgaactggtgcgcccggagccgagccgctttctactggagctgccgcaggatgatctgatttgggaacaggagcgcaaagtggtcagcgccgaagaacggatgcagaaagggcaaagccatctggcgaatctgaaagcgatgatggcggcaaaacgagggaaataatcaaggccggaaaagatgcgtcagcatcgcatccggcacttactcattaatgcacttccagcggccagtggacatagctctgccactggctttcctgagcaataatctctttacccagcggatgttgggttagccaaccttgcggaagcgttagggttagcagttcatggttagcctggagtgtcatctctggcacgagatcgtcacggcgacggctggcaaaaatgatggccaggcgtaggagacggcagagttgttctgcgacgcgcggcggtacggcattttgctgatgcaggctactgagatcgaccggattagtctggttgagcagtagcgtcgccagtagttttttctgtgcgggggtaaaaccgggaagatccagattacgcaccagataagcagcgtgttgcggcgcttgtttgaagtcaacgctcaggccgatttcatgaagctgacaggcgctgatgagtagatcgcggcttattgcttcaagatgccattcgttttccacctgatcgaagaagttagccgcaactttggctacgcgctgtgcctgatcaatatcgatcataaagcggcgttgaatattacgcagcgtacggctgcgaatatcctgctcgacggccagatgtagcatgccgtagaccaggccttcacgcagcgcaccgcccgccagggtcatacactgaatattcagttcggtaaaaatggcgatcaggatcgccagaccactcgggaacacgagcgcacgttccagcgtcagcccgtcaatctccagttcctccagccgaccgcaatgaatggctcgctgttttagttgctgtagcttttccagggtaatgcgttcatccatcccctgtgccatcatgatttcctggagcgcctgcacggtgccggaagcaccaacgcacactttccagccgtgataccggagttcatcggcaaccggacggagcacttcgcgtgccgctttttctgcagcatcaaaattttcctgccccagattacgatcggcaaaatagcgttccagccaggtgacgcagcccatgctcaggctgaataggctggtggtttgtgcacccgtgccggttaccagttcagtactggctccgcctatatccaccaccaggcgctgatcggcaccaccagtggtgtgagcaacgccctgataaatcagacgtgcttcctcttcaccgctgatcacctgtaccggacaaccgagaatttcctgcgctttggcaataaaatcacccgcattgacggcaaggcggagcgtcgccgtagcgacaacgcgaatttggctgggaggaatatcttgcagacgttcagcaaacaggcgtagacattgccaaccgcgctccattgcttcattggagagggcattttcgctattcaggccagcagccagacgcactttgcgttttattcgcgtcagcgtctggatgcttccagccacctcgcgcacaaccagcatatgaaaactattgctaccgagatcaatggctgcatacaggctgctggtggaacccatactcttcatccttcaatctgtctctgtgttggctgcattctttcacctgaatcacttactacagtaagctcatcgggattcattctcttgccatcttgatacagtttgaatgattttgagtatgacattttttatttaaccactacgacgacgattacgcggagcgccagtacgacgcggaccattgcctgtgcgcgggcgcgtgaggcgcagcggttttggcagatcggtcattagtgcgtccggattgtatttgcttaccggaatactgtgaccaatataggtctcaatagcaggtagatttagtgcatactcttcacacgccaggctgatagagtgaccgcttgcgcctgcgcgacctgtacgaccaatacggtgaacgtaatcttcacagtcatcggggagatcgtagttaaagacgtgcgtcactgccggaatatgtagaccacgcgcggcaacgtcggtggcaaccagaatatccagatcgccacgggtaaattcatcaagaatacgcagacgttttttctgcgcgacatcgcctgttaggagaccgacacgatgaccatctgctgccaggtggccccagatctcttcacaacggtgtttggtgttggcgaaaataatcgcgcggtctggccactcttcttcgatcagcgtttgcagtagacgcattttttcttcgttagaagggtagaaaagctcttctttaatacggtggcccgttttctgttccggttccacttcaatatattcggcattgttcatctgctcgaacgccagttcacgtacccggtagctaagcgtggcggagaacagcatgttgaggcgctggtttgcaggcggcatacggcggaacagccagcggatatctttaataaagcccagatcgtacatgcgatcggcttcgtccagtaccaccacctgaa

SEQ ID NO: 487 - DNA, 3718 bp, synthetic construct
  source 1..3718: mol_type = other DNA
aagcccagatcgtacatgcgatcggcttcgtccagtaccaccacctgaatggcaccgaggttaatgtggttctgcttggcgtagtcaattaaacgccccgtggtgccaatcagaatgtcaacgccgctttccagcactttcagctgtttgtcgtagccatcaccaccgtaagccagacccagcttcaggccagtagcttccgccagcggttctgcgtcggcatggatctgcacggcaagttcacgcgtcggtgccataatgagggcacgcggctgattcaccttgcgatcggcaatcgcaggatgagagagaagataatgaaacgtactcgtaagaaacgccatcgttttcccggtaccggtttgcgcctgcccggctacgtcacgacccgccagcgtcagcggaagggccagtgcctgaatgggcgtacagttatgaaacccttttttttcaagggcttctacaaccttcggatgcagggcgaagtcggaaaacttctgttctgtgagatgtgttttgctcatagtgtggtagaatatcagcttactattgctttacgaaagcgtatccggtgaaataaagtcaacctttagttggttaatgttacaccaacaacgaaaccaacacgccaggcttattcctgtggagttatatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagctaaaagagttcctcgacgctaacctggcgtaagggaatttcatgttcgggtgccccgtcgctaaaaactggacgcccggcgtgagtcatgctaacttagtgttgacttcgtattaaacataccttattaagtttgaatcttgtaatttccaacgcttcccgttttatcttaaatgcgaagtgaacagatttctggcagcagtctcaatccgtcttgtcgtttcagttctgcgtactctcctgtgacccgacagcgaaaagacatgagtcgatgaccgtaaacaggcatggatgatcctgccataccattcacaacattaagttcgagatttaccccaagtttaagaactcacaccactatgaatcttaccgaactcaagaatacgccggtttctgagctgatcactctcggcgaaaatatggggctggaaaacctggctcgtatgcgtaagcaggacattatttttgccatcctgaagcagcacgcaaagagtggcgaagatatctttggtgatggcgtactggagatactacaggatggatttggtttcctccgttccgcagacagctcctacctcgccggtcctgatgacatctacgtttcccctagccaaatccgccgtttcaacctccgcactggtgataccatctctggtaagattcgcccgccgaaagaaggtgaacgctattttgcgctgctgaaagttaacgaagttaacttcgacaaacctgaaaacgcccgcaacaaaatcctctttgagaacctcaccccgctgcacgcaaactctcgtctgcgtatggaacgtggtaacggttctactgaagatctcactgctcgcgtactggatctggcaagtcctatcggtcgtggtcagcgtggtctgattgtggcaccgccgaaagccggtaaaaccatgctgctgcagaacattgctcagagcattgcttacaaccacccggattgtgtgctgatggttctgctgatcgacgaacgtccggaagaagtaaccgagatgcagcgtctggtaaaaggtgaagttgttgcttctacctttgacgaacccgcatctcgccacgttcaggttgcggaaatggtgatcgagaaggccaaacgcctggttgagcacaagaaagacgttatcattctgctcgactccatcactcgtctggcgcgcgcttacaacaccgttgttccggcgagtggtaaagtgctaaccggtggtgtggatgccaacgccctgcatcgtccgaaacgcttctttggtgcggcgcgtaacgtggaagagggcggcagcctgaccattatcgcgacggcgcttatcgataccggttctaaaatggacgaagttatctacgaagagtttaaaggtacaggcaacatggaactgcacctctctcgtaagatcgctgaaaaacgcgtcttcccggctatcgactacaaccgttctggtacccgtaaagaagagctgctcacgactcaggaagaactgcagaaaatgtggatcctgcgcaaaatcattcacccgatgggcgaaatcgatgcaatggaattcctcattaataaactggcaatgaccaagaccaatgacgatttcttcgaaatgatgaaacgcagttaaatttgtcttatgccaaaaacgccacgtgtttacgtggcgttttgcttttatatctgtaatcttaatgccgcgctggcgatgttaggaaaattcctggaatttgctggcatgttatgcaatttgcatatcaaatggttaatttttgcacaggactggtgggtttggaacggactttcccttctgaataaaggtcttcgtggttatacttctgctaataattttctctgagagcatgcattgtgaatctcctgacagtgagtactgatctcatcagtatttttctcttcacgacactgtttctgttttttgcccgtaaggtggcaaaaaaagtcggtctcgtggataaaccaaacttccgcaaacgtcaccagggactaatacctctcgttggggggattagcgtttacgcagggatttgcttcacgttcggaattgtcgattactatattccgcatgcatctctctatctcgcttgtgccggtgtgcttgttttcattggcgcgctggatgaccgttttgatatcagcgtaaaaatccgtgccaccatacaggccgctgttggcattgttatgatggtgttcggcaagctttatctcagtagcctgggttatatctttggctcctgggagatggtgctcggaccgtttggttacttcctgacgctatttgccgtctgggcggccattaatgcgttcaacatggttgatggcattgatggcctactgggcgggctatcctgcgtcagctttgcagcaatcggtatgattttgtggttcgacgggcaaaccagcctcgcaatctggtgctttgcgatgatcgccgccatcctgccatacatcatgcttaaccttggtatcctgggtcgccgctacaaagtctttatgggtgatgcgggcagtacgctgattggttttaccgttatctggatcctgctcgaaacgacccagggcaaaacccatcccatcagcccggttaccgctttgtggataatcgccattccgctaatggatatggtggcgattatgtaccgtcgcctgcgtaaaggcatgagcccattctctcctgaccgtcagcatattcaccatttgatcatgcgtgccgggtttacttcccgtcaggcgtttgtgctgattacccttgccgcagca

SEQ ID NO: 488 - DNA, 3475 bp, synthetic construct
  source 1..3475: mol_type = other DNA
gcgtttgtgctgattacccttgccgcagcactgctcgcttccattggcgtgctggcagaatattctcattttgtcccggagtgggtcatgctggtgctctttctactagcattcttcctctatggatattgcattaagcgtgcctggaaagttgctcgctttattaagcgcgtaaaacgccgcctgcgtcgcaatcgtggtggcagccccaatctcaccaaataaatgaggatgtgatgacacaaccaatgcctgggaaaccggccgaagacgctgaaaatgaactggatattcgtgggctatttcgtaccctatgggctgggaagctatggattattggcatggggctggcgtttgcgctcatcgcgctggcgtatacgttttttgctcgtcaggagtggagcagcacggcgattaccgatcgtccaacggtgaatatgctggggggatattacagccagcagcaatttctacgtaacctggatgtccgtagtaacatggcttctgccgaccaaccaagcgtcatggacgaagcctataaagagtttgttatgcaactggccagctgggatacccgccgcgagttctggctgcaaaccgactattacaaacagcggatggtgggcaacagcaaagccgatgcggcgctactggatgaaatgattaacaacatccagtttatccccggagactttacccgcgcggtcaatgacagcgtgaagcttattgccgaaaccgcgcctgacgctaataacctgctccgtcagtatgttgcttttgccagccagcgtgcagccagccatctgaatgatgagctgaaaggcgcatgggcggcgcgtaccatccagatgaaagctcaggtgaagcgtcaggaagaggtggcgaaagccatctacgaccgccggatgaacagcattgagcaggcgctgaaaattgctgagcagcataatattagccgcagtgcgacagatgtacctgccgaggaactccctgatagtgaaatgttcctgcttgggcgtccaatgcttcaggctcgactggaaaatctccaggccgtcggtccggcctttgatctcgactatgatcagaatcgggccatgctcaacaccctgaatgttggtccaaccctggatccgcgttttcagacctatcgctatctacgtacgccggaagaaccggtaaaacgcgatagcccacgtcgtgccttcctgatgattatgtggggcattgtcggggggctgatcggggctggtgtcgcactcacccgccgttgcagcaaataacaacactgctgcggtgagcgcaaaggcgctcgccgcttattcgaagagaatcgatgtgaaagtactgactgtatttggtacgcgcccggaagccatcaagatggcgccgctagtgcatgcgctagcaaaagatcctttttttgaggctaaagtttgcgtcactgcgcagcatcgggagatgctcgatcaggtgctgaaactcttttccattgtacctgactacgatctcaacataatgcagccaggacagggcctgacagagataacctgtcggattctggaagggctaaaacctattcttgccgagttcaaaccagacgtcgtgctggttcacggcgatacgacgacgacgctggcaaccagcctggcggcgttttatcagcgtattcctgttggtcacgttgaggctggtctgcgcacgggcgatctctatagcccgtggccggaagaggctaaccgtacactaaccgggcatctggcgatgtatcacttctctccaaccgaaacttcccggcaaaacctactgcgtgaaaacgttgcggatagccgaatcttcattaccggtaatacagtcattgatgcactgctctgggtgcgtgaccaggtgatgagcagcgacaagctgcgtagtgaactggcggcaaattacccgtttatcgaccccgataaaaagatgattctggtgaccggtcaccgacgtgagagtttcggtcgtggctttgaagaaatctgccacgcgctggcagacatcgccaccacgcaccaggacatccagattgtctatccggtgcatctcaacccgaacgtccgcgaaccggtcaatcgcattctggggcatgtgaaaaatgtcattctgatcgatccccaggagtatctcccgtttgtctggctgatgaaccacgcctggctgattctaaccgacagtggcggcattcaggaagaagcgcctagcctggggaaacctgtgctggtgatgcgcgataccactgagcgtccggaagcggtgacggcgggtacggtgcgtctggtaggcacggataagcagcgaattgtcgaggaagtgacgcgtcttctcaaagacgaaaacgaatatcaagctatgagccgcgcccataacccgtatggtgatggtcaggcatgctctcgcattctggaagcgctcaaaaataatcggataagtctatgagttttgcgaccatttctgttatcggactgggttatatcgggctgccaacggcagcagcgtttgccagtcggcaaaaacaggtaattggtgtcgatatcaaccaacatgcggttgataccatcaatcgtggcgaaatccatatcgtcgaacctgatctagcgagtgtagtaaaaactgccgtagaaggcggttttctccgagcgagcacgacgccagttgaagcggatgcctggctgattgctgtacccacgccgtttaagggcgatcatgagccagatatgacctacgttgaaagcgctgctcgctccattgcgccagtgctgaaaaaaggcgcgctggtgatccttgaatccaccagcccggtggggagtaccgagaagatggcagaatggctcgcagagatgcgtccggatctcactttcccgcagcaggtgggcgagcaggcggacgtcaacattgcttactgcccggaacgcgtgctcccaggacaggtaatggtcgagctgattaaaaacgatcgcgtgattggtggtatgacgccggtttgtagcgcccgcgccagcgaactgtacaaaattttcctcgaaggtgagtgtgtcgtcactaacagccggacggcggaaatgtgtaagctcaccgaaaacagcttccgcgatgtgaatatcgcttttgctaatgaactaagcctgatttgtgccgatcaggggattaacgtctgggaactgattcgcctggcgaatcgtcaccctcgcgttaatattcttcagcctggccctggcgtgggcggtcactgcattgctgttgatccgtggtttatcgtggcacagaacccccagcaggcgcggcttatccgtaccgcgcgcgaagtgaacgatcacaaaccgttctgggttatcgatcaggtgaaagcggcggtggctgattgcctggcggctaccgataaacgcgccagtgaactgaaaatcgcctgctttggtctggcgtttaaaccgaatattgatgacct

SEQ ID NO: 489 - DNA, 3692 bp, synthetic construct
  source 1..3692: mol_type = other DNA
aaaatcgcctgctttggtctggcgtttaaaccgaatattgatgacctgcgcgaaagcccggcgatggaaatcgctgaactgatcgcccagtggcatagcggcgaaactctggttgttgagcctaacatccaccagctaccgaaaaaactgaccgggctttgtactctggcgcagcttgacgaggcgctggcaacggcagacgtgctggtgatgctggtcgatcatagtcagttcaaagttatcaatggcgacaatgtccatcagcagtatgtcgtcgatgccaaaggagtctggcgctgatgcgcaaaattctgataacaggtggtgccgggtttattggcagcgcgctggtgcgttatatcatcaacgaaacgagcgacgcggtggtagtggtcgataagctgacctacgccggaaacctgatgagcctggcaccggtcgcgcaaagcgagcgctttgcctttgagaaagttgatatctgcgatcgggcagaactggcacgcgtattcactgagcatcagccagactgtgtcatgcatctggcagccgaaagccatgttgaccgttctattgacggcccggcagcgtttattgaaaccaacattgtcgggacttatacactacttgaagcggcgcgggcttactggaatgcgctgacggaagataaaaaaagcgcgttccgttttcatcatatctccactgacgaagtatatggtgacctgcacagcacggatgatttcttcaccgaaaccacgccgtatgcgccgagcagcccttattccgcgagtaaagccagcagcgaccatctggtgcgcgcctggctgcggacctatggtctgccgacgcttatcaccaactgcagcaataactacggcccttaccactttccggaaaaactgatcccgctgatgatcctcaacgcgctggcgggtaaaagcctgccggtatatggcaacgggcagcaaatccgtgactggctgtatgtggaagatcacgcccgcgcgctgtattgcgtggcgaccaccgggaaagtcggtgaaacctataatattggtggtcacaacgagcgtaagaatctcgatgttgtggaaaccatttgcgagctgctggaagaactggctccgaacaagccgcacggcgtggcgcactatcgtgacctgatcacctttgtcgctgaccgtccggggcatgatctgcgttatgccattgatgctagcaaaattgcccgtgaacttggctggctgccgcaggaaacctttgaaagtggaatgcgtaaaacggttcagtggtatctggctaatgaaagctggtggaagcaggtgcaggacggcagctatcagggcgagcgtctcggtctgaaaggctaattttcagcggaggcaaacatgaaaggtattatcctggcgggcggttccggcacccgactacatccgattacgcgcggcgtaagcaagcaactgctaccgatttacgataagccaatgatttactatccgctgagcgtgctgatgctggccggtatccgcgaaattctcatcatcactacgccggaagataaaggttatttccagcgcctgctgggcgatggtagtgagttcggtatccagctggaatatgccgaacagcccagcccggacggtctggcgcaggcctttatcatcggtgaaaccttccttaatggtgaaccttcttgtctggtgctgggcgataacatcttcttcggtcagggcttcagtccgaagctgcgtcatgttgcggcgcgcaccgaaggggcgacggtttttggctatcaggtgatggacccggaacgctttggcgtggtggagtttgacgacaatttccgcgctatcagcctggaagaaaagccaaaacagccgaagagtaactgggcggtgaccgggctttatttctacgacagtaaagtcgtggagtacgcaaagcaggtgaagccgagcgagcgtggtgaactggagattacctccatcaaccagatgtacctcgaggcgggcaacctgaccgttgaactgctcgggcgcggatttgcctggctggataccggcactcacgacagcctgattgaagccagcacctttgtacagacggtggaaaaacgccagggctttaagattgcctgcctggaagagattgcctggcgtaacggctggctcgatgacgagggtgtgaagcgtgctgccagtagtctcgcgaaaactggctacggccaatatctgctggagctccttcgtgcccgtccgcgccagtattgagccactaacctgggaaaacgccttctttggtgttaacagcgccatcgtgcgcattacgtctgaagcaccgctcctgacgccagacgcgctcgcgccgtggagtcgggtgcaggccaaaattgcggcaagtaatacgggtgaactggatgccctgcaacagctgggattctccctggtagaaggtgaagttgatctagcgctacccgtgaacaatgccagtgatagcggtgctgtagtggcacaagagaccgatattcccgcactccgtcagctcgccagcgccgcatttgcgcaaagccgttttcgtgcgccgtggtatgcgcctgacgccagcagtcgcttttatgcacagtggattgaaaatgccgtgcgcggcacctttgatcatcaatgtctgattctccgtgcggcgtccggcgatattcgcggctatgtctctctccgggaactcaatgcgacagatgcgcgaattggcctgctggctggacgcggtgcaggtgctgagctgatgcaaacggcgctaaactgggcgtatgctcgcggtaaaacaactctacgggtggcgacccaaatgggcaacaccgccgcgcttaaacgatacatacaaagtggtgcgaatgtagaaagcaccgcgtactggctctaccgatgatcacatgattccatttaacgcaccgccggtggtgggaaccgaactcgactatatgcagagcgcaatgggtagcggcaaactgtgtggcgatggcggttttacccgtcgctgccagcagtggctggagcaacgttttggcagcgccaaagtgctcctgacgccgtcctgcaccgctagcctggagatggcggcgctgctgctcgatatccagcctggcgatgaagtgatcatgccgagctacacctttgtctccaccgccaatgcctttgtgctgcgtggcgcaaaaatcgtttttgtggatgttcgcccggacaccatgaacatcgacgaaacgctgattgaagcggcgatcaccgacaaaacgcgcgttatcgtgccggtccattacgcgggtgtggcctgcgaaatggacaccattatggcgctagcgaaaaagcataatctatttgtggtagaagatgccgctcagggcgtgatgtccacttacaaagggcgtgcactgggaaccattggtcatattggctgctttagcttccatgaaaccaaaaactacacggcgggcggtgaaggcggcgcgacgctgattaacgataaagcgctcatcgaacgagccgagatcatccgtgaaaagggcactaaccgcagccagttcttccgtggtcaggtcgataaatatacctggcgcgatattggctccagctatctaatgtccgatctgcaagctgcatacctgtgggcgcaactggaagcagcggatcgtatcaaccagcaac

SEQ ID NO: 490 - DNA, 3487 bp, synthetic construct
  source 1..3487: mol_type = other DNA
gcggatcgtatcaaccagcaacgtctggcgctgtggcaaaactactacgatgcgctcgcgcctctggcgaaagccgggcgtatcgagctgccgagcattcccgatggctgcgtgcagaacgcgcatatgttctacattaaactgcgggatattgatgaccggagcgcgctaattaactttctgaaagaagcggaaatcatggcggtgtttcattacattccgctgcacggttgccctgcgggggaacactttggtgagttccacggtgaagatcgctacaccaccaaagagagcgagcgcctgctgcgcctgccgctgttctacaacctgagccccgtcaatcagcgtacggtaattgcgactctactaaactacttttcctgatatgagcctagcaaaagcgtccctatggacggcggccagtacactggtcaagattggtgccgggctcctggtcggtaagctactggcggtgagttttggtccggcggggcttgggctggcggcaaatttccgccagctaattaccgtgctcggcgtgcttgccggggctggcatctttaacggtgtaaccaaatacgttgcccagtaccatgataatccgcaacagctgcgccgcgtggtcggcactagtagtgcgatggtacttggtttctctacgctgatggcgctggtttttgtgctggcagctgcgccaatcagccagggactatttggtaataccgactatcaggggctggtgcgtctcgtggcgctggtgcaaatggggatcgcctggggcaacctgttactggcgctgatgaaaggctttcgcgatgccgcaggtaatgcgctcagcctgattgtcggcagcctaattggcgttctcgcgtactacgtcagttaccgtctaggcggttatgaaggggcgctactgggtctggcgctgattcccgcgctggtggtaattcctgccgccatcatgctaatcaaacgtggtgtcatcccgctcagctatctgaaacccagctgggataacggtctggcagggcagctaagcaaatttacgctcatggcgctaattacgagcgtgaccttgcctgttgcttacatcatgatgcgtaaactgctggcggcgcagtatagctgggatgaggtggggatctggcaaggggtgagcagtatttccgatgcctacctgcaatttattacggcaagcttcagcgtatatctactgcccacgctaagccggctaacggaaaagcgcgatatcacccgggaagtggttaaaagcctgaaattcgtcctcccggcagtggcggcggcgagttttaccgtctggctgctgcgtgattttgctatctggctgctgctaagcaataaatttaccgctatgcgcgatctctttgcctggcagctcgtgggtgatgtgctcaaagtgggcgcttatgtctttggttatctggtgatcgccaaagcgagtctgcggttttatattctggcggaagtcagccagttcactctcctaatggtatttgcccactggctaatccctgcgcatggtgcactgggcgcggcgcaggcatatatggcaacttatatcgtctatttttctctttgttgtggcgtgtttctcctctggcgtcgacgggcatgactgtactgattcacgtactgggaagcgatatccctcaccataaccgaaccgttctacggtttttcaatgacgcgctggccgcgacgagcgagcacgcgcgcgagtttatggttgttggcaaggacgacggcctcagtgatagctgtccggcgctttctgtgcaatttttccctgggaaaaaaagcctggcggaagcggtcatcgcgaaagcaaaagctaaccgtcagcagcgttttttcttccacggtcagttcaatcccacactgtggctggctctgctgagtggtggcattaagcccagccagtttttctggcatatctggggggcagacctgtatgagctttccagtggcctacgctataagcttttttacccactacgtcgcctggcgcaaaagcgagtcggctgtgtatttgccacccgcggcgatctaagcttttttgccaaaacgcacccaaaggtgcggggcgaactgctgttcttcccgacgcggatggaccctagcctcaatacgatggcgaacgatcggcaacgtgaagggaaaatgaccattctggtggggaactccggcgaccgcagcaatgagcatattgctgccctacgcgccgttcatcagcaatttggcgatacggtaaaagtggtggtgccgatgggatatccgcctaataacgaagcgtacattgaggaagttcgtcaggcggggctggagctcttcagcgaagaaaatctacaaattctgagcgaaaaactggaatttgacgcctatctggcgctacttcgtcagtgcgatcttggttactttatttttgcccgccagcagggcattggtacgctgtgcctcctgattcaggcgggcattccttgtgtgcttaaccgggaaaatccgttctggcaggatatgacggaacaacatctaccggtgctgtttactaccgacgatctcaacgaggatattgtgcgtgaagcgcagcgccagctagcgagcgtggataaaaacaccattgccttctttagccctaactatctacaaggctggcagcgggcgctagcgattgccgcccgagaggtcgcatgagtctgctgcaattcagtggcctgtttgttgtctggctgctctgcacgctgtttattgccacgctgacctggtttgagtttcgccgtgtgcgctttaacttcaatgtcttctttagtctactgtttctactcacctttttcttcggcttcccgctgaccagcgtgctggtatttcgctttgatgttggtgtcgcgccgccagaaatcctactacaggcgctactttctgcgggctgcttctacgcggtttactatgtcacctacaaaacccgcctacgcaaacgcgttgctgatgtaccgcgccgtccgctgtttaccatgaaccgcgtggagaccaatcttacgtgggtgatcctgatgggtatcgcgctggtaagcgtcggcatcttcttcatgcacaacggctttctactgttccggcttaactcctacagtcagatcttttccagtgaagtctccggcgtggcgctcaaacgcttcttttactttttcatcccggcgatgctggtggtctactttctgcgccaggacagcaaagcgtggctgtttttcctcgtcagcacggtcgcctttggcctactgacttatatgattgtcggcggcactcgcgccaatatcatcatcgcattcgctatcttcctgtttattggcattattcgcggctggattagcctatggatgctggcggcggcgggcgtgctggggattgttggcatgttctggctggcactaaaacgctatggaatgaatgtgagcggcgatgaagcgttctatac

SEQ ID NO: 491 - DNA, 3583 bp, synthetic construct
  source 1..3583: mol_type = other DNA
gctggcactaaaacgctatggaatgaatgtgagcggcgatgaagcgttctatacgtttctctatctcactcgcgacaccttcagcccgtgggagaatctggcgctactgctacagaactacgacaacatcgacttccagggcctggctccaattgtccgcgatttctatgtctttatcccttcctggctgtggccgggtcgcccgagtatggtgctgaacagtgccaactactttacctgggaagtgctgaataaccactccggactggcgatcagccctacgcttataggcagtctggtggtgatgggcggcgcgctattcatcccgctcggggcgatcgtggttggtctgatcatcaaatggttcgactggctgtatgagctgggcaaccgcgagcctaatcgctataaagctgcgatactacacagtttctgctttggggcgatcttcaatatgatcgtgctggcgcgtgaagggctggatagctttgtcagtcgcgtggtcttttttatcgtggtcttcggcgcatgtctgatgatcgcaaaactgctatactggctttttgaaagcgccggactcattcataaacgtacaaaaagtagcctccggacgcaggttgaaggataacaatgaataacaacaccacggcaccaacctatacgctgcgtggcctccagctaattggttggcgtgatatgcagcacgccctcgattatctgtttgctgacgggcagcttaagcagggaacgctggttgccattaatgctgaaaaaatgctgactattgaagataacgccgaggtccgagagctcattaacgctgccgaatttaaatatgcggatggcatcagcgttgtacgtagtgtacgtaaaaagtacccgcaggcgcaggtttcccgcgttgccggtgccgatctctgggaagagctgatggcgcgcgcaggcaaagaagggacgccggtatttcttgtgggcggtaaacctgaagtgctggcgcaaactgaagctaaactgcgcaaccagtggaatgtgaatatcgttggcagtcaggatggttattttaaacccgagcagcgtcaggcgctgtttgaacgcattcatgccagcggtgcgcaaatcgtcaccgttgcgatgggaagcccaaagcaggagatcatcatgcgcgactgccgtctggtacatccagatgcgctgtatatgggcgttggcgggacttacgatgttttcaccggtcacgtaaaacgcgcaccgaaaatctggcaaacgctggggctggagtggctctaccgcctgcttagccagccgagccgcattaagcgtcagcttcgtctactgcgttatctccgctggcactacaccggcaacctatgattttcctctctttgtaaagcgacggagtggtcactccgtcgcctgtctgcttttttattacacaaagcattcaaatttttaatgctttatttgccatttctcctgaattacgaaaacatttgcaacactcgatgtacccataacgataaccggtaacaccggaaagcatgcaaacacaacacgaggatttatggcagataacaaaccagagctacagcgtgggctggaagctcgacatatcgaactcatcgccctggggggcaccattggcgtcggcctgtttatgggggccgccagtaccctgaaatgggccgggccatccgtactactagcctatatcatcgccgggctgttcgtctttttcatcatgcgtagtatgggcgaaatgctattcctcgaaccggttaccggtagcttcgccgtttatgcgcatcgttatatgagcccgttctttggctatctcaccgcctggtcttactggtttatgtggatggcggtggggatctctgaaatcaccgccattggcgtttatgtccagttctggttcccggagatggcgcagtggatacccgcactaatcgcagtggcgctggtggcgctagcgaatctggcggcggtgcggctatacggcgaaatcgagttctggttcgcgatgatcaaagtcaccacgattatcgtgatgattgtcattggcctgggcgtgattttctttggctttggcaatggcgggcagagcattggttttagcaatctcacagagcatggcggtttctttgcgggtggctggaaagggttcctgaccgctctgtgtattgtggtggcgtcctaccagggcgtggagctgattggcattactgccggtgaagcgaagaatccgcaggtgacgctgcgcagtgccgtaggcaaggtgctgtggcggatcctgattttctacgtaggcgcgattttcgttatcgtcaccatcttcccgtggaatgaaataggcagcaacggcagcccgttcgtactgacttttgccaaaatcggtattaccgcagcggcgggcattatcaactttgtggtgctgacggctgcgctctctggctgtaacagcggcatgtacagttgcggacgtatgctctacgcactggcgaaaaaccgtcagctcccggcggcaatggcgaaagtttcccgtcacggcgtaccggttgcgggtgtggcagtatctattgctattctgctaattggcagttgcctgaactacatcattcccaatccgcagcgtgtgtttgtctacgtctacagtgccagcgtgcttccggggatggtgccatggtttgtgatactaataagccagctgcgttttcggcgtgcacataaagcggcgattgccagccatccgttccgcagtatcctgttcccgtgggccaattacgtaacaatggcattcctgatttgcgttctaatcggcatgtactttaatgaagatacgcgtatgagcctgtttgttggcatcatctttatgctggcggtgacggcgatttataaagtttttggccttaatcgccacgggaaagcgcataaactggaggaataagcagcaaaacgcacaaaccgtaaccaaacgcgcaatttatttaaaaagggactagacagaggggtgggaagtccgtattatccacccccgcaacggcgctaagcgcccgtagctcagctggatagagcgctgccctccggaggcagaggtctcaggttcgaatcctgtcgggcgcgccatttagtcccggcgcttgagctgcggtggtagtaataccgcgtaacaagatttgtagtggtggctatagctcagttggtagagccctggattgtgattccagttgtcgtgggttcgaatcccattagccaccccattattagaagttgtgacaatgcgaaggtggcggaattggtagacgcgctagcttcaggtgttagtgtccttacggacgtgggggttcaagtcccccccctcgcaccacgactttaaagaattgaactaaaaattcaaaaagcagtatttcggcgagtagcgcagcttggtagcgcaactggtttgggaccagtgggtcggaggttcgaatcctctctcgccgaccaattttgaaccccgcttcggcggggttttttgttttctgtgcatttcgtcaccctcccttcgcaataaacgcccgtaataactcattgccccacggta

SEQ ID NO: 492 - DNA, 3736 bp, synthetic construct
  source 1..3736: mol_type = other DNA
cgcccgtaataactcattgccccacggtatgatttcgcccttaacgtattgaaggatgacttcaggcaaggagcgaccatgctgcaacaggttccaacgcgtgcttttcatgtgatggcgaaaccgagcggttccgattgtaatctgaactgtgactactgtttttatctcgaaaaacaatccctttaccgcgaaaagccagtcacgcatatggacgatgacacgctggaagcgtatgtccgtcactatatcgctgccagcgaaccgcaaaacgaagtggcttttacctggcagggcggcgaaccaacgctactcgggctggcgttttaccgccgtgccgtagcgctacaggcgaaatatggtgctggccgaaagataagtaacagcttccagactaacggcgtgctgctggatgacgaatggtgcgcgtttctcgcggagcatcattttcttgttggtctcagcctggatggcccgcctgagatccacaatcaatatcgcgtgactaaaggtggccgccccacgcataagctggtgatgcgtgccctgacgctcctgcaaaaacatcatgtcgactataacgtgctggtctgcgttaatcgcaccagcgcgcagcaaccgctacaggtatatgattttctatgcgatgcgggagtcgaattcatccagtttattccggtggtcgagcgcctggctgatgaaacaactgcccgcgatggacttaagctccatgcgcctggtgatattcagggtgagctaacggaatggagcgtgcgccccgaggagttcggtgagtttctggtggcgatattcgaccactggatcaaacgcgacgtcggcaagattttcgtgatgaatatcgaatgggcgtttgccaattttgtcggtgcgccgggtgcggtttgccatcatcagccaacctgtgggcgcagcgtgattgttgagcacaacggcgacgtttacgcctgtgatcactatgtttatccgcaatatcggctggggaatatgcaccagcaaacaattgcagaaatgatcgattccccgcaacagcaggcgtttggtgaagataaatttaagcagctcccggcgcagtgtcgcagttgtaacgtgctcaaagcgtgctggggaggctgcccgaaacaccgcttcatgctcgatgccagcggcaaaccgggactgaattatctatgtgccgggtatcagcgttatttccgccatctaccgccatatcttaaagcaatggctgatctactggcgcacggtcgcccggccagcgacattatgcatgcgcatctactggtggtgagtaagtaaaaatcggcggccgcctgcggttgattgccggatgcggcgtaaacgccttatccggcctacatgatcgtgcaaattcaataaattgcagcgttctgtaggctggataagatgcgtcagcatcgcatccggcaaaggcagatctcagcgatagcgccggcttagtcagatttaatctgcgcgcgtggtggatattttttcaggatctccatatacgcgtgcatttcggtctgtagcggtacacccatcggaatatggcgcacgccgatggagtcgctttcctgcggatcggtgtagaggttaaacacgctgcttcccgccgtttgcattactgtgccggtgaatccaccctgatatccgctctgggtataagcgtaaggttgctgaatcaggacgtgatacttgaactcatccatacgcacagcagcgagtttaccgttgaggaagtagtgctcggccttacggttagactgaccatttgttcccaggaagaaggatgtctggtccacaccatcgataaaggtggttttcggcacgagattcgccactttcgctccaggatgccctgccagatccagcgcggtagggaagagatctgccagatcgacaataccgtcagatttacgcggttggatcatccctttccagtaaacgaaagtcggtacgcgaacgccgccttcccaggtgctacctttcgcaccacggaacggggtgcgtccgtgcggcggtacttcggcttccggtccgttatcggaggtaaagacgatcagcgtgttatcaagctgaccgtttttctccagtgttttatacagattagcgaacacatcgttcatctccaccatgcagtcgccatagctggtgcgtgccggagagctacccgcatatttcgcatttgggtagttatcgaagtggcagccacgagtgccgtagtagaggaagaatggtttatcgctcttcgccatcttgtcgaggaacttaacgccatagtccatccagcgttgatccagatcttccatatatttcggcgtaatgtcggcaatggcctgttgttcgccgccgcgcaccgcatgaacgtcatctttgctgaacgggagttgcttgatgtattcagaacggtccggactcagggccacttccggattgacgtgaacgtcgcgccattcggtgtacatatcagacacgctgttaaagccacggaaatcatcaaagccaacgttctgcggctggctctctttgttttcccccatatgccattttccgatggcctgagtgacgtagccctgatcgtgcagtagctgcggcagcgtgggtgacccttgcagcccgcccggttgcccgtacattggcggcatcagaatgccgtggtggatggagtattgtccggtgagaatggtggcgcgggttggggaagagcttggttgagaatacgcgctagtgagaatcagcccctggctggcaacggcgtcgatatctggtgtagggttacccaccgccacgccgccaccgttaaaaccgacgtccatccagcccacatcgtccagtaggaaaacaaccacattcggtttcttaccggtttttttctcaagttctgccagcttctgctgggtttctttatcctgcgccggatgctgcattactggcatcatattgtcggcaatagtggtcgccggtttaaccagatactggtttgggtgatcgtatccggcaaagcctttgcgtgcggtggcagtactcggggtatctgctgcgctggccatgagaggaagagcggcggcgacagcaacaacaagacgtttgggactaaagctaaattccatgcaaaatgctccggtttcatgtcatcaaaatgatgacgtaattaagcattgataattgagatccctctccctgacaggatgattacataaataatagtgacaaaaataaattatttatttatccagaaaatgaattggaaaatcaggagagcgttttcaatcctacctctggcgcagttgatatgtaaggcaggtttattatatcgcgttgattattgatgctgtttttagttttaacggcaattaatatatgtgttattaattgaatgaattttatcattcataataagtatgtgtaggatcaagctcaggttaaatattcactcaggaagttattactcaggaagcaaagaggattacagaattatctcataacaagtgttaagggatgttatttcccgattctctgtggcataataaacgagtagatgctcattccatctcttatgttcgccttagtgcctcataaactccggaatgacgcagagccgtttacggtgcttatcgtccactgacagatgtcgcttatgcctcatcagacaccatggacacaacgttgagtgaagcacccacttgttgtcatacagacctgttttaacgcctgctcc

SEQ ID NO: 493 - DNA, 3709 bp, synthetic construct
  source 1..3709: mol_type = other DNA
gacctgttttaacgcctgctccgtaataagagcaggcgtttttttatgtatcaggaaggccccggaggtgcttgcctccgggtgagaaggaattactgtggcgggttattctgtagcgtgagcattagaccgtcgcgacgcatagctgcagcttcttccggcttgtgcaggcggtccagcgcgtcggcaagccatgcgtaatcgtaggcgtccggacgttgtttcagcgctgcgcggaaggcgaggcttgcttcctgccattctccgtgcttcatcagactctggcccagtgtgctccatagcagcgggcgatcgccgacgtttttgatttgctggcgcagcactttttccagctgttccggattgtttgttttcagtcgaggaatcggcagcagtaggcgatcgtcgtactggcgtttcaggccatcgatgataatttgctgggcagtatcatgatcgtcacattcaataagatgttccgccattgccacctgtagcgctacctgatgacgcgttttccggctttggtttttccaccagttacgtagaccttcgctaccgttatcggcacgcgcctgatccatcaggccaatccatgcctgttgttccagcattgcacgatgttcttcatcaccaacatgggctttcgccatacttgggataatatccagcaggctactccatgcacctgtgcggatatacgcctgttccgccagacggagtacttccggatggcgtggcgtaacttccagcagcttatccacgccgtggcgtgcagcatggttttcattacgggccagttgcagacgtacgcgggtgatttctaccggaatggtgtcgttgccggccagctccgctgcgcgttccagatgttggttggcgcgtgcttcatcaccacgttgttgcgcggcttcggcagccagtagatagttcaccaccggttgttccgcgtgatcggcatttttcgccatcagcttttcaacttgctgataatcgccttccgccagtttcagcagcgcctgttcggtctgcttacgtgcacggcgacgcttacgtccgacaaaccacccacgggtgtgcgcgccagtgcggaagatccgccgcagtagccactcaatggcaaacagcactaccatcgccagaattagtatgatcgccaggcccgtgacgctggtttcgatattgtagttgtcggtctggatcagcacataaccctgatggccggcaatcatcgggccaaccacgatccccgcaatcagtagcacaaagagtaggagcacttttagcatggttattctccttgcggcgcggctgccggagtatcagcttgcggtgcaggtgcaggtttagcttccgttgtccccgctgccggttgtgccagcaggttacgcacgcgagtctgcatcagtttttccagcatcgcctggctttgcagggtttccggaagatccatgctgatattttgctggctgagctggtccacctcgtcgaggaacgctttggtggtggcatcatcagtatcgtagtaagcacgtacccaggtggagacgttctccagcgcctggcgataagtctcttcctggtgacgcggtacagcttgtgctgcgaccagcaggcgagagcgaatattttcgcgcagatagatatcctgatttggcgcgagcagcggtacggcggtgtcatcacgacggcgaatcgtaatgaagttgtccataaagttctgccagcttttttgcagattgatacgccattcgctgatggaactggaaagctcttcaccgtcactatccatcgggctaccatcgctgtcattatcggccagacgcaggttatctacctgattactaagctgattaagcttaaggatgatgccgtcataatccacctggcttactgcagaaaggctggcgatatcatcggtaattgcccgacgaacggtaatcagactcgggtcattcatatccgccaggctggcgtctgcacttttcagtagcgctgcagcggtcgtgacgtcctgatcgctccacagcttccgtccggcgagtttcaccagaaaatcggcctgagccagcagccaggttttagcatcgctgccggaaatggtggcgaccttttgttggacttcatctagctgttttgccagcgtttcttgctgacgattcgcctgcttaagttgtgcagcttgttgcttaataatgccttccagctcggctttttggctctcctgggctttttgtagtgccgtcagttggttagccagggcatcgctggtggcggtctgattgacggcctgttgtttaccccagccatatagaccgatgcccgccgccagagcaatagcgatagccaccgcgctgagaattagtgcggtattgttcttactctttttttctgttgcgacaggttgactcgtggtgtccacggcctctcgggtctcttcaaccacggcggaggttttttcttgttccgtcattatggcttcctgttatgagagttattggagtgcccggagaagcgcatcgttgtcagcgttatcggcgaccttaatgtcttgccagcccagttcccgggcgagtttcgctagacgctcactgacgactagtagtcgacagtgtaggagccagtgctcacgataccattgtgggatcaggctccagagttgctgtagcatttcaccgctggtaacaacgaccatcgtcacctcgcgggcttgccagcgcatcgcttcttctgcaccatcgtaatggattgcgcatcgttgataacattcacaaaaagtgacctcagcaccgcgcgccgtcagggtatccccaattagctcacgaccaccattgccacggagtatcagcgcacgtttgcccgcaatattttggagttcagggagttgtagtaggacttcgctgatttcccgatcctgcgggtagagaatcttctgtccacttacggtatgtagtgccagtgcggtggtgcgtccaatggcgaaataatcaggtagtcggggccatttacgatcttgctgatgcagctgactttgggcaaaagcaaccgcgtgttggctgagggcaaatagcagatcgctctcccccagcgctgccagttgatcagcaagttgcgggagttgttgacccggagaaaactcaatcagcggaaaatgccaggccacctgccccagtgtgcgcagacggctcacgagctcttctccagcgggagacgggcgggtgacaaggatactcatgccggggcgtctccgttatagacttcagcgaggatctcgcgcgcgccgttattcagtagctcttctgccaggctaatccccatttgttcggcatcttgcggcgcaccgcggcgttcaccgcgaataatctggctaccgtccggcgcgccgaccagcgcacgcagccagatttcgccatcaataagctcggcgtagctaccaattggcacctgacatccgccttcgagacgggtattcatggcgcgttctgcggtaacgcgcagtgcagtttcgtggtgattcagcgcggcaagcagctcgcgagtgcgactatcatcaaggcggcattcaatacccaccgcaccttgtcctaccgccggaagagaaatctcgggtggtagcgcggcgcgaatacgactctccagaccgagacgttttagtccggctacggcaagaatgatggcatcgtattcgccgttatccagtttgctcagg

SEQ ID NO: 494 - DNA, 3531 bp, synthetic construct
  source 1..3531: mol_type = other DNA
gccgttatccagtttgctcaggcgagtgccgacgttgccgcgcagggagcggataatcagatccggacggcgttcagccagttggcactggcgacggagactggacgtcccgacgatactgcctgccgggagcgcatccagactgtcatagttattggacacaaaggcatcgcgaggatcttcacgctcacaaatagtgaccagtcccagaccttgcgggaattcaaccggcacatctttcatactgtgtacggcgatatcggcgcgattttcgaggagcgcgacttccagctcttttacaaagaggccttttccgcctactttcgccagcggcgtatcaagaatcacatcgccgcgcgtcaccatcggtaccagttcaacgaccaggcccggatggctcgccattagcttgtctttgacatagtgtgcctgccagagtgcaagtgggctttggcgtgtggcaatgcggagaacattgtcgagcatgcttgttaccgtcattatcatccgtggtccatcctaacatccttgccagagtgatgtcagtgttgtggtgaaacgtagacgcctgcgcaaaccgtaaaatgaggtctggcagtggatcctgacaggcgtttcacgccgttgtaataaggaatttacagagaataaacggtgctacacttgtatgtagcgcatctttctttacggtcaatcagcaaggtgttaaattgatcacgttttagaccattttttcgtcgtgaaactaaaaaaaccaggcgcgaaaagtggtaacggttacctttgacatacgaaatatcccgaatgccgcgtgttaccgttgatgttggcggaatcacagtcatgacgggtagcaaatcaggcgatacgtcttgtacctctatattgagactctgaaacagcgcctggatgccataaatcaactacgtgtggatcgcgcgcttgctgctatggggcctgcattccaacaggtctacagtctactgccgacactactacactatcaccatccgctaatgccgggttaccttgatggtaacgttcccaaaggcatttgcctttacacgcctgatgaaactcaacgccactacctgaacgagcttgaactgtatcgtggaatgagtgtacaggatccgccgaaaggtgagcttccaattactggtgtatacaccatgggcagcaccagctccgtagggcaaagttgttcctctgacctggatatctgggtctgtcatcaatcctggctcgatagcgaagagcgccaactactacaacgtaaatgtagcctgctggaaaactgggccgccagcctgggtgtggaagtcagcttcttcctgattgatgaaaaccgcttccgtcataatgaaagcggcagcctggggggcgaagattgtggctccacccagcatatactgctgcttgacgaattttatcgtaccgccgtgcgtctcgccggtaagcgtattctgtggaatatggtgccgtgcgacgaagaagagcattacgacgactatgtgatgacgctttacgcgcagggcgtgctgacgccaaatgaatggctggatctcggtggcctcagcagcctttctgctgaagagtactttggtgccagcctttggcagctctacaagagtatcgattccccatacaaagcggtactgaaaacactgctgctggaagcctattcctgggaatacccgaacccacgtctgctggcgaaagatatcaaacagcgtctacacgacggcgagattgtaagctttggtctcgatccatactgcatgatgctggagcgtgttactgaatacctgacggcgattgaagattttacccgtctggatctcgtacgtcgctgcttctatctcaaagtgtgcgaaaagctcagccgtgaacgcgcctgcgtaggctggcgtcgcgcagtgctaagccagctcgtgagcgagtggggttgggacgaagctcgtctggcaatgctcgataaccgcgctaactggaagattgatcaggtgcgtgaggcgcacaacgagctactcgacgcgatgatgcagagctaccgtaatctgatccgctttgcgcgtcgcaataaccttagcgtctccgccagtccgcaggatatcggcgtgctgacgcgtaagctgtatgccgcgtttgaagcactcccaggtaaagtgacgctggtaaacccgcagattagtcccgatctcagcgaaccgaatctgacctttatttatgtgccgccgggccgggctaaccgtagtggttggtatctgtataaccgcgcgccaaatattgagagcatcatcagccatcagccgctggaatataaccgttacctgaataaactggtggcgtgggcatggtttaacggcctgctgaccagccgcacccgtctatatattaaaggtaacggcattgtcgatctacctaagctacaggagatggtcgccgacgtgagccaccatttcccgctgcgcctccctgcaccgacaccgaaggcgctctacagcccgtgtgagatccgccatctggcgattatcgttaacctggaatatgacccgacagcggcgttccgcaatcaggtggtgcatttcgatttccgtaagctggatgtcttcagctttggcgagaatcaaaattgcctggtaggtagcgttgacctgctgtaccgcaacagctggaacgaagtgcgtacgctgcacttcaacggcgagcaaagcatgatcgaagccctgaaaactattctcggcaaaatgcatcaggacgccgcaccgccagatagcgtggaagtcttctgttatagccagcatctgcgcggcctcattcgtactcgcgtgcagcaactggtttctgagtgtattgaactacgtctttccagcacccgccaggaaaccgggcgtttcaaggcgctgcgcgtttctggtcaaacctgggggctattcttcgaacgcctgaatgtaagcgtacagaaactggaaaacgccatcgagttttatggcgcgattagccataacaaactgcacggcctgagtgtgcaggttgaaaccaatcacgtcaaactcccggcggtggtggacggctttgccagcgaagggatcatccagttctttttcgaagaaacgcaagacgagaatggctttaatatctacattctcgacgaaagcaaccgggttgaggtatatcaccactgcgaaggcagcaaagaggagctggtacgtgacgtcagtcgcttctacagcagtagccatgaccgttttacctacggcagtagcttcatcaacttcaacctgccgcagttctatcagattgtgaaggttgatggtcgtgaacaggtgattccgttccgcacaaaatctatcggtaacatgccgcctgccaatcaggatcacgatacgccgctactccagcaatattttagctgatgaacgtgccggaaagcgaggcttatccggcatgcaatcttagcggaaactgactgtttcacccgcctgctgcgtcgccgcctgttccagtagatcccagaaggtttcgccg

SEQ ID NO: 495 - DNA, 34 bp, synthetic construct
  source 1..34: mol_type = other DNA
ttgccaccggatagcgctaccttaagatgacgcg

SEQ ID NO: 496 - DNA, 34 bp, synthetic construct
  source 1..34: mol_type = other DNA
ttgccacctgatagcgttaccttaagatgacgcg

SEQ ID NO: 497 - DNA, 34 bp, synthetic construct
  source 1..34: mol_type = other DNA
ttgccacctgatagcgctaccttaagatgacgcg

SEQ ID NO: 498 - DNA, 34 bp, synthetic construct
  source 1..34: mol_type = other DNA
ttgccacctgatagcgctactttaagatgacgcg

SEQ ID NO: 499 - DNA, 18 bp, synthetic construct
  source 1..18: mol_type = other DNA
ttgccaccggatagcgct

SEQ ID NO: 500 - DNA, 18 bp, synthetic construct
  source 1..18: mol_type = other DNA
ttgccacctgatagcgtt

SEQ ID NO: 501 - DNA, 18 bp, synthetic construct
  source 1..18: mol_type = other DNA
ttgccacctgatagcgct

SEQ ID NO: 502 - DNA, 85 bp, synthetic construct
  source 1..85: mol_type = other DNA
ttgccacctggtagcgctacctggagatgacgagttgccacctgtcgaagctacctcgatgacgcgttgcgccaacctgtagcgc

SEQ ID NO: 503 - DNA, 82 bp, synthetic construct
  source 1..82: mol_type = other DNA
ttgccacctggtagcgctaccttaagatgacgagttgccacctgtggaagctaccttgatgacgcgttgcgacctgtagcgc

SEQ ID NO: 504 - DNA, 85 bp, synthetic construct
  source 1..85: mol_type = other DNA
ttgccacctggtagcgctacctggagatgacgcgttgccacctgtggaagctaccttgatgacgcgttgcgccaacctgtagcgc

SEQ ID NO: 505 - DNA, 82 bp, synthetic construct; identical to SEQ ID NO: 503
  source 1..82: mol_type = other DNA

SEQ ID NO: 506 - DNA, 82 bp, synthetic construct
  source 1..82: mol_type = other DNA
ttgccacctgatagcgctacctggagatgacgcgttgccacctgtggaagctaccttgatgacgcgttgcgacctgtagcgc

SEQ ID NO: 507 - DNA, 82 bp, synthetic construct
  source 1..82: mol_type = other DNA
ttgccacctggtagcgctaccttaagatgacgagttgccacctgtcgaagctaccttgatgacgcgttgcgacctgtagcgc

SEQ ID NO: 508 - DNA, 85 bp, synthetic construct
  source 1..85: mol_type = other DNA
ttgccacctgatagcgctaccttaagatgacgcgttgccacctgtcgaagctacctcgatgacgcgttgcgccaacctgtagcgc

SEQ ID NO: 509 - protein, 735 aa, Adeno-associated virus 2 [sequence omitted]

(19) United States
(12) Patent Application Publication
(10) Pub. No.: US 2025/0145983 A1
(43) Pub. Date: May 8, 2025

(54) TEMPLATED MUTAGENESIS AND NUCLEIC ACID SYNTHESIS

(71) Applicant: THE TRUSTEES OF COLUMBIA UNIVERSITY IN THE CITY OF NEW YORK, New York, NY (US)

(72) Inventors: Harris Wang, New York, NY (US); Liyuan Liu, New York, NY (US)

(21) Appl. No.: 18/837,509

(22) PCT Filed: Feb. 10, 2023
(86) PCT No.: PCT/US2023/062409

§ 371 Date: Aug. 9, 2024

Related U.S. Application Data
(60) Provisional application No. 63/476,305, filed on Dec. 20, 2022.
(60) Provisional application No. 63/331,022, filed on Apr. 14, 2022.
(60) Provisional application No. 63/309,417, filed on Feb. 11, 2022.

(51) Int. Cl. C12N 15/10; C07K 14/005; C12N 15/86

(52) U.S. Cl. CPC C12N 15/102; C07K 14/005; C12N 15/86; C12N 2750/14122; C12N 2750/14123; C12N 2750/14143

(57) ABSTRACT

The present disclosure provides to systems, methods, and compositions for template-based DNA, synthesis. Particularly the present disclosure provides systems, methods, and compositions for generating multi-site (e.g., three or more) sequence variants of template nucleic acid strands kilobases (e.g., greater than Ikb) m length.

### CROSS-REFERENCE TO RELATED APPLICATIONS

This application claims the benefit of U.S. Provisional Application Nos. 63/309,417, filed Feb. 11, 2022, 63/331,022, filed Apr. 14, 2022, and 63/476,305, filed Dec. 20, 2022, the contents of which are herein incorporated by reference in their entirety.

### STATEMENT REGARDING FEDERALLY SPONSORED RESEARCH OR DEVELOPMENT

This invention was made with government support under MCB-2032259 awarded by the National Science Foundation, AI132403 and DK118044 awarded by the National Institutes of Health, DE-AC52-07NA27344 awarded by the Department of Energy, and N00014-17-1-2353 awarded by the Office of Naval Research. The government has certain rights in the invention.

### FIELD

The present invention relates to systems, methods, and compositions for template-based DNA synthesis, particularly systems, methods, and compositions for making multi-site sequence variants of kilobases in length.

### SEQUENCE LISTING STATEMENT

The contents of the electronic sequence listing titled 40590_601_SequenceListing.xml (Size: 531,344 bytes; and Date of Creation: Feb. 9, 2023) is herein incorporated by reference in its entirety.

### BACKGROUND

Construction and manipulation of kilobase-sized DNA building blocks is foundational to synthetic biology and synthetic genomic. Accordingly, many applications in biological discovery and biotechnology call for the generation and testing of genetic variants or mutants stemming from a wild-type core sequence. At the gene and pathway level, hundreds to thousands of designed or mined sequences need to be iterated to optimize enzyme variants or viral mutants for a desired function. Many natural sequence variations often have unknown biological significance and thus require functional studies through de novo construction and detailed characterizations Large recoding and redesign projects have relied on de novo gene synthesis and genome assembly to test genome-scale designs, which are being further propelled by breakthroughs in computational protein prediction and design. From a conceptual perspective, de novo total DNA synthesis is fundamentally ill-suited for making genetic variants that are kilobases or larger because the majority of time and resources are wasted resynthesizing unchanged portions of a common core sequence from scratch. Despite advances in DNA synthesis over the last two decades, current methods still suffer from size limits, synthesis fidelity, and long lead times. As such, building synthetic sequences remain expensive, labor intensive, and impractical for gigabase genomes such as those of plants or mammals.

Unfortunately, current alternative methods also have various drawbacks. Strategies using cellular machineries such as double-stranded recombineering, base editing, or prime editing require assembling complicated constructs and lack scalability and efficiency at high multiplicities (e.g., greater than ten distinct mutations per gene) and operate within a cellular context, which is less amenable for scale up and automation compared to in vitro strategies. Commercial PCR mutagenesis kits using oligos, while simple and easy to obtain, can only make one or two mutations at a time. More multiplexable oligonucleotide-mediated allelic replacement methods rely on DNA transformation into cells and screening of colonies, which can add significant time, labor, and cost burdens. Currently, a large technology gap remains between genome editing, which is not practical at genome-scale, and de novo synthesis, which is too expensive at genome-scale. Given the various limitations in the field, a new synthesis paradigm is needed for making multi-site sequence variants, quickly, cheaply and in a scalable manner.

### SUMMARY

Provided herein are quick, inexpensive, and scalable synthesis methods for multi-site nucleic acid modification, particularly multi-site sequence variants, on large (e.g., greater than 1 kb) templates.

In some embodiments, the methods comprise annealing three or more mutagenic primers to a template nucleic acid, wherein each of the three or more mutagenic primers comprises at least one mutagenic nucleotide in comparison to template strand; and contacting primed template nucleic acid with a polymerase, a ligase, and nucleotide triphosphates under conditions suitable to extend and ligate a first mutant strand. In some embodiments, the methods comprise annealing at least 6 mutagenic primers. In some embodiments, the methods comprise annealing at least 9 mutagenic primers.

In some embodiments, the template nucleic acid comprises a length greater than 1 kilobases. In some embodiments, the template nucleic acid comprises a length greater than 4 kilobases. In some embodiments, the template nucleic acid is less than 10 kilobases. In some embodiments, the template nucleic acid is not in a circular plasmid.

In some embodiments, the at least one mutagenic nucleotide is each individually selected from a substitution, an insertion, or a deletion relative to the template nucleic acid.

In some embodiments, the template nucleic acid is or is derived from a genomic nucleic acid. In some embodiments, the genomic nucleic acid is from or is derived from a human. In some embodiments, the genomic nucleic acid is from or is derived from a microbial organism. In some embodiments, the genomic nucleic acid is from or is derived from a virus. In some embodiments, the genomic nucleic acid encodes a structural protein from the virus.

In some embodiments, the template nucleic acid comprises DNA. In some embodiments, the template nucleic acid comprises a single strand of DNA.

In some embodiments, the template nucleic acid comprises uracil. In some embodiments, the methods further comprise generating the template nucleic acid by amplifying a target polynucleotide in a reaction comprising deoxyuridine triphosphate and lacking deoxythymidine triphosphate. In some embodiments, the template nucleic acid comprises a modified or synthetic nucleotide. In some embodiments, the modified or synthetic nucleotide is a biotinylated nucleotide, a brominated nucleotide, and deoxyinosine monophosphate, or a combination thereof.

In some embodiments, the three or more mutagenic primers are provided in molar excess to the template nucleic acid. In some embodiments, the three or more mutagenic primers are phosphorylated. In some embodiments, the three or more mutagenic primers each have a 3′ end Guanosine or Cytidine. In some embodiments, the three or more mutagenic primers each have 5′ and 3′ ends having about 10 nucleotides with 100% complementarity to the template nucleic acid.

In some embodiments, the three or more mutagenic primers have the same melting temperatures. In some embodiments, the three or more mutagenic primers have different melting temperatures. In some embodiments, mutagenic primers at the 5′ end of the template nucleic acid have lower melting temperatures compared with mutagenic primers at the 3′ end of the template nucleic acid. In some embodiments, melting temperature for the three or more mutagenic primers have a gradation of melting temperatures from lowest to highest from 5′ to 3′ on the template nucleic acid. In some embodiments, the gradation of melting temperatures is 35° C. to 65° C.

In some embodiments, the annealing comprises decreasing the temperature from 95° C. to less than 20° C. at a rate of 3° C./sec. In some embodiments, wherein the annealing is completed while decreasing the temperature from 95° C. to 4° C. at a rate of 3° C./sec.

In some embodiments, the methods further comprise amplifying the first mutant strand. In some embodiments, the amplifying of the first mutant strand comprises contacting the first mutant strand with polymerase that is inactive for uracil-containing templates. In some embodiments, the methods further comprise generating a copy of the first mutant strand with a reaction mixture comprising deoxyuridine triphosphate and lacking deoxythymidine triphosphate.

In some embodiments, the methods further comprise modifying the first mutant strand, or an amplification product or copy thereof, to generate a second mutant strand. In some embodiments, modifying the first mutant strand, or amplification product thereof, comprises: annealing at least one mutagenic primer to the first mutant strand, or an amplification product or copy thereof, wherein the at least one mutagenic primer comprises at least one mutagenic nucleotide in comparison to the template nucleic acid; and contacting primed first mutant strand, or amplification product thereof, with polymerase, ligase, and nucleotide triphosphates to extend and ligate a second mutant strand.

In some embodiments, the methods further comprise amplifying the second mutant strand and/or generating a copy of the second mutant strand with a reaction mixture comprising deoxyuridine triphosphate and lacking deoxythymidine triphosphate. In some embodiments, the methods further comprise modifying the second mutant strand, or an amplification product or copy thereof, to generate a third mutant strand.

Also provided herein are methods for generating a modified viral genome. The methods comprise annealing three or more mutagenic primers to a template nucleic acid from or derived from a parent or wild-type virus genome, wherein each of the three or more mutagenic primers comprises at least one mutagenic nucleotide in comparison to the parent or wild-type virus genome; and contacting primed template nucleic acid with a polymerase, a ligase, and nucleotide triphosphates under conditions suitable to extend and ligate a mutant strand.

In some embodiments, the methods comprise annealing at least 6 mutagenic primers. In some embodiments, the methods comprise annealing at least 9 mutagenic primers. In some embodiments, the three or more mutagenic primers target sequences encoding structural proteins of the virus.

In some embodiments, the at least one mutagenic nucleotide is each individually selected from a substitution, an insertion, or a deletion relative to the template nucleic acid.

In some embodiments, the template nucleic acid comprises DNA. In some embodiments, the template nucleic acid comprises a single strand of DNA.

In some embodiments, the template nucleic acid comprises uracil. In some embodiments, the methods further comprise generating the template nucleic acid by amplifying a target polynucleotide in a reaction comprising deoxyuridine triphosphate and lacking deoxythymidine triphosphate. In some embodiments, the template nucleic acid comprises a modified or synthetic nucleotide. In some embodiments, the modified or synthetic nucleotide is a biotinylated nucleotide, a brominated nucleotide, and deoxyinosine monophosphate, or a combination thereof.

In some embodiments, the three or more mutagenic primers are provided in molar excess to the template nucleic acid. In some embodiments, the three or more mutagenic primers are phosphorylated. In some embodiments, the three or more mutagenic primers each have a 3′ end Guanosine or Cytidine. In some embodiments, the three or more mutagenic primers each have 5′ and 3′ ends having about 10 nucleotides with 100% complementarity to the template nucleic acid.

In some embodiments, the three or more mutagenic primers have the same melting temperatures. In some embodiments, the three or more mutagenic primers have different melting temperatures. In some embodiments, mutagenic primers at the 5′ end of the template nucleic acid have lower melting temperatures compared with mutagenic primers at the 3′ end of the template nucleic acid. In some embodiments, melting temperature for the three or more mutagenic primers have a gradation of melting temperatures from lowest to highest from 5′ to 3′ on the template nucleic acid. In some embodiments, the gradation of melting temperatures is 35° C. to 65° C.

In some embodiments, the annealing comprises decreasing the temperature from 95° C. to less than 20° C. at a rate of 3° C./sec. In some embodiments, wherein the annealing is completed while decreasing the temperature from 95° C. to 4° C. at a rate of 3° C./sec.

In some embodiments, the methods further comprise repeating the annealing and contacting at least once with each mutant strand, or an amplification product or copy thereof.

In some embodiments, the methods further comprise synthesizing the modified viral genome. In some embodiments, the methods further comprise inserting the modified viral genome into a cell to produce a variant virus or virus-like particle. As such, further provided are methods for making a modified virus or virus-like particle comprising: modifying a viral genome as described herein and inserting the modified viral genome into a cell to produce a modified virus or virus-like particle.

In some embodiments, the parent or wild-type virus genome is from or derived from a virus in the Parvoviridae family. In some embodiments, the parent or wild-type virus genome is derived from an adeno-associated virus.

Additionally provided are modified viral capsid proteins, nucleic acids (e.g., viral vectors) encoding the modified viral capsid proteins, compositions comprising the modified viral proteins, and engineered virus or virus-like particles (VLP) comprising the modified viral proteins. In some embodiments, the viral capsid protein comprises two or more amino acid substitutions and insertions in positions selected from 35-40, 132-152, 188-192, 445-460, 490-505, and 576-596 relative to SEQ ID NO: 509. In some embodiments, the viral capsid protein comprises two or more amino acid insertions between positions selected from: 37/38, 139/140, 190/191, 447/448, 501/502, and 591/592 relative to SEQ ID NO: 509. In some embodiments, each of the two or more amino acid insertions is individually a negatively charged amino acid. In some embodiments, the negatively charged amino acid is selected from aspartate and glutamate.

In some embodiments, the viral capsid proteins comprise an amino acid insertion between positions 591/592, 190/191, or combination thereof relative to SEQ ID NO: 509. In some embodiments, the viral capsid proteins comprise a glutamate insertion between positions 591/592, 190/191, or combination thereof relative to SEQ ID NO: 509. In some embodiments, the viral capsid proteins comprise amino acid insertions between positions 37/38 and 591/592 relative to SEQ ID NO: 509. In some embodiments, the viral capsid proteins between positions 190/191, 501/502, and 591/592 relative to SEQ ID NO: 509. In some embodiments, the viral capsid proteins between positions 37/38, 139/140, 190/191, 591/592 relative to SEQ ID NO: 509.

In some embodiments, the viral capsid proteins a glutamate insertion between positions 37/38 and 591/592 relative to SEQ ID NO: 509. In some embodiments, the viral capsid proteins an aspartate insertion between positions 190/191, 501/502, and 591/592 relative to SEQ ID NO: 509. In some embodiments, the viral capsid proteins an aspartate insertion between positions 37/38, 139/140, and 190/191, and a glutamate insertion between positions 591/592 relative to SEQ ID NO: 509.

Other aspects and embodiments of the disclosure will be apparent in light of the following detailed description.

### BRIEF DESCRIPTION OF THE DRAWINGS

FIGS. 1A-1D show the MEGAA method for DNA variant synthesis. FIG. 1A is a schematic overview of the MEGAA protocol. FIG. 1B is results from testing rsgA templates of different lengths and target positions. In the left panel, the numbers on the right side of the bar show the sizes of the fragments, and the span of the bar are the starting and ending positions of the fragments. Their corresponding MEGAA reaction products are shown in the middle panel with asterisks indicating U-containing templates. The right panel is a graph of variant generation efficiency in which the numbers on the right side of the bar show the percentages of fully complete variants. FIG. 1C is a chart of the efficiency of MEGAA per target site across different rsgA templates. FIG. 1D is a graph of the correlation between mean MEGAA efficiency across 9 targets versus rsgA template size.

FIGS. 2A-2D show MEGAA cycling, optimization, modeling, and automation. FIG. 2A is a schematic of MEGAA cycling to regenerate inputs for additional rounds. FIG. 2B is a graph of the variants generated across increasing number of MEGAA rounds, with random oligo annealing design (Design-1) and ordered oligo annealing design (Design-2). FIG. 2C is a graph of modeling MEGAA cycling efficiency using a binomial process to assess the fraction of all target sites converted at a given mean conversion rate (μ). Solid lines are population distributions at different conversion rates (0.1-0.95) predicted by the model. Dotted line is Design-1 data and Design-2 data over multiple MEGAA cycles.

FIG. 2D is a schematic and image of the MEGAAtron platform to automate the design and synthesis of variants and their validation by nanopore sequencing.

FIGS. 3A-3D show generation of SARS-CoV2 spike gene variants and E. coli codon compressed recoded fragment using MEGAA. FIG. 3A is a chart of 31 natural spike gene variant sequences individually made with MEGAA. MEGAA yield after 2 cycles measured by nanopore sequencing is shown. Asterisks indicate additional mutations in spike gene variants according to WHO.

FIG. 3B shows the generation of recoded genomes by systematically removing codons in E. coli genome.

FIG. 3C is a graph of MEGAA reaction results on 10 fragments showing fraction of recoded target sites in each fragment. MEGAA yield after 1 cycle measured by nanopore sequencing is shown. The numbers on the top of the bar show the percentages of fully complete variants above the percentages of greater than 90% complete variants for each fragment (N.A. indicate the value is less than 0.1%) FIG. 3D is graphs of the recoding efficiency across all target sites in 3 representative fragments.

FIGS. 4A-4D show AAV2 cap gene engineering using MEGAAtron. FIG. 4A is a schematic overview of AAV2 variant generation workflow. FIG. 4B shows package efficiency of 125 AAV2 variants normalized to wild-type levels. Six mutation sites VR-I to VR-VI are noted on the left. Dots in the plot represent individual barcoded replicates, and numbers next to the bars represent numbers of barcoded replicates for AAV variants used in this study. FIG. 4C is a violin plot showing packaging efficiency of variants based on number of mutations per variant. Numbers above the violin plot represent number of barcoded replicates (for WT) or number of variants (for AAV2 variants) shown in the plot. Definition of box-plot elements: center line: median; box limits: upper and lower 25th quartiles; whiskers: 1.5× interquartile range. FIG. 4D is a plot showing measured versus predicted packaging efficiency versus using a linear regression model.

FIG. 5 is a diagram of nanopore sequencing and variant pipeline for MEGAA product analysis. Sequences under full-length variants are SEQ ID NOs: 495-498 from top to bottom. Sequences in Variant-1, Variant-2 and Variant-3 are SEQ ID NOs: 499-501 from top to bottom. Sequences shown in alignment are SEQ ID NOs: 502-507 from top to bottom.

FIGS. 6A and 6B show MEGAA efficiency using different numbers of oligos in each pool against the rsgA6 template and comparison with commercial kits. FIG. 6A shows variant population generated with increasing number of oligos for MEGAA and 3 commercial kits. FIG. 6B are graphs showing the per target efficiency with different numbers of oligos per pool for MEGAA.

FIGS. 7A and 7B show MEGAA reaction using U-templates (pheS1-pheS16) generated near the pheS gene in E. coli K12 genome. A 12-pool oligo was used to target 12 sites spanning an approximately 1 kb region across pheS1-pheS16 templates (FIG. 7A). The numbers on the right side of the bar show the sizes of the fragments, and the span of the bar is the starting and ending positions of the fragments. FIG. 7B, left panel shows gel electrophoresis of the initial U-templates and their corresponding MEGAA products, asterisks indicate U-containing templates. Right panel shows the distribution of variants generated as determined by nanopore sequencing of the MEGAA products.

FIG. 8A shows MEGAA efficiency using improved oligo design. Design-1 does not consider order of oligo assembly to the template, while Design-2 provides an ordered annealing process for oligo assembly favoring 3′ regions first. Corresponding MEGAA efficiencies across 9 target sites are shown in the bottom panels for templates rsgA5, rsgA6 and rsgA7, indicating improved oligo incorporation efficiency using Design-2. FIG. 8B is a graph of the overall population of variants generated using Design-1 and Design-2 for different templates.

FIG. 9 shows MEGAA conversion efficiency of 9 target sites for the rsgA6 variant using oligo Design-1 pool, Design-2 pool or Design-3 pool. For each site, the % of converted sites (the MEGAA efficiency) quantified across all nanopore sequencing reads are shown for Design-1 (same Tm), Design-2 (increasing Tm from 5′ to 3′), and Design-3 (decreasing Tm from 5′ to 3′).

FIGS. 10A and 10B show MEGAA reactions using templates of different GC content. In FIG. 10A, a template with 29% GC content was generated from the sdpB gene of Bacillus subtilis 168 and the associated MEGAA efficiency based on 6 clones was analyzed by Sanger sequencing. Mean conversion efficiency per site (s1-s8) shown on bottom panel. In FIG. 10B, a template with 63% GC content was generated from the pheS gene of Pseudomonas aeruginosa PAO1 and the associated MEGAA efficiency based on 13 colonies was analyzed by Sanger sequencing. Mean conversion efficiency per site (s1-s6) shown on bottom panel.

FIG. 11 shows MEGAA conversion efficiency of 9 target sites for the rsgA6 variant using oligo Design-1 pool or Design-2 pool through iterative rounds of MEGAA cycling. Five rounds are performed with Design-1 pool and three rounds are performed with Design-2 pool. For each site, the % of converted sites (the MEGAA efficiency) quantified across all nanopore sequencing reads are shown for increasing rounds of MEGAA cycling.

FIG. 12 is an exemplary MEGAA design tool (MEGAA-dt) flow chart for oligo design. Template as shown is SEQ ID NO: 508.

FIG. 13 is a chart with cost and speed comparisons of MEGAA variant synthesis with commercial gene synthesis. Comparison is made with standard cost and delivery times for commercial vendor Twist Biosciences. MEGAA costs are estimated with reagent and raw material costs only.

FIGS. 14A-14D show NGS data of MEGAA reactions on SARS-CoV2 S gene using oligos containing degenerate bases (NNS) to produce Spike variant populations containing combinatorial mutations across multiple regions. FIGS. 14A and 14B show per-base nucleotide frequency of targeted sites for S′ genes variants with 6 combinatorial mutation regions (FIG. 14A) and 9 combinatorial mutation regions (FIG. 14B). Oligo degeneracy design and wild-type base at each site are shown above the frequency bar. FIGS. 14C and 14D show amino acid residues frequency of targeted sites for S genes variants with 6 combinatorial mutation regions (FIG. 14C) and 9 combinatorial mutation regions (FIG. 14D). Amino acid residues frequency were calculated based on trinucleotide frequency and the wild-type amino acid residue of targeted sites are indicated by arrow. Amino acid residues were classified into three groups based on number of codons they are mapped to in oligo degeneracy designs: 32 potential codons of NNS cover 13 single-codon residues, 5 double-codon residues and 3 triple-codon residues.

FIG. 15 is nanopore sequencing traces showing mutation efficiency per site across the 10 genomic recoding fragments generated by MEGAA.

FIG. 16A is a fitness map of all single-reside variants (amino acid substitutions or insertions) of AAV2 cap gene with regions of interest (ROI) highlighted. FIG. 16B is a heat map of packaging efficiency of an AAV saturation insertion mutagenesis library, with data from Ogden et al (PMID: 31780559). Regions of interest are surrounded by boxes, shown in the middle section. FIG. 16C is a graph of packaging efficiency (top) for the indicated tested variants (bottom).

FIGS. 17A and 17B are schematics showing AAV2 cap gene variants generation and particle preparation FIG. 17A is a schematic of MEGAA oligos design with GAT or GAA insertion mutations.

FIG. 17B is a schematic overview of AAV variants generation using MEGAA and subsequent cloning and packaging.

FIG. 18 is a comparison of packaging efficiency improvement relative to wild type of single insertion mutants measured in Ogden et al (PMID: 31780559).

FIGS. 19A and 19B show a linear model of AAV variants in packaging efficiency improvement. FIG. 19A shows the weights of individual mutation sites in the linear model with p-value shown on the right. The p-values were calculated by two-sided t-test on the linear regression to test if coefficient of the variable (presence or absence of mutations site) equals to zero in the model and no adjustment was performed on p-values. FIG. 19B is a boxplot of packaging efficiency of all variants with the mutation site. Numbers on the right represents numbers of variants with the mutation site. Definition of box-plot elements: center line: median, box limits: upper and lower 25th quartiles; whiskers: 1.5× interquartile range.

FIG. 20 is a graph of the estimation of correct DNA copies after 5 rounds of MEGAA reactions. Error rate of Q5 Hot Start High-Fidelity DNA Polymerase was previously estimated as 5E-07 substitution/base/doubling (PMID: 28060945) and the proportion of correct DNA copies, e.g., DNA without any accumulated amplification error, was calculated using the same model of NEB PCR Fidelity Estimator (pcrfidelityestimator.neb.com).

FIG. 21 is a comparison of workflows for MEGAA, Darwin Assembly (PMID: 29409059), and other commercial kits.

FIGS. 22A-22C are schematic overviews for exemplary methods to conducting the modification methods disclosed herein in emulsion droplets in which the mutagenic primers are tethered to a solid surface (e.g., plate, bead or particle, and the like) and/or suspended in droplets comprising the template and reagents necessary to carry out the disclosed methods. FIG. 22A provides an overview of key steps. FIG. 22B provides an outline of two exemplary strategies of oligo capture with barcoded beads and oligo release in emulsion droplet, followed by MEGAA reaction. FIG. 22C is an illustration of the strategy to individually capture two different templates (black or brown) in the same reaction.

### DETAILED DESCRIPTION

The disclosed systems, compositions, and methods advance methods for generating multi-site sequence variants, particularly on genome-scale. The disclosed methods, referred to herein as Mutagenesis by Template-guided Amplicon Assembly (MEGAA), combine aspects of de novo synthesis and mutagenesis to generate 10s to 100s to 1000s of defined sequence variations in kilobases of DNA in vitro rapidly and at high fidelity (greater than 90% efficiency per mutation).

Genetic variants are key for understanding biological function and evolution. The capacity to quickly and cheaply generate variants from an existing template or generate synthetic variants from a machine learning algorithm can greatly accelerate the path towards biological elucidation, prediction, and design. MEGAA offers an unprecedented capability to generate 10-100s of multi-site variations across kilobases of DNA at high efficiency in a matter of hours with automation. The methods may include generating and labeling the starting synthesis template to facilitate a dramatic decrease in background amplification during mutagenesis. The highly efficient scaffolded oligo assembly process that provides a high degree of scalability to allow incorporation of greater than 30 mutations at a time per kilobase. In contrast, other methods such as mutagenesis by integrated tiles (MITE) can support only 1 mutation per variant. In addition, as shown herein, the methods can be used to make diverse amplicons of greater than 1 kb in length.

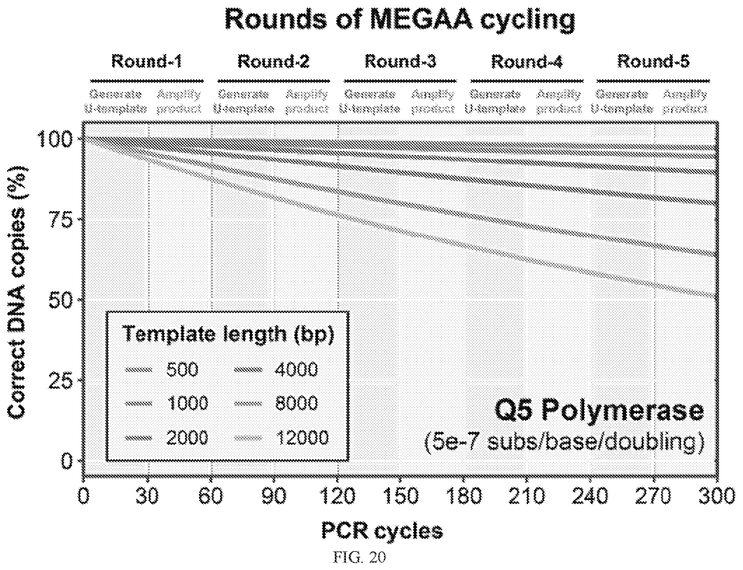

Although iterative PCRs during MEGAA cycling may potentially accumulate amplification errors, PCR-associated errors was minimal for over 5 MEGAA cycles (e.g., greater than 89% of a 2 kb template maintains the perfect sequence over 5 cycles) (FIG. 20). The distribution of variants can be reliably modeled to offer increased control of the in vitro mutagenesis reaction process. This variant synthesis platform can be more economic than de novo gene synthesis for long sequences. The 125-member AAV2 variant library cost approximately $33,000 to build through a commercial de novo gene synthesis vendor (2.2 kb at $0.12/bp) and can take up to 3 weeks to obtain, compared to approximately $2,900 (at $0.01/bp, reagents cost) using the MEGAAtron platform done in a few days by a single person.

Applications that may find use of the disclosed methods include, but are not limited to, plasmid recoding to avoid restriction modification systems, genome reengineering, viral variant studies, antibody repertoire generation, and de novo gene synthesis. Specific applications of variant generation include: genome-scale recoding of bacterial and eukaryotic genomes for genetic code expansion and pan-viral resistance; understanding the mutation selection path during pathogen evolution such as in flu or SARS-CoV2; and enhancing viral gene therapy vectors with improved performance and specificity. Particular applications include: phosphomimetic mutants (e.g., of EGFR) to map phospho-signaling networks, saturation mutagenesis of oncogenes (e.g., PIK3CA) to assess epistatic effects on oncogenicity and drug response, and generate combinatorial variants of therapeutic delivery vehicles (e.g., the capsid protein in Adeno-Associated Viruses) to explore possible improvements in viral packaging, stability, and tissue tropism for viral gene therapy applications. As described herein, the disclosed methods were used to make SARS-CoV-2 spike protein mutants for antibody neutralization studies of emerging variants of concern including the Omicron variant (B. 1.1.529) and to build recoded genome fragments with alternative codon assignments. In addition, six distinct regions of the AAV capsid (positions 35-40, 132-152, 188-192, 445-460, 490-500, 576-596) exhibited various levels of improvements when mutated (insertions or substitutes) in terms of packaging efficiency, thermal stability, and altered tissue tropism.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Although the terms “first,” “second,” “third,” etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first,” “second,” and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV218, AAV2g9, AAV-LK03, AAV7m8, AAV Anc80, AAV PHP.B, and any other AAV including chimeric AAV. See, e.g., BERNARD N. FIELDS et al., VIROLOGY, volume 2, chapter 69 (4th ed., Lippincott-Raven Publishers). A number of AAV serotypes and clades have been identified (see, e.g., Gao et al, (2004) J. Virology 78:6381-6388; Moris et al, (2004) Virology 33-: 375-383). “Adeno-associated virus” or AAV also encompasses chimeric AAV. The term “chimeric AAV” refers to an AAV comprising a protein capsid comprising capsid protein subunits with regions, domains, individual amino acids that are derived from two or more different serotypes of AAV or another virus, including for example, another parvovirus.

As used herein, the term “amplifying” or “amplification” in the context of nucleic acids refers to the production of at least one copy of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, cell, or tumor, may occur by any means of administration known to the skilled artisan.

The term “host cell,” as used herein, refers to a cell that can be infected by the viral vector, can replicate it, and can package it into viral particles that can infect fresh host cells. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. In some embodiments, the host cell is a prokaryotic cell, for example, a bacterial cell. In some embodiments, the host cell is an E. coli cell. In some embodiments, the host cell is a eukaryotic cell, for example, a yeast cell, an insect cell, or a mammalian cell. The type of host cell, depends on the viral vector employed, and suitable host cell/viral vector combinations will be readily apparent to those of skill in the art.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence” are used interchangeably. They refer to a polymeric form of nucleotides of any length, either deoxyribonucleotides or ribonucleotides, or analogs thereof. The term “oligonucleotide,” or “oligos,” as used herein, generally refers to a short nucleic acid sequence comprising from about 2 to about 100 nucleotides (e.g., about 5, 10, 15, 20, 25, 30, 35, 40, 45, 50, 55, 60, 65, 70, 75, 80, 85, 90, 95, 99, or 100 nucleotides, or a range defined by any of the foregoing values) Any of the oligonucleotide sequences described herein may comprise, consist essentially of, or consist of a complement of any of the sequences disclosed herein.

The terms “primer,” “primer sequence,” and “primer oligonucleotide,” as used herein, refer to an oligonucleotide which is capable of acting as a point of initiation of synthesis of an extension product complementary to the template nucleic acid (all types of DNA or RNA) when placed under suitable conditions (e.g., buffer, salt, temperature and pH) in the presence of nucleotides and an agent for nucleic acid polymerization (e.g., a polymerase). The terms “complement” or “complementary sequence,” as used herein, refers to a nucleic acid sequence that forms a stable duplex with an oligonucleotide described herein via Watson-Crick base pairing rules, and typically shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% greater complementarity with the disclosed oligonucleotide. Exact complements, or sequences or regions having 100% complementarity to a sequence form base pairs at each position in the sequence or target region.

The nucleic acids or primers described herein may be prepared using any suitable method, a variety of which are known in the art (see, for example, Sambrook et al., Molecular Cloning. A Laboratory Manual, 1989, 2. Supp. Ed., Cold Spring Harbour Laboratory Press: New York, N.Y.; M. A. Innis (Ed.), PCR Protocols. A Guide to Methods and Applications, Academic Press: New York, N. Y. (1990); P. Tijssen, Hybridization with Nucleic Acid Probes—Laboratory Techniques in Biochemistry and Molecular Biology (Parts I and II), Elsevier Science (1993); M. A. Innis (Ed.), PCR Strategies, Academic Press: New York, N. Y. (1995); and F. M. Ausubel (Ed.), Short Protocols in Molecular Biology, John Wiley & Sons: Secaucus, N.J. (2002); Narang et al., Meth. Enzymol., 68:90-98 (1979); Brown et al., Meth. Enzymol., 68:109-151 (1979); and Belousov et al., Nucleic Acids Res., 25:3440-3444 (1997), each of which is incorporated herein by reference in its entirety). Oligonucleotide synthesis may be performed on oligo synthesizers such as those commercially available from Perkin Elmer/Applied Biosystems, Inc. (Foster City, CA), DuPont (Wilmington, DE), or Milligen (Bedford, MA). Alternatively, oligonucleotides can be custom made and obtained from a variety of commercial sources well-known in the art, including, for example, the Midland Certified Reagent Company (Midland, TX), Eurofins Scientific (Louisville, KY), BioSearch Technologies, Inc. (Novato, CA), and the like. Oligonucleotides may be purified using any suitable method known in the art, such as, for example, native acrylamide gel electrophoresis, anion-exchange HPLC (see, e.g., Pearson et al., J. Chrom., 255:137-149 (1983), incorporated herein by reference), and reverse phase HPLC (see, e.g., McFarland et al., Nucleic Acids Res., 7:1067-1080 (1979), incorporated herein by reference).

The sequence of the oligonucleotides can be verified using any suitable sequencing method known in the art, including, but not limited to, chemical degradation (see, e.g., Maxam et al., Methods of Enzymology, 65:499-560 (1980), incorporated herein by reference), matrix-assisted laser desorption ionization time-of-flight (MALDI-TOF) mass spectrometry (see, e.g., Pieles et al., Nucleic Acids Res., 21: 3191-3196 (1993), incorporated herein by reference), mass spectrometry following a combination of alkaline phosphatase and exonuclease digestions (Wu et al, Anal. Biochem., 290:347-352 (2001), incorporated herein by reference), and the like.

The terms “protein,” “peptide,” and “polypeptide” are used interchangeably herein, and refer to a polymer of amino acid residues linked together by peptide bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, engineered, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N. Y. (2012)), the entire contents of which are incorporated herein by reference.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of proteins, nucleic acids, or compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

As used herein, the terms “providing,” “administering,” and “introducing,” are used interchangeably herein and refer to the placement into a cell, organism, or subject by a method or route which results in at least partial localization to a desired site. For example, the virus or virus-like particles, or compositions disclosed herein can be administered by any appropriate route which results in delivery to a desired location in the cell, organism, or subject.

A “vector” or “expression vector” is a replicon, such as plasmid, phage, virus, or cosmid, to which another DNA segment may be attached or incorporated so as to bring about the replication, transcription, or expression of the attached segment in a cell.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Templated Mutagenesis

Disclosed herein is a framework to build long-sequence variants that employs the concept of templated synthesis. At a high level, templated synthesis utilizes an existing nucleic acid (e.g., DNA) source as a starting point to build a single-stranded template that helps to anchor and anneal pools of short synthetic oligonucleotides encoding the desired changes (e.g., substitutions, insertions, deletions). Large gap regions between oligos are filled with the wild-type sequence of the template by polymerase (e.g., DNA polymerase) and ligated to form the final variant construct. The synthesized construct can be enriched from the mixed reaction and used directly in downstream applications such as assembly into longer constructs, plasmid cloning, or transformation into cells.

The methods comprise annealing three or more (e.g., three, four, five, six, seven, eight, nine, ten, or more) mutagenic primers to a template nucleic acid, wherein each of the three or more mutagenic primers comprises at least one mutagenic nucleotide (e.g., a substitution, an insertion, or a deletion) in comparison to template strand; and contacting the primed template nucleic acid with a polymerase, a ligase, and nucleotide triphosphates under conditions suitable to extend and ligate a first mutant strand. Such conditions are well-known in the art. The conditions encompass all reaction conditions including, but not limited to, temperature and/or temperature cycling, buffer, salt, ionic strength, pH, and the like.

In some embodiments, the annealing can be followed by the contacting (e.g., the extension and ligation reactions). In some embodiments, the annealing and contacting (e.g., the extension and ligation reactions) are simultaneous, e.g., proceed in the single-pot reaction.

The single-stranded template can be or be derived from any input nucleic acid source. The input nucleic acid may be DNA, RNA, or a combination thereof. For example, for input nucleic acids comprising RNA, the input may be reverse transcribed to DNA prior to use in the methods described herein. Thus, the input nucleic acid may be cDNA. The input nucleic acid can be from more than one individual or organism. The input nucleic acid can be synthetic. The input nucleic acid can be double stranded or single stranded. In some embodiments, the template nucleic acid is not in a circular plasmid.

In some embodiments, the template is or is derived from a genomic nucleic acid. The genomic nucleic acid may comprise an entire genome or a portion of a genome. Genomic nucleic acid can refer to actual nucleic acid material isolated from an organism, or alternatively, one or more copies of portions of the genome of an organism or one or more copies of the entire genome of an organism. For example, genomic nucleic acid can refer to a copy of a fragment of genomic nucleic acid that has been isolated from an organism. In some embodiments, genomic nucleic acid is isolated from a cell or other material and fragmented. The fragments are then copied or otherwise amplified. Although this amplified material may contain replica sequences rather than nucleic acid molecules isolated directly from the organism, this material is still referred to herein as genomic nucleic acid or nucleic acid obtained or derived from the genome of an organism. As such, the genomic nucleic acid described herein can include fragments or copies of fragments of genomic nucleic acid sequences.

The genomic nucleic acid may be from any source or organism including human genomic nucleic acids or microbial organism genomic nucleic acids. Genomic nucleic acids include those nucleic acids from all organellar sources (e.g., nucleus, mitochondria, chloroplasts), as well as linear or circular genomes.

In some embodiments, the template is or is derived from a microbial organism. In some embodiments, the microbial organism is a bacterium. In some embodiments, the microbial organism is a virus. In particular embodiments, the genomic nucleic acid encodes a structural protein (e.g., envelope, capsid or nucleocapsid, membrane, spike) from the virus.

In some embodiments, the template is or is derived from a plasmid, a cosmid, bacterial artificial chromosome (BAC), or yeast artificial chromosome (YAC).

In some embodiments, the template is associated with a disease or disorder.

In some embodiments, the template comprises DNA. In some embodiments, the template is generated by amplification of a nucleic acid with a buffer mix where dTTPs are replaced with dUTPs. A polymerase able to use dUTPs at the same fidelity as dTTPs is employed and, as a result, the template contains uracil bases, in the form of deoxyuridine monophosphate, instead of thymine bases. In some embodiments, the template comprises modified or synthetic nucleotides. In certain embodiments, modified or synthetic nucleotides other than uracil are employed in place of uracil and the cognate polymerase is utilized (See for example, Nucleic Acids Res. 2005 Sep. 28, 33 (17): 5640-6, incorporated herein by reference in its entirety). In some embodiments, the modified or synthetic nucleotide is a biotinylated nucleotide (e.g., biotinylated dUMP), a brominated nucleotide (e.g., bromo-dUMP), and deoxyinosine monophosphate (dIMP), or a combination thereof.

The disclosed methods are not limited by size of the template nucleic acid. In some embodiments the template nucleic acid has a length greater than 1 kilobases (kb). For example, the template nucleic acid may be greater that 1 kb, 2 kb, 3 kb, 4 kb, 5 kb, 6 kb, 7 kb, 8 kb, 9 kb, 10 kb, or more. In some embodiments the template nucleic acid has a length less than 10 kilobases (kb). For example, the length of the template nucleic acid may be 1-10 kb, 2-10 kb, 3-10 kb, 4-10 kb, 5-10 kb, 6-10 kb, 7-10 kb, 8-10 kb, 9-10 kb, 1-9 kb, 2-9 kb, 3-9 kb, 4-9 kb, 5-9 kb, 6-9 kb, 7-9 kb, 8-9 kb, 1-8 kb, 2-8 kb, 3-8 kb, 4-8 kb, 5-8 kb, 6-8 kb, 7-8 kb, 1-7 kb, 2-7 kb, 3-7 kb, 4-7 kb, 5-7 kb, 6-7 kb, 1-6 kb, 2-6 kb, 3-6 kb, 4-6 kb, 5-6 kb, 1-5 kb, 2-5 kb, 3-5 kb, 4-5 kb, 1-4 kb, 2-4 kb, 3-4 kb, 1-3 kb, 2-3 kb, or 1-2 kb.

The template and the mutagenic primers are incubated under conditions which facilitate annealing. Annealing is the formation of a double stranded polynucleotide between two single strands, e.g., a primer and a template. Annealing occurs through complementary base pairing between the two strands which are at least 50% or more (e.g., 60%, 70%, 80%, 90%, 95% or more) complementary to each other.

In some embodiments, the three or more mutagenic primers are provided in molar excess (e.g., about 50-, about 100-, about 250-, about 500-, about 750-, about 1,000-fold excess or more) to the template nucleic acid. In some embodiments, a forward extension primer is also added to the template for annealing. The forward extension primer may also be provided in a similar molar excess to the template as the mutagenic primers.

A fast-annealing step using excess oligos aids in limiting or preventing renaturation into a double-stranded template and accurate annealing of the oligos to the minus strand of the template. In some embodiments, the annealing is completed while decreasing the temperature from 95° C. to less than 20° C. (e.g., less than 15° C., less than 10° C., less than 5° C.). In some embodiments, the rate of decreasing is between 1 and 10° C./sec. In select embodiments, the rate of decreasing is 3° C./sec. In select embodiments, the annealing is completed while decreasing the temperature from 95° C. to 4° C. at a rate of 3° C./sec.

The mutagenic primers are so designed that they are not 100% complementary to the template nucleic acid. The mismatches due to insertions, deletions or substitutions are referred to as mutagenic nucleotides. During the disclosed methods, the mutagenic nucleotides are incorporated into the mutant strand. Each mutagenic primer comprises at least one mutagenic nucleotide (e.g., 2, 3, 4, 5, 6, 7, 8, 9, 10 or more).

The mutagenic primers are of sufficiently length to prime extension by the polymerase. The length depends on a variety of factors including template sequence surrounding the targeted site for mutation on the template, the reaction conditions, other reagents, presence of any nucleotide analogs in the sequence. Preferably the sites of the at least one mutagenic nucleotides are flanked by at least about 10 nucleotides of 100% complementarity to the template sequence. Determination of suitability of a single mutagenic primer for more than one mutagenic nucleotide is based on the distance or gap between the two mutagenic nucleotides. For example, if the mutagenic nucleotides are separated by fewer than twice the length of the flanking nucleotides of 100% complementarity, a single primer with two mutagenic nucleotides is applicable. For example, mutagenic nucleotides separated by less than 20 nucleotides may be in a single mutagenic primer. The sequence separating the two mutagenic nucleotides is preferably 100% complementary to the template.

In some embodiments, the three or more mutagenic primers are phosphorylated. 5′ phosphorylation may be achieved by a number of methods, for example, T-4 polynucleotide kinase treatment or synthetic addition, e.g., during synthesis of primers. The phosphorylated primers may be purified (e.g., chromatography (FPLC) or polyacrylamide gel electrophoresis) prior to use in the disclosed methods to remove contaminants. In some embodiments, the mutagenic primers may comprise at least one (e.g., 1, 2, 3, 4, etc.) Guanosine or Cytidine at the 3′ end.

Any of the primers described herein may be modified in any suitable manner so as to stabilize or enhance the binding affinity to the template. For example, the primers as described herein may comprise one or more modified oligonucleotide bases Any of the primers may include, for example, spacers, blocking groups, and modified nucleotides. Modified nucleotides are nucleotides or nucleotide triphosphates that differ in composition and/or structure from natural nucleotides and nucleotide triphosphates. Modifications include those naturally occurring that result from modification by enzymes that modify nucleotides, such as methyltransferases.

In some embodiments, the three or more mutagenic primers have different melting temperatures. Utilizing lower melting temperatures for upstream mutagenic primers compared to downstream primers can increase efficiency of the methods disclosed herein. As such, in some embodiments, the mutagenic primers at the 5′ end of the template nucleic acid have lower melting temperatures compared with mutagenic primers at the 3′ end of the template nucleic acid. In some embodiments, the mutagenic primers from the 5′ to 3′ ends are determined to have sequentially greater melting temperatures to facilitate ordered assembly and/or annealing on to the template. For example, in some embodiments, the 5′ mutagenic primer has a melting temperatures of at least 35° C. and the 3′ mutagenic primer has a melting temperature of at least 45° C. In some embodiments, the 3′ mutagenic primer has a melting temperature between 50 and 65° C. As such the gradation of melting temperatures for the mutagenic primers can range from 35-65° C.

Contacting the primed template with a polymerase, a ligase, and nucleotide triphosphates facilitates extension or gap filling. The polymerase is a non-strand displacing polymerase, for example, a polymerase lacking 5′ to 3′ exonuclease activity (e.g., T4 and T7 DNA Polymerases, Q5 High-Fidelity DNA Polymerase, Phusion High-Fidelity DNA Polymerase). The ligase ligates all newly synthesized segments into a first mutant strand product. Any known ligase is suitable for use in the disclosed methods. Non-limiting exemplary ligases include Taq DNA ligase and HiFi Taq DNA Ligase.

In some embodiments, the mutant strand, which has incorporated any or all mutagenic oligos, is amplified. Amplification can be performed using any suitable nucleic acid amplification method known in the art. In some embodiments, the amplification includes, but is not limited to, polymerase chain reaction (PCR), reverse-transcriptase PCR (RT-PCR), real-time PCR, transcription-mediated amplification (TMA), rolling circle amplification, nucleic acid sequence-based amplification (NASBA), strand displacement amplification (SDA), Transcription-Mediated Amplification (TMA), Single Primer Isothermal Amplification (SPIA), Helicase-dependent amplification (HDA), Loop mediated amplification (LAMP), Recombinase-Polymerase Amplification (RPA), and ligase chain reaction (LCR). In some embodiments, the polymerase utilized in the amplification does not polymerize off of U-containing templates Thus, the amplification enables the specific enrichment of the mutant amplicon products from the first round of the reaction.

The methods may further comprise modifying the first mutant strand or an amplification product or copy thereof, to generate a second mutant strand. The first mutant strand can be modified by any method know in the art.

The methods can be repeatedly cycled using the mutant strand output from one round as the template for the next round. In some embodiments, the methods as disclosed above can be repeated after the production of a first mutant strand using the same set of mutagenic primers with the desired mutagenic nucleotides as compared to the template to generate a second mutant strand. In some embodiments, the methods are repeated with each subsequent mutant strand. For example, the methods may further comprise repeating the above steps with the second mutant strand to generate a third mutant strand, with the third mutant strand to generate a fourth mutant strand, etc.

As such, the methods may further comprise generating a copy of the first/second/third mutant strand with a reaction mixture comprising deoxyuridine triphosphate and lacking deoxythymidine triphosphate for use as the starting template in the following repetition of the method. In some embodiments modifying the first/second/third mutant strand comprises annealing at least one mutagenic primer to the first mutant strand, or an amplification product or copy thereof, wherein the at least one mutagenic primer comprises at least one mutagenic nucleotide in comparison to the template nucleic acid as used in the first modification; and contacting primed first/second/third mutant strand, or amplification product thereof, with polymerase, ligase, and nucleotide triphosphates to extend and ligate a subsequent mutant strand.

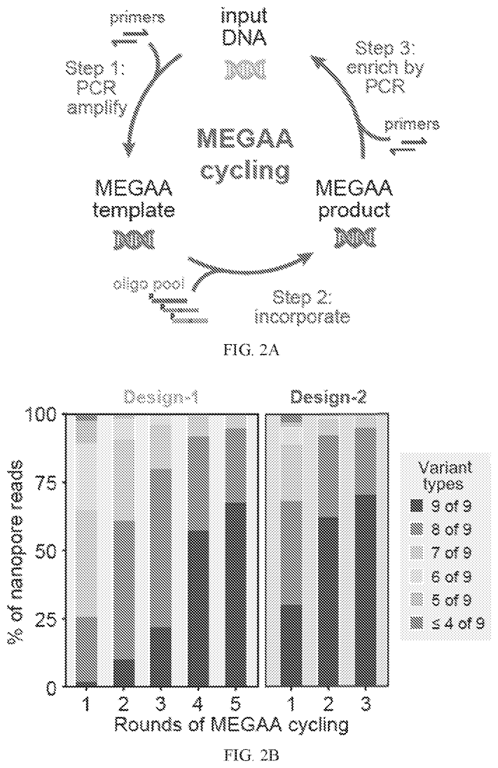

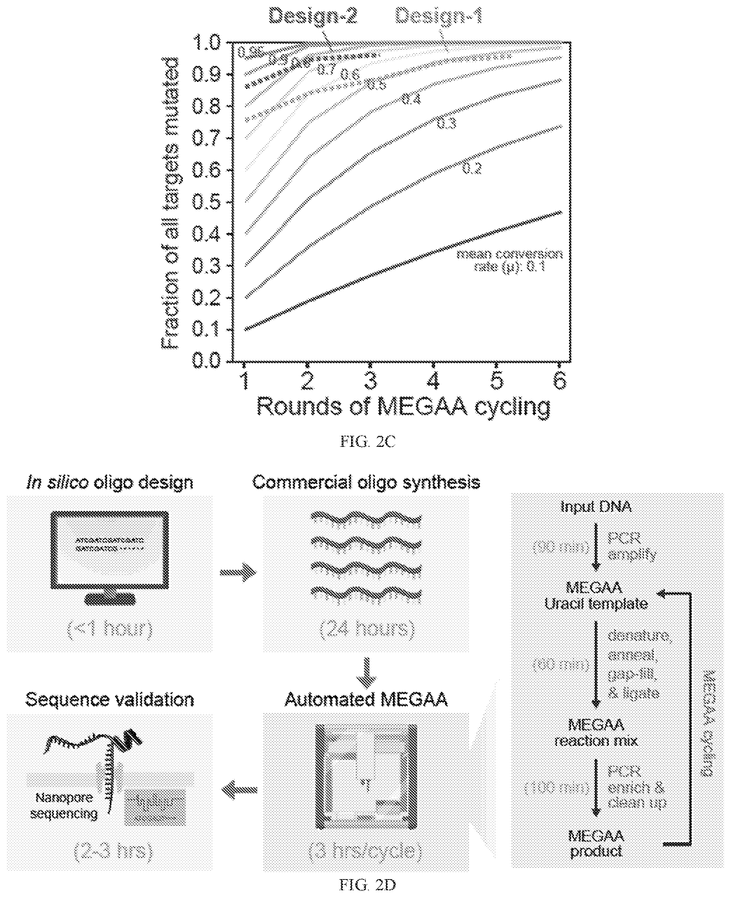

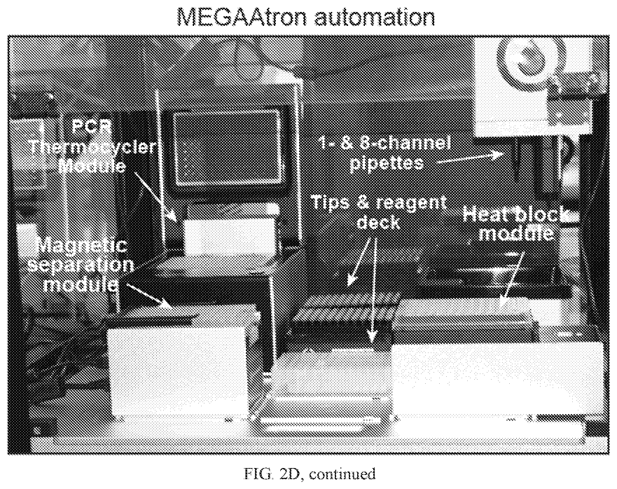

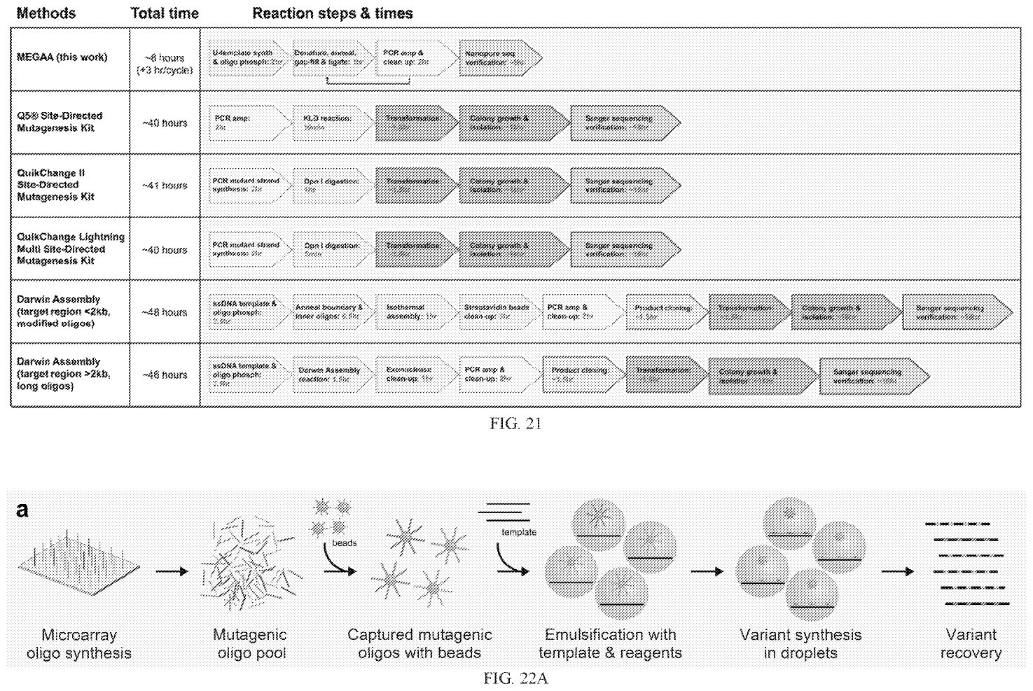

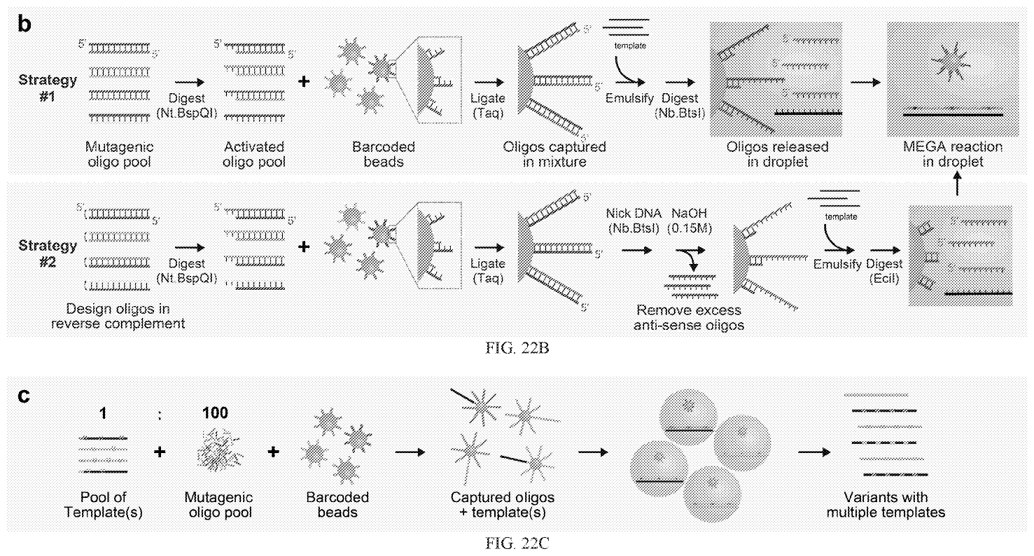

The methods described herein can be adapted for use in a variety of automated (e.g., as in FIG. 2D) and semi-automated systems and platforms, including those wherein the mutagenic oligos are tethered to a solid surface (e.g., bead or particle, as in FIG. 22) or those in which the entire method is carried out in solution.

### Modified Viral Genomes, Viral Proteins, and Virus and Virus-Like Particles

The disclosed methods may be employed to generate modified viral genomes. For example, the methods may be used with a template nucleic acid from or derived from a parent or wild-type virus genome targeted for modification (e.g., insertion, deletion, or substitution) by three or more mutagenic primers. In some embodiments, the three or more mutagenic primers target (e.g., bind to and modify) sequences encoding structural proteins (e.g., spike proteins, capsid or nucleocapsid proteins, membrane proteins, and envelope proteins) of the virus.

In some embodiments, the methods further comprise synthesizing the modified viral genome. This may comprise transcribing the mutant strands into RNA, creating single or double strand versions of the mutant strands, ligating the mutant strands into the remainder of the viral genome, and the like.

In some embodiments, the methods further comprise inserting the modified viral genome into a cell (e.g., a host cell) to produce a variant virus or virus-like particle. As such, the disclosure further provides methods of making a modified virus or virus-like particle comprising modifying a viral genome using the methods disclosed herein and inserting the modified viral genome into a cell to produce a modified virus or virus-like particle.

The methods disclosed herein may be employed to generate modified genomes of any virus of interest. The virus can be a dsDNA virus (e.g., Adenoviruses, Herpesviruses, Poxviruses), a single stranded “plus” sense DNA virus (e.g., Parvoviruses), a double stranded RNA virus (e.g., Reoviruses), a single stranded+sense RNA virus (e.g., Picornaviruses, Togaviruses), a single stranded “minus” sense RNA virus (e.g., Orthomyxoviruses, Rhabdoviruses), a single stranded+sense RNA virus with a DNA intermediate (e.g., Retroviruses), or a double stranded reverse transcribing virus (e.g., Hepadnaviruses).

In some embodiments, the parent or wild-type virus genome is from or derived from a parvovirus. Parvoviridae comprise a family of single-stranded DNA animal viruses. The parvoviruses and other members of the Parvoviridae family are generally described in Kenneth I. Berns, “Parvoviridae: The Viruses and Their Replication,” Chapter 69 in FIELDS VIROLOGY (3d Ed. 1996). Parvoviridae viruses include, for example, parvoviruses (e.g., chicken parvovirus, feline panleukopenia virus, hb parvovirus, h-1 parvovirus, killham rat virus, lapine parvovirus, luiii virus, minute virus of mice, mouse parvovirus 1, porcine parvovirus, rt parvovirus, tumor virus x, hamster parvovirus, rat minute virus 1, and rat parvovirus 1), erythroviruses (e.g., human parvovirus b19, pig-tailed macaque parvovirus, rhesus macaque parvovirus, simian parvovirus, bovine parvovirus type 3, and chipmunk parvovirus), dependoviruses (e.g., AAV-1, AAV-2, AAV-3, AAV-4, AAV-5, avian AAV, bovine AAV, canine AAV, duck AAV, equine AAV, goose parvovirus, ovine AAV, AAV-7, AAV-8, and bovine parvovirus 2), amdoviruses (e.g., aleutian mink disease virus), bocaviruses (e.g., bovine parvovirus and canine minute parvovirus), densoviruses (e.g., Galleria mellonella densovirus, Junonia coenia densovirus, Diatraea saccharalis densovirus, Pseudoplusia includens densovirus, and Toxorhynchites splendens densovirus), iteraviruses (e.g., Bombyx mori densovirus, Casphalia extranea densovirus, and Sibine fusca densovirus), brevidensoviruses (e.g., Aedes aegypti densovirus and Aedes albopictus densovirus), and pefudensoviruses (e.g., Periplaneta fuliginosa densovirus).

In some embodiments, the parent or wild-type virus genome is or is derived from an adeno-associated virus (AAV). The term covers all subtypes and both naturally occurring and recombinant forms, for example, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, or ovine AAV. “Primate AAV” refers to AAV that infect primates, “non-primate AAV” refers to AAV that infect non-primate mammals, “bovine AAV” refers to AAV that infect bovine mammals, etc.

The term “parent” is used herein to refer to viral genomes from which new sequences, which may be more or less attenuated, are derived. Parent viruses and sequences may include “wild type” or “naturally occurring” prototypes or isolates of variants. However, parent viruses also include mutants specifically created or selected in the laboratory on the basis of real or perceived desirable properties. Accordingly, parent viral genomes may have deletions, insertions, substitutions and the like compared to their wild type counterparts, and also include genomes which have codon substitutions.

Thus, in some embodiments, the parent or wild-type virus genome described herein, may be readily selected from among any virus. The genome may be readily isolated from any virus using techniques available to those of skill in the art. Such parental or wild-type virus genome may be isolated or obtained from academic, commercial, or public sources (e.g., the American Type Culture Collection, Manassas, Va.). Alternatively, parental or wild-type genomes may be obtained through synthetic or other suitable means by reference to published sequences such as are available in the literature or in databases such as, e.g., GenBank, PubMed, or the like.

All of the embodiments of the methods disclosed above are suitable for use in the methods to generate the modified viral genome.

Further disclosed herein are modified viral capsid proteins. In some embodiments, the modified viral capsid proteins comprise two or more (e.g., two, three, four, five, six or more) amino acid substitutions and insertions in positions selected from 35-40, 132-152, 188-192, 445-460, 490-505, and 576-596 relative to SEQ ID NO: 509. In some embodiments, the modified viral capsid proteins comprise two or more (e.g., two, three, four, five, six or more) amino acid insertions between positions selected from: 37/38, 139/140, 190/191, 447/448, 501/502, and 591/592 relative to SEQ ID NO: 509.

In select embodiments, each of the two or more amino acid insertions is individually a negatively charged amino acid. In some embodiments, the negatively charged amino acid is aspartate. In some embodiments, the negatively charged amino acid is glutamate.

In some embodiments, the modified viral capsid proteins comprise an amino acid insertion between positions 591/592 relative to SEQ ID NO: 509. In some embodiments, the modified viral capsid proteins comprise an amino acid insertion between positions 190/191 relative to SEQ ID NO: 509. In some embodiments, the modified viral capsid proteins comprise an amino acid insertion between positions 591/592 and 190/191 relative to SEQ ID NO: 509.

In some embodiments, the modified viral capsid proteins comprise a glutamate insertion between positions 591/592 relative to SEQ ID NO: 509. In some embodiments, the modified viral capsid proteins comprise a glutamate insertion between positions 190/191 relative to SEQ ID NO: 509. In some embodiments, the modified viral capsid proteins comprise a glutamate insertion between positions 591/592 and 190/191 relative to SEQ ID NO: 509. In some embodiments, the modified viral capsid proteins comprise an aspartate insertion between positions 591/592 relative to SEQ ID NO: 509. In some embodiments, the modified viral capsid proteins comprise an aspartate insertion between positions 190/191 relative to SEQ ID NO: 509. In some embodiments, the modified viral capsid proteins comprise an aspartate insertion between positions 591/592 and 190/191 relative to SEQ ID NO. 509.

In some embodiments, the modified viral capsid proteins comprise amino acid insertions between positions 37/38 and 591/592 relative to SEQ ID NO: 509. In some embodiments, the modified viral capsid proteins comprise a glutamate insertion between positions 37/38 relative to SEQ ID NO: 509. In some embodiments, the modified viral capsid proteins comprise a glutamate insertion between positions 591/592 relative to SEQ ID NO: 509. In some embodiments, the modified viral capsid proteins comprise glutamate insertions between positions 37/38 and 591/592 relative to SEQ ID NO: 509.

In some embodiments, the modified viral capsid proteins comprise amino acid insertions between positions 190/191, 501/502, and 591/592 relative to SEQ ID NO: 509. In some embodiments, the modified viral capsid proteins comprise an aspartate insertion between positions 190/191 relative to SEQ ID NO: 509. In some embodiments, the modified viral capsid proteins comprise an aspartate insertion between positions 501/502 relative to SEQ ID NO: 509. In some embodiments, the modified viral capsid proteins comprise an aspartate insertion between positions 591/592 relative to SEQ ID NO: 509. In some embodiments, the modified viral capsid proteins comprise aspartate insertions between positions 190/191, 501/502, and 591/592 relative to SEQ ID NO: 509.

In some embodiments, the modified viral capsid proteins comprise amino acid insertions between positions 37/38, 139/140, 190/191, 591/592 relative to SEQ ID NO: 509. In some embodiments, the modified viral capsid proteins comprise an aspartate insertion between positions 37/38 relative to SEQ ID NO: 509. In some embodiments, the modified viral capsid proteins comprise an aspartate insertion between positions 139/140 relative to SEQ ID NO: 509. In some embodiments, the modified viral capsid proteins comprise an aspartate insertion between positions 190/191 relative to SEQ ID NO: 509. In some embodiments, the modified viral capsid proteins comprise aspartate insertions between positions 37/38, 139/140, and 190/191 relative to SEQ ID NO: 509 In some embodiments, the modified viral capsid proteins comprise a glutamate insertion between positions 591/592 relative to SEQ ID NO: 509.

Any of the polypeptides described or referenced herein may comprise one or more additional amino acid substitutions, deletions or insertions as compared to the recited sequences. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

Nucleic acids which encode the modified capsid proteins are encompassed by this disclosure. In some embodiments, the nucleic acid is a viral vector. A viral vector is derived from or based upon one or more nucleic acid elements that comprise a viral genome. Particular viral vectors include lentivirus, pseudo-typed lentivirus, and parvovirus vectors, such as adeno-associated virus (AAV) vectors.

Also provided herein are virus and virus-like particles (VLPs) comprising modified capsid proteins or modified genomes disclosed herein. A “virus particle” refers to a single unit of virus comprising a capsid encapsulating or configured to encapsulate a polynucleotide, e.g., the viral genome (as in a wild-type virus) or a vector (as in a recombinant virus). “Virus-like particles” or “VLPs” refer to a structure that in at least one attribute resembles a virus but which has not been demonstrated to be infectious. Virus-like particles may or may not carry genetic information encoding for the proteins of the virus-like particle, but in general do not include the genetic materials required for viral replication and infection.

In some embodiments, the virus or virus-like particle is derived from a parvovirus, as described above. In some embodiments, the virus or virus-like particle is derived from an AAV virus. An AAV virus particle refers to a viral particle composed of at least one AAV capsid protein. If the particle comprises a heterologous viral vector, it would be referred to as an “rAAV vector particle.”

A rAAV virion can be constructed a variety of methods. For example, heterologous sequence(s) can be directly inserted into an AAV genome which has had the major AAV open reading frames (“ORFs”) excised therefrom. Other portions of the AAV genome can also be deleted, so long as a sufficient portion of the ITRs remain to allow for replication and packaging functions. In order to produce rAAV virions, an AAV expression vector can be introduced into a suitable host cell using known techniques, such as by transfection. Particularly suitable transfection methods include calcium phosphate co-, direct micro-injection into cultured cells, electroporation, liposome mediated gene transfer, lipid-mediated transduction, and nucleic acid delivery using high-velocity microprojectiles. Suitable cells for producing rAAV virions include microorganisms, yeast cells, insect cells, and mammalian cells, that can be, or have been, used as recipients of the viral vector.

An AAV virus that is produced may be replication competent or replication-incompetent. A “replication-competent” virus (e.g., a replication-competent AAV) refers to a phenotypically wild-type virus that is infectious and is also capable of being replicated in an infected cell (e.g., in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector.

Further disclosed herein are compositions comprising the disclosed viral vectors, viral particles, and virus-like particles. In some embodiments, the composition comprises a carrier, e.g., a pharmaceutically acceptable carrier. The phrase “pharmaceutically acceptable,” as used in connection with compositions and/or cells of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the viral vector or virus or virus-like particle and does not negatively affect the subject to which the composition(s) are administered.

Carriers may include any and all solvents, dispersion media, coatings, antibacterial and antifungal agents, isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil: glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, coating agents, sweetening, flavoring and perfuming agents, preservatives and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995).

The virus or virus-like particles, and pharmaceutical compositions disclosed herein provide a means for delivering nucleic acids and thus gene products into a broad range of cells, including dividing and non-dividing cells. The virus or virus-like particles, and pharmaceutical compositions can be employed to deliver to a cell in vitro, e.g., to produce a polypeptide in vitro or for ex vivo gene therapy.

The cell may be, a plant cell, an insect cell, a vertebrate cell, an invertebrate cell, an animal cell, a mammalian cell, or a human cell. In some embodiments, the cell is a plant cell. In some embodiments, the cell is an insect cell. In some embodiments, the cell is a vertebrate cell. In some embodiments, the cell is an invertebrate cell. In some embodiments, the cell is a mammalian cell. In some embodiments, the cell is a human cell. In some embodiments, the cell is a stem cell. In some cases, the cell is ex vivo (e.g., fresh isolate-early passage). In some cases, the cell is in vivo. In some cases, the cell is in culture in vitro (e.g., immortalized cell line).

The virus or virus-like particles, and pharmaceutical compositions are additionally useful in methods of delivering a gene product to cells in a subject, e.g., to express an immunogenic or therapeutic polypeptide or a functional RNA in the subject. The subject can be in need because the subject has a deficiency of the gene product or the production of the gene product in the subject may impart some beneficial effect, e.g., therapeutic or prophylactic benefit. More specifically, the virus or virus-like particles, and pharmaceutical compositions described herein can be used to deliver a desired gene product (e.g., polypeptide, protein, or functional RNA) to treat and/or prevent a disease state for which it is therapeutically or prophylactically beneficial to administer the gene product.

For example, the nucleic acid encapsulated in the virus or virus-like particles may encode one or more RNAs, including for example, an antisense nucleic acid, a ribozyme, RNAs that effect spliceosome-mediated/ram-splicing, interfering RNAs (RNAi) including siRNA, shRNA or miRNA that mediate gene silencing, and other non-translated RNAs.

Alternatively, or in addition, in some embodiments, the nucleic acid encapsulated in the virus or virus-like particles may encode one or more protein or polypeptides. Useful therapeutic protein or polypeptide products encoded by the expression cassette include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15 as well as TGFb proteins, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful gene products include proteins that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factors α and β, interferons α, β, TGFb and γ, stem cell factor, flk-2/flt3 ligand. Gene products produced by the immune system, or recombinant and engineered forms thereof, are also useful in the disclosed methods. These include, without limitation, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Still other useful gene products include any one of the receptors for the hormones, growth factors, cytokines, lymphokines, regulatory proteins and immune system proteins. For example, receptors for cholesterol regulation and/or lipid modulation, including the low density lipoprotein (LDL) receptor, high density lipoprotein (HDL) receptor, the very low density lipoprotein (VLDL) receptor, and scavenger receptors; glucocorticoid receptors and estrogen receptors: Vitamin D receptors; and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

In some embodiments, the virus or virus-like particles, and pharmaceutical compositions described herein can be used to deliver a gene editing system. For example, a zinc-finger nuclease, a homing endonuclease, a TALEN (transcription activator-like effector nuclease), a NgAgo (agronaute endonuclease), a SGN (structure-guided endonuclease), or one or more components of a CRISPR-Cas system.

A “CRISPR-Cas system” refers collectively to transcripts and other elements involved in the expression of and/or directing the activity of CRISPR-associated (“Cas”) genes, including sequences encoding a Cas gene, Cas protein, a cr (CRISPR) sequence (e.g., crRNA or an active partial crRNA), or other sequences and transcripts from a CRISPR locus. In some embodiments, one or more elements of a CRISPR system is derived from a type I, type II, or type III CRISPR system.

For example, the gene editing system may comprise one or more Cas proteins (e.g., Cas9), or other RNA-guided nucleases, and at least one guide RNA directed to a target nucleic acid. Typically, the RNA sequences employed in CRISPR/Cas systems are referred to collectively as “guide RNA” (gRNA) or single guide RNA (sgRNA). Thus, the terms “guide RNA,” “single guide RNA,” and “synthetic guide RNA,” are used interchangeably herein and may refer to a nucleic acid sequence comprising a tracrRNA and a pre-crRNA array containing a guide sequence. The terms “guide sequence,” “guide,” and “spacer,” are used interchangeably herein and refer to the nucleotide sequence within a guide RNA that specifies the target nucleic acid.

Also within the scope of the present disclosure are systems or kits comprising a polypeptide, a nucleic acid, a virus or virus-like particle, or composition as described herein. The systems or kits may further comprise one or more of: buffer or carrier constituents, transfection reagents, and cells for making the virus or virus-like particles, or expression of the polypeptide or nucleic acid.

| The kit may include instructions for use in any of the methods described herein or for methods of making or using the nucleic acids, polypeptides, or virus or virus-like particles. The instructions can comprise a description of administration of the virus or virus-like particles, or compositions to a subject to achieve the intended effect. The instructions generally include information as to dosage and administration.

The kits provided herein are in suitable packaging. Suitable packaging includes, but is not limited to, vials, bottles, jars, flexible packaging, and the like. Normally, the kit comprises a label or package insert(s) on or associated with the packaging. The packaging may be unit doses, bulk packages (e.g., multi-dose packages) or sub-unit doses Instructions supplied in the kits of the disclosure are typically written instructions on a label or package insert.

### EXAMPLES

The following are examples of the present invention and are not to be construed as limiting

### Materials and Methods

Chemical and Oligonucleotide Reagents All chemicals were purchased from Sigma-Aldrich unless otherwise noted. CloneJET PCR cloning system was purchased from Life Technologies. All enzymes were purchased from New England BioLabs. Mutagenic oligos and sequencing primers were purchased from Integrated DNA Technologies.

Strains, viruses, and culture conditions Genomic DNA from Escherichia coli strain K-12 MG1655 was used as the U-containing DNA template for MEGAA studies and codon replacement experiments. Plasmid pcDNA3.1 SARS-CoV-2 S D614G was obtained from Addgene to produce the SARS-CoV2 S gene variants. Plasmids pAAV-CMV vector, pRC2-mi342 vector and pHelper vector were purchased from Takara Bio Inc. (Cat. #6230) to produce the AAV2 capsid variants and for AAV packaging. NEB® Turbo Competent Escherichia coli was used for cloning reactions using standard protocols.

In silico design of MEGA oligos MEGAA oligos were designed to target a template sequence using a custom python script (MEGAA-dt) available at github.com hym0405 MEGAAdt. MEGAA-di script takes reference template sequences and desired mutations information as input and generates designs of mutagenesis oligos and sequences of final variants. Briefly, desired mutations of each variant are evaluated based on their proximity to determine potential oligo regions and mutations too close to each other will be covered by the same oligo. Next, the numbers of perfectly matched bases at 5′ end and 3′ end of oligos are determined sequentially based on their melting temperatures to make oligos assemble in order (lower melting temperatures for upstream oligos). Mutagenesis oligos are then evaluated for their length and distances to adjacent oligo and sequences of final oligo designs and variants are generated.

MEGAA protocol and MEGAAtron automation system In the first step, a MEGAA template is generated using an input DNA source (e.g., wild-type genomic DNA) by PCR amplification with a Q5U® hot start high-fidelity DNA polymerase (New England Biolabs, Cat. #M0515L) with a buffer mix where dTTPs are replaced with dUTPs. Q5U® hot start high-fidelity DNA polymerase is able to use dUTPs at the same fidelity as dTTPs, and as a result the MEGAA template contains uracil (U) bases instead of thymine (T) bases. In the second step, a mix of the MEGAA template, Q5U® hot start high-fidelity DNA polymerase, Taq DNA ligase (New England Biolabs, Cat. #M0208L), and dNTP is made Phosphorylated mutagenic oligos (approximately 30-40 nucleotides) containing the desired mutations (e.g., substitutions, insertions, and deletions) and a forward extension primer are also added to the mix at 500 to 1,000-fold excess of the template. Oligo annealing, extension, and ligation reactions then proceed in the single-pot reaction. Rapid oligo annealing (95° C.→4° C. at a rate of 3° C./sec) is performed on a standard thermal cycler. To increase MEGAA reactions throughput, a liquid handling robot (OT-2; Opentrons, Brooklyn, NY) equipped with magnetic, temperature, and PCR modules was used to automate the MEGAA reaction A detailed step-by-step protocol for the method is included below.

MEGAA characterization experiments A U-containing DNA template (rsgA6, 1,192 bp) was generated by PCR using a Q5U® hot start high-fidelity DNA polymerase with primers rsgA-F0/R0. In the meantime, 15 U-templates of different sizes (1.2-13 kb) were generated by amplifying the rsgA gene region of the E. coli genome with primers from rsgA-F1 to rsgA-F5 with rsgA-R0, rsgA-R1 to rsgA-R5 with rsgA-F0, rsgA-F1 to rsgA-F5 with rsgA-R1 to rsgA-R5 respectively. Another set of 16 U-templates (1.8-12 kb) were generated by amplifying the pheS gene region with a similar approach (Table 1). For rsgA6 fragment, 4 individual MEGAA reactions were performed with 1, 3, 6 and 9 phosphorylated mutantic oligos. For each rsgA pheS gene region mutagenesis, 9-target and 12-target phosphorylated mutantic oligo pool were added to MEGAA reactions. 3 rsgA6 variants (1, 3, and 6-target), rsgA1-rsgA15 and pheS1-pheS15 amplicons were prepared for nanopore sequencing.

MEGAA optimization experiments Oligo pools OP1 (OP1.1-OP1.9) and OP2 (OP2.1-OP2.9) were designed to target 9 sites in rsgA gene. Oligos in OP1 were designed with a similar melting temperature. However, Oligos in OP2 were designed to have a gradation of melting temperature from 47° C. to 64° C. 1,192 bp DNA U-containing template was by amplifying the rsgA gene region of the E. coli genome with primers rsgA-F0 and rsgA-R0. After the separate MEGAA reactions were performed, PCR amplicons (rsgA6) were cleaned up and performed via iteratively cycled MEGAA. 5- and 3-cycle MEGAA reactions were performed with OP1 and OP2 respectively. After each MEGAA reaction, rsgA6r1-r5 and rsgA6r1-r3 PCR amplicons from MEGAA OP1 and OP2 were prepared for nanopore sequence respectively.

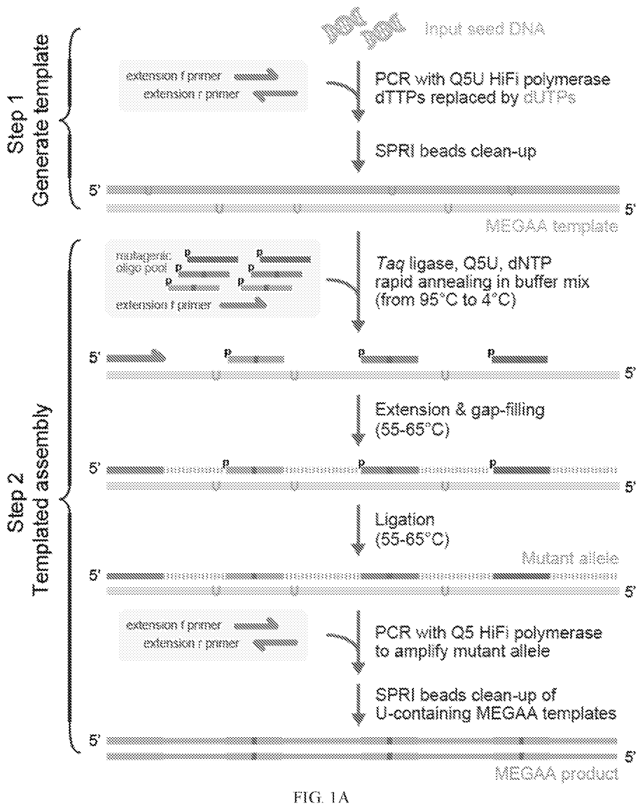

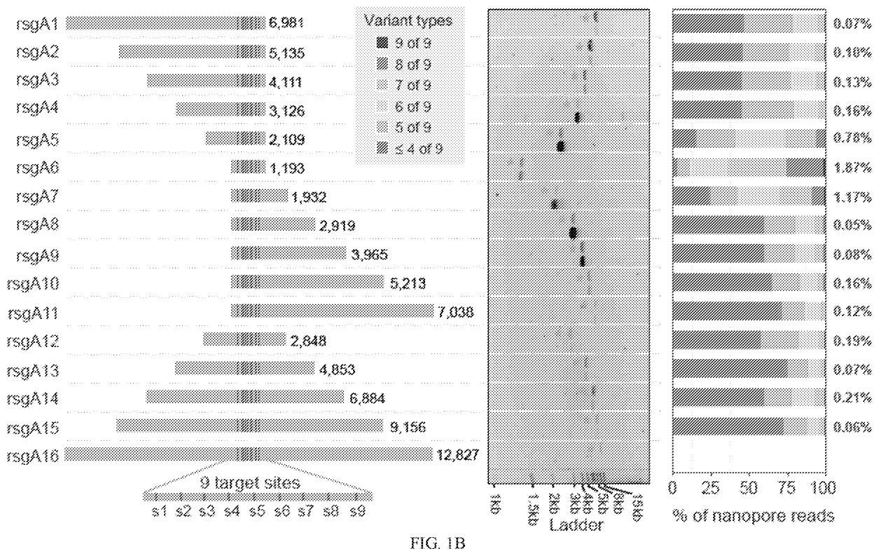

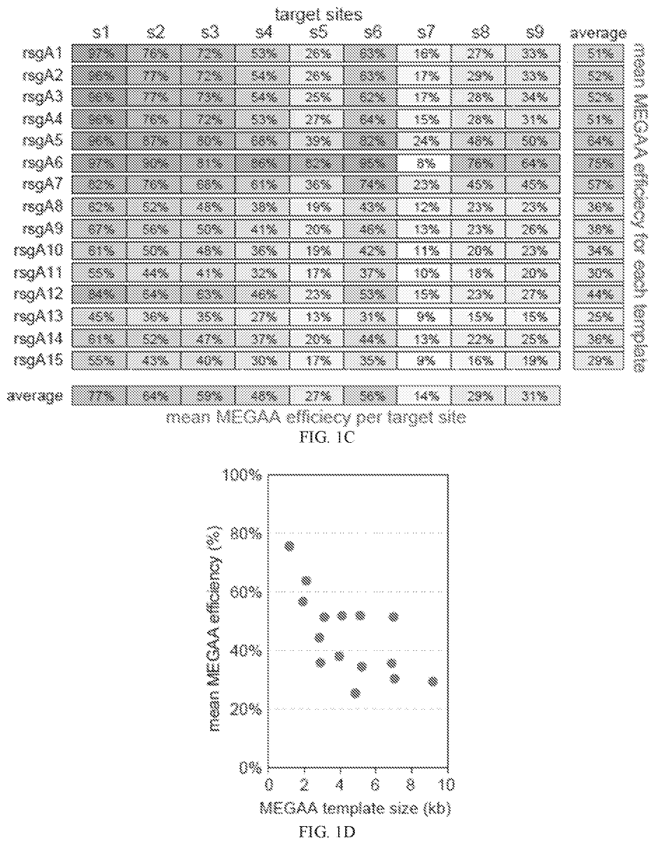

Comparison of the mutagenesis efficiency of MEGAA and commercial kit experiments MEGAA does not require the template to already be cloned into a circular plasmid DNA. Since circular plasmids are required for all these other methods as input, target plasmids (pJET1.2-rsgA6) were first generated by cloning the linear DNA fragments (rsgA6 template in FIG. 1B) into pJET1.2/blunt (Thermo Scientific #K1231). Mutagenesis was then performed according to the manufacturer's instructions. Subsequently, the mutagenesis efficiency of different methods was assessed by Nanopore sequencing. In brief, transformation was performed for mutated products and all colonies were scraped from plates and pooled together. On average, more than 500 colonies were obtained for commercial kits. Targeted 1.2 kb fragments were then amplified from the pooled colonies using uniquely barcoded primers targeting plasmid backbone region to avoid amplifying endogenous rsgA gene in E. coli genome. Finally, amplified products were subjected to gel examination and desired bands were excised for Nanopore sequencing.

SARS-CoV-2 S mutagenesis experiment U-containing S gene templates were PCR amplified using pcDNA3.1 SARS-CoV-2 S D614G plasmid40 as the DNA template with primers SARS-CoV-2 S_tempF and SARS-CoV-2 S_tempR Mutagenesis of the SARS-CoV-2 S was performed via a MEGAA reaction with the modification that primer lengths were adjusted to ensure ordered oligo annealing. Mutagenic oligos containing target codons were designed to generate all representative variants from alpha to lambda variants. Meanwhile, oligos containing degenerate bases (NNS) were designed to generate all combinations based on B.1.617.2 and AY.2 variants. Finally, thirty-three MEGAA reactions were carried out with sixty-four defined oligos and ten degenerated oligos (Table 1). All variants were prepared for Nanopore sequencing after cleaning up with SPRI beads.

Genome recoding mutagenesis experiment Approximately 36 kb DNA chunks in the E. coli K-12 genome were randomly chosen to be recoded with synonymous mutation DNA by compressed redundant codons (TTA→CTC, TTG→CTA, AGA→AGA, AGG→CGA, TCG→AGC, TCA→AGT). The DNA chucks were split into ten fragments with 17-54 bp overlaps. Ten paired primers 36K-F1/R1 to 36K-F10/R10 were designed and applied to amplify 10 U-containing DNA templates respectively. Meanwhile, 289 mutagenic oligos were designed with an ordered oligo annealing strategy to cover 1,015 mutated bases in the 36 kb DNA. Then 10 oligo pools, which contained 14 to 40 mutagenic oligos per pool rather than individual oligos were synthesized for MEGAA reactions. Following the MEGAA reaction steps, nanopore sequencing was applied to verify the recoding products.

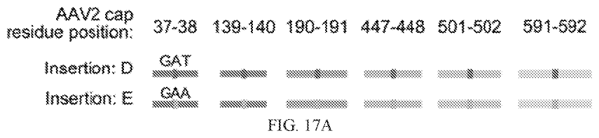

AAAV cap mutagenesis experiment U-containing DNA of the wildtype barcoded AAV2 cap gene were generated by two round PCR amplifications of the pRC2-mi342 vector (Takara Bio Inc, Cat. #6230) with primers AAV2-tempF/AAV2-tempR1 and AAV2-tempF/AAV2-tempR2. (FIG. 17) AAV2-tempR1 and AAV2-tempR2 included 12 bp random barcoding DNA. 12 oligos were designed to carry D or E insertions in the six variable regions (VR), VR-I to VR-VI, which positions were 35-40, 132-152, 188-192, 445-460, 490-500, 576-596 in capsid protein respectively. After oligo phosphorylation, MEGAA reactions were carried out with the oligo pool covering six variable regions. All cap variants were assessed on nanopore sequencing. The pAAV-CMV vector was linearized using EcoRI and BamHI restriction enzymes. Unique barcoded wildtype cap gene and MEGAA variants were digested using EcoRI and BamHI. The two products were purified using SPRI beads, ligated using T4 DNA ligase (New England Biolabs, Cat. #M0202M), and incubated at 16° C. overnight. A pooled plasmid was transformed into competent NEB Turbo cells and grown for 10 hours at 37° C. Individual colonies were picked for colony PCR with Oxford Nanopore sequencing barcoded primers. Indexed amplicons were pooled equimolar and sequenced on Oxford Nanopore platform to identify mutations sites as well as barcode sequences.

Virus was produced using AAVpro® Helper Free System (Takara Bio Inc, Cat. #6230), with minor adjustments. Briefly, a 150-mm cell culture dish (Thermo Scientific™ 150468) was inoculated with 6.0×10⁶ 293T cells in DMEM culture medium supplemented with 1× GlutaMAX™, 1× Pen/Strep antibiotic, and 5% FBS according to standard cell culture protocols. The 293T cells were split into 10×150-mm cell culture dishes for the experiment when cells were approximately 90% confluent. Two days after splitting the cells, PEI transfection was performed with PEI:DNA mass ratio of 3:1 with 36 μg pR2-mi342, 70 μg pHelper, and 0.25 μg pool variants, which included 125 unique barcoded pAAV-CMV-aav2cap variants along with 24 barcoded cap wildtype plasmids. The culture medium was completely replaced with fresh DMEM containing 1× GlutaMAX™, 1× Pen/Strep antibiotic, and 5% FBS at 12 hours after transfection. 50% media volume (200 mL) was added after 72 hours. After 5 days, isolation of AAV2 particles from AAV-producing cells was performed according to the AAVpro® Helper Free System instructions. Nuclease treatment was performed by adding 1/100 volume of 1 M MgCl₂ solution to the supernatant mixture obtained from AAVpro® Helper Free System and along with TURBO™ DNase (Thermo Scientific™ Cat. AM1907) to a final concentration of 0.4 U/μl. The supernatant was collected after 5,000×g centrifuge for 10 minutes at 4° C. and run purification of desalting and concentration of the AAV2 particles based on the protocol of AAVpro® Purification Kit (Takara Bio Inc, Cat. #6232).

To evaluate packaging efficiency of different variants, barcode regions of variants for input plasmids and virus particles were quantitatively amplified and sequenced on the NextSeq platform. Briefly, 1 μL purified AAV2 particles (1×108 GC/μL) and input plasmids pool were first subjected to a 12-cycle PCR amplification using AAV_bcRead primer pairs and SPRI beads cleanup to generate amplicon of barcodes regions. Next, a quantitative PCR reaction was performed to add indexed Illumina TruSeq adapters to the amplicon and advanced to the final extension step during exponential amplification. Yielding libraries were then purified by gel electrophoresis and sequenced on the Illumina NextSeq platform (2×75 paired-end mode) with 20% PhiX spike-in (Illumina FC-110-3001) according to manufacturer's instruction.

Raw sequencing reads of variants barcode amplicon were analyzed by in-house script to calculate the variants packaging efficiency. Briefly, barcode sequences of the variants were extracted from reads and matched to variant identity based on references from Nanopore sequencing. Reads mapped to each variant were then counted and the relative abundances were calculated as: RA (variant-X)=reads count mapped to variant-X/total mapped reads count Next, variant relative abundances between input plasmid and yielding virus particles were compared to quantify packaging efficiency:efficiency (variant-X)=RA (variant-X) in virus pool/RA (variant-X) in plasmids pool. The efficiency was then normalized by WT variants to generate final variant packaging efficiency used in downstream analysis normalized efficiency (variant-X)=efficiency (variant-X)/average of efficiency (WT variants). To explore the determinants of AAV2 packaging efficiency, a linear regression model was constructed in R 4.1.2 to predict packaging efficiency based on the binarized mutation profile of all 125 variants, and predicted efficiency as well as coefficients of mutation sites were extracted from the linear model to evaluate the overall performance and combinatorial effect of each site.

Nanopore sequencing and data analysis To determine overall variant generation efficiency, a barcoded Oxford Nanopore strategy was implemented to sequence the full length of generated variants. Briefly, unique dual 12-bp barcodes were added to both ends of the variants by PCR amplification, and yielded barcoded variants were pooled together and purified by gel electrophoresis. Approximately 300 fmol pooled variants after cleanup were subjected to Oxford Nanopore library preparation and sequencing following the manufacturer's instructions. Variants underwent Nanopore sequencing using the protocol ‘Amplicons by Ligation (SQK-LSK110)’ (Oxford Nanopore Technologies). Both MinION Flow Cell R9.4.1 (FLO-MIN106D) and R10.4 (FLO-MIN112) were used for sequencing on a MinION with the MinION software v.20.06.0 (ONT). Base-calling was performed with Guppy v.3.6.0 (ONT) in GPU mode. Full-length reads were first demultiplexed based on barcodes of both ends using an in-house Python script and subjected to quality filtering to only keep high-quality reads (no more than 3-bp mismatches and 1-bp gap in 20-bp region of both 5′ and 3′ ends). Demultiplexed reads were then aligned to reference sequence by MUSCLE41 v3.8.31 using default setting. Variant generation efficiency was then calculated based on reads alignment using an in-house Python script. In-house scripts used for Oxford Nanopore sequencing data analysis can be accessed at github.com/hym0405/MEGAAdt.

Analytical model of MEGAA cycling process Using a binomial distribution, the completeness of MEGAA reactions (C_{N}) at MEGAA cycle N can be predicted with the average oligo incorporation efficiency per locus (μ) through the formula C_{N}=1−(1−μ). The completeness C_{N} metric indicates the fraction (or % completeness) of all target sites mutated in the end-product mix with 1.0 meaning 100% of products have 100% mutations at all target sites. In this model, a C_{N} of 0.5 could mean that either 50% of products have all sites mutated or 100% of products have half of their sites mutated. Mapping the experimental data to this simple model gives an estimated average oligo incorporation efficiency μ of 0.8 to 0.9 for Design-2 oligos (i.e., 80-90% mutagenesis efficiency per site per MEGAA round), compared to 0.5 to 0.7 for Design-1 oligos (50-70% mutagenesis efficiency).

MEGAA mutagenesis protocol The MEGAA protocol was performed first by using a DNA seed (e.g., E. coli genomic DNA, pcDNA3.1 SARS-CoV-2 S D614G DNA, pRC2-mi342 Vector containing AAV2 cap gene) to generate an uracil-containing template, then by performing rounds of denaturing, ligation, and extension steps, followed by a final amplification step. Details steps are as follows.

### MEGAA Reaction Mixes and Buffers

| 10X MEGAA reaction enzyme mix | 2X MEGAA reaction master mix buffer |
|---|---|
| 50 mM potassium acetate | 1.7x Taq DNA Ligase Reaction Buffer (NEB# |
| | B0208SVIAL) |
| 20 mM tris-acetate | 1.7x Q5U Reaction Buffer (NEB# |
| | B9037SVIAL) |
| 10 mM magnesium acetate | 1.7x CutSmart Buffer |
| 200 μg/mL BSA | 0.17 mM dNTPs |
| 1 mM DTT | 0.33 mM Nicotinamide adenine dinucleotide |
| 50% glycerol | 6.67 mM DTT |
| 10 U/μL Taq DNA Ligase (NEB# M0208L) | 16.7% DMSO |
| 0.1 U/μL Q5U Host Start High-Fidelity DNA | pH 7.2 at 25° C. |
| Polymerase (NEB# M0515SVIAL) | |
| pH 7.4 at 25° C. | |

### MEGAA Reactions

Step 1: PCR generation of uracil-containing template and clean up To amplify the uracil containing template 1 μL of DNA template (e.g., 1 ng/μL E. coli genomic DNA) or diluted MEGAA product from last round of reaction, 1 μL of forward primer (20 μM), 1 μL of reverse primer (20 μM), 1 μL of 10 mM dNTPs (dATP, dCTP, dUTP and dGTP at 2.5 mM each, NEB #N0446S, #N0459S), 10 μL of 5×QSU® reaction buffer, 1 μL of Q5U® hot-start high-fidelity DNA polymerase, and 35 μL of nuclease-free water (Invitrogen #AM9937) were incubated under a PCR protocol comprising an initial denaturation step (98° C. for 30 seconds), 30 amplification cycles (98° C. for 10 seconds, 55-72° C. for 10 seconds (use of the NEB Tm Calculator highly recommended to determine temperature), 72° C. for X minutes (20-30 seconds/kb, and final elongation (72° C. for 5 min).

SPRI beads were used for the purification of the amplified uracil-containing template. Resuspended beads were added to the DNA sample at a 1× ratio of suspended SPRI beads to the uracil-containing template DNA and mixed. The mixture was incubated for 5 minutes at room temperature followed by separation of the supernatant from the beads (e.g., centrifugation and pelleting on a magnet) and removal of the supernatant. The pelleted beads were washed twice with 500 μL of freshly prepared 80% ethanol in nuclease free water without disturbing the pellet. Following second washing the beads were re-pelleted (e.g., centrifugation and pelleting on a magnet), residual ethanol was removed and the pellet was air dried but not to the point of cracking The pellet was resuspended 20-30 μL nuclease-free water and incubated for 2 minutes at room temperature. The beads were pelleted (e.g., centrifugation and pelleting on a magnet) and the eluate was retained and transferred to a new tube.

Step 2: Mutagenesis using MEGAA Oligos were prepared for phosphorylation (optional step for MEGAA cycling) by adding 100 μM of the oligos to 2.5 μL T4 DNA ligase buffer (10×), 5 μL T4 kinase (PNK) (NEB #M0201L), 2.5 μL PEG 8000 (50%) (Thermo Scientific™ #50-488-949) and enough nuclease-free water to reach 25 μL total. The reaction mixtures were incubated at 37° C. for 60-90 minutes followed by an incubation at 65° C. for 20 minutes to heat inactivate.

For one-pot MEGAA mutagenesis 0.5 pmol extension primer (non-phosphorylated), 0.5 fmol uracil-containing template, 0.5 pmol individual mutagenic oligo or pool oligos (0.5 pmol each), 2 μL 10×MEGAA reaction enzyme mix, 10 μL 2×MEGAA reaction master mix buffer, and a quantity of nuclease-free water to 20 μL final volume were mixed and incubated in a MEGAA program (95° C. for 90 seconds denaturation, 4° C.′ for 60 seconds annealing, 55-65° C. for 3 minutes extension/ligation and 65° C. for 60-90 minutes final ligation).

Step 3: Amplification of MEGAA product To amplify the MEGAA product, 2 μL of MEGAA product, 1 μL of forward primer 20 μM, 1 μL of reverse primer 20 μM, 25 μL of 2×Q5® Hot Start High-Fidelity Master Mix (NEB #M0494L), 21 μL of nuclease-free water (Invitrogen) were mixed and amplified using a PCR protocol comprising an initial denaturation (8° C. for 30 seconds), 30 cycles (98° C. for 10 seconds, 55-72° C. for 10 seconds (use of the NEB Tm Calculator is highly recommended to determine precise temperature), 72° C. for X minutes (20-30 seconds/kb), and a final elongation of 72° C. for 5 minutes.

The resulting MEGAA product can be used in downstream applications. Additional MEGAA cycling can be performed if the efficiency was not ideal. MEGAA cycling comprises repeating MEGAA Steps 1-3 with 1:40000 diluted product as input to minimize the contamination in uracil-containing template.

### MEGAAtron Automation System Components and Operation

MEGAAtron template generation master mix (49.5 μL in total) comprises 1 μL of forward primer 5 μM (5 μmol), 1 μL of reverse primer 5 μM (5 μmol), 1 μL of 10 mM dNTPs (dATP, dCTP, dUTP and dGTP at 2.5 mM each), 10 μL of 5×QSUR reaction buffer, 1 μL of Q5UR hot-start high-fidelity DNA polymerase, and 35.5 μL of nuclease-free water.

MEGAAtron reaction master mix (2.8 μL in total) comprises 0.2 μL of extension primer 0.5 μM (non-phosphorylated), 0.4 μL 10×MEGAA reaction enzyme mix, 2 L 2×MEGAA reaction master mix buffer, and 0.2 μL of nuclease-free water.

MEGAAtron amplification master mix (46 μL in total) comprises 2 μL of forward primer 20 μM (40 μmol), 2 μL of reverse primer 20 μM (40 μmol), 25 μL of 2×Q5® Hot Start High-Fidelity Master Mix, and 17 μL of nuclease-free water.

Step 1: PCR generation of uracil-containing template and clean up To amplify the uracil-containing template 0.5 μL of DNA template (e.g., 1 ng/μL. E. coli genomic DNA) or diluted MEGAA 1007 product from last round of reaction and 49.5 μL premade MEGAAtron template generation master mix are amplified in a PCR protocol comprising an initial denaturation (98° C. for 30 seconds), 30 cycles of 98° C. for 10 seconds, 55-72° C. for 10 seconds (use of the NEB Tm Calculator is highly recommended to determine temperature), and 72° C. for X minutes (20-30 seconds/kb), and a final elongation of 72° C. for 5 min.

SPRI beads were used for the purification of the amplified uracil-containing template. Resuspended beads were added to the generated template at a 1× ratio of suspended SPRI beads to the generated template and mixed. The SPRI cleanup used the same protocol as above, with reduced volumes, as scaled for PCR plates. Templates were eluted into 80 μL nuclease-free water, yielding 0.0005 UM uracil-containing template.

Step 2: Mutagenesis using MEGAA For one-pot MEGAA mutagenesis 2.8 μL premade MEGAAtron reaction master mix. 0.2 μL 0.0005 μM uracil-containing template, and 1 μL 0.1 μM corresponding oligos pool were transferred into each well and incubated in a MEGAA program (95° C. for 90 seconds denaturation, 4° C. for 60 seconds annealing, 55-65° C. for 3 minutes extension/ligation, and 65° C. for 60-90 minutes final ligation).

Step 3: Amplification of MEGAA product To amplify the MEGAA product, 46 μL premade MEGAAtron amplification master mix was added to the plate and amplified in a PCR protocol comprising an initial denaturation (98° C. for 30 seconds), 30 cycles of 98° C. for 10 seconds, 55-72° C. for 10 seconds (use of the NEB Tm Calculator highly recommended to determine temperature), and 72° C. for X minutes (20-30 seconds/kb), and a final elongation of 72° C. for 5 min.

The resulting MEGAA product can be used in downstream applications. Additional MEGAA cycling can be performed if the efficiency was not ideal. MEGAA cycling comprises repeating MEGAA Steps 1-3 with 1:40000 diluted product as input to minimize the contamination in uracil-containing template.

### Example 1

### Template Mediated Variant Synthesis

Mutagenesis by Template-guided Amplicon Assembly (MEGAA) uses a seed DNA material to generate an initial template for subsequent annealing, extension, and ligation of oligo pools that carry mutations of interest (FIG. 1A). The generated variant is then specifically amplified against the initial template to yield the final high-fidelity product. In the first step, the input seed DNA is amplified by PCR using a Q5U® hot start high-fidelity DNA polymerase where dTTPs are substituted with dUTPs. This results in MEGAA templates where all thymine (T) bases are replaced by uracil (U) bases. In the second step, the U-containing template is combined with Taq DNA ligase, QSU® hot start high-fidelity DNA polymerase, dNTP, and the desired mutagenic pool of oligos and a forward extension primer at 500 to 1,000-fold molar excess of the template as a single-pot reaction in a compatible buffer. Then oligo annealing, extension, and ligation reactions proceed. Since the Q5U® polymerase does not exhibit strand displacement activity nor 5′ to 3′ exonuclease activity, once mutagenic oligos are annealed to the template, the polymerase will only gap fill between oligos and allow subsequent ligation by Taq DNA ligase. Rapid oligo annealing is performed from 95° C. down to 4° C.′ at a rate of 3° C./sec. Fast annealing with excess oligos avoids renaturation of the U-template DNA. Furthermore, this prevents Taq ligase from unwarranted ligation before the single-stranded variant allele is fully gap-filled. The assembled single-stranded variant allele, which has incorporated the mutagenic oligos, is then amplified by PCR using a Q5® hot-start high-fidelity DNA polymerase that cannot extend off of U-containing templates. Archaeal polymerases such as Q5 bind tightly to uracil nucleotides, which stall DNA polymerization. Q5U® is a modified Q5 DNA polymerase that contains a mutation in the uracil-binding pocket to enable amplification of templates containing uracil and inosine bases. This enables specific amplification of the variant amplicon from the MEGAA reaction for direct downstream applications (e.g., cloning, sequencing, or transformation).

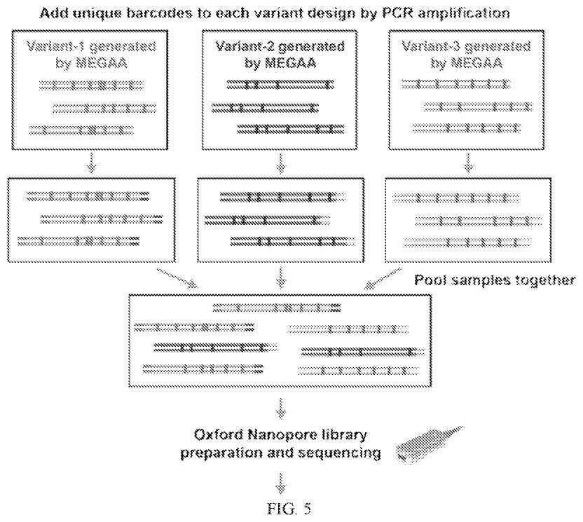

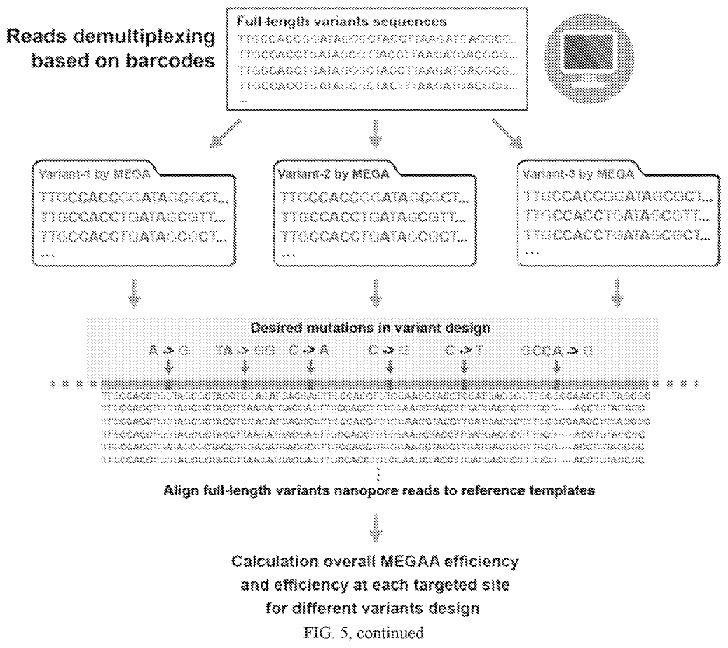

To accurately and rapidly analyze the full-length MEGAA products in parallel, a low cost long-read sequencing pipeline was developed using the Oxford Nanopore MinION platform with a PCR barcoding scheme that allowed multiplexing of up to 96 samples per run (FIG. 5). A custom variant pipeline was used to assess MEGAA efficiency across target sites. With this setup, MEGAA products can be analyzed cost-effectively (approximately $2.90 per sample), with sufficient accuracy for unique variant identification, and with a reduced turnaround time (from 2 days by Sanger sequencing to only approximately 2 hours).

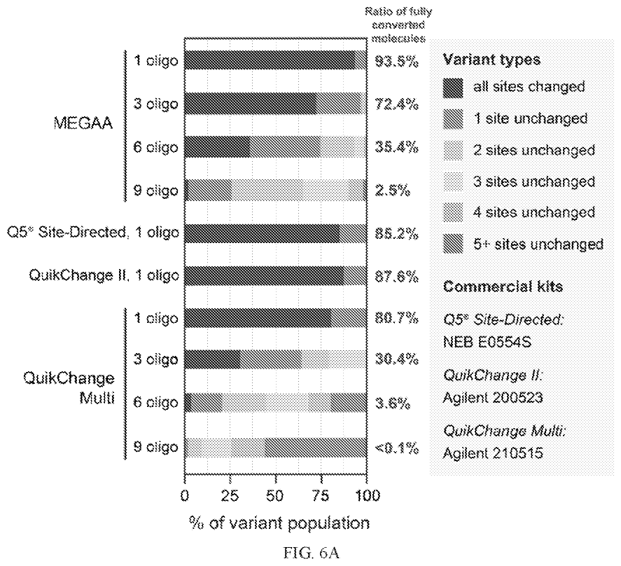

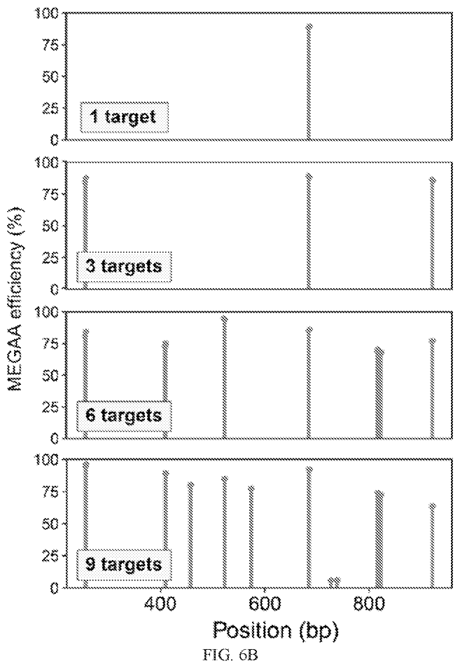

The ability of MEGAA to generate variants was piloted using oligo pools containing 1, 3, 6 or 9 oligos for a 1,192 bp DNA template (rsgA gene from E. coli K-12), with each oligo (20-39 nt) containing a 2-5 base substitution of the template sequence (Table 1). The efficiency and completeness of each variant synthesis by MEGAA was assessed by nanopore sequencing. Variants from oligo pools containing 1 oligo were generated at greater than 90% efficiency, while greater than 70% were generated completely in a 3-pool reaction, 35% were generated completely in a 6-pool reaction, and 2.5% were generated completely in a 9-pool reaction with approximately 25% of variants having 8 or 9 mutations. (FIG. 6A). In larger oligo pools (e.g., 9-pool), targets near the 5′ region were generally more efficiently converted than those at the 3′ region (FIG. 6B). A head-to-head experiment was included to compare MEGAA efficiency with other commercial directed mutagenesis kits. For single-site mutagenesis, MEGAA efficiency was higher than commercial kits, (93.5% for MEGAA versus 85.2%, 87.6%, and 80.7% for Q5® Site-Directed Mutagenesis Kit, QuikChange II Site-Directed Mutagenesis Kit, and QuikChange Lightning Multi Site-Directed Mutagenesis Kit, respectively) (FIG. 6A). Importantly, MEGAA was substantially more efficient than the commercial kits in multiplex target reactions. While only 3.6% of sequences were completely mutated for a 6-oligo pool reaction in the QuikChange Lighting reaction, 35.4% of sequences in MEGAA were completely mutated.

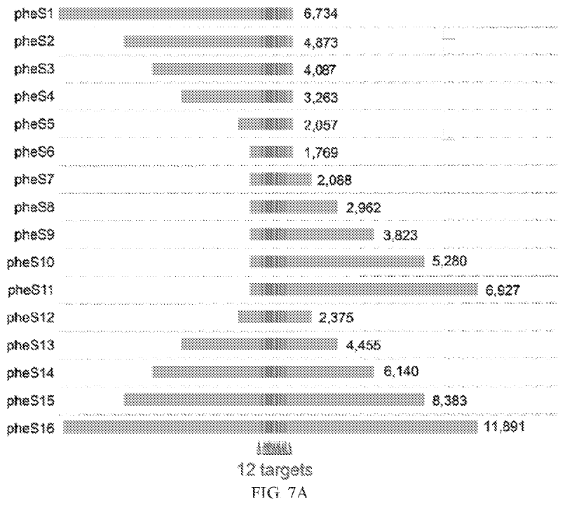

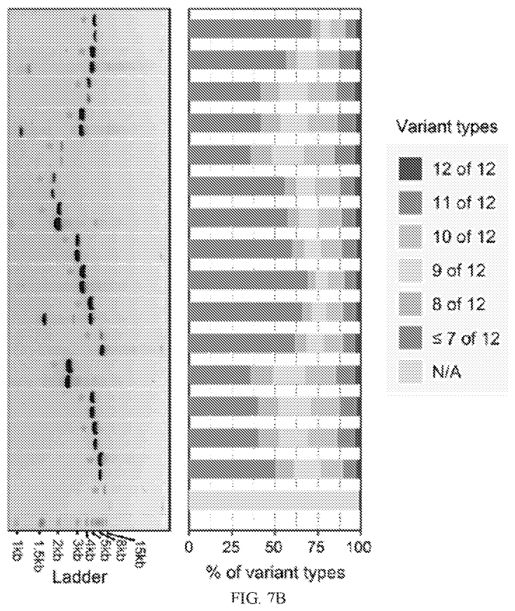

Next, the capacity of MEGAA to work on templates of different sizes ranging from 1 kb to 13 kb was tested. Sixteen U-templates of different sizes (rsgA1-rsgA16) were generated by amplifying the rsgA gene region of the E. coli K-12 genome. Then, separate MEGAA reactions were performed using the same 9-oligo pool designed against a shared 1 kb region across the different sized U-templates (FIG. 1B). In general, MEGAA products had robust amplicon bands at the expected sizes. Templates larger than 10 kb (e.g., rsgA16) did not produce a detectable amplicon. From nanopore sequencing of MEGAA products, varying levels of completeness in MEGAA product yield were observed, with more than 40% of all products having at least S of 9 targets converted for almost all templates (FIG. 1B). The mean MEGAA efficiency across all target sites reached as high as 75% for the rsgA6 template (1,192 bp) and as low as 29% for the rsgA15 template (9,156 bp) (FIG. 1C). Target-specific differences in MEGAA efficiency were observed that were consistent across all template sizes. In general, 5′ targets were more efficiently generated than 3′ targets, suggesting global factors governing oligo assembly (FIG. 1C). Targets s5 and s7 were less efficiently generated than expected, which implies that local oligo annealing factors are also at play. Overall, the size of the template correlated with MEGAA efficiency, with shorter templates more efficiently converted (FIG. 1D). To verify that these results hold true for another template, the experiment was repeated on 16 additional templates (pheS1-pheS16) derived from the E. coli K-12 genome near the pheS gene using a 12-oligo pool. The same trends were observed-most target sites were made at high efficiency with some variation in some targets (FIG. 7). Together, these findings indicated that MEGAA is efficient and multiplexable across different templates of kilobases in length and can be amenable for further improvements.

### Example 2

### Optimization of Variant Synthesis and Iterative Cycling

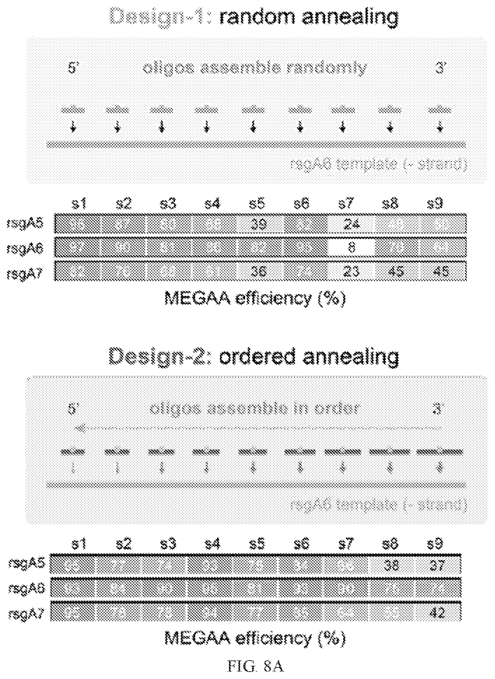

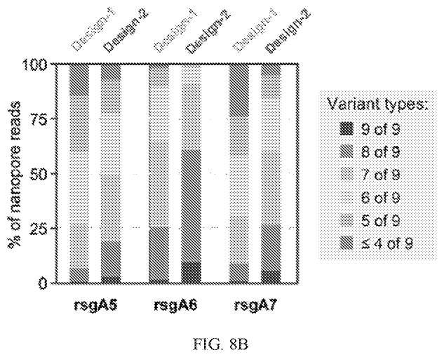

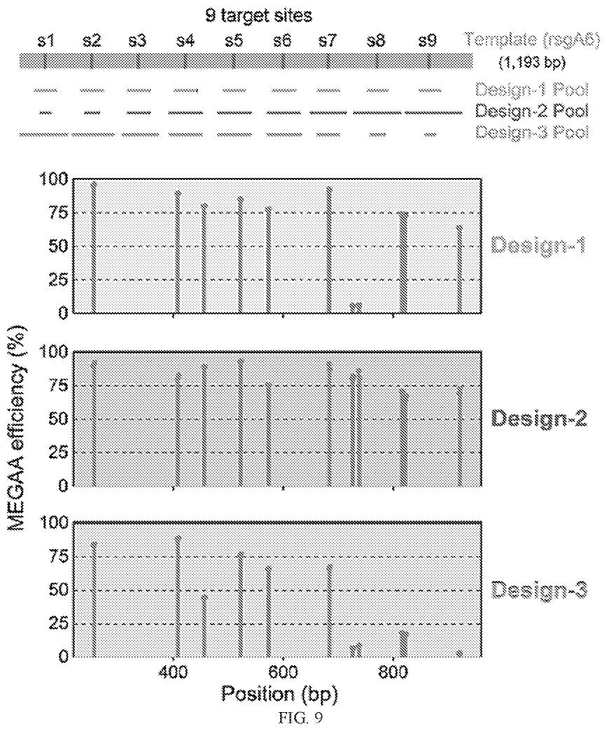

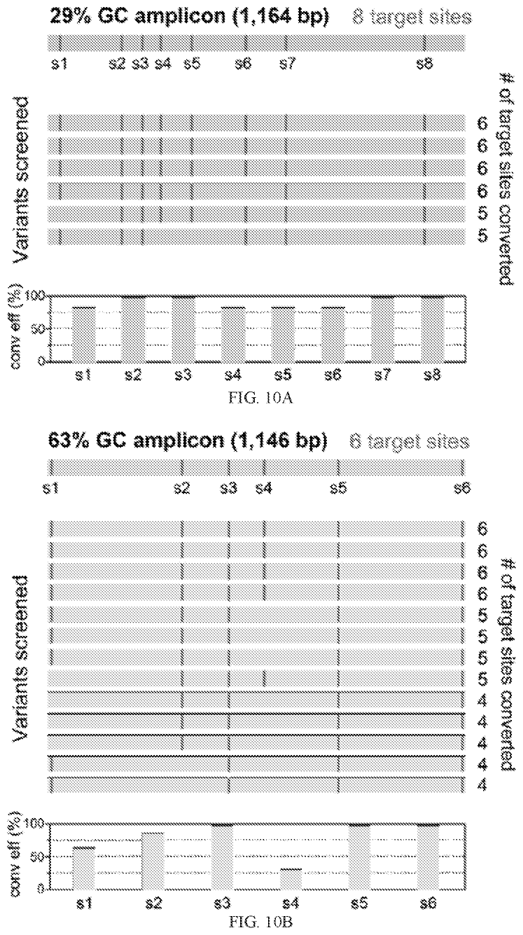

The reduced MEGAA efficiency near the 3′ region of templates was hypothesized to be due to extension of the template without having the oligos annealed in their proper place. Therefore, a strategy was devised where oligos were designed to have a gradation of melting temperatures (Tm), with 5′ oligos having the lowest Tm (47° C.) and 3′ oligos having the highest Tm (64° C.) (FIG. 8). This strategy should support a more ordered assembly process whereby 3′ oligos first anneal to the U-template before 5′ oligos, which would increase the likelihood of generating a more fully converted variant. A head-to-head comparison of this new oligo design (Design-2) with the prior oligo design (Design-1) was performed where all oligos had the same Tm. With Design-2 oligos, the resulting MEGAA variants had a notably improved mean MEGAA efficiency of 86% per target (versus 75% for Design-1), with nearly all target positions performing better, specially s7 (FIG. 8). Also tested was the opposite oligo design (Design-3) with 5′ oligos having the highest Tm and 3′ oligos having the lowest Tm, which yielded even lower oligo incorporation at the 3′ region and thus further confirmed the oligo design principle (FIG. 9). Using the Design-2 strategy, MEGAA could operate on templates with GC contents ranging from 29% to 63%, yielding mean conversion rates per target of 91.7% and 81.0% respectively (FIG. 10). To characterize off-target mutations in MEGAA products, products were cloned into shuttle vectors, transformed them into cells, and isolated selected colonies for Sanger sequencing, which did not reveal any additional mutations outside of MEGAA target sites.

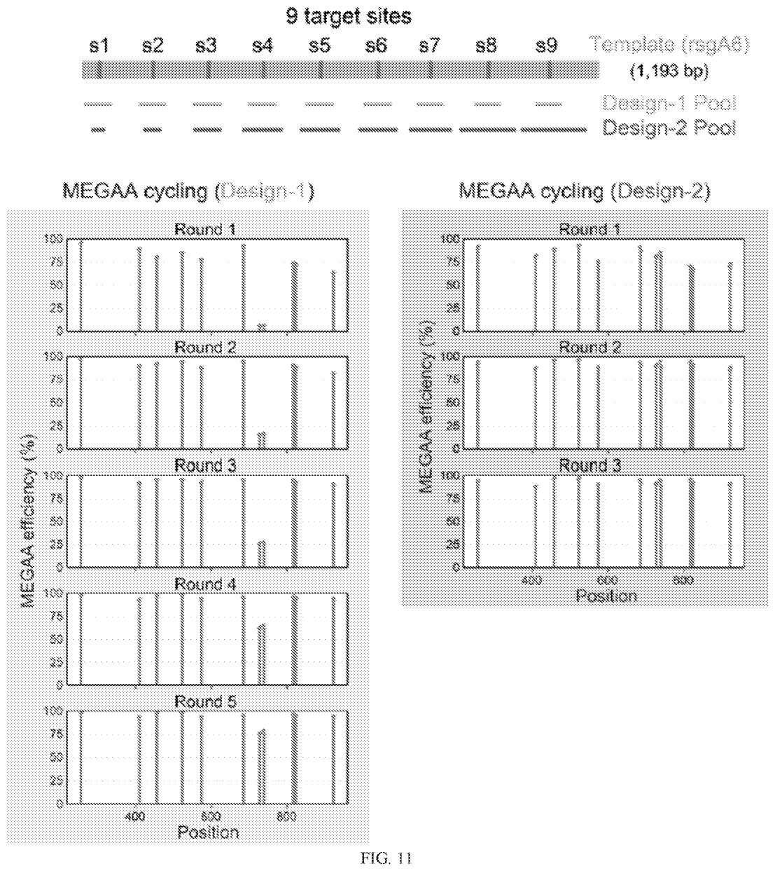

Conceptually, MEGAA could be repeatedly cycled such that the output from one round is used as the direct input of the next round, which could further enhance MEGAA product conversion towards the target genotype (FIG. 2A). Using the rsgA6 template and the Design-1 or Design-2 for 9-oligo pools, a protocol was developed and tested whereby the MEGAA product from the prior round is reamplified into U-containing templates for the next round of MEGAA reactions without any laborious cell transformation nor clonal purification steps. For Design-I oligos, as more MEGAA rounds are performed, the fraction of fully converted variants increased, reaching near completion after the 5th round (FIGS. 2B and 11). For Design-2 oligos, the desired variant was almost completely generated after just 2 or 3 rounds, highlighting the substantially improved performance using the more optimized oligo design. Importantly, the conversion state of the variant product over multiple MEGAA cycles can be modeled using a simple binominal distribution. For Design-2 oligos, the experimental data matched the model prediction of an overall MEGAA efficiency per site between 80-90% per cycle, while Design-1 oligos gave a more varying MEGAA efficiency between 50-70% (FIG. 2C). Therefore, MEGAA can be computationally modeled and experimentally tuned to generate variants of different levels of mutational saturation across a population.

### Example 3

### Automated Construction of DNA Sequence Variants

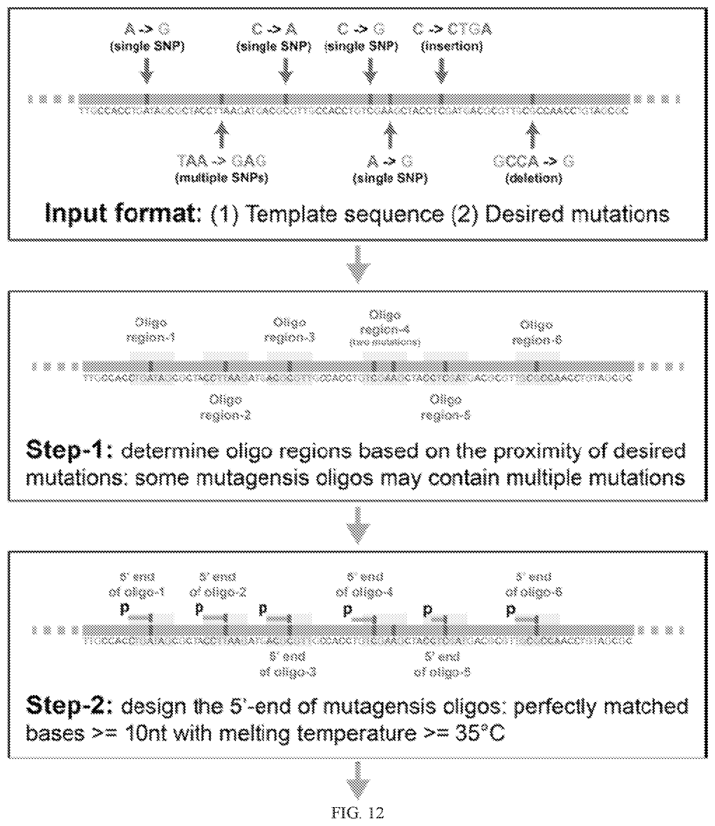

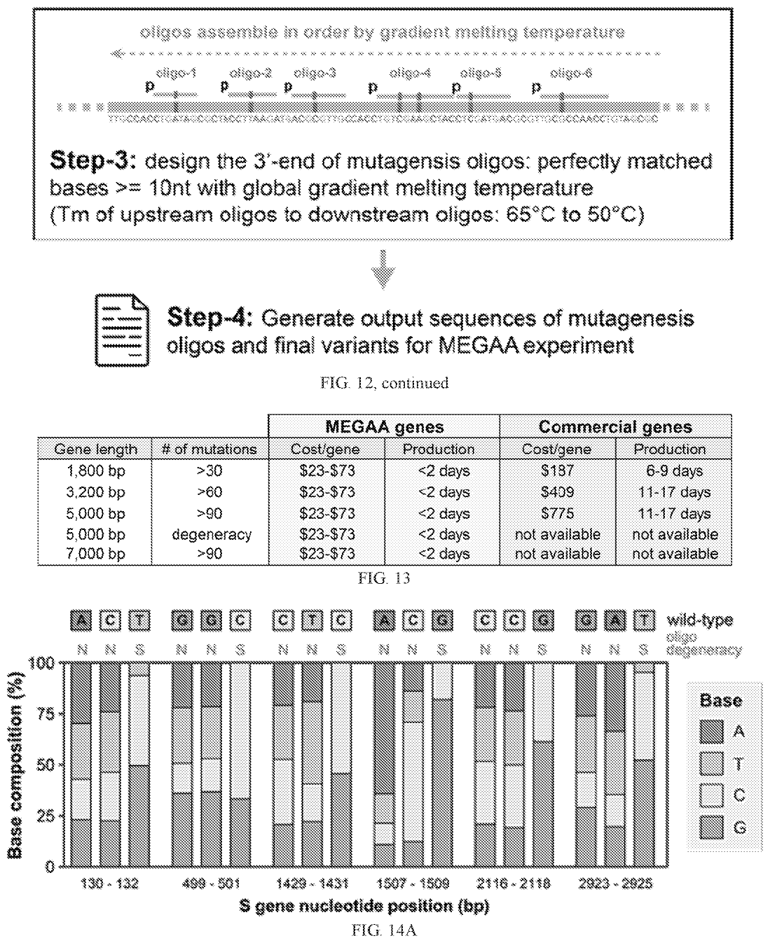

To generalize and standardize the variant synthesis platform, a low-cost open-source liquid handling and nucleic acid amplification workstation (Opentrons OT-2) was used to execute MEGAA reactions in an automated end-to-end pipeline dubbed MEGAAtron (FIG. 2D). First, a MEGAA design tool (MEGAA-dt) was developed to generate sequences of mutagenesis oligos based on input templates and desired changes by automatically optimizing for high MEGAA efficiency and low resource requirements (FIG. 12). MEGAA oligos are ordered from commercial vendors individually or as premixed pools. Reagents, templates, and oligos are loaded onto the MEGAAtron robotic system, which can produce 24 different variants in a single run, including all steps of the protocol through a MEGAA round (or multiple rounds) from PCR amplification to product purification (FIG. 2D). The resulting MEGAA products are assessed by nanopore sequencing for quality control and efficiency characterization. The overall turnaround time of the pipeline once all inputs are ready (e.g., oligo pool, initial template) is less than 6 hours with a cost ranging from approximately $20 per variant (depending on variant type) including oligos, consumables, and sequencing, which is 10 times cheaper than commercialized gene synthesis (FIG. 13). To obtain truly 100% clonal variants, an additional cloning step can be performed, and a minimal amount of colony sequencing used to identify the desired variant based on nanopore sequencing analysis (e.g., 3 out of 4 colonies picked are expected to contain the perfect variant from nanopore reads).

### Example 4

### Gene and Genome-Scale Templated Variant Synthesis

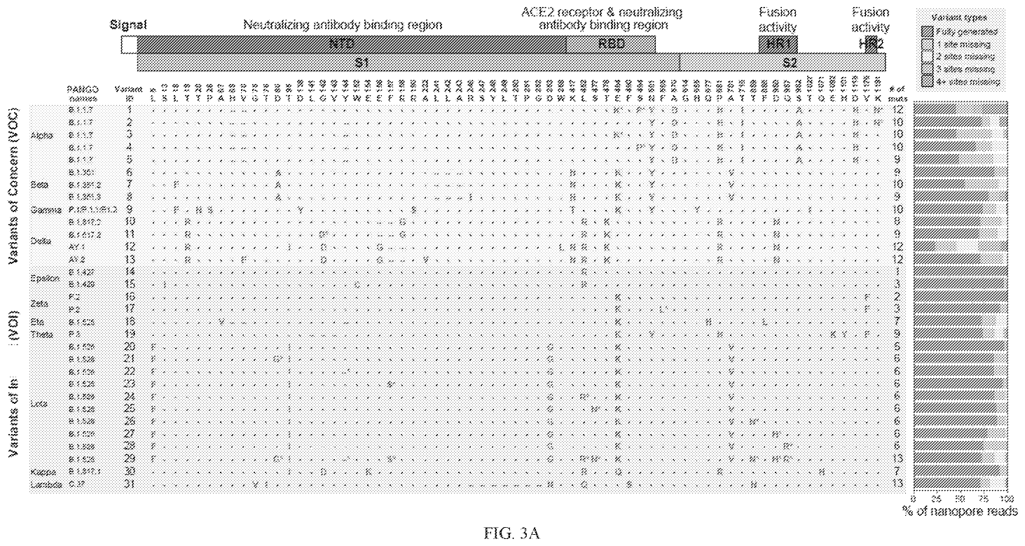

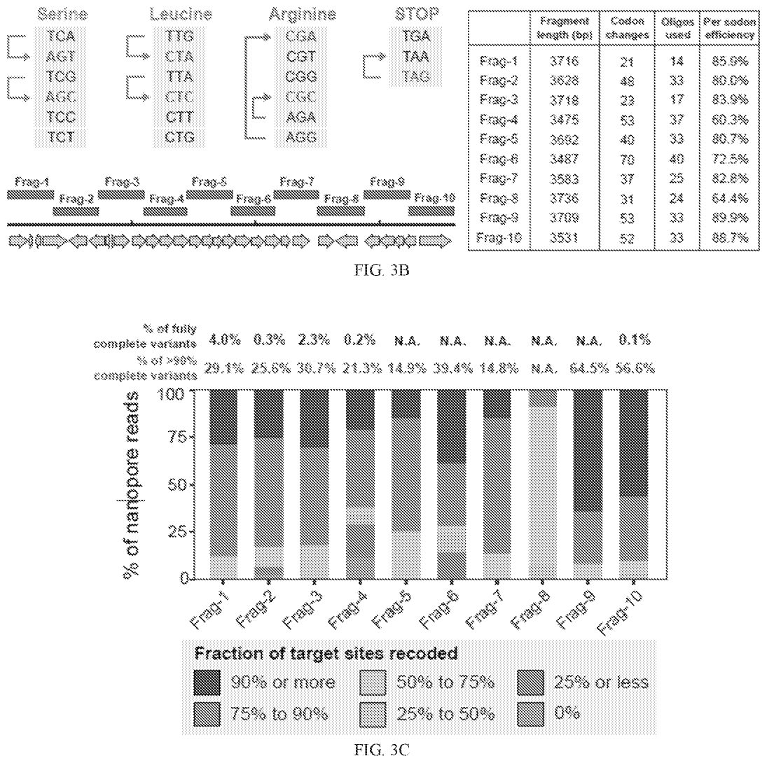

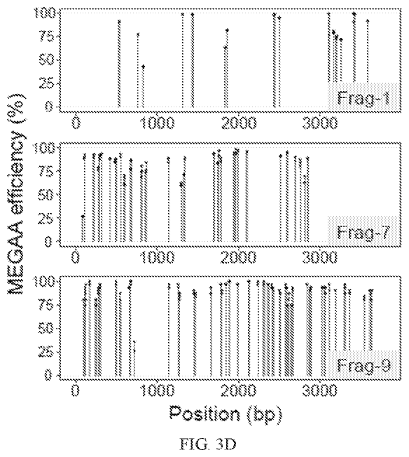

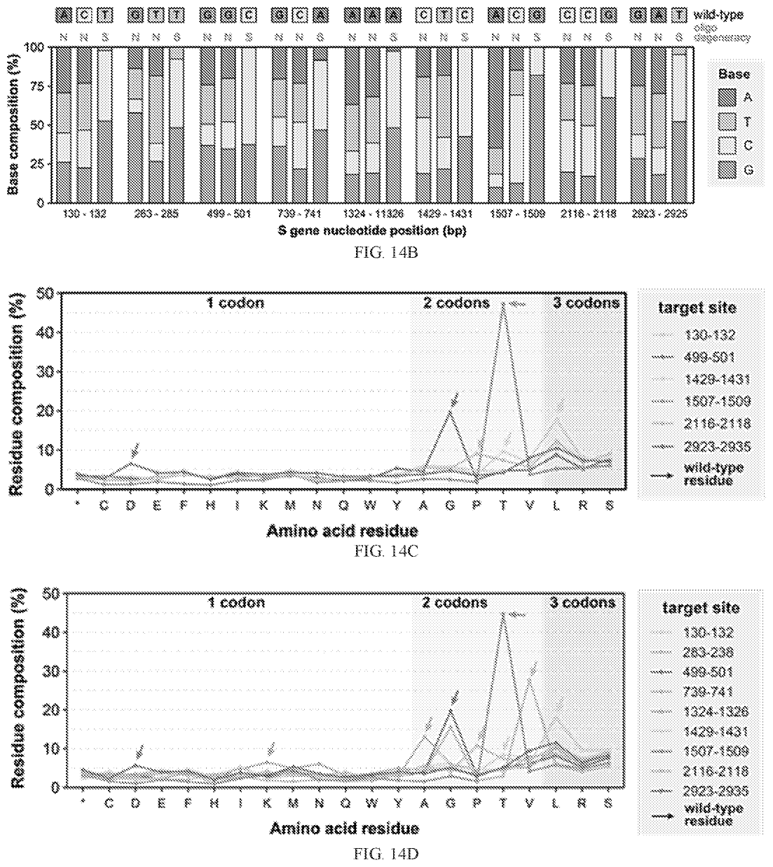

Fast variant production of key viral components can facilitate the testing of neutralizing antibodies and therapies against variants and help establish zoonotic transmission paths for better pandemic preparedness. The 3,822 bp S gene that encodes the Spike protein from the SARS-CoV-2 virus, which has been extensively characterized by surveillance sequencing during the ongoing global pandemic since late 2019, was chosen. A set of 31 representative natural S gene variants was assimilated from different SARS-CoV2 lineages from around the world, encompassing major variants of interest (VOI) and variants of concern (VOC) as of fall 2021 (FIG. 3A) Across the 31 S gene variants, 66 unique mutations are present, with some variants containing up to 13 substitutions and deletions. Oligos were commercially synthesized for each target site and separately pooled to produce their respective variants on the MEGAAtron system. Within 12 hours and two round of MEGAA reactions, all S gene variants were successfully generated to a high degree of saturation as assessed by full-length nanopore sequencing (FIG. 3A). In 27 of 31 variants, the correct complete variant sequence was observed in greater than 50% of single-molecule reads from nanopore sequencing. This means that 1 of every 2 molecules in each MEGAA product had the perfect sequence, confirmed by Sanger sequencing of select cloned variants. Furthermore, for 14 variants with residue substitutions, 89.3±8.6% of the nanopore reads were fully mutated. Of the 17 deletion-containing variants, 65.5±16.6% showed fully mutated nanopore reads. Notably, variant ID31, which contains a 21 bp deletion along with 6 separate residue substitutions, exhibits complete variant generation in 70% of the reads, thus demonstrating the versatility of the method in making different mutation types. Beyond defined variants, the generation of complex variant populations by MEGAA was explored using oligos with degenerate bases to target multiple sites. Using a 6-pool or a 9-pool oligo set with NNS base degeneracies on the B.1.617.2 (Delta) S gene template, complex yet even variant pools estimated to contain greater than 1E9 and greater than 3.5E13 unique sequences for the 6-pool and 9-pool MEGAA reactions were produced, respectively (FIG. 14).

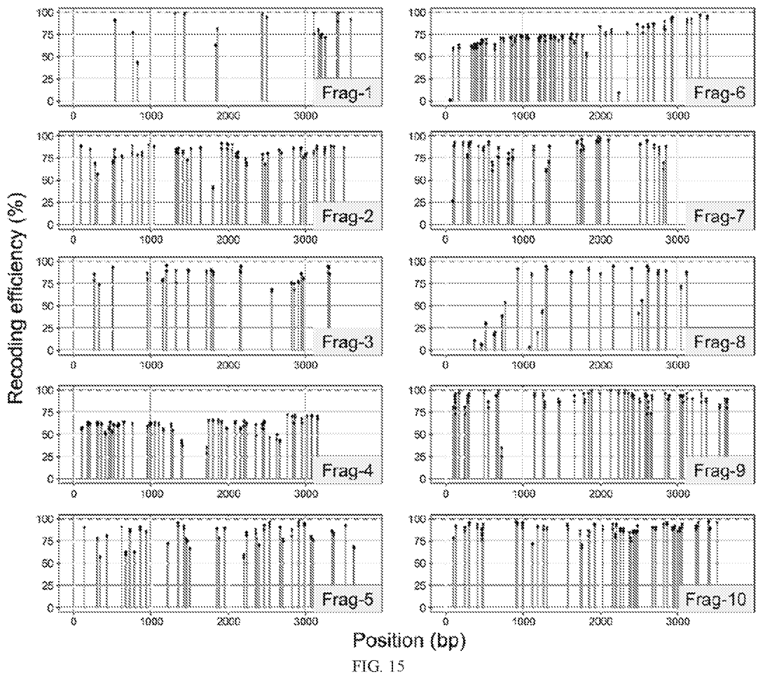

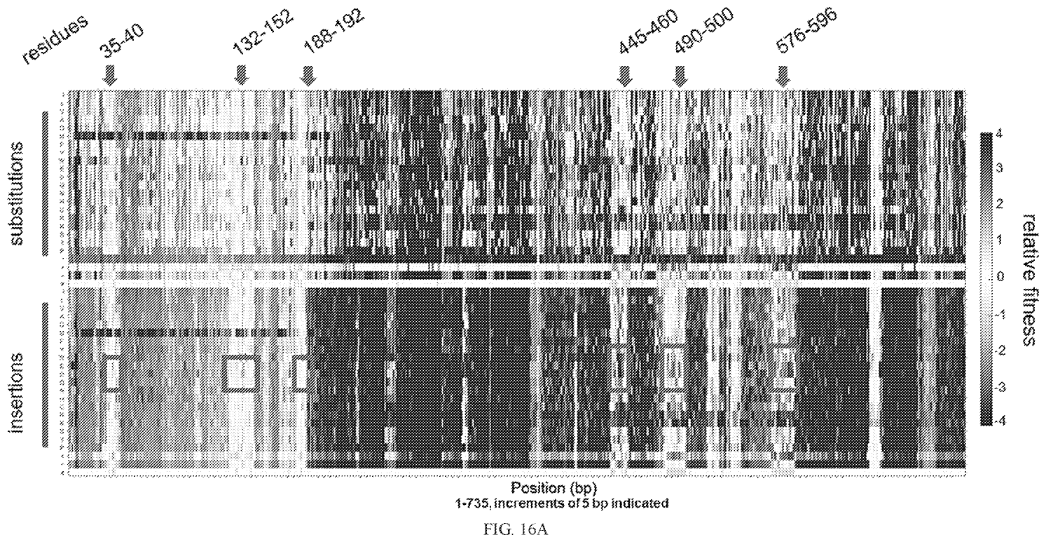

Next, MEGAAtron was applied in a synthetic biology application involving genome-scale codon replacement (FIG. 3B). Several recent studies explored the generation of synthetic genomes with recoded and reduced codon assignments that could provide biocontainment to viral infections and expansion of the genetic code with non-natural amino acids. Thus far, these efforts required either multiplex oligo-recombineering or de novo synthesis of kilobase-sized fragments and subsequent hierarchical assemblies into full genomes, which are highly resource intensive approaches. Conversely, MEGAA is a new “templated synthetic genome synthesis” framework that is facile, less expensive, and more scalable. A codon replacement scheme (TTA→CTC, TTG→CTA, AGA→AGA, AGG→CGA, TCG→AGC, TCA→AGT) was adopted for the E. coli K-12 genome based on prior recoding strategies (FIG. 3B). MEGAAtron was used to generate 10 recoded fragments each at approximately 3.6 kb in length using E. coli K12 genomic DNA as the seed sequence (Table 2). A total of 428 codon changes were made across this 36 kb genomic region. The resulting MEGAA products were pooled and analyzed by nanopore sequencing. Impressively, many fragments with greater than 50 codon changes (e.g., Frag-9 and Frag-10) had greater than 70% of products with greater than 75% of targets recoded from a single cycle of MEGAA (FIG. 3C). In general, most sites were efficiently targeted (78.8% efficiency) although some outliers were observed (FIGS. 3D and 15). Importantly, these fragments were generated in less than 3 days at 20 times lower cost than by commercial de novo gene synthesis Once generated, these 3.6 kb fragments could then be combined into larger blocks by established genome assembly methods. This approach finds use for recoding bacterial and eukaryotic genomes.

### Example 5

### Generating Gene Therapy Carrier Variants Using MEGAA

Adeno-associated viruses (AA Vs) have emerged as a safe and promising viral vector for DNA-based gene therapy, with over 149 past or ongoing clinical trials. The AAV capsid consists of 60 molecules of viral proteins encoded by the cap gene in the 4.8 kb single-stranded DNA genome of AAV. Mutations in the cap gene can lead to a variety of altered viral properties including changes in tissue tropism, packaging efficiency, thermal stability, and neutralization escape by canonical antibodies. A study generated a comprehensive single-residue saturation mutagenesis library of the cap gene in AAV2 and found many variable regions of the cap gene that individually modified AAV properties. However, the combinatorial effects of multiple distant mutations were not explored.

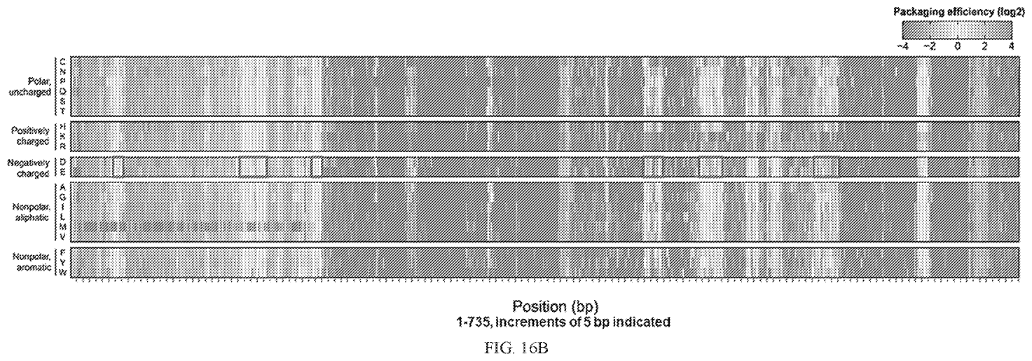

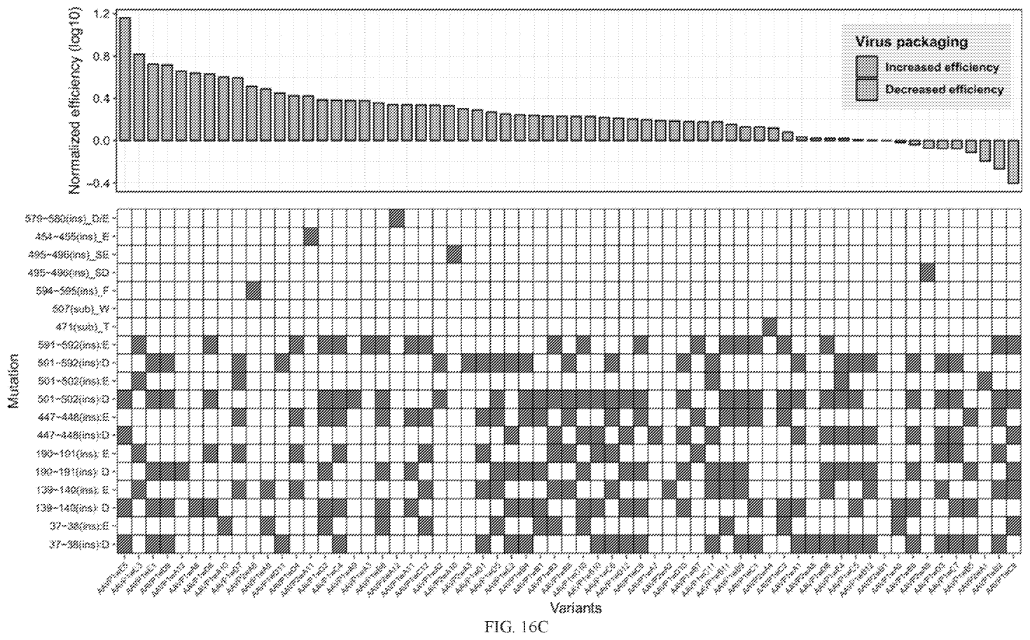

In preliminary analysis, 6 distinct regions (positions 35-40, 132-152, 188-192, 445-460, 490-500, 576-596) were found to exhibit various levels of improvements when mutated (insertions or substitutes) in terms of packaging efficiency, thermal stability, and altered tissue tropism (FIG. 16A). Interestingly, residue changes to negatively charged aspartic acid (D) or glutamic acid (E) at regions 445-460, 490-500, or 576-596, which are located at the surface of the capsid, gave particularly improved fitness compared to the wild-type.

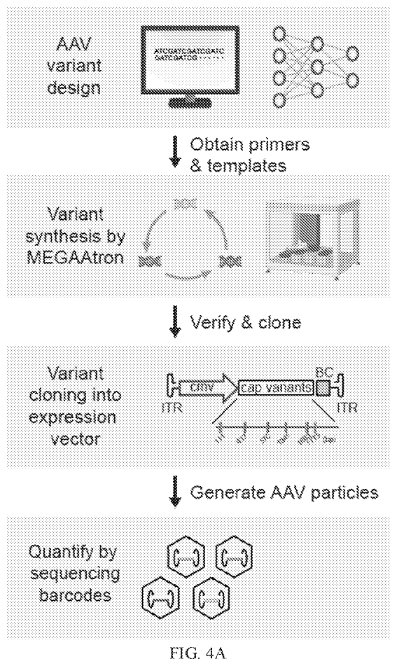

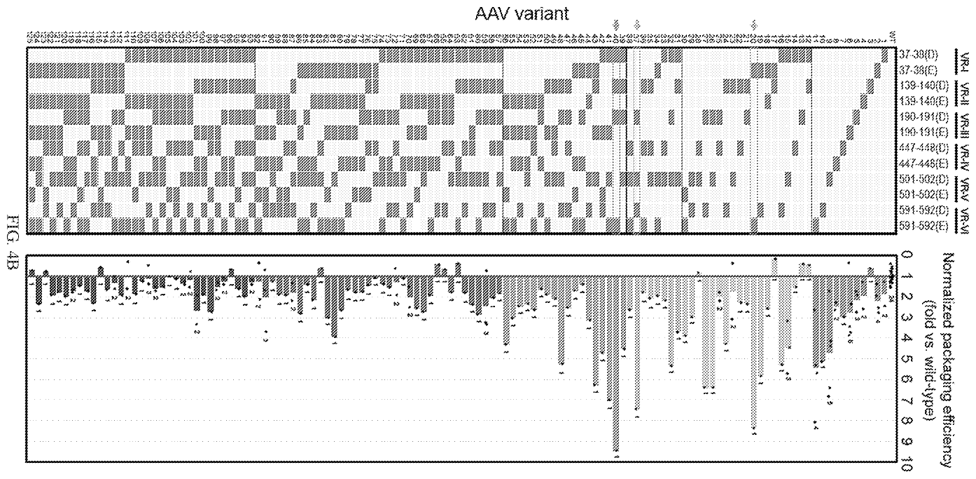

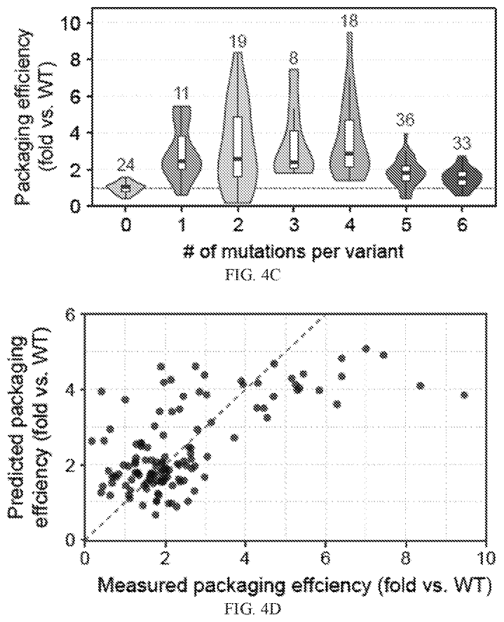

MEGAAtron was used to build AAV variants each containing up to 6 insertions at selected sites along the capsid protein that individually showed enhanced packaging efficiency based on saturation insertion data (FIGS. 4A and 16). In particular, negatively charged residues of aspartic acid (D) or glutamic acid (E) were chosen to insert into the capsid protein at residue positions 37/38, 139/140, 190/191, 447/448, 501/502, or 591/592, which in general are surface-facing on the capsid. Altering the AAV surface charge can impact various viral particle properties and enhance purification by ion exchange during manufacturing. Each variant also contained a unique 24 bp barcode that enable rapid identification and quantification by short-read Illumina sequencing. Variants were produced using MEGAAtron in an arrayed format with defined 1 to 12 mutant oligo combinations and cloned into pAAV-CMV plasmid. Isolates were verified by nanopore sequencing (Table 3, FIG. 17). In total, 192 barcoded clones were verified corresponding to 125 unique variants, including 24 wild-type barcoded variants (Table 3). Plasmids carrying each variant were equally pooled and transfected together into HEK293T cells to assess viral packaging efficiency by Illumina barcodes sequencing. Packaging efficiency was quantified as the abundance of variants in the virus pool relative to the plasmid pool.

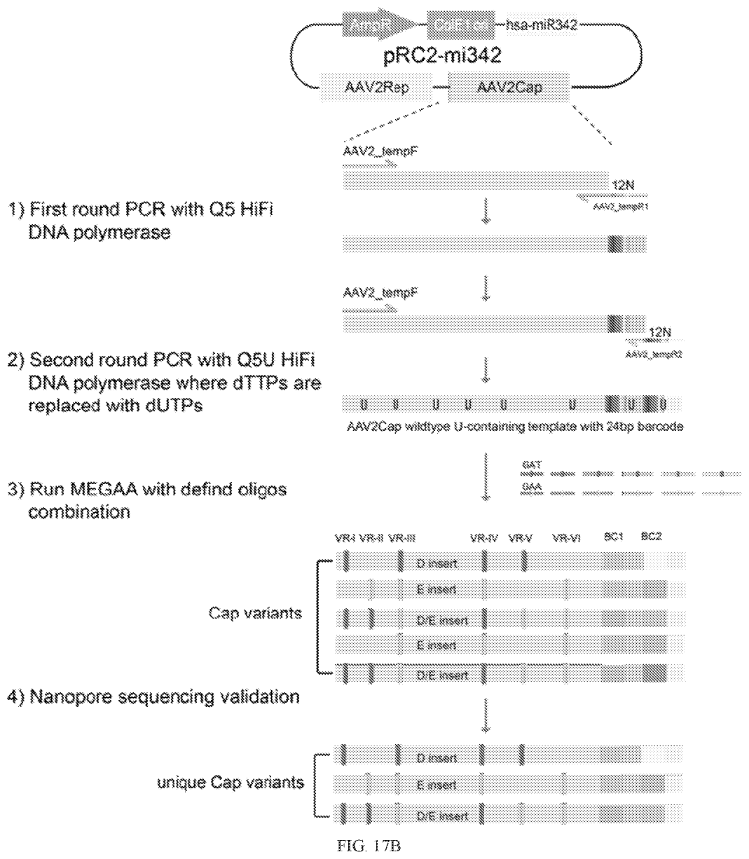

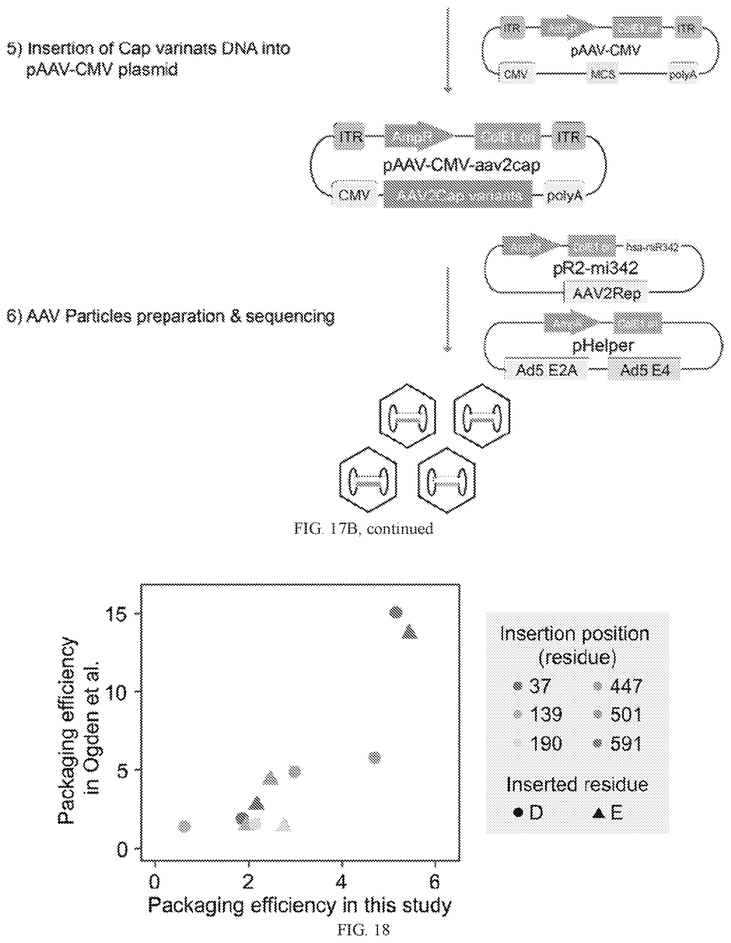

In general, single residue D or E insertions at each of the 6 chosen sites showed improvements in AAV2 packaging efficiency compared to the wild-type, which correlated well with previous data and thus verified the quantitative assay (FIG. 18). Next, the combinatorial variants in the library were explored and several were identified that exhibited substantially improved packaging efficiency (FIG. 4B). Notably, Var20 (37/38E, 591/592E), Var37 (190/191D, 501/502D, 591/592D) and Var40 (37/38D, 139/140D, 190/191D, 591/592E) showed 8.4-fold, 7.4-fold and 9.5-fold improvement over wild-type, respectively. Interestingly, variants containing 5 or 6 insertions had much poorer packaging efficiencies overall (mean of 1.9-fold and 1.5-fold, respectively) than variants with 1 to 4 mutations (mean of 2.9 to 3.7-fold, respectively) (FIG. 4C). These results suggest that an excess in negative surface charge residues substantially reduced improvements in AAV packaging and indicate an upper limit to guiding combinatorial optimizations of this set of variant designs. Nevertheless, even with a limited combinatorial survey of 4-site or fewer variants, high-performing mutants were identified within the AAV2 library.

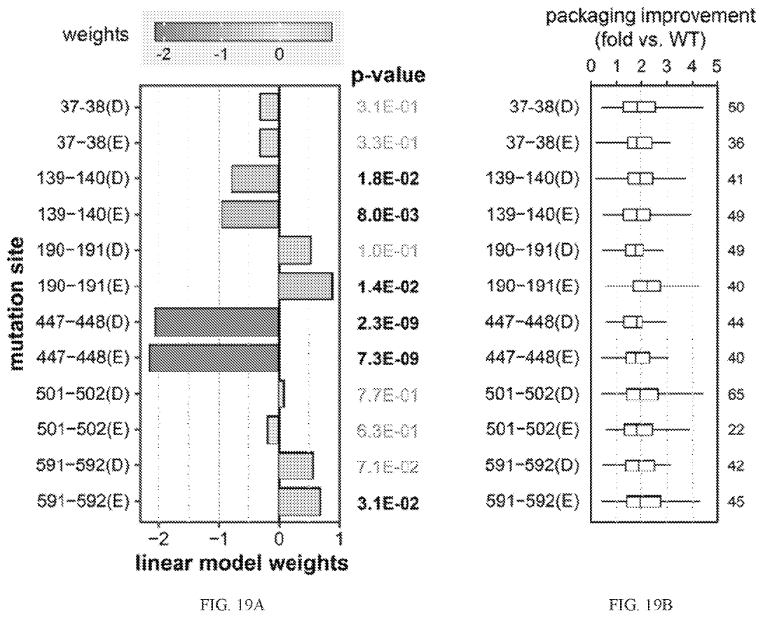

Finally, the library data was applied to a linear regression model to investigate mutation determinants of AAV2 packaging. In general, the linear model was able to predict improved packaging efficiency to a reasonable level (adjusted R2 of 0.383, p-value of 5.7e-10) (FIGS. 4D and 19). Interestingly, 591/592 (E) and 190/191 (E) mutations had statistically significant positive coefficients in the linear model (p<0.05). On the other hand, 447/448 (D/E) mutations had large negative coefficients and 139/140 (D/E) had small negative coefficients that were both statistically significant (p<0.05).

### Example 6

Scaling the Multiplex Synthesis of Greater than 1,000 Gene Variants

To scale MEGAA to thousands of variants, a strategy (MEGAAdrop) using barcoded beads to capture subpools of oligos into picoliter emulsion droplets where MEGA reactions can take place can be used (FIG. 22). The strategy shares similar steps to DropSynth but with key differences. First, an oligo pool is commercially synthesized that contains greater than 104 oligos divided into 100s-1000s distinct subpools where each subpool encodes a single variant. Each oligo has the target sequence to its template and a capture barcode unique to its subpool. Second, beads conjugated with a DNA barcode containing the complementary capture sequence will be generated (similar to DropSynth or Dropseq) and mixed with the MEGAA U-template and the oligo pool. The barcoded beads will capture unique oligo subpools into individual droplets along with the template. MEGAA single-pot reaction mixes are then added to the mixture and picoliter aqueous droplets are generated in oil emulsions where each droplet has a single bead. Oligos are released from each bead and MEGAA reactions can proceed in the droplet Upon completion, the variant pool is collected by breaking up the droplets for downstream use.

To generate capturable oligos by barcoded beads, oligos can be amplified to higher concentrations from the oligo library synthesis using universal primers to produce double-stranded (ds) oligos, which when digested generate a 5′ overhang that can hybridize to a unique 3′ overhang barcode corresponding to their encoded beads. Bead-bound ds-oligos are ligated to stabilize the captured oligos for droplet emulsion.

Two strategies (FIG. 22B) may release the oligos in emulsions. In the first strategy, the bead-bound oligo mixture is emulsified after oligo binding. Oligos are released in the emulsion by nicking the positive strand using Nb.BtsI and denaturing prior to MEGA steps. In the second strategy, bead-bound oligos are digested with Nb. BtsI and then incubated with 0.15 M NaOH to remove the negative strand. After neutralizing the NaOH, the bead pool may be emulsified and MEGA oligos can be released using Type-IIS restriction enzyme EciI. The key differences between these strategies are that in option 1, the strands complementary to the ss MEGA oligos are retained in the emulsion whereas option 2 requires an additional step which may reduce oligo yields within emulsions. Different barcode strategies, sequence orthogonality, barcode length, and capture and release efficiencies can be used. Purity of the oligos captured for individual beads can assessed by sequencing to determine levels of off-target capture.

To further increase the number of different templates that can be used in a single mixed reaction, with an aim of reaching up to 96 at a time, modified templates, each containing a unique barcode that will be exposed for capture by their corresponding subpool capture beads, will be used (FIG. 22C). Ligation of templates to the beads will form stable bead-template complexes that can be used to subsequently capture oligos as above. Ninety-six template-bead capture reactions can be performed on a single 96-well plate to enable multiplexing of MEGAdrop to 100 unique templates.

TABLE 1
Oligonucleotides

| | SEQ |
|---|---|
| | ID |
| Name | NO: |
| rsgA-F0 | 1 |
| rsgA-R0 | 2 |
| OP1.1 | 3 |
| OP1.2 | 4 |
| OP1.3 | 5 |
| OP1.4 | 6 |
| OP1.5 | 7 |
| OP1.6 | 8 |
| OP1.7 | 9 |
| OP1.8 | 10 |
| OP1.9 | 11 |
| pheS-F0 | 12 |
| pheS-F1 | 13 |
| pheS-F2 | 14 |
| pheS-F3 | 15 |
| pheS-F4 | 16 |
| pheS-F5 | 17 |
| pheS-R0 | 18 |
| pheS-R1 | 19 |
| pheS-R2 | 20 |
| pheS-R3 | 21 |
| pheS-R4 | 22 |
| pheS-R5 | 23 |
| rsgA-F1 | 24 |
| rsgA-F2 | 25 |
| rsgA-F3 | 26 |
| rsgA-F4 | 27 |
| rsgA-F5 | 28 |
| rsgA-R1 | 29 |
| rsgA-R2 | 30 |
| rsgA-R3 | 31 |
| rsgA-R4 | 32 |
| rsgA-R5 | 33 |
| rsgA-F0 | 34 |
| rsgA-R0 | 35 |
| pheS-oligo-1 | 36 |
| pheS-oligo-2 | 37 |
| pheS-oligo-3 | 38 |
| pheS-oligo-4 | 39 |
| pheS-oligo-5 | 40 |
| pheS-oligo-6 | 41 |
| pheS-oligo-7 | 42 |
| pheS-oligo-8 | 43 |
| pheS-oligo-9 | 44 |
| pheS-oligo-10 | 45 |
| pheS-oligo-11 | 46 |
| pheS-oligo-12 | 47 |
| OP2.1 | 48 |
| OP2.2 | 49 |
| OP2.3 | 50 |
| OP2.4 | 51 |
| OP2.5 | 52 |
| OP2.6 | 53 |
| OP2.7 | 54 |
| OP2.8 | 55 |
| OP2.9 | 56 |
| Bacillus subtilis | 57 |
| 168 sdpB-F | |
| Bacillus subtilis | 58 |
| 168 sdpB-R | |
| Bacillus subtilis | 59 |
| 168 sdpB oligo 1 | |
| Bacillus subtilis | 60 |
| 168 sdpB oligo 2 | |
| Bacillus subtilis | 61 |
| 168 sdpB oligo 3 | |
| Bacillus subtilis | 62 |
| 168 sdpB oligo 4 | |
| Bacillus subtilis | 63 |
| 168 sdpB oligo 5 | |
| Bacillus subtilis | 64 |
| 168 sdpB oligo 6 | |
| Bacillus subtilis | 65 |
| 168 sdpB oligo 7 | |
| Bacillus subtilis | 66 |
| 168 sdpB oligo 8 | |
| P. aeruginosa | 67 |
| PAO1 phes-F | |
| P. aeruginosa | 68 |
| PAO1 phes-R | |
| P. aeruginosa | 69 |
| PAO1 phes-oligo1 | |
| P. aeruginosa | 70 |
| PAO1 phes-oligo2 | |
| P. aeruginosa | 71 |
| PAO1 phes-oligo3 | |
| P. aeruginosa | 72 |
| PAO1 phes-oligo4 | |
| P. aeruginosa | 73 |
| PAO1 phes-oligo5 | |
| P. aeruginosa | 74 |
| PAO1 phes-oligo6 | |
| SARS-CoV-2 S- | 75 |
| tempF | |
| SARS-CoV-2 S- | 76 |
| tempR | |
| SARS-CoV-2 S- | 77 |
| L5F | |
| SARS-CoV-2 S- | 78 |
| S13I | |
| SARS-CoV-2 S- | 79 |
| L18F, T20N, P26S | |
| SARS-CoV-2 S- | 80 |
| T19R | |
| SARS-CoV-2 S- | 81 |
| L18F | |
| SARS-CoV-2 S- | 82 |
| A67V, 69del, 70del | |
| SARS-CoV-2 S- | 83 |
| 69del, 70del | |
| SARS-CoV-2 S- | 84 |
| V70F | |
| SARS-CoV-2 S- | 85 |
| G75V, T76I | |
| SARS-CoV-2 S- | 86 |
| D80A | |
| SARS-CoV-2 S- | 87 |
| D80G | |
| SARS-CoV-2 S- | 88 |
| T95I | |
| SARS-CoV-2 S- | 89 |
| D138Y | |
| SARS-CoV-2 S- | 90 |
| L141del, G142del, | |
| V143del | |
| SARS-CoV-2 S- | 91 |
| G142D | |
| SARS-CoV-2 S- | 92 |
| Y144del | |
| SARS-CoV-2 S- | 93 |
| G142D E154K | |
| SARS-CoV-2 S- | 94 |
| 144del F157S | |
| SARS-CoV-2 S- | 95 |
| W152C | |
| SARS-CoV-2 S- | 96 |
| E154K | |
| SARS-CoV-2 S- | 97 |
| E156G, 157del, | |
| 158del | |
| SARS-CoV-2 S- | 98 |
| 156del, 157de, l | |
| R158G | |
| SARS-CoV-2 S- | 99 |
| F157S | |
| SARS-CoV-2 S- | 100 |
| R190S | |
| SARS-CoV-2 S- | 101 |
| D215G | |
| SARS-CoV-2 S- | 102 |
| A222V | |
| SARS-CoV-2 S- | 103 |
| 241del 242del | |
| 243del R246I | |
| SARS-CoV-2 S- | 104 |
| 241del 242del | |
| 243del | |
| SARS-CoV-2 S- | 105 |
| 246 247 248 249 | |
| 250 251 252 | |
| D253N | |
| SARS-CoV-2 S- | 106 |
| D253G | |
| SARS-CoV-2 S- | 107 |
| W258L | |
| SARS-CoV-2 S- | 108 |
| K417N | |
| SARS-CoV-2 S- | 109 |
| K417T | |
| SARS-CoV-2 S- | 110 |
| L452Q | |
| SARS-CoV-2 S- | 111 |
| L452R | |
| SARS-CoV-2 S- | 112 |
| S477N E484K | |
| SARS-CoV-2 S- | 113 |
| S477N | |
| SARS-CoV-2 S- | 114 |
| T478K | |
| SARS-CoV-2 S- | 115 |
| E484K | |
| SARS-CoV-2 S- | 116 |
| E484Q | |
| SARS-CoV-2 S- | 117 |
| F490S | |
| SARS-CoV-2 S- | 118 |
| S494P N501Y | |
| SARS-CoV-2 S- | 119 |
| N501Y | |
| SARS-CoV-2 S- | 120 |
| F565L | |
| SARS-CoV-2 S- | 121 |
| A570D | |
| SARS-CoV-2 S- | 122 |
| H655Y | |
| SARS-CoV-2 S- | 123 |
| Q677H | |
| SARS-CoV-2 S- | 124 |
| P681H | |
| SARS-CoV-2 S- | 125 |
| P681R | |
| SARS-CoV-2 S- | 126 |
| A701V | |
| SARS-CoV-2 S- | 127 |
| T716I | |
| SARS-CoV-2 S- | 128 |
| T859N | |
| SARS-CoV-2 S- | 129 |
| F888L | |
| SARS-CoV-2 S- | 130 |
| D950H Q957R | |
| SARS-CoV-2 S- | 131 |
| D950H | |
| SARS-CoV-2 S- | 132 |
| D950N | |
| SARS-CoV-2 S- | 133 |
| Q957R | |
| SARS-CoV-2 S- | 134 |
| S982A | |
| SARS-CoV-2 S- | 135 |
| T1027I | |
| SARS-CoV-2 S- | 136 |
| Q1071H | |
| SARS-CoV-2 S- | 137 |
| E1092K H1101Y | |
| SARS-CoV-2 S- | 138 |
| D1118H | |
| SARS-CoV-2 S- | 139 |
| V1176F | |
| SARS-CoV-2 S- | 140 |
| K1191N | |
| SARS-CoV-2 S- | 141 |
| NNS-1 | |
| SARS-CoV-2 S- | 142 |
| NNS-2 | |
| SARS-CoV-2 S- | 143 |
| NNS-3 | |
| SARS-CoV-2 S- | 144 |
| NNS-4 | |
| SARS-CoV-2 S- | 145 |
| NNS-5 | |
| SARS-CoV-2 S- | 146 |
| NNS-6 | |
| SARS-CoV-2 S- | 147 |
| NNS-7 | |
| SARS-CoV-2 S- | 148 |
| NNS-8 | |
| SARS-CoV-2 S- | 149 |
| NNS-9 | |
| SARS-CoV-2 S- | 150 |
| NNS-10 | |
| 36K-F1 | 151 |
| 36K-F2 | 152 |
| 36K-F3 | 153 |
| 36K-F4 | 154 |
| 36K-F5 | 155 |
| 36K-F6 | 156 |
| 36K-F7 | 157 |
| 36K-F8 | 158 |
| 36K-F9 | 159 |
| 36K-F10 | 160 |
| 36K-R1 | 161 |
| 36K-R2 | 162 |
| 36K-R3 | 163 |
| 36K-R4 | 164 |
| 36K-R5 | 165 |
| 36K-R6 | 166 |
| 36K-R7 | 167 |
| 36K-R8 | 168 |
| 36K-R9 | 169 |
| 36K-R10 | 170 |
| 36K-oligo-1 | 171 |
| 36K-oligo-2 | 172 |
| 36K-oligo-3 | 173 |
| 36K-oligo-4 | 174 |
| 36K-oligo-5 | 175 |
| 36K-oligo-6 | 176 |
| 36K-oligo-7 | 177 |
| 36K-oligo-8 | 178 |
| 36K-oligo-9 | 179 |
| 36K-oligo-10 | 180 |
| 36K-oligo-11 | 181 |
| 36K-oligo-12 | 182 |
| 36K-oligo-13 | 183 |
| 36K-oligo-14 | 184 |
| 36K-oligo-15 | 185 |
| 36K-oligo-16 | 186 |
| 36K-oligo-17 | 187 |
| 36K-oligo-18 | 188 |
| 36K-oligo-19 | 189 |
| 36K-oligo-20 | 190 |
| 36K-oligo-21 | 191 |
| 36K-oligo-22 | 192 |
| 36K-oligo-23 | 193 |
| 36K-oligo-24 | 194 |
| 36K-oligo-25 | 195 |
| 36K-oligo-26 | 196 |
| 36K-oligo-27 | 197 |
| 36K-oligo-28 | 198 |
| 36K-oligo-29 | 199 |
| 36K-oligo-30 | 200 |
| 36K-oligo-31 | 201 |
| 36K-oligo-32 | 202 |
| 36K-oligo-33 | 203 |
| 36K-oligo-34 | 204 |
| 36K-oligo-35 | 205 |
| 36K-oligo-36 | 206 |
| 36K-oligo-37 | 207 |
| 36K-oligo-38 | 208 |
| 36K-oligo-39 | 209 |
| 36K-oligo-40 | 210 |
| 36K-oligo-41 | 211 |
| 36K-oligo-42 | 212 |
| 36K-oligo-43 | 213 |
| 36K-oligo-44 | 214 |
| 36K-oligo-45 | 215 |
| 36K-oligo-46 | 216 |
| 36K-oligo-47 | 217 |
| 36K-oligo-48 | 218 |
| 36K-oligo-49 | 219 |
| 36K-oligo-50 | 220 |
| 36K-oligo-51 | 221 |
| 36K-oligo-52 | 222 |
| 36K-oligo-53 | 223 |
| 36K-oligo-54 | 224 |
| 36K-oligo-55 | 225 |
| 36K-oligo-56 | 226 |
| 36K-oligo-57 | 227 |
| 36K-oligo-58 | 228 |
| 36K-oligo-59 | 229 |
| 36K-oligo-60 | 230 |
| 36K-oligo-61 | 231 |
| 36K-oligo-62 | 232 |
| 36K-oligo-63 | 233 |
| 36K-oligo-64 | 234 |
| 36K-oligo-65 | 235 |
| 36K-oligo-66 | 236 |
| 36K-oligo-67 | 237 |
| 36K-oligo-68 | 238 |
| 36K-oligo-69 | 239 |
| 36K-oligo-70 | 240 |
| 36K-oligo-71 | 241 |
| 36K-oligo-72 | 242 |
| 36K-oligo-73 | 243 |
| 36K-oligo-74 | 244 |
| 36K-oligo-75 | 245 |
| 36K-oligo-76 | 246 |
| 36K-oligo-77 | 247 |
| 36K-oligo-78 | 248 |
| 36K-oligo-79 | 249 |
| 36K-oligo-80 | 250 |
| 36K-oligo-81 | 251 |
| 36K-oligo-82 | 252 |
| 36K-oligo-83 | 253 |
| 36K-oligo-84 | 254 |
| 36K-oligo-85 | 255 |
| 36K-oligo-86 | 256 |
| 36K-oligo-87 | 257 |
| 36K-oligo-88 | 258 |
| 36K-oligo-89 | 259 |
| 36K-oligo-90 | 260 |
| 36K-oligo-91 | 261 |
| 36K-oligo-92 | 262 |
| 36K-oligo-93 | 263 |
| 36K-oligo-94 | 264 |
| 36K-oligo-95 | 265 |
| 36K-oligo-96 | 266 |
| 36K-oligo-97 | 267 |
| 36K-oligo-98 | 268 |
| 36K-oligo-99 | 269 |
| 36K-oligo-100 | 270 |
| 36K-oligo-101 | 271 |
| 36K-oligo-102 | 272 |
| 36K-oligo-103 | 273 |
| 36K-oligo-104 | 274 |
| 36K-oligo-105 | 275 |
| 36K-oligo-106 | 276 |
| 36K-oligo-107 | 277 |
| 36K-oligo-108 | 278 |
| 36K-oligo-109 | 279 |
| 36K-oligo-110 | 280 |
| 36K-oligo-111 | 281 |
| 36K-oligo-112 | 282 |
| 36K-oligo-113 | 283 |
| 36K-oligo-114 | 284 |
| 36K-oligo-115 | 285 |
| 36K-oligo-116 | 286 |
| 36K-oligo-117 | 287 |
| 36K-oligo-118 | 288 |
| 36K-oligo-119 | 289 |
| 36K-oligo-120 | 290 |
| 36K-oligo-121 | 291 |
| 36K-oligo-122 | 292 |
| 36K-oligo-123 | 293 |
| 36K-oligo-124 | 294 |
| 36K-oligo-125 | 295 |
| 36K-oligo-126 | 296 |
| 36K-oligo-127 | 297 |
| 36K-oligo-128 | 298 |
| 36K-oligo-129 | 299 |
| 36K-oligo-130 | 300 |
| 36K-oligo-131 | 301 |
| 36K-oligo-132 | 302 |
| 36K-oligo-133 | 303 |
| 36K-oligo-134 | 304 |
| 36K-oligo-135 | 305 |
| 36K-oligo-136 | 306 |
| 36K-oligo-137 | 307 |
| 36K-oligo-138 | 308 |
| 36K-oligo-139 | 309 |
| 36K-oligo-140 | 310 |
| 36K-oligo-141 | 311 |
| 36K-oligo-142 | 312 |
| 36K-oligo-143 | 313 |
| 36K-oligo-144 | 314 |
| 36K-oligo-145 | 315 |
| 36K-oligo-146 | 316 |
| 36K-oligo-147 | 317 |
| 36K-oligo-148 | 318 |
| 36K-oligo-149 | 319 |
| 36K-oligo-150 | 320 |
| 36K-oligo-151 | 321 |
| 36K-oligo-152 | 322 |
| 36K-oligo-153 | 323 |
| 36K-oligo-154 | 324 |
| 36K-oligo-155 | 325 |
| 36K-oligo-156 | 326 |
| 36K-oligo-157 | 327 |
| 36K-oligo-158 | 328 |
| 36K-oligo-159 | 329 |
| 36K-oligo-160 | 330 |
| 36K-oligo-161 | 331 |
| 36K-oligo-162 | 332 |
| 36K-oligo-163 | 333 |
| 36K-oligo-164 | 334 |
| 36K-oligo-165 | 335 |
| 36K-oligo-166 | 336 |
| 36K-oligo-167 | 337 |
| 36K-oligo-168 | 338 |
| 36K-oligo-169 | 339 |
| 36K-oligo-170 | 340 |
| 36K-oligo-171 | 341 |
| 36K-oligo-172 | 342 |
| 36K-oligo-173 | 343 |
| 36K-oligo-174 | 344 |
| 36K-oligo-175 | 345 |
| 36K-oligo-176 | 346 |
| 36K-oligo-177 | 347 |
| 36K-oligo-178 | 348 |
| 36K-oligo-179 | 349 |
| 36K-oligo-180 | 350 |
| 36K-oligo-181 | 351 |
| 36K-oligo-182 | 352 |
| 36K-oligo-183 | 353 |
| 36K-oligo-184 | 354 |
| 36K-oligo-185 | 355 |
| 36K-oligo-186 | 356 |
| 36K-oligo-187 | 357 |
| 36K-oligo-188 | 358 |
| 36K-oligo-189 | 359 |
| 36K-oligo-190 | 360 |
| 36K-oligo-191 | 361 |
| 36K-oligo-192 | 362 |
| 36K-oligo-193 | 363 |
| 36K-oligo-194 | 364 |
| 36K-oligo-195 | 365 |
| 36K-oligo-196 | 366 |
| 36K-oligo-197 | 367 |
| 36K-oligo-198 | 368 |
| 36K-oligo-199 | 369 |
| 36K-oligo-200 | 370 |
| 36K-oligo-201 | 371 |
| 36K-oligo-202 | 372 |
| 36K-oligo-203 | 373 |
| 36K-oligo-204 | 374 |
| 36K-oligo-205 | 375 |
| 36K-oligo-206 | 376 |
| 36K-oligo-207 | 377 |
| 36K-oligo-208 | 378 |
| 36K-oligo-209 | 379 |
| 36K-oligo-210 | 380 |
| 36K-oligo-211 | 381 |
| 36K-oligo-212 | 382 |
| 36K-oligo-213 | 383 |
| 36K-oligo-214 | 384 |
| 36K-oligo-215 | 385 |
| 36K-oligo-216 | 386 |
| 36K-oligo-217 | 387 |
| 36K-oligo-218 | 388 |
| 36K-oligo-219 | 389 |
| 36K-oligo-220 | 390 |
| 36K-oligo-221 | 391 |
| 36K-oligo-222 | 392 |
| 36K-oligo-223 | 393 |
| 36K-oligo-224 | 394 |
| 36K-oligo-225 | 395 |
| 36K-oligo-226 | 396 |
| 36K-oligo-227 | 397 |
| 36K-oligo-228 | 398 |
| 36K-oligo-229 | 399 |
| 36K-oligo-230 | 400 |
| 36K-oligo-231 | 401 |
| 36K-oligo-232 | 402 |
| 36K-oligo-233 | 403 |
| 36K-oligo-234 | 404 |
| 36K-oligo-235 | 405 |
| 36K-oligo-236 | 406 |
| 36K-oligo-237 | 407 |
| 36K-oligo-238 | 408 |
| 36K-oligo-239 | 409 |
| 36K-oligo-240 | 410 |
| 36K-oligo-241 | 411 |
| 36K-oligo-242 | 412 |
| 36K-oligo-243 | 413 |
| 36K-oligo-244 | 414 |
| 36K-oligo-245 | 415 |
| 36K-oligo-246 | 416 |
| 36K-oligo-247 | 417 |
| 36K-oligo-248 | 418 |
| 36K-oligo-249 | 419 |
| 36K-oligo-250 | 420 |
| 36K-oligo-251 | 421 |
| 36K-oligo-252 | 422 |
| 36K-oligo-253 | 423 |
| 36K-oligo-254 | 424 |
| 36K-oligo-255 | 425 |
| 36K-oligo-256 | 426 |
| 36K-oligo-257 | 427 |
| 36K-oligo-258 | 428 |
| 36K-oligo-259 | 429 |
| 36K-oligo-260 | 430 |
| 36K-oligo-261 | 431 |
| 36K-oligo-262 | 432 |
| 36K-oligo-263 | 433 |
| 36K-oligo-264 | 434 |
| 36K-oligo-265 | 435 |
| 36K-oligo-266 | 436 |
| 36K-oligo-267 | 437 |
| 36K-oligo-268 | 438 |
| 36K-oligo-269 | 439 |
| 36K-oligo-270 | 440 |
| 36K-oligo-271 | 441 |
| 36K-oligo-272 | 442 |
| 36K-oligo-273 | 443 |
| 36K-oligo-274 | 444 |
| 36K-oligo-275 | 445 |
| 36K-oligo-276 | 446 |
| 36K-oligo-277 | 447 |
| 36K-oligo-278 | 448 |
| 36K-oligo-279 | 449 |
| 36K-oligo-280 | 450 |
| 36K-oligo-281 | 451 |
| 36K-oligo-282 | 452 |
| 36K-oligo-283 | 453 |
| 36K-oligo-284 | 454 |
| 36K-oligo-285 | 455 |
| 36K-oligo-286 | 456 |
| 36K-oligo-287 | 457 |
| 36K-oligo-288 | 458 |
| 36K-oligo-289 | 459 |
| AAV2-tempF | 460 |
| AAV2-tempR1 | 461 |
| AAV2-tempR2 | 462 |
| AAV2cap-37- | 463 |
| 38(ins)-D | |
| AAV2cap-37- | 464 |
| 38(ins)-E | |
| AAV2cap-139- | 465 |
| 140(ins)-D | |
| AAV2cap-139- | 466 |
| 140(ins)-E | |
| AAV2cap-190- | 467 |
| 191(ins)-D | |
| AAV2cap-190- | 468 |
| 191(ins)-E | |
| AAV2cap-447- | 469 |
| 448(ins)-D | |
| AAV2cap-447- | 470 |
| 448(ins)-E | |
| AAV2cap-501- | 471 |
| 502(ins)-D | |
| AAV2cap-501- | 472 |
| 502(ins)-E | |
| AAV2cap-591- | 473 |
| 592(ins)-D | |
| AAV2cap-591- | 474 |
| 592(ins)-E | |

TABLE 2
E. coli Recoding Genome Segments

| | Wildtype sequence | Recoding sequence | | | |
|---|---|---|---|---|---|
| 36 Kb segments | (SEQ ID NO) | (SEQ ID NO) | Length (bp) | mutantic oligos ID | # mutagenic oligos |
| Fragment1 | 475 | 485 | 3,716 | 36K-oligo-1 to 36K-oligo-14 | 14 |
| Fragment2 | 476 | 486 | 3,628 | 36K-oligo-15 to 36K-oligo-47 | 33 |
| Fragment3 | 477 | 487 | 3,718 | 36K-oligo-48 to 36K-oligo-64 | 17 |
| Fragment4 | 478 | 488 | 3,475 | 36K-oligo-65 to 36K-oligo-101 | 37 |
| Fragment5 | 479 | 489 | 3,692 | 36K-oligo-102 to 36K-oligo-134 | 33 |
| Fragment6 | 480 | 490 | 3,487 | 36K-oligo-135 to 36K-oligo-174 | 40 |
| Fragment7 | 481 | 491 | 3,583 | 36K-oligo-175 to 36K-oligo-199 | 25 |
| Fragment8 | 482 | 492 | 3,736 | 36K-oligo-200 to 36K-oligo-223 | 24 |
| Fragment9 | 483 | 493 | 3,709 | 36K-oligo-224 to 36K-oligo-256 | 33 |
| Fragment10 | 484 | 494 | 3,531 | 36K-oligo-257 to 36K-oligo-289 | 33 |

TABLE 3
AAV2 Variants

| Cap | | | |
|---|---|---|---|
| variant | | | # mutagenic |
| ID | Genotype | mutantic oligos ID | oligos |
| 1 | 111(ins): GAT | AAV2cap-37-38(ins): D | 1 |
| 2 | 111(ins): GAA | AAV2cap-37-38(ins): E | 1 |
| 3 | 147(ins): GAT | AAV2cap-139-140(ins): D | 1 |
| 4 | 147(ins): GAA | AAV2cap-139-140(ins): E | 1 |
| 5 | 570(ins): GAT | AAV2cap-190-191(ins): D | 1 |
| 6 | 570(ins): GAA | AAV2cap-190-191(ins): E | 1 |
| 7 | 1341(ins): GAT | AAV2cap-447-448(ins): D | 1 |
| 8 | 1341(ins): GAA | AAV2cap-447-448(ins): E | 1 |
| 9 | 1503(ins): GAT | AAV2cap-501-502(ins): D | 1 |
| 10 | 1733(ins): GAT | AAV2cap-591-592(ins): D | 1 |
| 11 | 1733(ins): GAA | AAV2cap-591-592(ins): E | 1 |
| 12 | 111(ins): GAT; 147(ins): GAT | AAV2cap-37-38(ins): D; AAV2cap-139-140(ins): D | 2 |
| 13 | 111(ins): GAT; 570(ins): GAT | AAV2cap-37-38(ins): D; AAV2cap-190-191(ins): D | 2 |
| 14 | 111(ins): GAT; 1341(ins): GAT | AAV2cap-37-38(ins): D; AAV2cap-447-448(ins): D | 2 |
| 15 | 111(ins): GAT; 1503(ins): GAT | AAV2cap-37-38(ins): D; AAV2cap-501-502(ins): D | 2 |
| 16 | 111(ins): GAT; 1733(ins): GAT | AAV2cap-37-38(ins): D; AAV2cap-591-592(ins): D | 2 |
| 17 | 111(ins): GAA; 147(ins): GAT | AAV2cap-37-38(ins): E; AAV2cap-139-140(ins): D | 2 |
| 18 | 111(ins): GAA; 147(ins): GAA | AAV2cap-37-38(ins): E; AAV2cap-139-140(ins): E | 2 |
| 19 | 111(ins): GAA; 1733(ins): GAT | AAV2cap-37-38(ins): E; AAV2cap-591-592(ins): D | 2 |
| 20 | 111(ins): GAA; 1733(ins): GAA | AAV2cap-37-38(ins): E; AAV2cap-591-592(ins): E | 2 |
| 21 | 147(ins): GAT; 570(ins): GAT | AAV2cap-139-140(ins): D; AAV2cap-190-191(ins): D | 2 |
| 22 | 147(ins): GAT; 1341(ins): GAT | AAV2cap-139-140(ins): D; AAV2cap-447-448(ins): D | 2 |
| 23 | 147(ins): GAT; 1503(ins): GAT | AAV2cap-139-140(ins): D; AAV2cap-501-502(ins): D | 2 |
| 24 | 147(ins): GAT; 1733(ins): GAT | AAV2cap-139-140(ins): D; AAV2cap-591-592(ins): D | 2 |
| 25 | 570(ins): GAT; 1341(ins): GAT | AAV2cap-190-191(ins): D; AAV2cap-447-448(ins): D | 2 |
| 26 | 570(ins): GAT; 1503(ins): GAT | AAV2cap-190-191(ins): D; AAV2cap-501-502(ins): D | 2 |
| 27 | 570(ins): GAT; 1733(ins): GAT | AAV2cap-190-191(ins): D; AAV2cap-591-592(ins): D | 2 |
| 28 | 1341(ins): GAT; 1503(ins): GAT | AAV2cap-447-448(ins): D; AAV2cap-501-502(ins): D | 2 |
| 29 | 1503(ins): GAT; 1733(ins): GAT | AAV2cap-501-502(ins): D; AAV2cap-591-592(ins): D | 2 |
| 30 | 1503(ins): GAA; 1733(ins): GAA | AAV2cap-501-502(ins): E; AAV2cap-591-592(ins): E | 2 |
| 31 | 111(ins): GAT; 147(ins): | AAV2cap-37-38(ins): D; AAV2cap-139-140(ins): D; AAV2cap-501- | 3 |
| | GAT; 1503(ins): GAT | 502(ins): D | |
| 32 | 111(ins): GAT; 570(ins): | AAV2cap-37-38(ins): D; AAV2cap-190-191(ins): D; AAV2cap-501- | 3 |
| | GAT; 1503(ins): GAT | 502(ins): D | |
| 33 | 111(ins): GAT; 1341(ins): | AAV2cap-37-38(ins): D; AAV2cap-447-448(ins): D; AAV2cap-501- | 3 |
| | GAT; 1503(ins): GAT | 502(ins): D | |
| 34 | 111(ins): GAA; 1503(ins): | AAV2cap-37-38(ins): E; AAV2cap-501-502(ins): D; AAV2cap-591- | 3 |
| | GAT; 1733(ins): GAA | 592(ins): E | |
| 35 | 147(ins): GAT; 1341(ins): | AAV2cap-139-140(ins): D; AAV2cap-447-448(ins): D; AAV2cap-501- | 3 |
| | GAT; 1503(ins): GAT | 502(ins): D | |
| 36 | 147(ins): GAT; 1341(ins): | AAV2cap-139-140(ins): D; AAV2cap-447-448(ins): D; AAV2cap-591- | 3 |
| | GAT; 1733(ins): GAA | 592(ins): E | |
| 37 | 570(ins): GAT; 1503(ins): | AAV2cap-190-191(ins): D; AAV2cap-501-502(ins): D; AAV2cap-591- | 3 |
| | GAT; 1733(ins): GAT | 592(ins): D | |
| 38 | 1341(ins): GAT; 1503(ins): | AAV2cap-447-448(ins): D; AAV2cap-501-502(ins): D; AAV2cap-591- | 3 |
| | GAT; 1733(ins): GAA | 592(ins): E | |
| 39 | 111(ins): GAT; 147(ins): GAT; | AAV2cap-37-38(ins): D; AAV2cap-139-140(ins): D; AAV2cap-190- | 4 |
| | 570(ins): GAT; 1503(ins): GAT | 191(ins): D; AAV2cap-501-502(ins): D | |
| 40 | 111(ins): GAT; 147(ins): GAT; | AAV2cap-37-38(ins): D; AAV2cap-139-140(ins): D; AAV2cap-190- | 4 |
| | 570(ins): GAT; 1733(ins): GAA | 191(ins): D; AAV2cap-591-592(ins): E | |
| 41 | 111(ins): GAT; 570(ins): GAA; | AAV2cap-37-38(ins): D; AAV2cap-190-191(ins): E; AAV2cap-501- | 4 |
| | 1503(ins): GAT; 1733(ins): GAA | 502(ins): D; AAV2cap-591-592(ins): E | |
| 42 | 111(ins): GAT; 570(ins): GAA; | AAV2cap-37-38(ins): D; AAV2cap-190-191(ins): E; AAV2cap-501- | 4 |
| | 1503(ins): GAA; 1733(ins): GAT | 502(ins): E; AAV2cap-591-592(ins): D | |
| 43 | 111(ins): GAA; 147(ins): GAT; | AAV2cap-37-38(ins): E; AAV2cap-139-140(ins): D; AAV2cap-190- | 4 |
| | 570(ins): GAA; 1503(ins): GAT | 191(ins): E; AAV2cap-501-502(ins): D | |
| 44 | 111(ins): GAA; 147(ins): GAA; | AAV2cap-37-38(ins): E; AAV2cap-139-140(ins): E; AAV2cap-501- | 4 |
| | 1503(ins): GAT; 1733(ins): GAT | 502(ins): D; AAV2cap-591-592(ins): D; | |
| 45 | 111(ins): GAA; 570(ins): GAT; | AAV2cap-37-38(ins): E; AAV2cap-190-191(ins): D; AAV2cap-447- | 4 |
| | 1341(ins): GAA; 1733(ins): GAA | 448(ins): E; AAV2cap-591-592(ins): E | |
| 46 | 111(ins): GAA; 1341(ins): GAA; | AAV2cap-37-38(ins): E; AAV2cap-447-448(ins): E; AAV2cap-501- | 4 |
| | 1503(ins): GAA; 1733(ins): GAA | 502(ins): E; AAV2cap-591-592(ins): E | |
| 47 | 147(ins): GAT; 570(ins): GAT; | AAV2cap-139-140(ins): D; AAV2cap-190-191(ins): D; AAV2cap-447- | 4 |
| | 1341(ins): GAT; 1733(ins): GAT | 448(ins): D; AAV2cap-591-592(ins): D | |
| 48 | 147(ins): GAT; 570(ins): GAT; | AAV2cap-139-140(ins): D; AAV2cap-190-191(ins): D; AAV2cap-501- | 4 |
| | 1503(ins): GAT; 1733(ins): GAT | 502(ins): D; AAV2cap-591-592(ins): D | |
| 49 | 147(ins): GAT; 570(ins): GAA; | AAV2cap-139-140(ins): D; AAV2cap-190-191(ins): E; AAV2cap-447- | 4 |
| | 1341(ins): GAT; 1503(ins): GAT | 448(ins): D; AAV2cap-501-502(ins): D | |
| 50 | 147(ins): GAT; 570(ins): GAA; | AAV2cap-139-140(ins): D; AAV2cap-190-191(ins): E; AAV2cap-501- | 4 |
| | 1503(ins): GAT; 1733(ins): GAA | 502(ins): D; AAV2cap-591-592(ins): E | |
| 51 | 147(ins): GAA; 570(ins): GAT; | AAV2cap-139-140(ins): E; AAV2cap-190-191(ins): D; AAV2cap-447- | 4 |
| | 1341(ins): GAA; 1733(ins): GAT | 448(ins): E; AAV2cap-591-592(ins): D | |
| 52 | 147(ins): GAA; 570(ins): GAA; | AAV2cap-139-140(ins): E; AAV2cap-190-191(ins): E; AAV2cap-447- | 4 |
| | 1341(ins): GAT; 1733(ins): GAA | 448(ins): D; AAV2cap-591-592(ins): E | |
| 53 | 147(ins): GAA; 570(ins): GAA; | AAV2cap-139-140(ins): E; AAV2cap-190-191(ins): E; AAV2cap-447- | 4 |
| | 1341(ins): GAA; 1503(ins): GAT | 448(ins): E; AAV2cap-501-502(ins): D | |
| 54 | 147(ins): GAA; 570(ins): GAA; | AAV2cap-139-140(ins): E; AAV2cap-190-191(ins): E; AAV2cap-447- | 4 |
| | 1341(ins): GAA; 1733(ins): GAT | 448(ins): E; AAV2cap-591-592(ins): D | |
| 55 | 147(ins): GAA; 570(ins): GAA; | AAV2cap-139-140(ins): E; AAV2cap-190-191(ins): E; AAV2cap-447- | 4 |
| | 1341(ins): GAA; 1733(ins): GAA | 448(ins): E; AAV2cap-591-592(ins): E | |
| 56 | 147(ins): GAA; 570(ins): GAA; | AAV2cap-139-140(ins): E; AAV2cap-190-191(ins): E; AAV2cap-501- | 4 |
| | 1503(ins): GAA; 1733(ins): GAA | 502(ins): E; AAV2cap-591-592(ins): E | |
| 57 | 111(ins): GAT; 147(ins): GAT; 570(ins): | AAV2cap-37-38(ins): D; AAV2cap-139-140(ins): D; AAV2cap-190- | 5 |
| | GAT; 1341(ins): GAT; 1503(ins): GAT | 191(ins): D; AAV2cap-447-448(ins): D; AAV2cap-501-502(ins): D | |
| 58 | 111(ins): GAT; 147(ins): GAT; 570(ins): | AAV2cap-37-38(ins): D; AAV2cap-139-140(ins): D; AAV2cap-190- | 5 |
| | GAT; 1341(ins): GAT; 1733(ins): GAT | 191(ins): D; AAV2cap-447-448(ins): D; AAV2cap-591-592(ins): D | |
| 59 | 111(ins): GAT; 147(ins): GAT; 570(ins): | AAV2cap-37-38(ins): D; AAV2cap-139-140(ins): D; AAV2cap-190- | 5 |
| | GAT; 1503(ins): GAT; 1733(ins): GAT | 191(ins): D; AAV2cap-501-502(ins): D; AAV2cap-591-592(ins): D | |
| 60 | 111(ins): GAT; 147(ins): GAT; 570(ins): | AAV2cap-37-38(ins): D; AAV2cap-139-140(ins): D; AAV2cap-190- | 5 |
| | GAT; 1503(ins): GAT; 1733(ins): GAA | 191(ins): D; AAV2cap-501-502(ins): D; AAV2cap-591-592(ins): E | |
| 61 | 111(ins): GAT; 147(ins): GAT; | AAV2cap-37-38(ins): D; AAV2cap-139-140(ins): D; AAV2cap-190- | 5 |
| | 570(ins): GAA; 1341(ins): GAT; 1503(ins): GAT | 191(ins): E; AAV2cap-447-448(ins): D; AAV2cap-501-502(ins): D | |
| 62 | 111(ins): GAT; 147(ins): GAT; 1341(ins): | AAV2cap-37-38(ins): D; AAV2cap-139-140(ins): D; AAV2cap-447- | 5 |
| | GAT; 1503(ins): GAT; 1733(ins): GAT | 448(ins): D; AAV2cap-501-502(ins): D; AAV2cap-591-592(ins): D | |
| 63 | 111(ins): GAT; 147(ins): GAT; 1341(ins): | AAV2cap-37-38(ins): D; AAV2cap-139-140(ins): D; AAV2cap-447- | 5 |
| | GAA; 1503(ins): GAT; 1733(ins): GAA | 448(ins): E: AAV2cap-501-502(ins): D; AAV2cap-591-592(ins): E | |
| 64 | 111(ins): GAT; 147(ins): GAA; 570(ins): | AAV2cap-37-38(ins): D; AAV2cap-139-140(ins): E; AAV2cap-190- | 5 |
| | GAT; 1341(ins): GAT; 1503(ins): GAT | 191(ins): D; AAV2cap-447-448(ins): D; AAV2cap-501-502(ins): D | |
| 65 | 111(ins): GAT; 147(ins): GAA; 570(ins): | AAV2cap-37-38(ins): D; AAV2cap-139-140(ins): E; AAV2cap-190- | 5 |
| | GAT; 1341(ins): GAT; 1733(ins): GAT | 191(ins): D; AAV2cap-447-448(ins): D; AAV2cap-591-592(ins): D | |
| 66 | 111(ins): GAT; 147(ins): GAA; 570(ins): | AAV2cap-37-38(ins): D; AAV2cap-139-140(ins): E; AAV2cap-190- | 5 |
| | GAT; 1341(ins): GAA; 1733(ins): GAT | 191(ins): D; AAV2cap-447-448(ins): E; AAV2cap-591-592(ins): D | |
| 67 | 111(ins): GAT; 147(ins): GAA; 570(ins): | AAV2cap-37-38(ins): D; AAV2cap-139-140(ins): E; AAV2cap-190- | 5 |
| | GAT; 1341(ins): GAA; 1733(ins): GAA | 191(ins): D; AAV2cap-447-448(ins): E; AAV2cap-591-592(ins): E | |
| 68 | 111(ins): GAT; 147(ins): GAA; 570(ins): | AAV2cap-37-38(ins): D; AAV2cap-139-140(ins): E; AAV2cap-190- | 5 |
| | GAA; 1341(ins): GAA; 1503(ins): GAT | 191(ins): E; AAV2cap-447-448(ins): E; AAV2cap-501-502(ins): D | |
| 69 | 111(ins): GAT; 147(ins): GAA; 570(ins): | AAV2cap-37-38(ins): D; AAV2cap-139-140(ins): E; AAV2cap-190- | 5 |
| | GAA; 1341(ins): GAA; 1503(ins): GAA | 191(ins): E; AAV2cap-447-448(ins): E; AAV2cap-501-502(ins): E | |
| 70 | 111(ins): GAT; 147(ins): GAA; 570(ins): | AAV2cap-37-38(ins): D; AAV2cap-139-140(ins): E; AAV2cap-190- | 5 |
| | GAA; 1341(ins): GAA; 1733(ins): GAT | 191(ins): E; AAV2cap-447-448(ins): E; AAV2cap-591-592(ins): D | |
| 71 | 111(ins): GAT; 147(ins): GAA; 1341(ins): | AAV2cap-37-38(ins): D; AAV2cap-139-140(ins): E; AAV2cap-447- | 5 |
| | GAA; 1503(ins): GAT; 1733(ins): GAA | 448(ins): E; AAV2cap-501-502(ins): D; AAV2cap-591-592(ins): E | |
| 72 | 111(ins): GAT; 570(ins): GAT; 1341(ins): | AAV2cap-37-38(ins): D; AAV2cap-190-191(ins): D; AAV2cap-447- | 5 |
| | GAT; 1503(ins): GAT; 1733(ins): GAT | 448(ins): D; AAV2cap-501-502(ins): D; AAV2cap-591-592(ins): D | |
| 73 | 111(ins): GAT; 570(ins): GAT; 1341(ins): | AAV2cap-37-38(ins): D; AAV2cap-190-191(ins): D; AAV2cap-447- | 5 |
| | GAA; 1503(ins): GAT; 1733(ins): GAT | 448(ins): E; AAV2cap-501-502(ins): D; AAV2cap-591-592(ins): D | |
| 74 | 111(ins): GAT; 570(ins): GAT; 1341(ins): | AAV2cap-37-38(ins): D; AAV2cap-190-191(ins): D; AAV2cap-447- | 5 |
| | GAA; 1503(ins): GAT; 1733(ins): GAA | 448(ins): E; AAV2cap-501-502(ins): D; AAV2cap-591-592(ins): E | |
| 75 | 111(ins): GAA; 147(ins): GAT; 570(ins): | AAV2cap-37-38(ins): E; AAV2cap-139-140(ins): D; AAV2cap-190- | 5 |
| | GAT; 1341(ins): GAT; 1503(ins): GAT | 191(ins): D; AAV2cap-447-448(ins): D; AAV2cap-501-502(ins): D | |
| 76 | 111(ins): GAA; 147(ins): GAT; 570(ins): | AAV2cap-37-38(ins): E; AAV2cap-139-140(ins): D; AAV2cap-190- | 5 |
| | GAT; 1341(ins): GAT; 1503(ins): GAA | 191(ins): D; AAV2cap-447-448(ins): D; AAV2cap-501-502(ins): E | |
| 77 | 111(ins): GAA; 147(ins): GAA; 570(ins): | AAV2cap-37-38(ins): E; AAV2cap-139-140(ins): E; AAV2cap-190- | 5 |
| | GAT; 1341(ins): GAA; 1503(ins): GAA | 191(ins): D; AAV2cap-447-448(ins): E; AAV2cap-501-502(ins): E | |
| 78 | 111(ins): GAA; 147(ins): GAA; 570(ins): | AAV2cap-37-38(ins): E; AAV2cap-139-140(ins): E; AAV2cap-190- | 5 |
| | GAA; 1341(ins): GAA; 1503(ins): GAA | 191(ins): E; AAV2cap-447-448(ins): E; AAV2cap-501-502(ins): E | |
| 79 | 111(ins): GAA; 147(ins): GAA; 570(ins): | AAV2cap-37-38(ins): E; AAV2cap-139-140(ins): E; AAV2cap-190- | 5 |
| | GAA; 1341(ins): GAA; 1733(ins): GAT | 191(ins): E; AAV2cap-447-448(ins): E; AAV2cap-591-592(ins): D | |
| 80 | 111(ins): GAA; 147(ins): GAA; 570(ins): | AAV2cap-37-38(ins): E; AAV2cap-139-140(ins): E; AAV2cap-190- | 5 |
| | GAA; 1341(ins): GAA; 1733(ins): GAA | 191(ins): E; AAV2cap-447-448(ins): E; AAV2cap-591-592(ins): E | |
| 81 | 111(ins): GAA; 147(ins): GAA; 570(ins): | AAV2cap-37-38(ins): E; AAV2cap-139-140(ins): E; AAV2cap-190- | 5 |
| | GAA; 1503(ins): GAT; 1733(ins): GAA | 191(ins): E; AAV2cap-501-502(ins): D; AAV2cap-591-592(ins): E | |
| 82 | 111(ins): GAA; 147(ins): GAA; 570(ins): | AAV2cap-37-38(ins): E; AAV2cap-139-140(ins): E; AAV2cap-190- | 5 |
| | GAA; 1503(ins): GAA; 1733(ins): GAA | 191(ins): E; AAV2cap-501-502(ins): E; AAV2cap-591-592(ins): E | |
| 83 | 111(ins): GAA; 147(ins): GAA; 1341(ins): | AAV2cap-37-38(ins): E; AAV2cap-139-140(ins): E; AAV2cap-447- | 5 |
| | GAT; 1503(ins): GAT; 1733(ins): GAA | 448(ins): D; AAV2cap-501-502(ins): D; AAV2cap-591-592(ins): E | |
| 84 | 111(ins): GAA; 147(ins): GAA; 1341(ins): | AAV2cap-37-38(ins): E; AAV2cap-139-140(ins): E; AAV2cap-447- | 5 |
| | GAA; 1503(ins): GAT; 1733(ins): GAT | 448(ins): E; AAV2cap-501-502(ins): D; AAV2cap-591-592(ins): D | |
| 85 | 111(ins): GAA; 570(ins): GAT; 1341(ins): | AAV2cap-37-38(ins): E; AAV2cap-190-191(ins): D; AAV2cap-447- | 5 |
| | GAA; 1503(ins): GAT; 1733(ins): GAA | 448(ins): E; AAV2cap-501-502(ins): D; AAV2cap-591-592(ins): E | |
| 86 | 111(ins): GAA; 570(ins): GAA; 1341(ins): | AAV2cap-37-38(ins): E; AAV2cap-190-191(ins): E; AAV2cap-447- | 5 |
| | GAA; 1503(ins): GAT; 1733(ins): GAA | 448(ins): E; AAV2cap-501-502(ins): D; AAV2cap-591-592(ins): E | |
| 87 | 147(ins): GAT; 570(ins): GAT; 1341(ins): | AAV2cap-139-140(ins): D; AAV2cap-190-191(ins): D; AAV2cap-447- | 5 |
| | GAT; 1503(ins): GAT; 1733(ins): GAT | 448(ins): D; AAV2cap-501-502(ins): D; AAV2cap-591-592(ins): D | |
| 88 | 147(ins): GAA; 570(ins): GAT; 1341(ins): | AAV2cap-139-140(ins): E; AAV2cap-190-191(ins): D; AAV2cap-447- | 5 |
| | GAT; 1503(ins): GAA; 1733(ins): GAT | 448(ins): D; AAV2cap-501-502(ins): E; AAV2cap-591-592(ins): D | |
| 89 | 147(ins): GAA; 570(ins): GAT; 1341(ins): | AAV2cap-139-140(ins): E; AAV2cap-190-191(ins): D; AAV2cap-447- | 5 |
| | GAA; 1503(ins): GAA; 1733(ins): GAT | 448(ins): E; AAV2cap-501-502(ins): E; AAV2cap-591-592(ins): D | |
| 90 | 147(ins): GAA; 570(ins): GAA; 1341(ins): | AAV2cap-139-140(ins): E; AAV2cap-190-191(ins): E; AAV2cap-447- | 5 |
| | GAA; 1503(ins): GAT; 1733(ins): GAT | 448(ins): E; AAV2cap-501-502(ins): D; AAV2cap-591-592(ins): D | |
| 91 | 147(ins): GAA; 570(ins): GAA; 1341(ins): | AAV2cap-139-140(ins): E; AAV2cap-190-191(ins): E; AAV2cap-447- | 5 |
| | GAA; 1503(ins): GAA; 1733(ins): GAT | 448(ins): E; AAV2cap-501-502(ins): E; AAV2cap-591-592(ins): D | |
| 92 | 147(ins): GAA; 570(ins): GAA; 1341(ins): | AAV2cap-139-140(ins): E; AAV2cap-190-191(ins): E; AAV2cap-447- | 5 |
| | GAA; 1503(ins): GAA; 1733(ins): GAA | 448(ins): E; AAV2cap-501-502(ins): E; AAV2cap-591-592(ins): E | |
| 93 | 111(ins): GAT; 147(ins): GAT; 570(ins): | AAV2cap-37-38(ins): D; AAV2cap-139-140(ins): D; AAV2cap-190- | 6 |
| | GAT; 1341(ins): GAT; 1503(ins): | 191(ins): D; AAV2cap-447-448(ins): D; AAV2cap-501- | |
| | GAT; 1733(ins): GAT | 502(ins): D; AAV2cap-591-592(ins): D | |
| 94 | 111(ins): GAT; 147(ins): GAT; 570(ins): | AAV2cap-37-38(ins): D; AAV2cap-139-140(ins): D; AAV2cap-190- | 6 |
| | GAT; 1341(ins): GAT; 1503(ins): | 191(ins): D; AAV2cap-447-448(ins): D; AAV2cap-501- | |
| | GAT; 1733(ins): GAA | 502(ins): D; AAV2cap-591-592(ins): E | |
| 95 | 111(ins): GAT; 147(ins): GAT; 570(ins): | AAV2cap-37-38(ins): D; AAV2cap-139-140(ins): D; AAV2cap-190- | 6 |
| | GAT; 1341(ins): GAT; 1503(ins): | 191(ins): D; AAV2cap-447-448(ins): D; AAV2cap-501- | |
| | GAA; 1733(ins): GAA | 502(ins): E; AAV2cap-591-592(ins): E | |
| 96 | 111(ins): GAT; 147(ins): GAT; 570(ins): | AAV2cap-37-38(ins): D; AAV2cap-139-140(ins): D; AAV2cap-190- | 6 |
| | GAT; 1341(ins): GAA; 1503(ins): | 191(ins): D; AAV2cap-447-448(ins): E; AAV2cap-501- | |
| | GAT; 1733(ins): GAT | 502(ins): D; AAV2cap-591-592(ins): D | |
| 97 | 111(ins): GAT; 147(ins): GAT; 570(ins): | AAV2cap-37-38(ins): D; AAV2cap-139-140(ins): D; AAV2cap-190- | 6 |
| | GAT; 1341(ins): GAA; 1503(ins): | 191(ins): D; AAV2cap-447-448(ins): E; AAV2cap-501- | |
| | GAT; 1733(ins): GAA | 502(ins): D; AAV2cap-591-592(ins): E | |
| 98 | 111(ins): GAT; 147(ins): GAT; 570(ins): | AAV2cap-37-38(ins): D; AAV2cap-139-140(ins): D; AAV2cap-190- | 6 |
| | GAA; 1341(ins): GAT; 1503(ins): | 191(ins): E; AAV2cap-447-448(ins): D; AAV2cap-501- | |
| | GAT; 1733(ins): GAT | 502(ins): D; AAV2cap-591-592(ins): D | |
| 99 | 111(ins): GAT; 147(ins): GAT; 570(ins): | AAV2cap-37-38(ins): D; AAV2cap-139-140(ins): D; AAV2cap-190- | 6 |
| | GAA; 1341(ins): GAT; 1503(ins): | 191(ins): E; AAV2cap-447-448(ins): D; AAV2cap-501- | |
| | GAA; 1733(ins): GAA | 502(ins): E; AAV2cap-591-592(ins): E | |
| 100 | 111(ins): GAT; 147(ins): GAT; 570(ins): | AAV2cap-37-38(ins): D; AAV2cap-139-140(ins): D; AAV2cap-190- | 6 |
| | GAA; 1341(ins): GAA; 1503(ins): | 191(ins): E; AAV2cap-447-448(ins): E; AAV2cap-501- | |
| | GAT; 1733(ins): GAT | 502(ins): D; AAV2cap-591-592(ins): D | |
| 101 | 111(ins): GAT; 147(ins): GAT; 570(ins): | AAV2cap-37-38(ins): D; AAV2cap-139-140(ins): D; AAV2cap-190- | 6 |
| | GAA; 1341(ins): GAA; 1503(ins): | 191(ins): E; AAV2cap-447-448(ins): E; AAV2cap-501- | |
| | GAT; 1733(ins): GAA | 502(ins): D; AAV2cap-591-592(ins): E | |
| 102 | 111(ins): GAT; 147(ins): GAA; 570(ins): | AAV2cap-37-38(ins): D; AAV2cap-139-140(ins): E; AAV2cap-190- | 6 |
| | GAT; 1341(ins): GAT; 1503(ins): | 191(ins): D; AAV2cap-447-448(ins): D; AAV2cap-501- | |
| | GAT; 1733(ins): GAT | 502(ins): D; AAV2cap-591-592(ins): D | |
| 103 | 111(ins): GAT; 147(ins): GAA; 570(ins): | AAV2cap-37-38(ins): D; AAV2cap-139-140(ins): E; AAV2cap-190- | 6 |
| | GAT; 1341(ins): GAT; 1503(ins): | 191(ins): D; AAV2cap-447-448(ins): D; AAV2cap-501- | |
| | GAT; 1733(ins): GAA | 502(ins): D; AAV2cap-591-592(ins): E | |
| 104 | 111(ins): GAT; 147(ins): GAA; 570(ins): | AAV2cap-37-38(ins): D; AAV2cap-139-140(ins): E; AAV2cap-190- | 6 |
| | GAT; 1341(ins): GAT; 1503(ins): | 191(ins): D; AAV2cap-447-448(ins): D; AAV2cap-501- | |
| | GAA; 1733(ins): GAT | 502(ins): E; AAV2cap-591-592(ins): D | |
| 105 | 111(ins): GAT; 147(ins): GAA; 570(ins): | AAV2cap-37-38(ins): D; AAV2cap-139-140(ins): E; AAV2cap-190- | 6 |
| | GAT; 1341(ins): GAT; 1503(ins): | 191(ins): D; AAV2cap-447-448(ins): D; AAV2cap-501- | |
| | GAA; 1733(ins): GAA | 502(ins): E; AAV2cap-591-592(ins): E | |
| 106 | 111(ins): GAT; 147(ins): GAA; 570(ins): | AAV2cap-37-38(ins): D; AAV2cap-139-140(ins): E; AAV2cap-190- | 6 |
| | GAT; 1341(ins): GAA; 1503(ins): | 191(ins): D; AAV2cap-447-448(ins): E; AAV2cap-501- | |
| | GAT; 1733(ins): GAT | 502(ins): D; AAV2cap-591-592(ins): D | |
| 107 | 111(ins): GAT; 147(ins): GAA; 570(ins): | AAV2cap-37-38(ins): D; AAV2cap-139-140(ins): E; AAV2cap-190- | 6 |
| | GAT; 1341(ins): GAA; 1503(ins): | 191(ins): D; AAV2cap-447-448(ins): E; AAV2cap-501- | |
| | GAT; 1733(ins): GAA | 502(ins): D; AAV2cap-591-592(ins): E | |
| 108 | 111(ins): GAT; 147(ins): GAA; 570(ins): | AAV2cap-37-38(ins): D; AAV2cap-139-140(ins): E; AAV2cap-190- | 6 |
| | GAA; 1341(ins): GAT; 1503(ins): | 191(ins): E; AAV2cap-447-448(ins): D; AAV2cap-501- | |
| | GAT; 1733(ins): GAT | 502(ins): D; AAV2cap-591-592(ins): D | |
| 109 | 111(ins): GAT; 147(ins): GAA; 570(ins): | AAV2cap-37-38(ins): D; AAV2cap-139-140(ins): E; AAV2cap-190- | 6 |
| | GAA; 1341(ins): GAT; 1503(ins): | 191(ins): E; AAV2cap-447-448(ins): D; AAV2cap-501- | |
| | GAT; 1733(ins): GAA | 502(ins): D; AAV2cap-591-592(ins): E | |
| 110 | 111(ins): GAT; 147(ins): GAA; 570(ins): | AAV2cap-37-38(ins): D; AAV2cap-139-140(ins): E; AAV2cap-190- | 6 |
| | GAA; 1341(ins): GAT; 1503(ins): | 191(ins): E; AAV2cap-447-448(ins): D; AAV2cap-501- | |
| | GAA; 1733(ins): GAA | 502(ins): E; AAV2cap-591-592(ins): E | |
| 111 | 111(ins): GAT; 147(ins): GAA; 570(ins): | AAV2cap-37-38(ins): D; AAV2cap-139-140(ins): E; AAV2cap-190- | 6 |
| | GAA; 1341(ins): GAA; 1503(ins): | 191(ins): E; AAV2cap-447-448(ins): E; AAV2cap-501- | |
| | GAT; 1733(ins): GAA | 502(ins): D; AAV2cap-591-592(ins): E | |
| 112 | 111(ins): GAA; 147(ins): GAT; 570(ins): | AAV2cap-37-38(ins): E; AAV2cap-139-140(ins): D; AAV2cap-190- | 6 |
| | GAT; 1341(ins): GAT; 1503(ins): | 191(ins): D; AAV2cap-447-448(ins): D; AAV2cap-501- | |
| | GAT; 1733(ins): GAA | 502(ins): D; AAV2cap-591-592(ins): E | |
| 113 | 111(ins): GAA; 147(ins): GAT; 570(ins): | AAV2cap-37-38(ins): E; AAV2cap-139-140(ins): D; AAV2cap-190- | 6 |
| | GAT; 1341(ins): GAA; 1503(ins): | 191(ins): D; AAV2cap-447-448(ins): E; AAV2cap-501- | |
| | GAT; 1733(ins): GAA | 502(ins): D; AAV2cap-591-592(ins): E | |
| 114 | 111(ins): GAA; 147(ins): GAT; 570(ins): | AAV2cap-37-38(ins): E; AAV2cap-139-140(ins): D; AAV2cap-190- | 6 |
| | GAA; 1341(ins): GAT; 1503(ins): | 191(ins): E; AAV2cap-447-448(ins): D; AAV2cap-501- | |
| | GAT; 1733(ins): GAT | 502(ins): D; AAV2cap-591-592(ins): D | |
| 115 | 111(ins): GAA; 147(ins): GAT; 570(ins): | AAV2cap-37-38(ins): E; AAV2cap-139-140(ins): D; AAV2cap-190- | 6 |
| | GAA; 1341(ins): GAT; 1503(ins): | 191(ins): E; AAV2cap-447-448(ins): D; AAV2cap-501- | |
| | GAA; 1733(ins): GAT | 502(ins): E; AAV2cap-591-592(ins): D | |
| 116 | 111(ins): GAA; 147(ins): GAT; 570(ins): | AAV2cap-37-38(ins): E; AAV2cap-139-140(ins): D; AAV2cap-190- | 6 |
| | GAA; 1341(ins): GAA; 1503(ins): | 191(ins): E; AAV2cap-447-448(ins): E; AAV2cap-501- | |
| | GAT; 1733(ins): GAT | 502(ins): D; AAV2cap-591-592(ins): D | |
| 117 | 111(ins): GAA; 147(ins): GAA; 570(ins): | AAV2cap-37-38(ins): E; AAV2cap-139-140(ins): E; AAV2cap-190- | 6 |
| | GAT; 1341(ins): GAT; 1503(ins): | 191(ins): D; AAV2cap-447-448(ins): D; AAV2cap-501- | |
| | GAT; 1733(ins): GAA | 502(ins): D; AAV2cap-591-592(ins): E | |
| 118 | 111(ins): GAA; 147(ins): GAA; 570(ins): | AAV2cap-37-38(ins): E; AAV2cap-139-140(ins): E; AAV2cap-190- | 6 |
| | GAT; 1341(ins): GAT; 1503(ins): | 191(ins): D; AAV2cap-447-448(ins): D; AAV2cap-501- | |
| | GAA; 1733(ins): GAA | 502(ins): E; AAV2cap-591-592(ins): E | |
| 119 | 111(ins): GAA; 147(ins): GAA; 570(ins): | AAV2cap-37-38(ins): E; AAV2cap-139-140(ins): E; AAV2cap-190- | 6 |
| | GAT; 1341(ins): GAA; 1503(ins): | 191(ins): D; AAV2cap-447-448(ins): E; AAV2cap-501- | |
| | GAT; 1733(ins): GAT | 502(ins): D; AAV2cap-591-592(ins): D | |
| 120 | 111(ins): GAA; 147(ins): GAA; 570(ins): | AAV2cap-37-38(ins): E; AAV2cap-139-140(ins): E; AAV2cap-190- | 6 |
| | GAT; 1341(ins): GAA; 1503(ins): | 191(ins): D; AAV2cap-447-448(ins): E; AAV2cap-501- | |
| | GAT; 1733(ins): GAA | 502(ins): D; AAV2cap-591-592(ins): E | |
| 121 | 111(ins): GAA; 147(ins): GAA; 570(ins): | AAV2cap-37-38(ins): E; AAV2cap-139-140(ins): E; AAV2cap-190- | 6 |
| | GAA; 1341(ins): GAT; 1503(ins): | 191(ins): E; AAV2cap-447-448(ins): D; AAV2cap-501- | |
| | GAT; 1733(ins): GAT | 502(ins): D; AAV2cap-591-592(ins): D | |
| 122 | 111(ins): GAA; 147(ins): GAA; 570(ins): | AAV2cap-37-38(ins): E; AAV2cap-139-140(ins): E; AAV2cap-190- | 6 |
| | GAA; 1341(ins): GAT; 1503(ins): | 191(ins): E; AAV2cap-447-448(ins): D; AAV2cap-501- | |
| | GAT; 1733(ins): GAA | 502(ins): D; AAV2cap-591-592(ins): E | |
| 123 | 111(ins): GAA; 147(ins): GAA; 570(ins): | AAV2cap-37-38(ins): E; AAV2cap-139-140(ins): E; AAV2cap-190- | 6 |
| | GAA; 1341(ins): GAT; 1503(ins): | 191(ins): E; AAV2cap-447-448(ins): D; AAV2cap-501- | |
| | GAA; 1733(ins): GAT | 502(ins): E; AAV2cap-591-592(ins): D | |
| 124 | 111(ins): GAA; 147(ins): GAA; 570(ins): | AAV2cap-37-38(ins): E; AAV2cap-139-140(ins): E; AAV2cap-190- | 6 |
| | GAA; 1341(ins): GAA; 1503(ins): | 191(ins): E; AAV2cap-447-448(ins): E; AAV2cap-501- | |
| | GAT; 1733(ins): GAA | 502(ins): D; AAV2cap-591-592(ins): E | |
| 125 | 111(ins): GAA; 147(ins): GAA; 570(ins): | AAV2cap-37-38(ins): E; AAV2cap-139-140(ins): E; AAV2cap-190- | 6 |
| | GAA; 1341(ins): GAA; 1503(ins): | 191(ins): E; AAV2cap-447-448(ins): E; AAV2cap-501- | |
| | GAA; 1733(ins): GAA | 502(ins): E; AAV2cap-591-592(ins): E | |

TABLE 4
Summary of Mutagenic Methods

| | | | % perfect variants made | | | | | |
|---|---|---|---|---|---|---|---|---|
| | | Multi- | 1 | 3 | 6 | 9 | Input | |
| Methods | Strategy | site | oligo | oligo | oligo | oligo | Template | Oligos |
| MEGAA | Uracil- | Yes, | 93.5 | 72.4 | 35.4 | 2.5 | Linear | 30-40 nt |
| | containing | up to | | | | (30.1 | DNA | standard |
| | dsDNA, | 20-30 | | | | opt.) | | oligos |
| | random | oligos | | | | | | (~24 hour |
| | annealing, | | | | | | | synthesis) |
| | polymerase | | | | | | | |
| | extension, | | | | | | | |
| | and nicks | | | | | | | |
| | ligation | | | | | | | |
| Q5 ® Site- | polymerase | No | 85.2 | NA | NA | NA | Circular | 30-40 nt |
| Directed | extension, | | | | | | plasmid | standard |
| Mutagenesis Kit | KLD | | | | | | | oligos |
| [NEB E0554S] | digestion, | | | | | | | (~24 hour |
| | and in vitro | | | | | | | synthesis) |
| | ligation | | | | | | | |
| QuikChange II | polymerase | No | 87.6 | NA | NA | NA | Circular | 30-40 nt |
| Site-Directed | extension, | | | | | | plasmid | standard |
| Mutagenesis Kit | Dpn I | | | | | | | oligos |
| [Agilent 200523] | digestion, | | | | | | | (~24 hour |
| | and in vivo | | | | | | | synthesis) |
| | ligation | | | | | | | |
| QuikChange | polymerase | Yes, | 80.7 | 30.4 | 3.6 | <0.1 | Circular | 30-40 nt |
| Lightning Multi | extension, | up to | | | | | plasmid | standard |
| Site-Directed | Dpn I | 3 | | | | | | oligos |
| Mutagenesis Kit | digestion, | | | | | | | (~24 hour |
| [Agilent 210515] | and in | | | | | | | synthesis) |
| | vitro | | | | | | | |
| | ligation | | | | | | | |
| Darwin assembly | ssDNA, | Yes, | 12.5- | NA | NA | NA | Circular | Chemically |
| [PMID: 29409059] | modified | up to | 99.65 * | | | | plasmid, | modified |
| | oligos, | 6 | | | | | target | oligos |
| | polymerase | | | | | | region < | needed |
| | extension, | | | | | | 2 Kb | (~5 day |
| | and nicks | | | | | | | synthesis) |
| | ligation | | | | | | | |
| | SSDNA, | Yes, | >99.76 * | NA | NA | NA | Circular | Long |
| | long oligos | up to | | | | | plasmid, | oligos |
| | (>130 nt), | 5 | | | | | target | (>130 nt) |
| | polymerase | | | | | | region > | needed |
| | extension, | | | | | | 2 Kb | (~3 day |
| | and nicks | | | | | | | synthesis) |
| | for long | | | | | | | |

| | | | Total | | |
|---|---|---|---|---|---|
| | | Total | time | | Automation |
| | Bacterial | Protocol | from | Cost per | and Primer |
| Methods | Transformation | Time | design | sample | design tool |
| MEGAA | Not needed; | ~8 hours | ~32 | ~$20 | Yes; |
| | Compatible | (+3 | hours | | Mutagenesis |
| | with iterative | hours/ | | | oligo |
| | cycling | cycle) | | | design tool |
| | | | | | (MEGAAdt) |
| | | | | | is available |
| | | | | | online |
| Q5 ® Site- | Needed for | ~40 | ~3 | ~$20 | NA; NA |
| Directed | purification | hours | days | | |
| Mutagenesis Kit | | | | | |
| [NEB E0554S] | | | | | |
| QuikChange II | Needed for | ~41 | ~3 | ~$35 | NA; NA |
| Site-Directed | purification | hours | days | | |
| Mutagenesis Kit | | | | | |
| [Agilent 200523] | | | | | |
| QuikChange | Needed for | ~40 | ~3 | ~$45 | NA; NA |
| Lightning Multi | purification | hours | days | | |
| Site-Directed | | | | | |
| Mutagenesis Kit | | | | | |
| [Agilent 210515] | | | | | |
| Darwin assembly | Not needed for | ~48 | ~7 | >$160; | Yes; NA |
| [PMID: 29409059] | product; Not | hours | days | biotinylated | |
| | compatible with | | | oligo | |
| | iterative cycling | | | needed | |
| | Not needed for | ~46 | ~5 | >$130; | NA; NA |
| | product; Not | hours | days | special | |
| | compatible with | | | purification | |
| | iterative cycling | | | needed | |
| | | | | ligation | |
| | | | | oligos | |

* From PMID: 29409059, unverified.

The scope of the present invention is not limited by what has been specifically shown and described herein. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A method for modifying a nucleic acid comprising:
annealing three or more mutagenic primers to a template nucleic acid, wherein each of the three or more mutagenic primers comprises at least one mutagenic nucleotide in comparison to template strand; and
contacting primed template nucleic acid with a polymerase, a ligase, and nucleotide triphosphates under conditions suitable to extend and ligate a first mutant strand.

2. The method of claim 1, comprising annealing at least 6 mutagenic primers.

3. (canceled)

4. The method of claim 1, wherein the template nucleic acid is or is derived from a genomic nucleic acid.

5. The method of claim 4, wherein the genomic nucleic acid is from or is derived from a human, a microbial organism, or a virus.

6-10. (canceled)

11. The method of claim 1, further comprising generating the template nucleic acid by amplifying a target polynucleotide in a reaction comprising deoxyuridine triphosphate and lacking deoxythymidine triphosphate.

12. The method of claim 1, wherein the template nucleic acid comprises a modified or synthetic nucleotide selected from a biotinylated nucleotide, a brominated nucleotide, and deoxyinosine monophosphate, or a combination thereof.

13. (canceled)

14. The method of claim 1, wherein the three or more mutagenic primers are provided in molar excess to the template nucleic acid.

15. The method of claim 1, wherein the three or more mutagenic primers are phosphorylated, have a 3′ end Guanosine or Cytidine, have 5′ and 3′ ends having about 10 nucleotides with 100% complementarity to the template nucleic acid, or a combination thereof.

16-19. (canceled)

20. The method of claim 1, wherein mutagenic primers at the 5′ end of the template nucleic acid have lower melting temperatures compared with mutagenic primers at the 3′ end of the template nucleic acid.

21. The method of claim 20, wherein the three or more mutagenic primers have a gradation of melting temperatures from lowest to highest from 5′ to 3′ on the template nucleic acid.

22-25. (canceled)

26. The method of claim 1, wherein the template nucleic acid comprises a length greater than 1 kilobases.

27-28. (canceled)

29. The method of claim 1, wherein the template nucleic acid is not in a circular plasmid.

30. The method of claim 1, further comprising amplifying the first mutant strand, wherein the amplifying of the first mutant strand comprises contacting the first mutant strand with polymerase that is inactive for uracil-containing templates.

31. (canceled)

32. The method of claim 1, wherein method further comprises generating a copy of the first mutant strand with a reaction mixture comprising deoxyuridine triphosphate and lacking deoxythymidine triphosphate.

33. The method of claim 1, further comprising modifying the first mutant strand, or an amplification product or copy thereof, to generate a second mutant strand.

34. (canceled)

35. The method of claim 33, further comprising amplifying the second mutant strand and/or generating a copy of the second mutant strand with a reaction mixture comprising deoxyuridine triphosphate and lacking deoxythymidine triphosphate.

36. The method of claim 35, further comprising modifying the second mutant strand, or an amplification product or copy thereof, to generate a third mutant strand.

37-58. (canceled)

59. A polypeptide comprising a viral capsid protein having two or more amino acid insertions between positions selected from: 37/38, 139/140, 190/191, 447/448, 501/502, and 591/592 relative to SEQ ID NO: 509,
wherein each of the two or more amino acid insertions is individually a negatively charged amino acid.

60-69. (canceled)

70. An engineered virus or virus-like particle (VLP) comprising the polypeptide of claim 59 or a nucleic acid encoding thereof.

71. (canceled)

72. A method of delivering a nucleic acid to a cell comprising contacting the cell with an effective amount of the engineered virus or VLP of claim 70 or a composition comprising thereof, wherein the nucleic acid is encapsulated in the engineered virus or VLP

73-76. (canceled)
